(19) United States
(12) Patent Application Publication
(10) Pub. No.: US 2025/0177570 A1
(43) Pub. Date: Jun. 5, 2025

(54) MULTIPLEXED REPRESSION OF IMMUNOSUPPRESSIVE GENES

(71) Applicant: Yale University, New Haven, CT (US)

(72) Inventors: Sidi Chen, New Haven, CT (US); Feifei Zhang, New Haven, CT (US); Guangchuan Wang, New Haven, CT (US); Ryan Chow, New Haven, CT (US)

(21) Appl. No.: 18/840,303

(22) PCT Filed: Feb. 17, 2023
(86) PCT No.: PCT/US2023/062809

§ 371 Date: Aug. 21, 2024

Related U.S. Application Data
(60) Provisional application No. 63/312,730, filed on Feb. 22, 2022.

(51) Int. Cl. A61K 48/00; C12N 9/22; C12N 15/11; C12N 15/86

(52) U.S. Cl. CPC A61K 48/0058; C12N 9/22; C12N 15/11; C12N 15/111; C12N 15/86; C12N 2310/20; C12N 2750/14143

(57) ABSTRACT

The present disclosure provides in one aspect a method of enhancing an immune response in a subject in need thereof. In certain embodiments, the method comprises administering to the subject an effective amount of a gene silencing system comprising a plurality of gRNAs, wherein the gene silencing system decreases expression of at least one endogenous immunosuppressive gene in a target cell, thereby enhancing the immune response. Also included are methods and compositions for enhancing anti-tumor immune responses in subject in need thereof.

### CROSS-REFERENCE TO RELATED APPLICATION

The present application claims priority under 35 U.S.C. § 119(e) to U.S. Provisional Patent Application No. 63/312,730, filed Feb. 22, 2022, which is hereby incorporated by reference in its entirety herein.

### STATEMENT REGARDING FEDERALLY SPONSORED RESEARCH OR DEVELOPMENT

This invention was made with government support under CA238295, CA231112 and CA225498 awarded by the National Institutes of Health and under W81XWH-20-1-0072 awarded by the United States Army. The government has certain rights in the invention.

### BACKGROUND

Checkpoint blockade immunotherapy has transformed cancer medicine. Cancer patients that used to have little to no options can now benefit from this class of powerful drugs that may substantially enhance survival.

However, single agent checkpoint antibodies usually have low response rates in patients. Combinatorial immunotherapy involving single antibodies may improve the therapeutic efficacy compared to single agents. However, the difficulties for the approach of combining more and more antibodies scale exponentially, as development of each specific and potent therapeutic antibody is a daunting task by itself.

A more flexible, versatile, and effective means for combinatorial immunotherapy is urgently needed. The present disclosure addresses this need.

### BRIEF SUMMARY

As described herein, the present disclosure relates to methods and compositions useful for providing means for combinatorial immunotherapy through the administration of CRISPR-based gene silencing systems which target immunosuppressive factors expressed by both immune cells and tissues including tumor cells.

As such, in one aspect, the present disclosure includes a method of enhancing an immune response in a subject in need thereof, the method comprising administering to the subject an effective amount of a gene silencing system, wherein the gene silencing system decreases expression of at least one endogenous immunosuppressive gene in a target cell, thereby enhancing the immune response.

In another aspect, the present disclosure includes a method of enhancing an anti-tumor immune response in a subject in need thereof, the method comprising administering to the subject an effective amount of a gene silencing system, wherein the gene silencing system decreases expression of at least one endogenous immunosuppressive gene in a target cell, thereby enhancing the anti-tumor immune response.

In certain embodiments, the gene silencing system is a CRISPR-based gene silencing system which comprises a plurality of AAV-CRISPR vectors, wherein the plurality of AAV-CRISPR vectors comprises a Cas nuclease and a plurality of guide RNAs (gRNAs) homologous to mRNA from a plurality of target genes associated with immune suppression.

In certain embodiments, the gRNA sequences comprise at least one nucleic acid sequence selected from the group consisting of SEQ ID NOs: 1-1657.

In certain embodiments, the plurality of gRNAs comprise the nucleotide sequences consisting of SEQ ID NOs: 1-1657.

In certain embodiments, the gRNA sequences comprise at least one nucleic acid sequence selected from the group consisting of SEQ ID NOs: 1-92.

In certain embodiments, the plurality of gRNAs comprise the nucleotide sequences consisting of SEQ ID NOs: 1-92.

In certain embodiments, the target genes are selected from the group consisting of Pdl1, Galectin9, Galectin3, and Cd47, or any combination thereof.

In certain embodiments, the CRISPR-based gene silencing system is selected from the group consisting of a type III (Cmr/Csm) system, a type VI system, and a type II system.

In certain embodiments, the type VI system comprises a Cas13 nuclease.

In certain embodiments, the Cas nuclease is a Cas13 nuclease.

In certain embodiments, the Cas13 nuclease is selected from the group consisting of Cas13a, Cas13b, Cas13c, and Cas13d.

In certain embodiments, the Cas13 nuclease is Cas13d.

In certain embodiments, the target cell is an immune cell.

In certain embodiments, the target cell is a T cell.

In certain embodiments, the target cell is a tumor cell.

In certain embodiments, the target cell is a immune cell and a tumor cell.

In certain embodiments, the gene silencing system comprises an RNA interference (RNAi) system.

In certain embodiments, the RNAi system is selected from a shRNA-based system, an siRNA-based system, and a miRNA-based system.

In certain embodiments, the RNAi system targets an endogenous RNA sequence comprising the nucleic acid sequence set forth in SEQ ID NOs: 1658-1665.

In certain embodiments, the RNAi system targets a gene selected from the group consisting of CD200, CD66, Galectin 3, CD47, or any combination thereof.

In certain embodiments, the RNAi system is an shRNA system.

In certain embodiments, the shRNA system comprises at least one nucleic acid selected from the group consisting of SEQ ID NO: 1666-1681.

In certain embodiments, the AAV-CRISPR vector is selected from the group consisting of AAV1, AAV2, AAV3, AAV3B, AAV4, AAV5, AAV6, AAV7, AAV8, AAV9, AAV12, AAV-B1, AAV-DJ, AAV-Retro, AAVrh8, AAVrh10, AAVrh25, Anc80L65, LK03, AAVrh18, AAVrh74, AAVrh32.33, AAVrh39, AAVrh43, Oligo001, PHP-B, and Spark 100.

In certain embodiments, the AAV-CRISPR vector is AAV9.

In certain embodiments, administering the effective amount of the gene silencing system comprises a one dose, a two dose, a three dose, a four dose, or a multi-dose treatment.

In certain embodiments, the tumor is a cancer selected from the group consisting of breast cancer, lung cancer, pancreatic cancer, melanoma, glioma, hepatoma, colon cancer, and brain cancer.

In certain embodiments, the administration of the gene silencing system results in increased CD8+ T cell infiltration into the tumor.

In certain embodiments, the gene silencing system is administered intratumoraly.

In certain embodiments, further comprising administering an additional anti-tumor treatment.

In certain embodiments, the additional anti-tumor treatment is selected from the group consisting of chemotherapy, radiation, surgery, an immune checkpoint inhibitor, and an immune checkpoint blockade antibody.

In certain embodiments, the subject is a mammal.

In certain embodiments, the subject is a human.

In another aspect, the present disclosure includes a vector comprising an adeno-associated virus (AAV) genome, a U6 promoter sequence, a gRNA sequence, an EFS promoter sequence, and a Cas nuclease gene.

In certain embodiments, the gRNA sequence comprises at least one nucleic acid sequence selected from the group consisting of SEQ ID NOs: 1-1657.

In certain embodiments, the Cas nuclease is a RNA-targeting nuclease.

In certain embodiments, the Cas nuclease is a Cas13 nuclease.

In certain embodiments, the Cas13 nuclease is selected from the group consisting of Cas13a, Cas13b, Cas13c, and Cas13d.

In certain embodiments, the Cas13 nuclease is Cas13d.

In another aspect, the present disclosure includes a composition comprising a gRNA library, wherein the gRNA library comprises a plurality of gRNAs that target a plurality of immunosuppressive genes in a cell.

In certain embodiments, the plurality of gRNAs comprise at least one gRNA selected from the group consisting of SEQ ID NOs: 1-1657.

In certain embodiments, the plurality of gRNAs comprise the nucleic acid sequences of SEQ ID NOs: 1-1657.

In certain embodiments, the plurality of gRNAs comprise the nucleic acid sequences of SEQ ID NOs: 3-92.

In certain embodiments, the plurality of gRNAs comprise the nucleic acid sequences of SEQ ID NOs: 93-1657.

In certain embodiments, the gRNA library is packaged into an AAV vector.

### BRIEF DESCRIPTION OF THE DRAWINGS

The following detailed description of specific embodiments of the present disclosure will be better understood when read in conjunction with the appended drawings. For the purpose of illustrating the present disclosure, exemplary embodiments are show in the drawings. It should be understood, however, that the present disclosure is not limited to the precise arrangements and instrumentalities of the embodiments shown in the drawings.

FIGS. 1A-1D illustrate multiplexed Cas13d repression of immunosuppressive genes as combinatorial cancer immunotherapy. FIG. 1A: Schematics of the experimental design for evaluating multiplexed repression of immunosuppressive genes (MUCIG) as immunotherapy.

FIG. 1B: Criteria used to design four different gRNA libraries targeting immunosuppressive gene combinations. FIG. 1C: Growth curves of orthotopic E0771 tumors in C57BL/6 mice. Mice were intratumorally injected with PBS (n=4), AAV-Vector (n=5), AAV-MUCIG Lib1 (n=4), Lib2 (n=4), Lib3 (n=4), or Lib4 (n=5) at days 5, 9 and 14. Statistical significance was assessed by two-way ANOVA, (**p<0.01, ****p<0.0001). FIG. 1D: Spider plots of (FIG. 1C) separated by treatment group for visibility.

FIGS. 2A-2H illustrate rational optimization of MUCIG generates AAV-PGGC, an effective four-gene combination immunotherapy. FIGS. 2A-2D: Protein-level characterization of immunosuppressive factors across a panel of syngeneic cancer cell lines. FIG. 2A: FACS analysis of PGGC library targets (PD-L1, Galectin9, Galectin3, and CD47) in different murine cancer cell lines, either by surface (FIG. 2A) or intracellular (FIG. 2B) staining. FIGS. 2C-2D: Heat maps detailing surface (FIG. 2C) and intracellular (FIG. 2D) expression of several immunosuppressive factors, determined by FACS. Data are expressed in terms of the percentage of total cells that express each marker. FIG. 2E: C57BL/6 mice (n=3) were orthotopically injected with 2×e⁶ E0771-GFP cells. The tumors were harvested at 23 days post injection. Tumor tissues were dissociated for FACS analysis of the indicated markers in each compartment. FIG. 2F: Schematics of the experimental design for intratumoral delivery of the four-gene AAV-PGGC cocktail. FIG. 2G: Growth curves of orthotopic E0771 tumors in C57BL/6 mice. Mice were intratumorally injected with PBS (n=5), AAV-Vector (n=5), AAV-MUCIG Lib4 (n=5), and AAV-PGGC (n=5) at days 5, 9 and 14. Statistical significance was assessed by two-way ANOVA, (****p<0.0001). FIG. 2H: Spider plots of (FIG. 2C), separated by treatment group for visibility.

FIGS. 3A-3F illustrate that AAV-PGGC therapy demonstrates broader anti-tumor activity in syngeneic models of different cancer types. C57BL/6 mice were subcutaneously injected with B16F10 melanoma cells (FIGS. 3A-3B), CT26 colon cancer cells (FIGS. 3C-3D), or Pan02 pancreatic cancer cells (FIGS. 3E-3F). Mice were intratumorally injected with PBS, AAV-Vector, AAV-MUCIG Lib4 or AAV-PGGC at the timepoints indicated by black arrowheads. FIG. 3A: Growth curves of B16F10 tumors treated with PBS (n=5), AAV-Vector (n=5), AAV-MUCIG Lib4 (n=5), and AAV-PGGC (n=5). FIG. 3B: Spider plots of growth curves in (A), separated for visibility. FIG. 3C: Growth curves of CT26 tumors treated with PBS (n=5), AAV-Vector (n=5), AAV-MUCIG Lib4 (n=5), and AAV-PGGC (n=5). FIG. 3D: Spider plots of growth curves in (FIG. 3C), separated for visibility. FIG. 3E: Growth curves of Pan02 tumors treated with PBS (n=5), AAV-Vector (n=5), AAV-MUCIG Lib4 (n=5), and AAV-PGGC (n=5). FIG. 3F: Spider plots of growth curves in (FIG. 3E), separated for visibility. Statistical significance was assessed by two-way ANOVA, (**p<0.01, ****p<0.0001).

FIGS. 4A-4I illustrate that AAV-PGGC treatment remodels the immunosuppressive tumor microenvironment. FIG. 4A: Schematic of experimental design for analyzing of the composition of tumor infiltrating immune populations after AAV-PGGC therapy. FIG. 4B: Relative abundances of several immune populations in orthotopic E0771 (top panels) and CT26 (bottom panels) tumors, at the endpoint of tumor study (35 days post tumor induction). For the E0771 model, mice were treated with PBS (n=3), AAV-Vector (n=4) or AAV-PGGC (n=4) at days 4, 9 and 14. For the CT26 model, mice were treated with PBS (n=4), AAV-Vector (n=4) or AAV-PGGC (n=4) at days 4, 9 and 14. Statistical significance was assessed by two-tailed unpaired/test (*p<0.05, **p<0.01, ***p<0.001). FIG. 4C: UMAP visualization of single tumor-infiltrating immune cells, profiled by scRNA-seq. Mice bearing orthotopic E0771 tumors were treated with PBS, AAV-Vector or AAV-PGGC at days 4, 9 and 14. Tumors were harvested at day 29, and live CD45⁺ cells were sorted for scRNA-seq. FIG. 4D: Violin plots showing the expression levels of representative marker genes across the main cell clusters. FIG. 4E: Relative proportions of each cell type, across treatment groups. Statistical analysis between groups was performed by two-tailed Fisher's exact test. FIG. 4F: UMAP visualization of single tumor-infiltrating immune cells, profiled by scRNA-seq. Mice bearing subcutaneous CT26 tumors were treated with PBS, AAV-Vector or AAV-PGGC at days 4, 9 and 14. Tumors were harvested at day 29, and live CD45⁺ cells were sorted for scRNA-seq. FIG. 4G: Violin plots showing the expression levels of representative marker genes across the main cell clusters. FIG. 4H: Relative proportions of each cell type, across treatment groups. Statistical analysis between groups was performed by two-tailed Fisher's exact test. FIG. 4I: Schematic illustration of AAV-PGGC therapy as a new mode of combinatorial immunotherapy.

FIGS. 5A-5D illustrate the identification of efficient Cas13d gRNAs against Pdl1 and Galectin9. FIG. 5A: Schematic of the experimental approach to identify efficient gRNAs targeting Pdl1 and Galectin9. FIG. 5B: Target sites of the 40 Cas13d gRNAs located along the Pdl1 transcript. FIGS. 5C&5D: FACS analysis of Pdl1 (FIG. 5C) and Galectin9 (FIG. 5D) knockdown efficacy by Cas13d-gRNAs. The gRNA with the highest knockdown efficacy is highlighted in red. Data are expressed as the relative mean of fluorescent intensity (MFI).

FIGS. 6A-6D illustrate the Cas13d-mediated silencing of endogenous immunosuppressive genes in cancer cells. FIG. 6A: Schematic of the experimental approach to identify efficient Cas13d gRNAs targeting various immunosuppressive genes. FIG. 6B: Knockdown efficiency of gRNAs targeting different immunosuppressive genes in cancer cell lines. Data are expressed as the relative mean of fluorescent intensity (MFI). FIG. 6C: Comparison of WT-DR and mut-DR knockdown efficiency. FIG. 6D: Comparison of the Cas13d gRNA-mediated and shRNA-mediated target knockdown. Statistical significance was assessed by two-tailed unpaired/test (*p<0.05, **p<0.01, ***p<0.001, ****p<0.0001).

FIG. 7 illustrates persistent ectopic gene expression in tumors after intratumoral AAV injection. C57BL/6 mice were orthotopically injected with 2×10e⁶ E0771 cells. AAV-luciferase-GFP was then intratumorally injected at the indicated time points. In vivo bioluminescence imaging was performed to visualize luciferase activity.

FIGS. 8A-8B illustrate that AAV-MUCIG treatment alters the composition of immune populations in the tumor microenvironment. FIGS. 8A-8B: FACS characterization of immune populations in orthotopic E0771 tumors. Mice were treated with PBS (n=4), AAV-Vector (n=5) or AAV-MUCIG Lib1 (n=4), AAV-MUCIG Lib2 (n=4), AAV-MUCIG Lib3 (n=4), and AAV-MUCIG Lib4 (n=5) at days 4, 9 and 14. Tumors were harvested at day 37 and analyzed by FACS for T cells (FIG. 8A) and myeloid cells (FIG. 8B). Statistical significance was assessed by two-tailed unpaired/test (*p<0.05, **p<0.01, ***p<0.001).

FIGS. 9A-9C illustrate common signatures of downregulated genes in immune cell populations upon AAV-PGGC treatment. Overlap of the down-regulated genes in CD8⁺ T cells (FIG. 9A), neutrophils (FIG. 9B) and macrophages (FIG. 9C), comparing AAV-PGGC vs AAV-Vector in both the CT26 and E0771 tumor models.

FIGS. 10A-10D illustrate in vivo studies demonstrating multiplexed Cas13d repression of immunosuppressive genes as combinatorial cancer immunotherapy. FIG. 10A: Growth curves of E0771 tumors in C57BL/6 mice. 2×10e⁶ E0771 cells were orthotopically injected into C57BL/6 mice. Mice were intratumorally injected with PBS (n=9), AAV-MUCIG-Vector (Cas13d) (n=10), AAV-MUCIG Pool1 (n=9), Pool2 (n=9), Pool3 (n=9), or Pool4 (n=10) at days 5, 9 and 14 with 2e¹¹ AAV per dose. FIG. 10B: spider plots of (FIG. 10A) separated by treatment group for visibility. FIG. 10C: Growth curves of orthotopic E0771 tumors in C57BL/6 mice. Mice were intratumorally injected with AAV-Vector (Cas13d) (n=10), AAV-MUCIG Pool4 (n=5), and AAV-PGGC (n=10) at days 5, 9 and 14 with 2e¹¹ AAV per dose. FIG. 10D: Spider plots of (FIG. 10C), separated by treatment group for visibility.

FIG. 11 illustrate the relative abundances of CD45%+ populations in orthotopic E0771 (top panels) and subcutaneous Colon26 (bottom panels) tumors, at the endpoint of tumor study (35 days post tumor induction). For the E0771 model, mice were intratumorally treated with PBS (n=3), AAV-Vector (n=4) or AAV-PGGC (n=4) at days 4, 9 and 14. For the Colon26 model, mice were intratumorally treated with PBS (n=4), AAV-Vector (n=4) or AAV-PGGC (n=4) at days 4, 9 and 14. Statistical significance was assessed by one-way ANOVA Tukey's multiple comparisons test, adjusted P Value. (*p<0.05, **p<0.01, * p<0.001).

FIGS. 12A-12L illustrate that AAV-PGGC therapy is dependent on CD8+ T cells, and inhibits metastatic cancer and extends survival. FIG. 12A: Schematic of experimental design for AAV-PGGC and antibody treatment. C57BL/6 mice were orthotopically injected with 2×e6 E0771 cells. Mice were intratumorally injected with AAV-Vector or AAV-PGGC at the indicated timepoint. The tumor bearing mice were intraperitoneally (IP) treated with 100 ug per dose of isotype control, anti-CD8 or anti-GR1 antibody at the indicated time points. FIG. 12B: Growth curves of orthotopic E0771 tumors in C57BL/6 mice after different combinations of AAV with antibodies. FIGS. 12C & 12D: Plot split from FIG. 12B. Analysis of specifical CD8+ T cell (FIG. 12C) or MDSC/neutrophil (FIG. 12D) depletion on AAV-PGGC treatment. FIG. 12E: Schematic of experimental design for a dual site tumor model. C57BL/6 mice were orthotopically injected with 2×e6 E0771 cells at left and 0.2×e6 E0771 cells at right fat pad. Mice were intratumorally injected with AAV-Vector or AAV-PGGC (2e11 AAV per dose) at the indicated timepoint only at the left site. FIGS. 12F & 12G: Growth curves of primary tumor site (FIG. 12F) and distant tumor site (FIG. 12G) in C57BL/6 mice after AAV-PGGC treatment. Statistical significance was assessed by two-way ANOVA. FIG. 12H: Schematic of experimental design for E0771 orthotopic tumor and lung distant metastasis model. C57BL/6 mice were orthotopically injected with 2×e6 E0771-lucifese expressing cells, and a day later intravenous (IV) injection of 0.2×e6 E0771-luciferse expressing cells. Mice were intratumorally and intravenously injected with AAV-Vector or AAV-PGGC (4e11 AAV per dose), and intraperitoneally (IP) treated with 100 μg per dose of isotype or anti-Gr1 antibody at the indicated time points. FIG. 12I: Growth curves of primary tumor in C57BL/6 mice after AAV-PGGC plus anti-Gr1 antibody treatment. Statistical significance was assessed by two-way ANOVA. FIG. 12J: Lung metastatic progression was measured by bioluminescent imaging using IVIS. FIG. 12K: Lung metastasis free survival. The metastasis free survival is defined by the luciferase signal when first showed up in the lung. To standardize across mice, the luciferase signal was firstly normalized from min 500 to max 70,000, then the lung luciferase signal was checked whether showed up or not. Survival curves were analyzed by Log-rank (Mantel-Cox) test. FIG. 12L: Overall survival. Survival curves were analyzed by Log-rank (Mantel-Cox) test. Data points in this figure are presented as mean+s.e.m. Statistical significance was assessed by two-way ANOVA, or Log-rank test as indicated in each panel. *p<0.05, **p<0.01, ***p<0.001, ****p<0.0001. Non-significant comparisons not shown.

FIG. 13 is a histogram illustrating Flow cytometry analysis of PDL1 knockdown efficacy by Cas13d-gRNAs. The gRNA with the highest knockdown efficacy is highlighted in red. Data are expressed as the relative mean of fluorescent intensity (MFI). The gene expression level of Vector was normalized to 1.

FIGS. 14A-14B illustrate further examples of Cas13d-mediated silencing of endogenous immunosuppressive genes in cancer cells. FIG. 14A: Schematic of the experimental approach to compare the knockdown efficient of wild type director repeat (WT-DR) and mutant (Mut-DR). E0771 tumor cells were transduced with Cas13d-EGFP lentivirus. Then GFP⁺ cells were sorted and transfected with WT-DR gRNA or Mut-DR gRNA plasmids. Two days after transfection, target gene expression was tested by flow cytometry analysis. FIG. 14B: Schematic of the experimental approach to compare knockdown efficient between Cas13d gRNAs and shRNA. The cas13d all-in-one (gRNA-Cas13-EGFP) or shRNA plasmid was transfected into Hepa1-6 or MC38 cells. Two days after transfection, target gene expression was tested by flow cytometry analysis. The gRNA successful transfected cells were gated by GFP+ cells.

FIGS. 15A-15C illustrate cas13d on-target and collateral activity testing when targeting endogenous immunosuppressive genes. FIG. 15A: Diagram of Cas13d collateral activity and on-target activity by a dual-GFP and mCherry reporter system. E0711 cell line was co-transduced with three lentiviruses (Cas13d-blasticidin, GFP and mCherry). The Cas13d-expressing dual reporter E0771 cells was selected with blasticidin and then sorted by GFP+mCherry+double positive cells. The dual reporter cells were then transduced with Cas13d-guideRNA lentivirus. Then the GFP and mCherry fluorescent signal was determined by flow cytometry. The on-target gene expression was tested by flow cytometry and qPCR.

FIG. 15B: Flow cytometry analysis of E0771 dual-reporter cells after transduced with different guide RNAs. NTC (Non-Transduced-Control), EV (Empty Vector), SCRg (scramble guideRNA). FIG. 15C: RT-qPCR analysis of the target gene expression. The gene mRNA expression level of SCRg was normalized to 1. Data points in this figure are presented as mean+s.e.m. Statistical significance was assessed by one-way ANOVA Tukey's multiple comparisons test, adjusted P Value. Multiple comparisons were summarized in the bellowing table. *p<0.05, **p<0.01, ***p<0.001.

FIGS. 16A-16F illustrate that AAV-PGGC therapy demonstrates broader anti-tumor activity in syngeneic models of different cancer types. FIG. 16A: Melanoma model. C57BL/6 mice were subcutaneously injected with 1×e⁶ B16F10 melanoma cells. Growth curves of B16F10 tumors intratumorally treated with PBS (n=5), AAV-Vector (n=5), AAV-MUCIG Pool4 (n=5), and AAV-PGGC (n=5) (2e11 AAV per dose) at the timepoints indicated by black arrowheads. FIG. 16B: Spider plots of growth curves in (A), separated for visibility. FIG. 16C: Colon cancer model. BALB/C mice were subcutaneously injected with 2×e⁶ Colon26 colon cancer cells. Growth curves of Colon26 tumors intratumorally injected with PBS (n=5), AAV-Vector (n=5), AAV-MUCIG Pool4 (n=5), and AAV-PGGC (n=5) (2e11 AAV per dose) at the timepoints indicated by black arrowheads. FIG. 16D: Spider plots of growth curves in (FIG. 15C), separated for visibility. FIG. 16E: Pancreatic cancer model. C57BL/6 mice were subcutaneously injected with 2×e⁶ Pan02 pancreatic cancer cells. Growth curves of Pan02 tumors intratumorally treated with PBS (n=5), AAV-Vector (n=5), AAV-MUCIG Pool4 (n=5), and AAV-PGGC (n=5) (2e11 AAV per dose) at the timepoints indicated by black arrowheads. FIG. 16F: Spider plots of growth curves in (FIG. 16E), separated for visibility. Data points in this figure are presented as mean+s.e.m. Statistical significance was assessed by two-way ANOVA. **p<0.01, ****p<0.0001. Non-significant comparisons not shown.

FIG. 17 is a series of dot plots illustrating the gating strategy for myeloid and lymphocyte cells flow cytometry staining. Arrows indicate the parent population that the subsequent plot is gated on. CD8⁺ T=CD8 CD45⁺, CD4⁺=CD4 CD45⁺, Macrophage=CD11b⁺F4/80⁺, Dendric cell (DC)=CD11c⁺MHCII⁺, MDSC=CD11b⁺Ly6G (PMN-MDSC)+CD11b⁺Ly6C⁺(M-MDSC).

FIGS. 18A-18E illustrate that AAV-PGGC local treatment moderately inhibits lung metastasis. FIG. 18A: Schematic of experimental design for E0771 orthotopic tumor and lung distant metastasis model. C57BL/6 mice were orthotopically injected with 2×e6 E0771-luciferase expressing cells, and a day later 0.2×e6 E0771-luciferase expressing cells were intravenously (I.V.) injected. Then mice were intratumorally injected with AAV-Vector or AAV-PGGC (2e11 AAV per dose) at the indicated time points. FIG. 18B: Growth curves of primary tumor in C57BL/6 mice after AAV-PGGC treatment. FIG. 18C: Lung metastatic progression was measured by bioluminescent imaging using IVIS. Representative IVIS imaging are shown. FIG. 18D: Lung metastatic free survival. The metastasis free survival is defined by the luciferase signal when first showed up in the lung. To standardize across mice, the luciferase signal was firstly normalized from min 100 to max 10,000, then the lung luciferase signal was checked whether showed up or not. Survival curves were analyzed by Log-rank (Mantel-Cox) test. FIG. 18E: Overall survival. Survival curves were analyzed by Log-rank (Mantel-Cox) test. Data points in this figure are presented as mean±s.e.m. Statistical significance was assessed by two-way ANOVA, or Log-rank test as indicated in each panel. *p<0.05, ***p<0.001. Non-significant comparisons not shown.

### DETAILED DESCRIPTION

### Definitions

Unless defined otherwise, all technical and scientific terms used herein have the same meaning as commonly understood by one of ordinary skill in the art to which the present disclosure pertains. Although any methods and materials similar or equivalent to those described herein can be used in the practice for testing of the present disclosure, the preferred materials and methods are described herein. In describing and claiming the present disclosure, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20% or +10%, more preferably +5%, even more preferably +1%, and still more preferably +0.1% from the “Allogeneic” refers to any material derived from a different animal of the same species.

As used herein, the term “autologous” is meant to refer to any material derived from the same individual to which it is later to be re-introduced into the individual.

As used herein, the term “bp” refers to base pair.

The term “complementary” refers to the degree of anti-parallel alignment between two nucleic acid strands. Complete complementarity requires that each nucleotide be across from its opposite. No complementarity requires that each nucleotide is not across from its opposite. The degree of complementarity determines the stability of the sequences to be together or anneal/hybridize. Furthermore various DNA repair functions as well as regulatory functions are based on base pair complementarity.

The term “CRISPR/Cas” or “clustered regularly interspaced short palindromic repeats” or “CRISPR” refers to DNA loci containing short repetitions of base sequences followed by short segments of spacer DNA from previous exposures to a virus or plasmid. Bacteria and archaea have evolved adaptive immune defenses termed CRISPR/CRISPR-associated (Cas) systems that use short RNA to direct degradation of foreign nucleic acids. In bacteria, the CRISPR system provides acquired immunity against invading foreign DNA via RNA-guided DNA cleavage.

The term “CRISPR-Cas13d” system or “CRISPR/Cas13d” system as used herein refers to a type IV-D CRISPR/Cas system that has been modified for editing/engineering gene expression. It is typically comprised of a “guide” RNA (gRNA) and a non-specific CRISPR-associated endonuclease (Cas13d). “Guide RNA (gRNA)” is used interchangeably herein with “short guide RNA (sgRNA)” or “single guide RNA (sgRNA). The gRNA is a short synthetic RNA composed of a “scaffold” sequence necessary for Cas13d-binding and a user-defined ˜20 nucleotide “spacer” or “targeting” sequence which defines the target nucleic acid. Cas13d systems differ from genome-editing CRISPR/Cas systems in that they target RNA (e.g. mRNA) for nucleolytic activity. In this way, the activity of the CRISPR-Cas13d system is to reduce gene expression via degradation of mRNA transcripts rather than mutation of the DNA sequene of the target gene. The RNA target of Cas13d can be changed by changing the targeting sequence present in the gRNA.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

The term “therapeutic” as used herein means a treatment and/or prophylaxis. A therapeutic effect is obtained by suppression, remission, or eradication of a disease state.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression which can be used to communicate the usefulness of the compositions and methods of the present disclosure. The instructional material of the kit of the present disclosure may, for example, be affixed to a container which contains the nucleic acid, peptide, and/or composition of the present disclosure or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockout” as used herein refers to the ablation of gene expression of one or more genes.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient vectors for gene delivery. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the present disclosure. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

A “mutation” as used herein is a change in a DNA sequence resulting in an alteration from a given reference sequence (which may be, for example, an earlier collected DNA sample from the same subject). The mutation can comprise deletion and/or insertion and/or duplication and/or substitution of at least one deoxyribonucleic acid base such as a purine (adenine and/or thymine) and/or a pyrimidine (guanine and/or cytosine). Mutations may or may not produce discernible changes in the observable characteristics (phenotype) of an organism (subject).

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T”.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Parenteral” administration of an immunogenic composition includes, e.g., subcutaneous (s.c.), intravenous (i.v.), intramuscular (i.m.), or intrasternal injection, or infusion techniques.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

As used herein, a DNA or RNA nucleotide sequence as recited refers to a polynucleotide molecule comprising the indicated bases in a 5′ to 3′ direction, from left to right.

A “sample” or “biological sample” as used herein means a biological material from a subject, including but is not limited to organ, tissue, exosome, blood, plasma, saliva, urine and other body fluid. A sample can be any source of material obtained from a subject.

As used herein, the terms “sequencing” or “nucleotide sequencing” refer to determining the order of nucleotides (base sequences) in a nucleic acid sample, e.g. DNA or RNA. Many techniques are available such as Sanger sequencing and high-throughput sequencing technologies (also known as next-generation sequencing technologies) such as Illumina's HiSeq and MiSeq platforms or the GS FLX platform offered by Roche Applied Science.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in α/β and γ/δ 30 forms, which are structurally similar but have distinct anatomical locations and functions. Each chain is composed of two extracellular domains, a variable and constant domain. In some embodiments, the TCR can be modified on any cell comprising a TCR, including, for example, a helper T cell, a cytotoxic T cell, a memory T cell, regulatory T cell, natural killer T cell, and/or gamma delta T cell.

The term “immune response” as used herein is defined as a cellular response to an antigen that occurs when lymphocytes identify antigenic molecules as foreign and induce the formation of antibodies and/or activate lymphocytes to remove the antigen.

“Chimeric antigen receptor” or “CAR” refers to an engineered receptor that is expressed on a T cell or any other effector cell type capable of cell-mediated cytotoxicity. The CAR comprises an extracellular domain having an antigen binding domain that is specific for a ligand or receptor. The CAR optionally also includes a transmembrane domain, and a costimulatory signaling domain. In some embodiments, the CAR comprises a hinge. In some embodiments, the antigen binding domain is specific for EGFRvIII. In some embodiments, the costimulatory signaling domain is a 4-1BB signaling domain. In some embodiments, the CAR further comprises a CD3 zeta signaling domain. A CAR-T cell is a T cell engineered to express a CAR.

“Costimulatory ligand,” as the term is used herein, includes a molecule on an antigen presenting cell (e.g., an aAPC, dendritic cell, B cell, and the like) that specifically binds a cognate costimulatory molecule on a T cell, thereby providing a “second” signal which, in addition to the primary signal provided by, for instance, binding of a TCR/CD3 complex with an MHC molecule loaded with peptide, mediates a T cell response, including, but not limited to, proliferation, activation, differentiation, and the like.

A “costimulatory molecule” refers to the cognate binding partner on a T cell that specifically binds with a costimulatory ligand, thereby mediating a costimulatory response by the T cell, such as, but not limited to, proliferation. Costimulatory molecules include, but are not limited to CD28, CD27, and OX40.

Ranges: throughout the present disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Effective immune responses are a fundamental aspect of immunotherapeutic approaches for the treatment of various diseases. However, clinically-important immune responses are often limited by endogenous inhibitory mechanisms such as the expression of so-called “immune checkpoint” or “immune checkpoint inhibitor” genes and proteins by both immune cells (e.g. T cells) and surrounding tissue/tumor cells. While these complex sets of inhibitory mechanisms normally function to protect self-tissues from harmful, inappropriate autoimmune reactions, or to limit the extent of typical, productive immune responses, these mechanisms can also blunt therapeutic immune responses. This situation applies particularly to anti-tumor immune responses, in which immune tolerance mechanisms, among other inhibitory phenomena, have traditionally limited the efficacy of immunotherapeutic approaches such as cancer vaccines and T cell adoptive transfer.

Development of drugs and biologic molecules (e.g. antibodies) which block and prevent the transduction of inhibitory signals via immune checkpoint proteins has demonstrated significant success in enhancing therapeutic immune responses, particularly antibodies against PD-1 and CTLA-4. However current therapies focus on single or a few gene or protein targets, which may contribute to low response rates in some patients, given the complexity and heterogeneity of immune signaling networks. Likewise, while the use of two or more checkpoint-blockade antibodies has demonstrated clinical effectiveness, the difficulties for the approach of combining more and more antibodies scale exponentially, as development of each specific therapeutic antibody is a daunting task by itself.

The present disclosure provides a more flexible, versatile, and effective means for combinatorial immunotherapy through the administration of CRISPR-based gene silencing systems which target immunosuppressive factors expressed by both immune cells and tissues including tumor cells.

### Methods

The present disclosure includes methods for enhancing immune responses in vivo. One aspect of the method comprises administering to a subject an effective amount of a gene silencing system, wherein the gene silencing system decreases expression of at least one endogenous immunosuppressive gene in a target cell, thereby enhancing the immune response.

In another aspect, the present disclosure includes a method of enhancing an anti-tumor immune response in a subject in need thereof, comprising administering to the subject an effective amount of a gene silencing system, wherein the gene silencing system decreases expression of at least one endogenous immunosuppressive gene in a target cell, thereby enhancing the anti-tumor immune response.

In certain embodiments, the gene silencing system of the present disclosure comprises a CRISPR-based gene silencing system which comprises a plurality of AAV-CRISPR vectors. In certain embodiments, the AAV-CRISPR vectors comprise a Cas nuclease and a plurality of guide RNAs (gRNAs) homologous to mRNA transcribed from a plurality of target genes associated with immune suppression. In this way, the administration of the AAV-CRISPR vectors of the present disclosure results in the down-regulation and/or silencing of the target genes. In certain embodiments, the AAV-CRISPR vectors of the present disclosure comprise at least one gRNA sequence selected from the group consisting of SEQ ID NOs: 1-1657 or any combination thereof. In certain embodiments, the plurality of gRNAs of the present disclosure comprise the nucleic sequences comprising SEQ ID NOs: 1-1657 or any combination thereof. In certain embodiments, the plurality of gRNAs of the present disclosure are selected from the nucleic acid sequences set forth in SEQ ID NOs: 1-92 or any combination thereof. In certain embodiments, the target genes are selected from the group comprising CD200, CD66, Pdl1, Galectin9, Galectin3, CD47, and any combination thereof.

In certain embodiments, the gene silencing system comprises an RNAi or RNA interference system which comprises a plurality of short-hairpin RNAs (shRNAs) homologous to mRNA transcribed from a plurality of target genes associated with immune suppression. Administration of the plurality of shRNAs to target cells results in the degradation of mRNA transcribed from target genes, thereby silencing or reducing the expression of target genes. In certain preferred embodiments, the target genes are selected from the group comprising CD200, CD66, Pdl1, Galectin9, Galectin3, CD47, and any combination thereof.

### CRISPR/Cas13d Systems

Type VI CRISPR-Cas systems such as Cas13d systems are relatively simple CRISPR systems which require only one Cas13 protein and a crispr RNA (crRNA), which is a component of the guide RNA (gRNA), for activity. The RNA-specific nucleolytic activity of these systems is provided by the Cas13 protein, and is mediated by the presence of two HEPN domains within this protein. The guide RNA (gRNA) and the Cas protein form a complex that identifies and cleaves target sequences, with the sequence of the gRNA being complementary to the target sequence. In the case of Cas13-based systems, the target sequences are RNAs including mRNAs, and recognition by the Cas13/gRNA complex cleaves and degrades the target RNA molecules. In this way, Cas13-based CRISPR systems can be used to silence the expression of specific genes via the degradation of mRNA transcribed from those genes. Likewise, CRISPR-Cas13 systems can be used to target one or many genes simultaneously by the inclusion or administration of any number of gRNAs with the Cas13 protein. It is also contemplated that any RNA-targeting CRISPR system can be used in the methods of the present disclosure. Non-exclusive examples of RNA-targeting CRISPR systems include but are not limited to type III (Cmr/Csm) systems, type VI systems including Cas13 proteins Cas13a, Cas13b, Cas13c, and Cas13d, and type II systems.

The guide RNA is specific for a nucleic acid sequence of interest and targets that region for Cas endonuclease-induced double strand breaks and degradation. The target sequence of the guide RNA sequence may be within a loci of a gene or within a non-coding region of the genome or may be RNA molecules including mRNA transcribed from specific genes. In certain embodiments, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

Guide RNA (gRNA), also referred to as “short guide RNA” or “sgRNA”, provides both targeting specificity and scaffolding/binding ability for the Cas nuclease. The gRNA can be a synthetic RNA composed of a targeting sequence and scaffold sequence derived from endogenous bacterial crRNA and tracrRNA. gRNA is used to target Cas to a specific target sequence. Guide RNAs can be designed using standard tools well known in the art.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR gene silencing system are introduced into a target cell, such that expression of the elements of the CRISPR system direct formation of a CRISPR complex at one or more target sites. For example, a Cas enzyme, a guide sequence linked to a tracr-mate sequence, and a tracr sequence could each 30 be operably linked to separate regulatory elements on separate vectors. Alternatively, two or more of the elements expressed from the same or different regulatory elements may be combined in a single vector, with one or more additional vectors providing any components of the CRISPR gene silencing system not included in the first vector. CRISPR gene silencing system elements that are combined in a single vector may be arranged in any suitable orientation, such as one element located 5′ with respect to (“upstream” of) or 3′ with respect to (“downstream” of) a second element. The coding sequence 5 of one element may be located on the same or opposite strand of the coding sequence of a second element, and oriented in the same or opposite direction. In certain embodiments, a single promoter drives expression of a transcript encoding a CRISPR enzyme (e.g. Cas13d) and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In certain embodiments, a vector drives the expression of the CRISPR system. The art is replete with suitable vectors that are useful in the present disclosure. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present disclosure may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4th Edition, Molecular Cloning: A Laboratory Manual, Cold Spring Harbor Laboratory, New York, 2012), and in other virology and molecular biology manuals. Viruses, which are useful as 10 vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193). In certain preferred embodiments, the vector of the present disclosure is an adeno-associated virus (AAV) which comprises the CRISPR gene silencing system of the present disclosure.

### AAV Vector Systems

AAV are relatively small, non-enveloped viruses with a ˜4 kb genome that is flanked by inverted terminal repeats (ITRs). The genome contains two open reading frames, one of which provides proteins necessary for replication and the other provides components required for construction of the viral capsid. Wild-type AAV is typically found in the presence of adenovirus as the adenoviruses provide helper proteins that are essential for packaging of the AAV genome into virions. Therefore, AAV production piggy-backs on co-infection with adenovirus and relies on three key elements: the ITR-flanked genome, the open-reading frames, and adeno-helper genes. Due to their non-pathogenic ability to readily infect human cells, AAV is well-studied as a vector for gene delivery. AAV may be readily obtained and their use as vectors for gene delivery has been described in, for example, Muzyczka, 1992; U.S. Pat. No. 4,797,368, and PCT Publication WO 91/18088. Construction of AAV vectors is described in a number of publications, including Lebkowski et al., 1988; Tratschin et al., 1985; Hermonat and Muzyczka, 1984; vectors is described in a number of publications, including Lebkowski et al., 1988; Tratschin et al., 1985; Hermonat and Muzyczka, 1984.

AAV-based vector systems typically separate the viral AAV genes, Adenovirus-derived helper genes, and the transgene payload onto two or three separate plasmids. Three plasmid systems consist of an AAV helper plasmid comprising the rep (replication) and cap (capsid) genes, an adenoviral helper plasmid comprising at least the E2a gene, E4 gene, and VA (viral associated) RNA, and a payload plasmid comprising the transgene and associated promoters and enhancers flanked by ITR sequences. The helper plasmid or plasmids do not comprise ITRs in order to prevent packaging of a functional, infectious viral genome. In certain embodiments, the AAV-CRISPR vectors of the present disclosure comprise AAV particles which comprise a transgene payload comprising a nucleic acid encoding a U6, a DR

In certain embodiments, the CRISPR-based gene silencing system of the present disclosure comprises a plurality of AAV-CRISPR vectors, wherein the AAV-CRISPR vectors comprise a Cas nuclease (e.g. a Cas13d nuclease) and a plurality of gRNAs homologous to mRNA from a plurality of target genes associated with immune suppression. In certain embodiments, the AAV-CRISPR vectors comprise AAV particles which comprise a transgene payload comprising an AAV genome, a U6 promoter sequence, a gRNA sequence, an EFS promoter sequence, and a Cas nuclease gene (e.g. a Cas13d nuclease gene).

The tissue tropism of AAV vector particles is influenced by the serotype of the capsid protein, though the receptors and co-receptors that the capsid proteins bind to are often poorly understood and can be expressed by multiple tissue types. For example, AAV2, one of the most well-studied serotypes, has a binding affinity largely for heparan sulfate proteoglycan (HSPG) and as such has a tropism in humans for eye, brain, lung, liver, muscle, and joint tissues. Likewise, AAVs 1, 4, 5, and 6 have a binding affinity largely for sialic acid and a tropism for neuronal tissues and AAVs 5 and 8 which share a tropism for skeletal muscle cell. In this way, the serotype of the AAV capsid protein can be selected to target the payload nucleic acid of the AAV vector to a specific tissue or cell type. Alteration or modification of capsid protein structure can also alter the tissue or cellular tropism and affinity of the resulting AAV vector particles.

In certain embodiments of the present disclosure, the AAV-CRISPR vectors target immune cells including CD4+ T cells, CD8+ T cells, B cells, antigen presenting cells, and the like. In certain embodiments, the AAV-CRISPR vectors target non-immune tissue cells including but not limited to endothelial cells, mesenchymal cells, fibroblasts, and the like, as these cells also express immunosuppressive factors which contribute to immune suppression. In certain embodiments, the target cells are tumor cells. Suppression of anti-tumor immune responses have been demonstrated to be a key factor in tumor development and progression and contribute to poor prognosis and patient survival and tumor cells and tissues have been found to express a complex network of factors including checkpoint inhibitor proteins, which contribute to the immunosuppressive nature of the tumor microenvironment (TME). In certain embodiments, the AAV-CRISPR vectors of the present disclosure target both immune and non-immune cells withing a particular microenvironment simultaneously. By way of a non-exclusive example, the AAV-CRISPR vectors of the present disclosure can be introduced into a tumor microenvironment, where the CRISPR-based gene silencing system of the present disclosure suppresses the expression of one or more immunosuppressive genes expressed by both immune and non-immune cells including tumor cells, the overall effect being the improvement of anti-tumor immune responses.

It is contemplated that the AAV-CRISPR vectors of the present disclosure can be used with any naturally occurring, modified, hybrid, or engineered AAV capsid protein including, but not limited to AAV1, AAV2, AAV3, AAV3B, AAV4, AAV5, AAV6, AAV7, AAV8, AAV9, AAV12, AAV-B1, AAV-DJ, AAV-Retro, AAVrh8, AAVrh10, AAVrh25, Anc80L65, LK03, AAVrh18, AAVrh74, AAVrh32.33, AAVrh39, AAVrh43, Oligo001, PHP-B, and Spark 100 among others. In certain embodiments, the AAV-CRISPR vector is AAV9. The skilled artisan would be able to select an appropriate capsid protein for use with the present disclosure based on the desired target tissue or cell type.

### Compositions

In certain embodiments, the present disclosure includes use of “guide RNAs” (gRNAs) which can be utilized in combination with RNA-targeting CRISPR systems to modulate gene expression by targeting and degrading specific mRNA sequences.

In one aspect, the present disclosure includes a guide RNA (gRNA) library that target a plurality of immunosuppressive genes in a target cell. In some embodiments, the gRNA library comprises a plurality of nucleic acids comprising one or more nucleotide sequences selected from the group consisting of SEQ ID NOs. 1-1,657. In further embodiments, the library further comprises at least one nucleotide sequence selected from the group consisting of SEQ ID NOs. 3-92. In further embodiments, the library further comprises at least one nucleotide sequence selected from the group consisting of SEQ ID NOs. 93-1,657. In some embodiments, the gRNA library comprises a plurality of nucleic acids comprising the nucleotide sequences of SEQ ID NOs. 1-1,657. In further embodiments, the library comprises a plurality of nucleic acids comprising the nucleotide sequences of SEQ ID NOs. 3-92. In further embodiments, the library comprises a plurality of nucleic acids comprising the nucleotide sequences of SEQ ID NOs. 93-1,657. In certain embodiments, the library can be packaged into a vector. Any vector known to one of ordinary skill in the art can be used, including but not limited to lentiviral vectors, adenoviral vectors, and adeno-associated viral (AAV) vectors.

With regard to any of the gRNA libraries or lentiviral libraries comprising the SEQ ID NOs. 1-1,657 or any combination thereof, it should be understood by one of ordinary skill in the art that the present disclosure is construed to encompassing every individual SEQ ID NO. in the range and all combinations thereof.

Also included in the present disclosure is a vector, e.g. an AAV-CRISPR vector. The vector comprises a adeno-associated virus (AAV) genome, a U6 promoter sequence, a gRNA sequence, an EFS promoter sequence, and a Cas nuclease gene. The vector can include additional components, including but not limited ot an LTR sequence, aT2A sequence, a linker sequence, an NLS sequence, and a short PA sequence. In certain embodiments, the first promoter is a U6 promoter and/or the second promoter is an EFS promoter.

Any promoter known to one of ordinary skill in the art can be incorporated into any of the vectors of the present disclosure. Suitable promoter and enhancer elements are known to those of skill in the art. For expression in a bacterial cell, suitable promoters include, but are not limited to, lacl, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences may also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the present disclosure should not be limited to the use of constitutive promoters. Inducible promoters are also contemplated as part of the present disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In certain embodiments, the vector comprises a U6 promoter and/or an EFS promoter. Certain embodiments of the present disclosure include more than one promoter per vector. It should be known to one of ordinary skill in the art that the when a vector comprises more than one promoter, said promoters can include two or more of the same promoter or two or more different promoters. For example, the vector may comprise a first promoter comprising a U6 promoter and a second promoter comprising an EFS promoter.

In addition, any of the vectors/plasmids of the present disclosure can include additional components. For example, an antibiotic resistance gene/sequence. Any antibiotic resistance gene/sequence or selection marker known to one of ordinary skill in the art can be include in the vector.

The present disclosure should be construed to encompass any type of vector known to one of ordinary skill in the art. For example, the vector can comprise an adeno-associated virus, but can also comprise other viral vectors including but not limited to adenovirus, lentivirus, retrovirus, hybrid viral vectors, or any combinations thereof.

### Cancer and Other Diseases/Disorders

Certain embodiments of the present disclosure include compositions and methods for enhancing an immune response. In other aspects, the present disclosure include compositions and methods for enhancing anti-tumor immune Reponses. It is contemplated that any disease which can be targeted by an immune response can be treated with the compositions of the present disclosure which enhance such immune responses. Diseases/disorder/conditions that can be treated include but are not limited to autoimmune diseases, inflammation, neuroimmune disorders, and other immune system disorders.

The present disclosure includes compositions and methods for enhancing anti-tumor immune responses. Enhancement of such responses results in greater killing of tumor cells by the patient's immune system or by adoptively transferred immune cells which are specific for the cancer, thereby treating the cancer. Types of cancer that can be treated include, but are not limited to, Acute Lymphoblastic Leukemia (ALL), Acute Myeloid Leukemia (AML), Adrenocortical Carcinoma, AIDS-Related Cancers, Kaposi Sarcoma, AIDS-Related Lymphoma, Primary CNS Lymphoma, Anal Cancer, Appendix Cancer (Gastrointestinal Carcinoid Tumors), Astrocytomas, Atypical Teratoid/Rhabdoid Tumor, Brain Cancer, Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors, Burkitt Lymphoma, Non-Hodgkin Lymphoma, Carcinoid Tumors, Carcinoma of Unknown Primary, Cardiac (Heart) Tumors, Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma (Mycosis Fungoides and Sezary Syndrome), Ductal Carcinoma In Situ (DCIS), Endometrial Cancer, Ependymoma, Esophageal Cancer, Esthesioneuroblastoma, Ewing Sarcoma, Extracranial Germ Cell Tumor, Eye Cancer, Intraocular Melanoma, Fallopian Tube Cancer, Fibrous Histiocytoma of Bone, Osteosarcoma, Gallbladder Cancer, Gastric Cancer, Stomach Cancer, Gastrointestinal Carcinoid Tumor, Gastrointestinal Stromal Tumors (GIST), Central Nervous System Germ Cell Tumors, Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis (Langerhans Cell), Hodgkin Lymphoma, Hypopharyngeal Cancer, Intraocular Melanoma, Islet Cell Tumors, Pancreatic Neuroendocrine Tumors, Kidney Cancer, Renal Cell Cancer, Langerhans Cell Histiocytosis, Laryngeal Cancer, Leukemia, Lip and Oral Cavity Cancer, Liver Cancer, Lung Cancer (Non-Small Cell and Small Cell), Lymphoma, Male Breast Cancer, Malignant Fibrous Histiocytoma of Bone and Osteosarcoma, Melanoma, Intraocular (Eye) Melanoma, Merkel Cell Carcinoma (Skin Cancer), Malignant Mesothelioma, Metastatic Cancer, Metastatic Squamous Neck Cancer with Occult Primary, Midline Tract Carcinoma With NUT Gene Changes, Mouth Cancer, Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Nasal Cavity and Paranasal Sinus Cancer, Nasopharyngeal Cancer, Neuroblastoma, Non-Small Cell Lung Cancer, Oral Cancer, and Oropharyngeal Cancer, Ovarian Cancer, Pancreatic Cancer, Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System (CNS) Lymphoma, Primary Peritoneal Cancer, Prostate Cancer, Rectal Cancer, Recurrent Cancer, Retinoblastoma, Rhabdomyosarcoma, Salivary Gland Cancer, Sarcoma, Vascular Tumors, Uterine Sarcoma, Sezary Syndrome (Lymphoma), Small Cell Lung Cancer, Small Intestine Cancer, Soft Tissue Sarcoma, Squamous Cell Carcinoma, Stomach (Gastric) Cancer, Throat Cancer, Thymoma, Thymic Carcinoma, Thyroid Cancer, Transitional Cell Cancer of the Renal Pelvis and Ureter, Carcinoma of Unknown Primary, Ureter and Renal Pelvis, Transitional Cell Cancer, Urethral Cancer, Uterine Cancer, Vaginal Cancer, Vulvar Cancer, Wilms Tumor, and combinations thereof.

In certain embodiments, the subject can be administered an additional treatment, such as but not limited to an anti-tumor treatment. For example, the subject can be administered a combination of a composition of the present disclosure and an additional treatment, such as but not limited to an anti-tumor treatment. Examples of additional treatments include but are not limited to, chemotherapy, radiation, surgery, medication, immune checkpoint inhibitors, immune checkpoint blockade (ICB) antibodies, immune checkpoint inhibitors that block CTLA-4 or PD1, anti-CTLA4 monoclonal antibody, anti-PD1 monoclonal antibody, anti-PD-LI monoclonal antibody, adoptive cell transfer, human recombinant cytokines, cancer vaccines, immunotherapy, targeted therapy, hormone therapy, stem cell transplant, precision medicine, non-specific immunotherapy (e.g. cytokines and chemokines, such as IL-2, IFNa, IFNb, IFNg), oncolytic virus therapy, T-cell therapy (e.g. adoptive transfer of TILs, CAR-T therapy), cancer vaccines (e.g. conventional DC vaccine), Ipilimumab (Yervoy), Nivolumab (Opdivo), Pembrolizumab (Keytruda), Atezolizumab (Tecentriq), Avelumab (Bavencio), Durvalumab (Imfinzi), Anti-LAG-3, anti-TIM1, Anti-TIM3, Anti-CSF-R, IDO inhibitor, OX-40 agonist, GITR agonist, CD80 agonist, CD86 agonist, ICOS agonist, ICOSLG agonist, CD276 agonist, VTCN1 agonist, TNFSF14 agonist, TNFSF9 agonist, TNFSF4 agonist, CD70 agonist, CD40 agonist, LGALS9 agonist, CD80 inhibitor, CD86 inhibitor, ICOS inhibitor, ICOSLG inhibitor, CD276 inhibitor, VTCN1 inhibitor, TNFSF14 inhibitor, TNFSF9 inhibitor, TNFSF4 inhibitor, CD70 inhibitor, CD40 inhibitor, LGALS9 inhibitor, TLR9 agonist, CD20 antibody, CD80 antibody, TIGIT antibody, B7-H1 antibody, B7-H2 antibody, B7-H3 antibody, B7-H4 antibody, CD28 antibody, CD47 antibody, anti-BTLA, anti-Galetin9, anti-IL 15R, anti-GD2. In some embodiments the monoclonal antibody is fully human, humanized or chimeric.

### Introduction of Nucleic Acids

In certain embodiments an expression system is used for the introduction of gRNAs and Cas13d proteins into the cells of interest. Typically employed options include but are not limited to plasmids and viral vectors such as adeno-associated virus (AAV) vector or lentivirus vector.

Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8): 861-70 (2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5:505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present disclosure, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the present disclosure.

Moreover, the nucleic acids may be introduced by any means, such as transducing the cells, transfecting the cells, and electroporating the cells. One nucleic acid may be introduced by one method and another nucleic acid may be introduced into the cell by a different method.

### RNA

In certain embodiments, the nucleic acids introduced into the cell are RNA. In another embodiment, the RNA is mRNA that comprises in vitro transcribed RNA or synthetic RNA.

The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA.

PCR can be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary”, as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In certain embodiments, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In certain embodiments, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In certain embodiments, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In certain embodiments, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In certain embodiments, the mRNA has a cap on the 5′ end and a 3′ poly(A) tail which determine ribosome binding, initiation of translation and stability mRNA in the cell. On a circular DNA template, for instance, plasmid DNA, RNA polymerase produces a long concatameric product which may not be suitable for expression in eukaryotic cells. The transcription of plasmid DNA linearized at the end of the 3′ UTR results in normal sized mRNA which may not be effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003)).

The conventional method of integration of polyA/T stretches into a DNA template is by molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The poly A/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In certain embodiments, the poly(A) tail is between 100 and 5000 adenosines.

Poly(A) tails of RNAs can be further extended following in vitro transcription with the use of a poly(A) polymerase, such as E. coli polyA polymerase (E-PAP). In certain embodiments, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

In some embodiments, the RNA is electroporated into the cells, such as in vitro transcribed RNA.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

One advantage of RNA transfection methods of the present disclosure is that RNA transfection is essentially transient and vector-free. A RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

RNA has several advantages over more traditional plasmid or viral approaches. Gene expression from an RNA source does not require transcription and the protein product is produced rapidly after the transfection. Further, since the RNA has to only gain access to the cytoplasm, rather than the nucleus, and therefore typical transfection methods result in an extremely high rate of transfection. In addition, plasmid based approaches require that the promoter driving the expression of the gene of interest be active in the cells under study.

In another aspect, the RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, U.S. Pat. Nos. 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

### Pharmaceutical Compositions

Pharmaceutical compositions of the present disclosure may comprise an AAV-CRISPR vector or a gene silencing system, as described herein, in combination with one or more pharmaceutically or physiologically acceptable carriers, diluents or excipients. Also provided are pharmaceutical compositions comprising an engineered immune cell of the present disclosure.

Compositions of the present disclosure may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

The administration of the composition of the present disclosure may be carried out in any convenient manner known to those of skill in the art. The composition of the present disclosure may be administered to a subject by aerosol inhalation, injection, ingestion, transfusion, implantation or transplantation. The compositions described herein may be administered to a patient transarterially, subcutaneously, intradermally, intratumorally, intranodally, intramedullarly, intracystically intramuscularly, by intravenous (i.v.) injection, parenterally or intraperitoneally. In other instances, the composition of the present disclosure are injected directly into a site of inflammation in the subject, a local disease site in the subject, a lymph node, an organ, a tumor, and the like.

It should be understood that the methods and compositions that would be useful in the present disclosure are not limited to the particular formulations set forth in the examples. The following examples are put forth so as to provide those of ordinary skill in the art with a complete disclosure and description of how to make and use the cells, expansion and culture methods, and therapeutic methods of the present disclosure, and are not intended to limit the scope of what the inventors regard as their disclosure.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the present disclosure, and, as such, may be considered in making and practicing the present disclosure. Particularly useful techniques for particular embodiments will be discussed in the sections that follow.

### Experimental Examples

The present disclosure is now described with reference to the following Examples. These Examples are provided for the purpose of illustration only, and the present disclosure is not limited to these Examples, but rather encompasses all variations that are evident as a result of the teachings provided herein.

The materials and methods employed in these experiments are now described.

Cell lines. E0771 cell was from CH3. Hepa1-6, MC38, Colon26, B16F10, Pan02 were from ATCC. HEK293FT cell was purchased from Thermo Fisher Scientific for producing virus. All cell lines were maintained at 37C with 5% CO2 in D10 medium (Dulbecco's modified Eagle's medium supplemented with 10% fetal bovine serum).

Mice. Mice of both sexes, between age 6 and 12 weeks, were used for the study. 6-8-week-old C57BL/6Nr mice were purchase from Charles River lab. Female mice were used for breast cancer (E0771) models. Male mice were used for B16F10 and Pan02 mouse model. 6-8-week-old BALB/C mice were purchased from Jackson lab. All animals were housed in standard, individually ventilated, pathogen-free conditions, with a 12 h: 12 h or a 13 h: 11 h light cycle, at room temperature (21-23° C.) and 40-60% relative humidity.

Cas13d cancer cell line generation. For lentivirus production, 20 ug plasmid of PXR001 (EF1a-Cas13d-2A-EGFP, addgene #109049) together with 10 μg pMD2.G and 15 μg psPAX2 were co-transfected into HEK293FT cells in a 150 mm cell culture dish at 80-90% confluency using 135 μl LipoD293 transfection reagent (Signage, SL100668). Virus supernatant was collected 48h post transfection, centrifuged at 3000 g for 15 min to remove the cell debris. The supernatant was then concentrated with Amicon Ultra-15 filter from 20 ml to 2 ml. The virus was aliquoted and stored at −80 C. To generate Cas13d overexpression cell line, the cancer cells were transduced with lentivirus PXR001, and the positive cells which were GFP expressing were flow cytometry sorted.

Transfection and flow cytometry knockdown efficacy test. To test each gRNA knockdown efficacy, gRNAs were cloned into BbsI site of PXR003 plasmid (Cas13d gRNA cloning backbone, addgene #109053) and were transient transfected into Cas13d expressing cancer cell. For the transfection experiments, 5×10⁴ cells per well of a 48 well plate was seeded 12h before transfection. 500 ng gRNA plasmid together with a 1:1 ratio of Lipofectamine 2000 to DNA were transfected into cells. Flow cytometry was performed at 48h post transfection.

Generation of AAV-MUCIG library. An AAV version plasmid expressing U6-mutation direct repeat-gRNA clone site-EFS-Cas13d (pAAV-U6-EFS-Cas13d) was cloned into AAV backbone. All pooled gRNA library were synthesized as single stranded oligonucleotides from Genescript or IDT. The oligos were amplified by PCR and Gibson cloned into pAAV-U6-EFS-Cas13d. The purification and electroporation of Gibson products into Endura electrocompetent cells were performed as previously described, with at least ×100 coverage of colonies represented per sgRNAs. AAV was produced by co-transfecting HEK293FT cells with AAV-MUCIG library together with AAV9 serotype plasmid and helper plasmid PDF6. Briefly, HEK293FT cells were seeded in 150 cm dish or hyper flask 12-18h before transfection. When cells got 80-90% confluency, 6.2 ug AAV-vector or AAV-MUCIG library, 8.7 ug AAV9 serotype, and 10.4 ug PDF6 were transfected with 130 μl PEI, incubating 10-15 min before adding into cells. Replicates collected multiple dishes were pooled to enhance production yield. Cells were collected 72 h post transfection. For AAV purification, chloroform (1:10 by volume) was added and was shaken vigorously for 1 h at 37° C. NaCl was added to a final concentration of IM and shaken until dissolved. The mixture was centrifuges at 20,000 g for 15 min at 4 C. The aqueous layer was transferred to a new tube, and then PEG 8000 (10%, w/v) was added and shaken until dissolved. The mixture was incubated on ice for 1 h. The pellet was spun down at 20,000 g for 15 min at 4° C. The supernatant was discarded, and the pellet was resuspended in DPBS. The resuspension was treated with Benzonase and MgCl2 AT 37 C for 30 min. Chloroform (1:1 by volume) was then added, shaken and spun down at 12,000 g for 15 min at 4° C. The aqueous layer was isolated and concentrated through Ambion Ultra-15 tube. The concentrated solution was washed with PBS and the filtration process repeated. Then AAV was treated with DNase I for 30 min at 37° C. Genomic copy number (GC) of AAV was determined by real-time qPCR using custom TaqMan assays (Thermo Fisher Scientific) targeted to EFS promoter.

Therapeutic testing of AAV-g-MUCIG in syngeneic tumor models. Syngeneic orthotopic breast tumor was established by transplanting 2×10⁶ E0771 cells into mammary fat pad of 6-8-week-old female C57BL/6Nr mice. Then 5, 9, and 14 days after transplantation, 2e11 AAV particles of vector or MUCIG, or PBS were injected intratumorally into tumor bearing mice. The tumor volume was measured every 3-4 days. For the B16F10 melanoma model, 1×10⁶ B16F10 cancer cells were subcutaneously injected into the male left flank of C57BL/6Nr mice. 5, 9, 13 days post transplantation, 2×10¹¹ AAV particles of vector or MUCIG, or PBS were intratumorally administrated into tumor bearing mice. The tumor volume was measured every 2 days. For the pancreatic tumor model, 2×10⁶ Pan02 cells were subcutaneously injected into the left flank of C57BL/6Nr mice. Then, 5, 14, 18 days after transplantation, 2e11 AAV particles of vector or MUCIG, or PBS were intratumorally administrated into tumor bearing mice. The tumor volume was measured every 3-4 days. For the colon tumor model, 2×10⁶ CT26 cells were subcutaneously injected into the left flank of BALB/C mice. Then, 5, 9, 14 days after transplantation, 2e¹¹ AAV particles of vector or MUCIG, or PBS were intratumorally administrated into tumor bearing mice. The tumor volume was measured every 3 days. Tumor volume was calculated with the formula: volume =π/6*xyz. Two-way ANOVA was used to compare growth curves between treatment groups.

In vivo luciferase imaging. The bioluminescent imaging was performed to detect AAV delivery gene expression. Mice were injected with luciferin (150 mg/kg) by intraperitoneal injection and activity quantified in live animal for 10 min later following with 1 min exposure. The photon flux was monitored by the PE IVIS Spectrum in vivo imaging system. The signaling was monitored and quantified by the IVIS software.

Isolation of TILs. Tumors were minced into 1 mm size pieces and then digested with 100U/ml collagenase IV and DNase I for 60 min at 37° C. Tumor suspensions were filtered through 100-μm cell strainer to remove large bulk masses. The cells were washed twice with wash buffer (PBS plus 2% FBS). 1 ml ACK lysis buffer was added to lysis red blood cell by incubating 2-5 min at room temperature. The suspension was then diluted with wash buffer and spin down at 400 g for 5 min at 4° C. Cell pellet was resuspended with wash buffer and followed by passing through a 40 μm cell strainer. Cells were spin down and washed twice with wash buffer. At last, cell pellet was resuspended in MACS buffer (PBS with 0.5% BSA and 2 mM EDTA). The single cell suspensions were used for flow cytometry staining and FACS sorting. TILs were labeled as Cd45 positive cells.

Flow cytometry. For the TILs FACS analysis, single cell suspension from tumor were prepared as described above. For the myeloid cell staining panel, anti-CD45-Percp-Cy5.5, anti-CD11b-FITC, anti-CD11c-PE/Dazzle, anti-F4/80-PE, anti-Ly6G-BV605, anti-Ly6C-APC, and anti-MHCII-PE/Cy7 were used. For lymphoid cell staining panel, anti-CD45-Percp-Cy5.5, anti-CD8-BV605, anti-CD4-PE. All flow antibodies were used at 1:100 dilutions for staining. The LIVE/DEAD Near-IR was diluted 1:1000 to distinguish live or dead cells.

For the in vitro cancer cell line staining, cancer cells were incubated with trypsin and washed twice with PBS. For cell surface staining, surface antibody was diluted 1:100 and stained in MACS buffer on ice for 15 min. Cells were washed twice with MACS buffer. For intracellular staining, Intracellular Fixation & Permeabilization Buffer Set (eBioscience) was used to fix and permeabilize cells. Briefly, after the surface marker staining, cells were resuspended in 100 μl Fixation/Permeabilization working solution, and incubated on ice for 15 min. Then cells were washed with 1× permeabilization buffer by centrifugation at 600 g for 5 min. Then the cell pellet was resuspended in 100 μl of 1× permeabilization buffer with 1:100 intracellular staining antibodies and incubating on ice for 15 min. After staining, cells were centrifuged at 600 g for 5 min, and washed twice with staining buffer before being analyzed or sorted on a BD FACSAria. The data were analyzed using FlowJo software.

Immune cell profiling by scRNA-seq. E0771 or CT26 tumors were collected at the indicated time point post injection. Single cell suspensions were collected as described above. The cells were labeled with Cd45-Percp-Cy5.5 antibody and live/dead dye. FACS sorted cells were gated on CD45⁺ live cells. Sorted cells were washed with PBS, and cell numbers and viabilities were assessed by trypan blue staining. The 10,000 CD45⁺ cells isolated from tumors were used for scRNA-seq library prep by following the protocol from 10× Genomics Chromium Next GEM Single Cell 5′ Reagent Kits V2.

scRNA-seq data analysis. Analysis of scRNA-seq was performed using the Seurat v4 package in R. All cells from the three treatment groups (PBS, AAV-Vector, and AAV-PGGC) were merged and integrated by tumor type (E0771 or CT26). The data was filtered to retain cells with <15% mitochondrial counts and 200-3500 unique expressed features. The expression data for each cell was normalized by the total reads and log-transformed. Harmony was utilized to integrate datasets from the same tumor type for the purpose of identifying cell clusters. Each cell cluster was annotated by cell type using canonical marker genes, with higher-resolution subclustering of the lymphocyte populations. To determine differences in cell type frequencies, 2×2 contingency tables were constructed for each cell type, comparing AAV-Vector and AAV-PGGC treatment groups. A two-tailed Fisher's exact test was performed on the contingency table for each cell type. Differentially expressed genes were identified by comparing cells from AAV-Vector vs AAV-PGGC treatment groups using the default settings in Seurat, with statistical significance set at adjusted p<0.05.

Statistical analysis. Data analysis was performed using GraphPad Prism v.9 and R 3.5. The unpaired, two-sided, unpaired/test was used to compare two groups unless indicated otherwise. Two-way ANOVA was used to compare multiple groups in the tumor growth curves with two independent variables. P<0.05 was considered statistically significant.

### Example 1: Efficient Knockdown of Endogenous Immune Suppressive Genes Using Cas13d

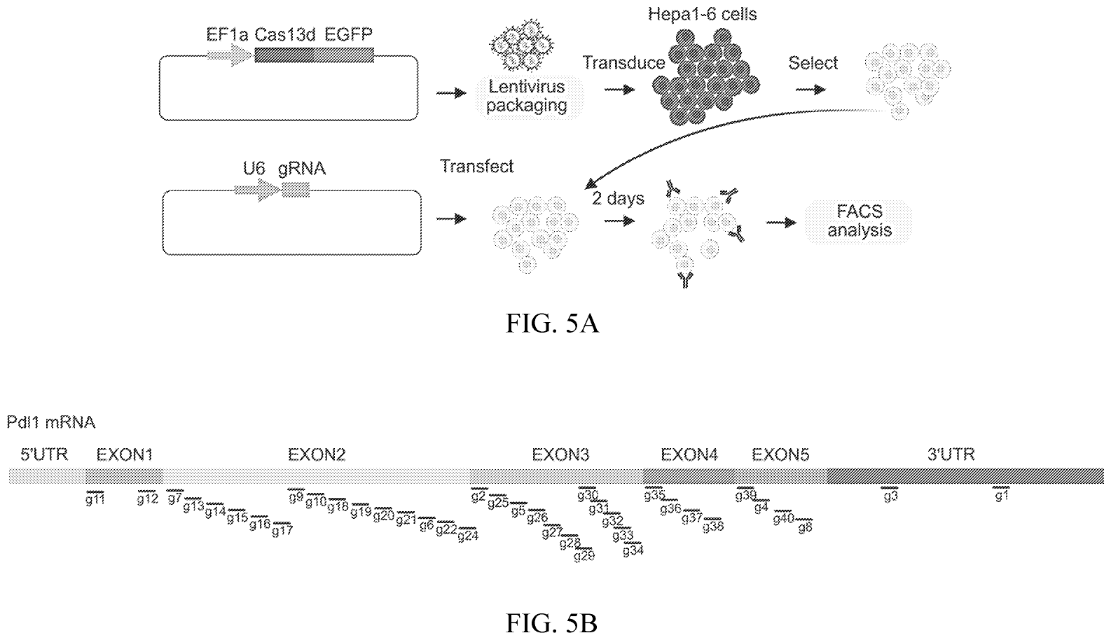

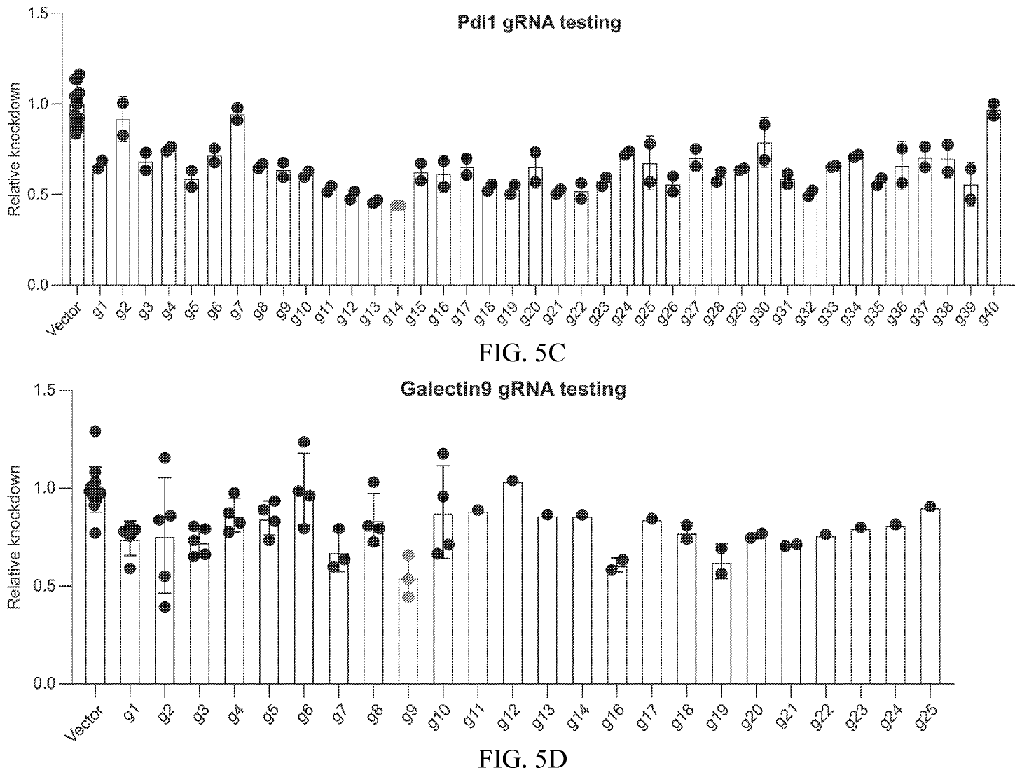

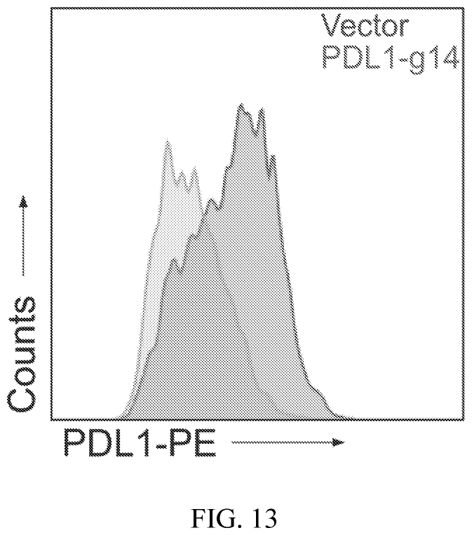

To assess the efficiency of Cas13d-mediated RNA knockdown, a Hepa1-6 tumor cell line was established which stably expressed Cas13d-GFP. gRNAs were then transfected into the cells, and FACS analysis of gene expression was performed 2 days after transfection (FIG. 5A). To identify effective gRNAs for Cas13d-mediated knockdown efficiency of Pdl1 ((′d274), 40 gRNAs that targeted the Pdl1 mRNA sequence (FIG. 5B and Table 1) were screened. FACS analysis showed that 29 out of 40 gRNAs could successfully knock down Pdl1 (FIGS. 5C and 13). Among all the gRNAs, g14 showed the best knockdown efficiency, resulting in 56%±0.079% reduction of Pdl1. Similarly, 25 gRNAs were designed targeting Galectin9 and assessed or knockdown efficiency. It was found that transfection of g9 could successfully knock down Galectin9 by 45%±0.073% (FIG. 5D).

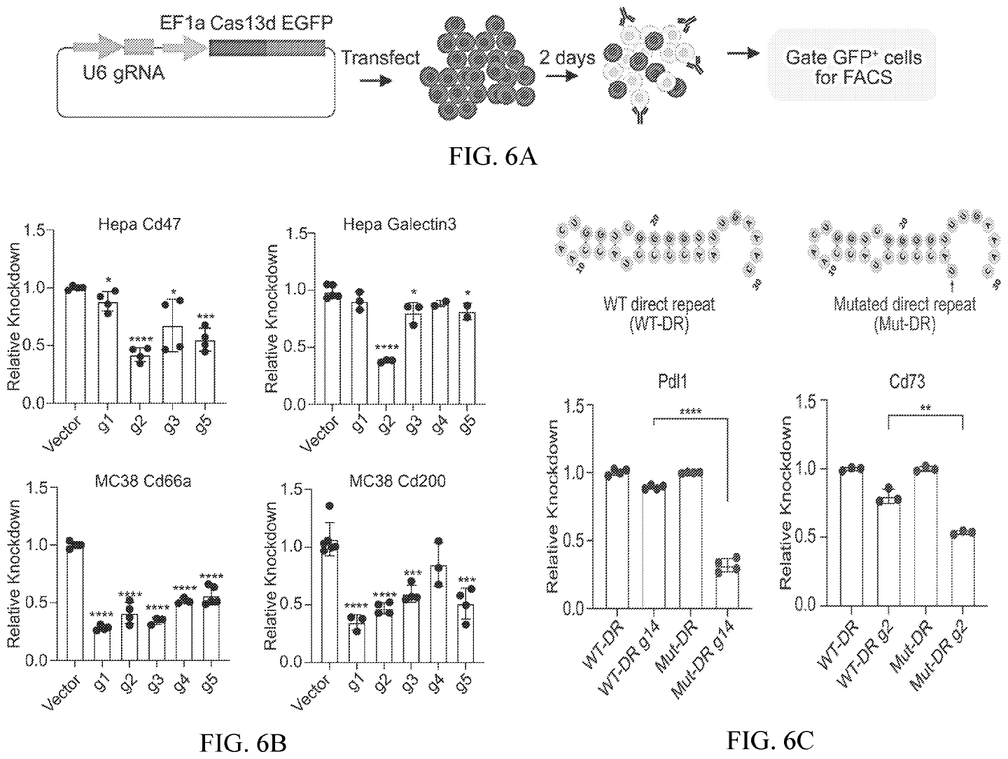

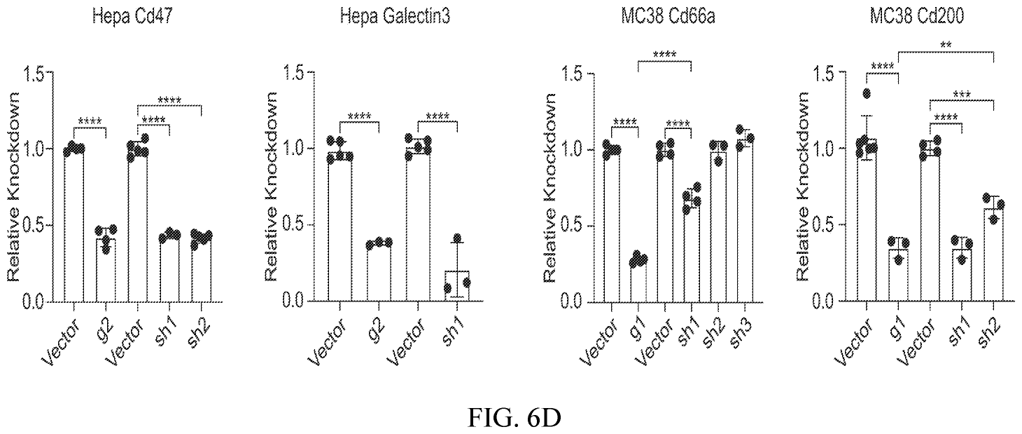

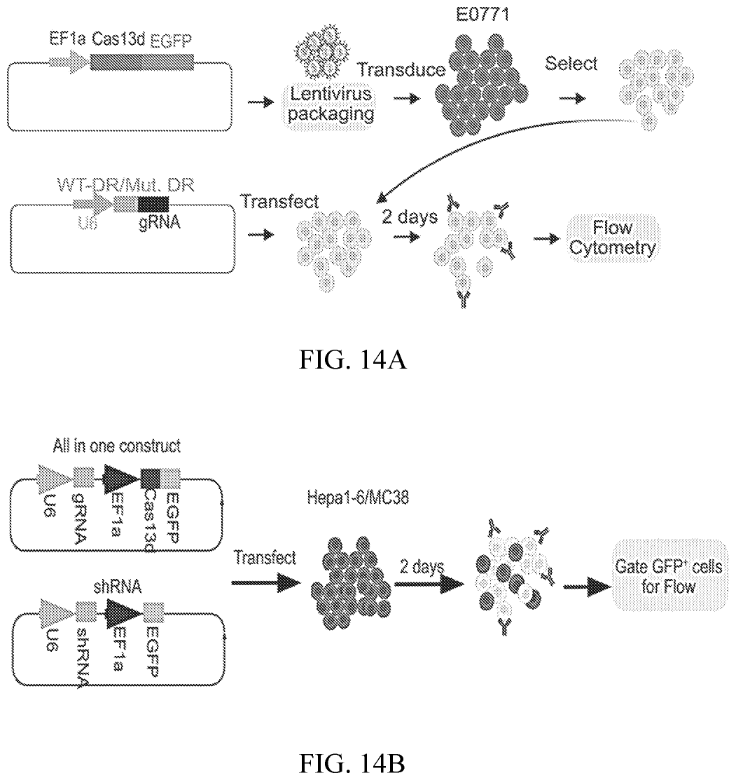

A computational model to predict Cas13d gRNAs was recently developed (H. H. Wessels et al., Nat Biotechnol 38, 722-727 (2020)). To further improve the Cas13d gRNA design for immune genes, this design tool was applied to design 4 to 5 gRNAs for 4 different immunosuppressive genes of interest: (′d47, Galectin-3, Cd66a, and (d200 (Table 1) To assess the efficiency of these tool-designed gRNAs, an all-in-one vector was generated including gRNA, Cas13d and selection marker EGFP (FIG. 6A). FACS was performed in order to gate the GFP positive cells, and then gRNA knockdown efficacy was analyzed by fluorescence intensity. It was found that the designed gRNAs could efficiently knock down the target genes (FIG. 6B). For all 4 targeted genes, at least one gRNA was identified for each target gene that achieved over 50% knockdown efficiency. For Cd66a, all 5 designed gRNAs showed robust knockdown. These data thus indicated that the Cas13d gRNA design tool is predictive and reliable for the following multiplex genes targeting. To achieve stronger gene repression, the knockdown efficiency of gRNAs bearing the wildtype direct repeat (WT-DR) was compared to a mutant DR, as previously described. The WT-DR or the Mut-DR-gRNA plasmid was transfected into E0771-Cas13d overexpressing cells (FIG. 14A). The FACS analysis showed that using a mutated DR with the Pdl1 gRNA improved the knockdown efficacy by 57%±0.026% when compared to WT-DR (FIG. 6C). For Cd73 gRNA, it was similarly observed 26.4±0.031% improvement with mut-DR. Studies also compared the knockdown efficacy between Cas13d-mediated gRNAs and shRNAs (Table 2), illustrating that Cas13d-mediated gRNA had better or similar knockdown than shRNAs, for the same genes in the same cell types, even if the WT-DR was used (FIGS. 6D and 14B). These data indicate that Cas13d-gRNA-mediated knockdown is an effective approach to repress multiplex tumor instinct immune suppressive gene expressions.

TABLE 1
Example 1 gRNA sequences

| SEQ | | | SEQ | | |
|---|---|---|---|---|---|
| ID NO: | Name | gRNA Sequence | ID NO: | Name | gRNA Sequence |
| 1 | Scramble- | CGAGGGCGACTTAACCTTAGG | 47 | Galectin9 | GTGGGCAGGACGAAAGTTCTGAG |
| | g1 | AT | | g5 | |
| 2 | Scramble- | GTATCCCATAGTCCTTAAATT | 48 | Galectin9 | GAACCATATGGATGGTAGTTTGA |
| | g2 | GG | | g6 | |
| 3 | Pdl1g1 | AATGAGGTAAATGTAATGCTA | 49 | Galectin9 | GGGTACACCACAGGAGGGATTCC |
| | | G | | g7 | |
| 4 | Pdl1g2 | GTTGATTTTGCGGTATGGGGC | 50 | Galectin9 | CCATTTGGAATGGGGGTGTAGAA |
| | | A | | g8 | |
| 5 | Pdl1g3 | AATGAGATGAGATGTTGAGTG | 51 | Galectin9 | ATATCATGATGGACTTGGACGGG |
| | | C | | g9 | |
| 6 | Pdl1g4 | TTTGAGCTTGTATCTTCAACG | 52 | Galectin9 | GGCAAGACATTGCCTGATATCAT |
| | | C | | g10 | |
| 7 | Pdl1g5 | TTAGTTCATGCTCAGAAGTGG | 53 | Galectin9 | ccttcacatatgatccacaccga |
| | | C | | g11 | |
| 8 | Pdl1g6 | TTATGCAGCAGTAAACGCCTG | 54 | Galectin9 | agctaggaaacagaaaccacagc |
| | | C | | g12 | |
| 9 | Pdl1g7 | CTTTGGAGCCGTGATAGTAAA | 55 | Galectin9 | ataaaggacagcttcaagcagcc |
| | | C | | g13 | |
| 10 | Pdl1g8 | CTCGAATTGTGTATCATTTCG | 56 | Galectin9 | agaatttcttgttcaccatcacc |
| | | G | | g14 | |
| 11 | Pdl1g9 | GAATCACTTGCTCATCTTCCTT | 57 | Galectin9 | gaaagcaatgtcacctccacagc |
| | | | | g15 | |
| 12 | Pdl1g10 | AAGGTCCTCCTCTCCTGCCAC | 58 | Galectin9 | ggaaagataagacacaggcagag |
| | | A | | g16 | |
| 13 | Pdl1g11 | TATAATGCCAGCAAATATCCT | 59 | Galectin9 | ttgaactctgacctctgcaccag |
| | | CA | | g17 | |
| 14 | Pdl1g12 | CGTAGCAAGTGACAGCAGGCT | 60 | Galectin9 | cgatcagagatctgagcaaaccc |
| | | GT | | g18 | |
| 15 | Pdl1g13 | GCTGCCATACTCCACCACGTA | 61 | Galectin9 | tagagtgttgatatcctgcaagt |
| | | CA | | g19 | |
| 16 | Pdl1g14 | CCATCGTGACGTTGCTGCCAT | 62 | Galectin9 | acacagcctagaaaacccccttt |
| | | AC | | g20 | |
| 17 | Pdl1g15 | TAACGCAAGCAGGTCCAGCTC | 63 | Galectin9 | ttactgtggacattgtgggtcagt |
| | | CC | | g21 | |
| 18 | Pdl1g16 | TTTCCCAGTACACCACTAACG | 64 | Galectin9 | atcctcccaagcagacttcgctct |
| | | CA | | g22 | |
| 19 | Pdl1g17 | ACTTGCTCATCTTCCTTTTCCC | 65 | Galectin9 | aaaggtatggtataggctggggt |
| | | A | | g23 | |
| 20 | Pdl1g18 | CCCTGAAGTTGCTGTGCTGAG | 66 | Galectin9 | tgaaagttcaccacaaacctttg |
| | | GC | | g24 | |
| 21 | Pdl1g19 | CTGGTCCTTTGGCAGCGAGGC | 67 | Galectin9 | aactcttggtagtcccctggagg |
| | | TC | | g25 | |
| 22 | Pdl1g20 | TGATCTGAAGGGCAGCATTTC | 68 | Cd73-g1 | TACAATTACAAGATAGTCCAAGG |
| | | CC | | | |
| 23 | Pdl1g21 | CTGCGTCCTGCAGCTTGACGT | 69 | Cd73-g2 | AGATGTATTCAGAAACCACGCTG |
| | | CT | | | |
| 24 | Pdl1g22 | CTGATTATGCAGCAGTAAACG | 70 | Cd73-g3 | CTTTCGGTTAATATCGTACACCA |
| | | CC | | | |
| 25 | Pdl1g23 | CGCTTGTAGTCCGCACCACCG | 71 | Cd73-g4 | ATCTCAAAACCAGAGTGCCCCAG |
| | | TA | | | |
| 26 | Pdl1g24 | TCAGCGTGATTCGCTTGTAGT | 72 | Cd73-g5 | CTGAGAGACAACAAGAGCCCAAA |
| | | CC | | | |
| 27 | Pdl1g25 | TCTGGTTGATTTTGCGGTATG | 73 | Cd200-g1 | ACATCAGATTCAAGTAGACCAGG |
| | | GG | | | |
| 28 | Pdl1g26 | GCCTGACATATTAGTTCATGC | 74 | Cd200-g2 | AAGAGACACATGTAGCAGCCCTC |
| | | TC | | | |
| 29 | Pdl1g27 | CTCGGCCTGACATATTAGTTC | 75 | Cd200-g3 | TCATAGAAGCACATAGAGAACGG |
| | | AT | | | |
| 30 | Pdl1g28 | GGTTGGTGGTCACTGTTTGTC | 76 | Cd200-g4 | AAAGTCACAGAAAAAGAAGCTGG |
| | | CA | | | |
| 31 | Pdl1g29 | TGGTGACACTTCTCTTCCCACT | 77 | Cd200-g5 | CCATCAGCAGAACAGTAGCAGGT |
| | | C | | | |
| 32 | Pdl1g30 | TCTGTCCGGGAAGTGGTGACA | 78 | Cd66a-g1 | CAAACAGACAGTAGCAGAGGCCA |
| | | CT | | | |
| 33 | Pdl1g31 | GACTGCTGGTCACATTGAGAA | 79 | Cd66a-g2 | GAAGCAAAATATACACAGGCTGT |
| | | GC | | | |
| 34 | Pdl1g32 | TAGAAAACATCATTCGCTGTG | 80 | Cd66a-g3 | GTACATGAAATCGCACAGTCGCC |
| | | GC | | | |
| 35 | Pdl1g33 | TGTGATCTCCAAAACGTACAG | 81 | Cd66a-g4 | TTAAAAGAAACACAAGAAGGCAG |
| | | TA | | | |
| 36 | Pdl1g34 | CTCCGCTGTGTGGTTTTGCCCT | 82 | Cd66a-g5 | GACAGAAATCTAGTGGCCCTAAG |
| | | G | | | |
| 37 | Pdl1g35 | TGTTCTGTGGAGGATGTGTTG | 83 | Galectin3- | CCAGTATCATAAAAACCCCCAAA |
| | | CA | | g1 | |
| 38 | Pdl1g36 | TGGATCCCAGAAGCACCCAGT | 84 | Galectin3- | TTAAGCGAAAAGCTGTCTGCCAT |
| | | GA | | g2 | |
| 39 | Pdl1g37 | AAGAGGAGGACCGTGGACAC | 85 | Galectin3- | CAGTCCTAAGATTTGACATCCAG |
| | | TAC | | g3 | |
| 40 | Pdl1g38 | TCTCAAGAAGAGGAGGACCG | 86 | Galectin3- | CACACAGCTTACTTACAACCACC |
| | | TGG | | g4 | |
| 41 | Pdl1g39 | ATTTCTCCACATCTAGCATTCT | 87 | Galectin3- | AGGGCATATAAAGACAGGCAGCT |
| | | C | | g5 | |
| 42 | Pdl1g40 | GGTTTTTTGAGCTTGTATCTTC | 88 | Cd47-g1 | CAAGCAAGACAGAAGCGCCAAGT |
| | | A | | | |
| 43 | Galectin9 | TTAATGTATGGAGACTGGGCA | 89 | Cd47-g2 | TAGAGATTACAATGAGGCCAAGT |
| | g1 | C | | | |
| 44 | Galectin9 | GAGTTTTCTGTTTGCGCCCCTT | 90 | Cd47-g3 | ACCAAAGCAAGGACGTAGCCCAG |
| | g2 | | | | |
| 45 | Galectin9 | ATATAGCTAGCTTGGGGGCAA | 91 | Cd47-g4 | CCACGATGACTGTGAGCACCAGC |
| | g3 | T | | | |
| 46 | Galectin9 | GGGTTAATGTATGGAGACTGG | 92 | Cd47-g5 | TAAACAGTAGTTGAGCTGAACCT |
| | g4 | GC | | | |

TABLE 2
shRNA Sequences

| | | SEQ | Full Sequence |
|---|---|---|---|
| | Target Sequence | ID | (Underline: Target sequence, Bold: Loop, |
| Name: | (SEQ ID NO:) | NO: | Italic: Target sequence, reverse complement) |
| msCd200sh1F | CAGAGTCTGGACAAAGGATTT | 1666 | ACCGGCAGAGTCTGGACAAAGGATTTCTCGA |
| | (1658) | | GAAATCCTTTGTCCAGACTCTGTTTTTG |
| msCd200sh1R | | 1667 | GATCCAAAAACAGAGTCTGGACAAAGGATTT |
| | | | CTCGAGAAATCCTTTGTCCAGACTCTGCC |
| msCd200sh2F | GCCCATAGTACACCTTCACTA | 1668 | ACCGGGCCCATAGTACACCTTCACTACTCGA |
| | (1659) | | GTAGTGAAGGTGTACTATGGGCTTTTTG |
| msCd200sh2R | | 1669 | GATCCAAAAAGCCCATAGTACACCTTCACTA |
| | | | CTCGAGTAGTGAAGGTGTACTATGGGCCC |
| msCd66ash1F | GCGACTGTGCAATTTCATGTA | 1670 | ACCGGGCGACTGTGCAATTTCATGTACTCGA |
| | (1660) | | GTACATGAAATTGCACAGTCGCTTTTTG |
| msCd66ash1R | | 1671 | GATCCAAAAAGCGACTGTGCAATTTCATGTA |
| | | | CTCGAGTACATGAAATTGCACAGTCGCCC |
| msCd66ash2F | CCTGTGTCTACAAACGCTGAA | 1672 | ACCGGCCTGTGTCTACAAACGCTGAACTCGA |
| | (1661) | | GTTCAGCGTTTGTAGACACAGGTTTTTG |
| msCd66ash2R | | 1673 | GATCCAAAAACCTGTGTCTACAAACGCTGAA |
| | | | CTCGAGTTCAGCGTTTGTAGACACAGGCC |
| msCd66ash3F | CTTTGCTTGGTACAAGGGAAA | 1674 | ACCGGCTTTGCTTGGTACAAGGGAAACTCGA |
| | (1662) | | GTTTCCCTTGTACCAAGCAAAGTTTTTG |
| msCd66ash3R | | 1675 | GATCCAAAAACTTTGCTTGGTACAAGGGAAA |
| | | | CTCGAGTTTCCCTTGTACCAAGCAAAGCC |
| msGalectin3sh | CCGCATGCTGATCACAATCAT | 1676 | ACCGGCCGCATGCTGATCACAATCATCTCGA |
| 1F | (1663) | | GATGATTGTGATCAGCATGCGGTTTTTG |
| msGalectin3sh | | 1677 | GATCCAAAAACCGCATGCTGATCACAATCAT |
| 1R | | | CTCGAGATGATTGTGATCAGCATGCGGCC |
| mCD47sh1F | GAAGTTGAACAAATCGTATAT | 1678 | ACCGGGAAGTTGAACAAATCGTATATCTCGA |
| | (1664) | | GATATACGATTTGTTCAACTTCTTTTTG |
| mCD47sh1R | | 1679 | GATCCAAAAAGAAGTTGAACAAATCGTATAT |
| | | | CTCGAGATATACGATTTGTTCAACTTCCC |
| mCD47sh2F | ATCTCAGTCTCAGACTTAATC | 1680 | ACCGGATCTCAGTCTCAGACTTAATCCTCGA |
| | (1665) | | GGATTAAGTCTGAGACTGAGATTTTTTG |
| mCD47sh2R | | 1681 | GATCCAAAAAATCTCAGTCTCAGACTTAATCC |
| | | | TCGAGGATTAAGTCTGAGACTGAGATCC |

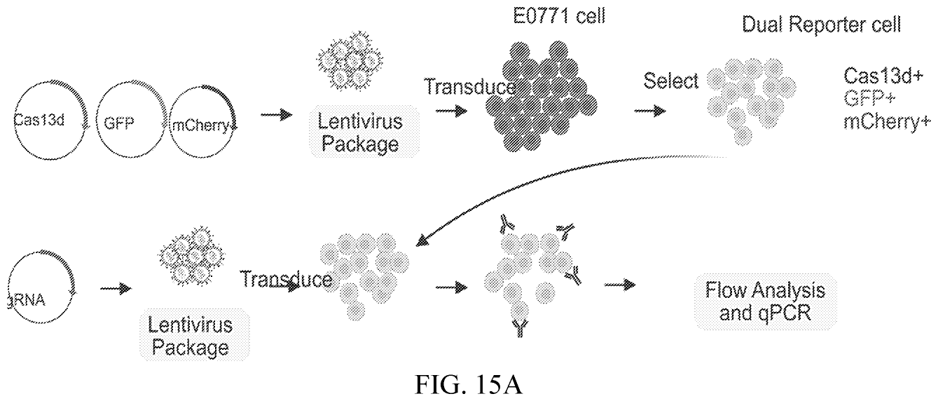

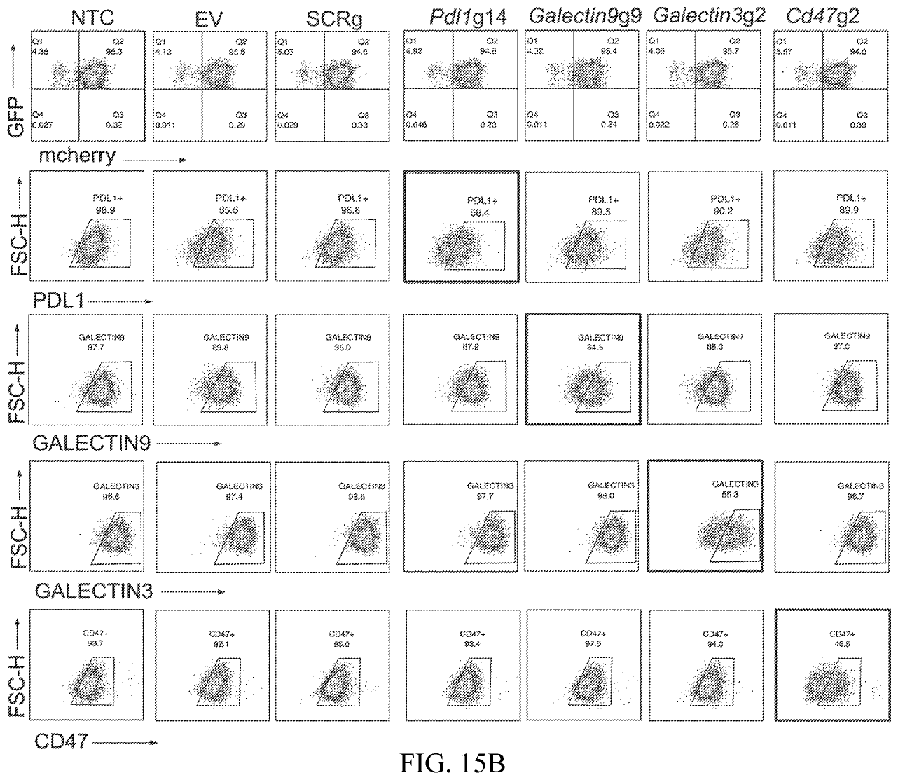

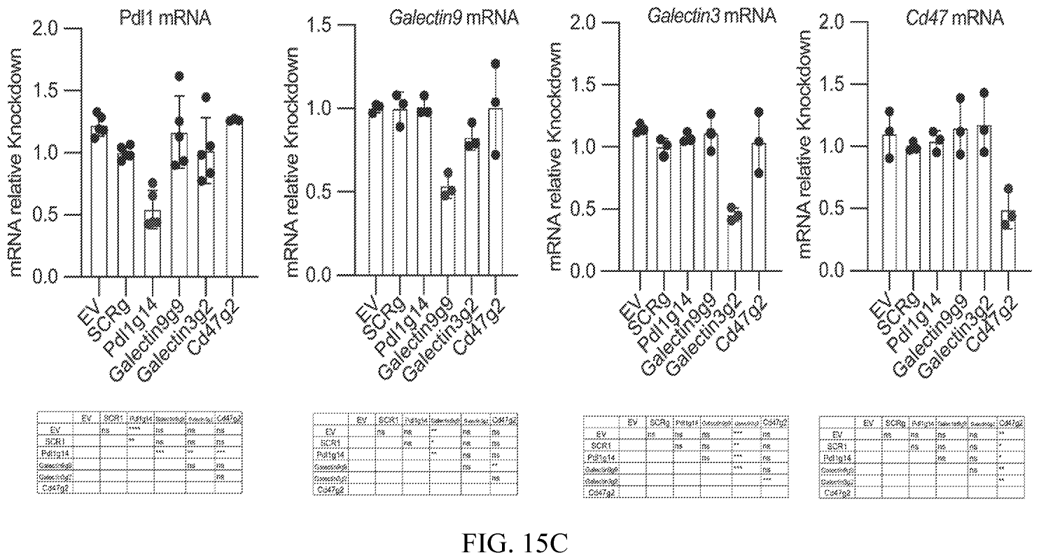

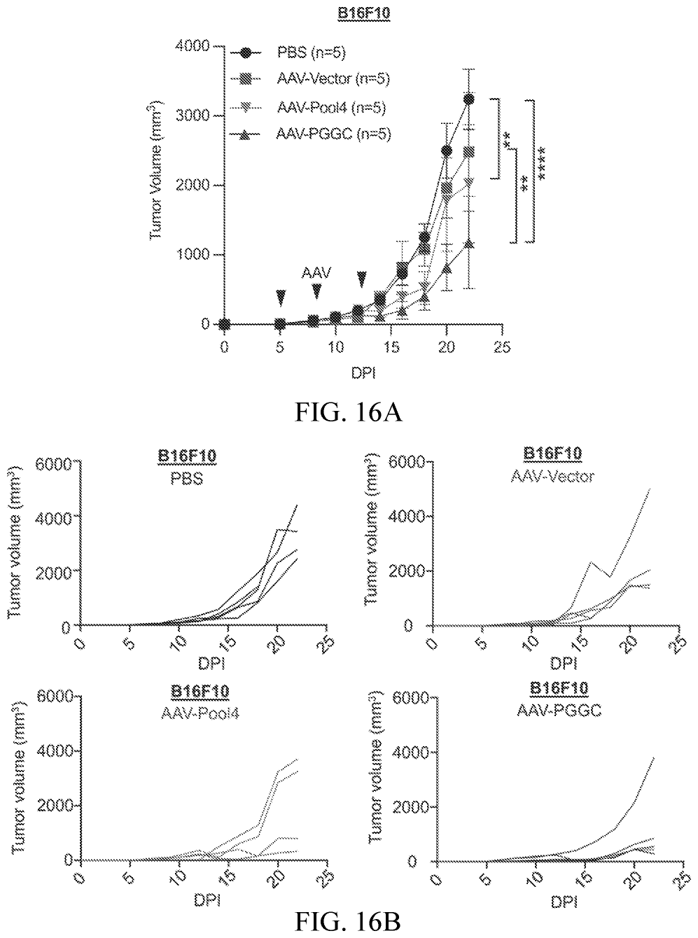

Recently, Cas13d was reported to have collateral activity in human cells. It was reported that when targeting the transfected DsRed in HEK cells, the co-transfected reporter gene GFP would be markedly down-regulated. However, when targeting the endogenous RNAs, the extent of collateral activity could be influenced by the abundance of the target RNA. To test how strong the collateral activity when targeting the endogenous immunosuppressive genes, a GFP and mCherry dual reporter system was generated that would indicate the collateral activity of Cas13d (FIG. 15A). Instead of transient transfection of the reporter gene plasmids, an E0771 cell line was established which stably expressed Cas13d, GFP and mCherry protein by lentivirus transduction in order to better mimic the endogenous gene expression. According to the flow cytometry results, both the GFP and mCherry reporters showed stable expression among all the tested guide RNAs targeting the immunosuppressive genes, including non-transduced control (NTC) and empty vector (EV) (FIG. 15B). Subsequent studies then tested the specific gene targeting of these guide RNAs at protein by flow cytometry (FIG. 15B). Even though scramble control caused a very mild background knockdown of PDL1 or GALECTIN9, the on-target knockdown is still much stronger (FIG. 15B). Then RT-qPCR was performed to test the gene knockdown at RNA level. The data and statistical test among groups showed specific targeting of all the guide RNAs (FIG. 15C). These data suggested specific on-target of the Cas13d guide RNAs when targeting the endogenous immunosuppressive genes.

### Example 2: AAV-Mediated Immunosuppressive Gene Repression as an Immunotherapeutic Modality

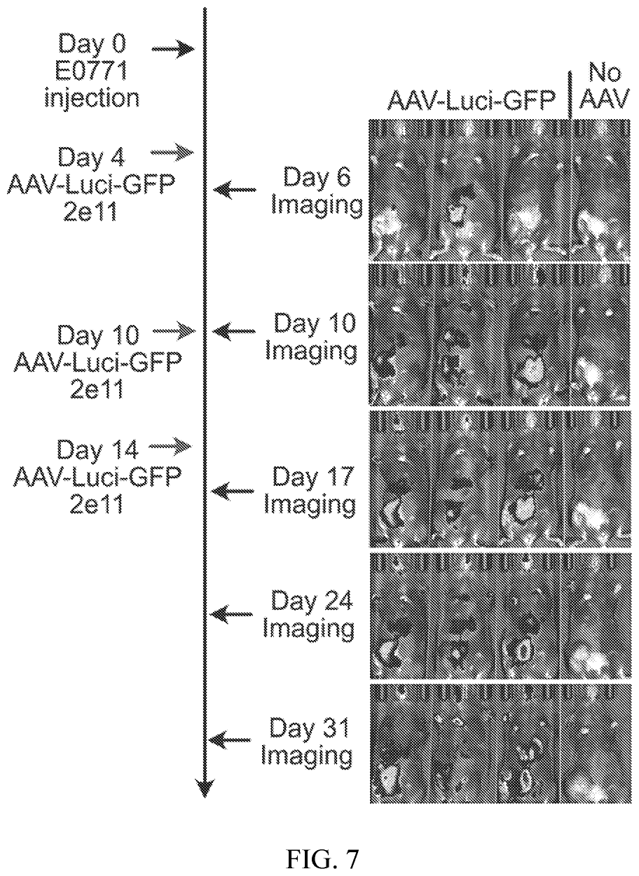

Given that gene knockdown is not complete by Cas13d, the natural question is whether such degree of knockdown can lead to effective immune modulation, and thereby anti-tumor immunity, in vivo. Adeno-associated virus (AAVs) is one of the leading vehicles for transgene delivery. To evaluate the feasibility of in vivo Cas13d and gRNA intratumoral delivery, studies first generated an AAV vector expressing firefly luciferase and GFP (AAV-Luci-GFP). AAV-Luci-GFP was intratumorally injected into E0771 tumor-bearing mice and analyzed for luciferase activity by in vivo bioluminescent imaging (FIG. 7). The time course imaging showed that luciferase was persistently expressed in the tumor and, unsurprisingly, also in the liver (FIG. 7). These data indicate that intratumoral AAV injection can successfully deliver genetic cargo into the TME.

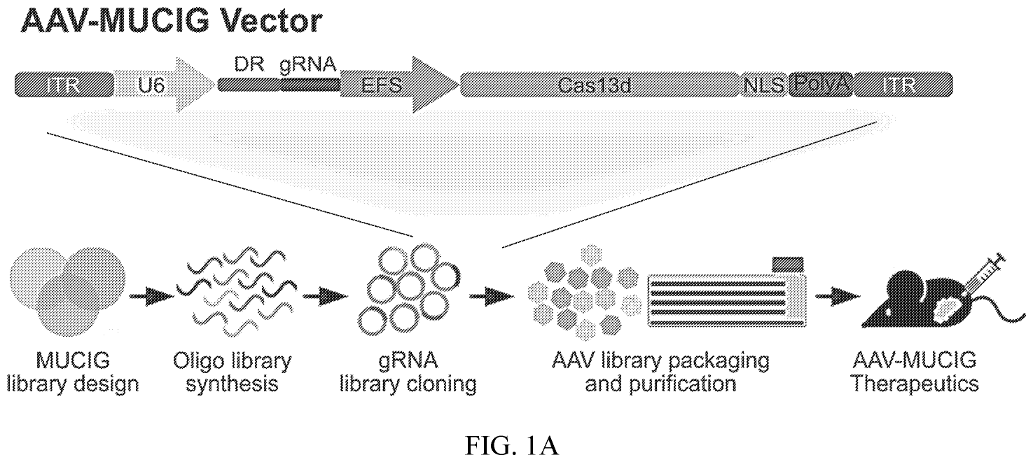

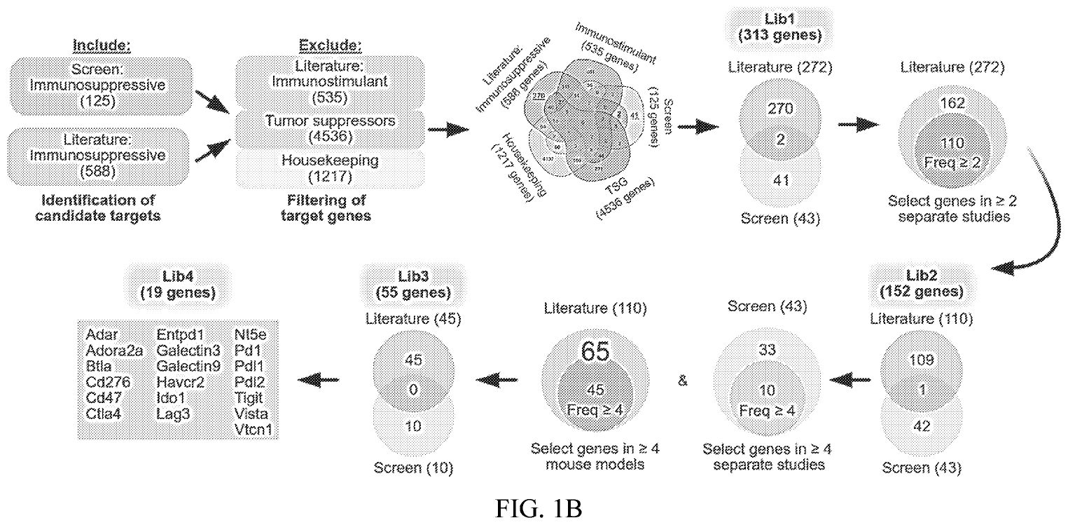

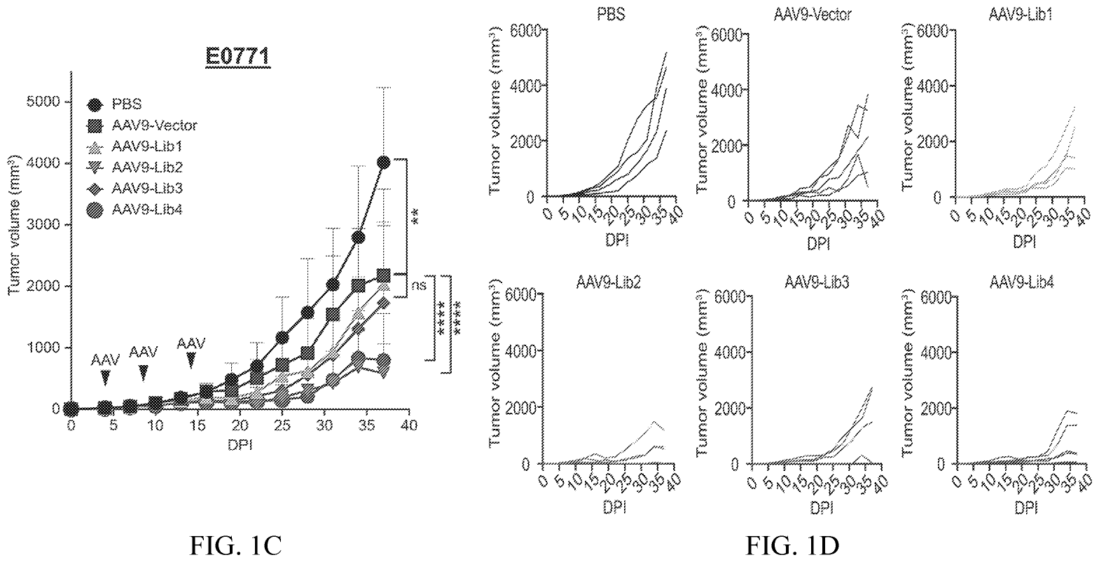

Having evaluated the feasibility of the Cas13d gRNA knockdown system, studies next sought to investigate whether silencing multiple immunosuppressive genes in the TME via AAV delivery of Cas13d and gRNAs could function as a combinatorial immunotherapy. This approach was termed MUCIG (Multiplex Universal Combinatorial Immunotherapy via Gene silencing). First, libraries of different scales were designed which target combinations of immunosuppressive genes (FIG. 1A, B). The first library was designed on the basis of several criteria. By leveraging the knowledge from the literature and the immunogenomic databases such as TISIDB, 588 tumor immunosuppressive genes and 535 tumor immunostimulative genes were identified (FIG. 1B). In order to avoid undesired side effects, sets of genes were identified to be excluded, including tumor suppressor genes (TSGs) and house-keeping genes. The top hits identified from functional screens were further considered for genetic factors that enable cancer cells to escape the immune system, selecting genes that have been experimentally validated to be cancer immunotherapy targets. Next, a core set of genes were identified which were recently demonstrated to be cancer-intrinsic T cell killing evasion genes across at least 3 cancer models. Thus, a total of 125 genes were identified from screen data. With a tiered approach, four initial Cas13d gRNA libraries were designed for MUCIG experiments (MUCIG-Lib1: 313 genes (Table 3), Lib2: 152 genes (Table 4); Lib3: 55 genes (Table 5); Lib4, 19 genes (Table 6)) (FIG. 1B).

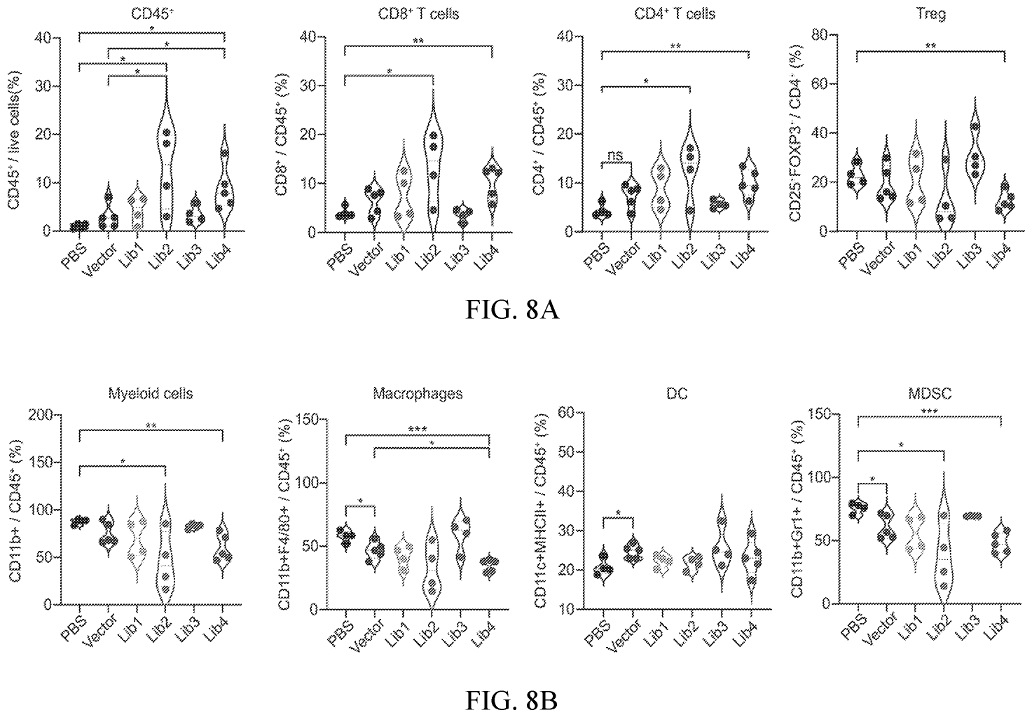

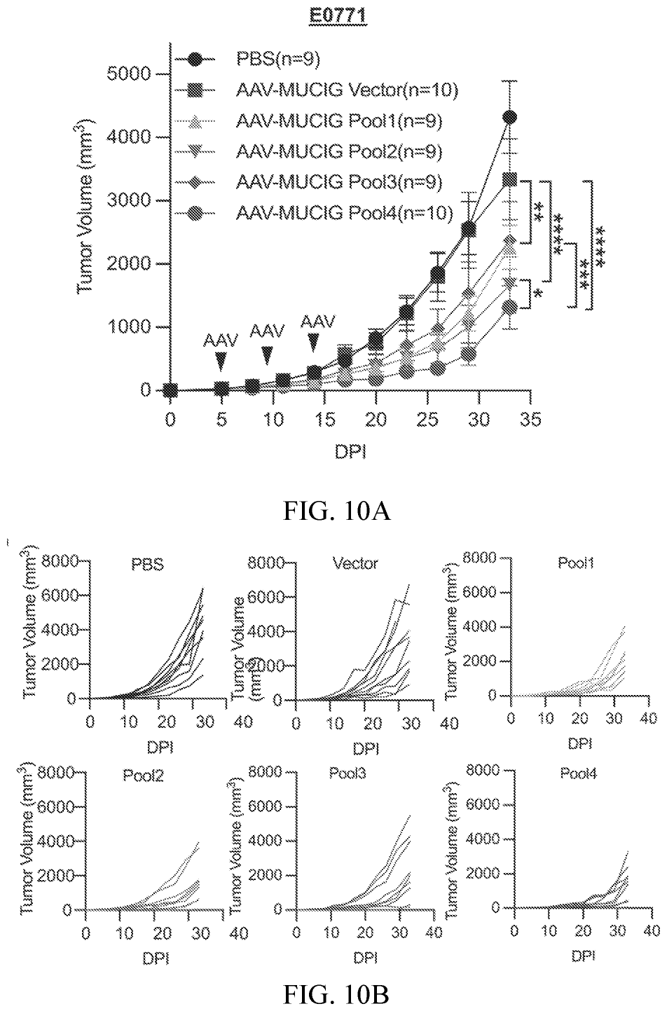

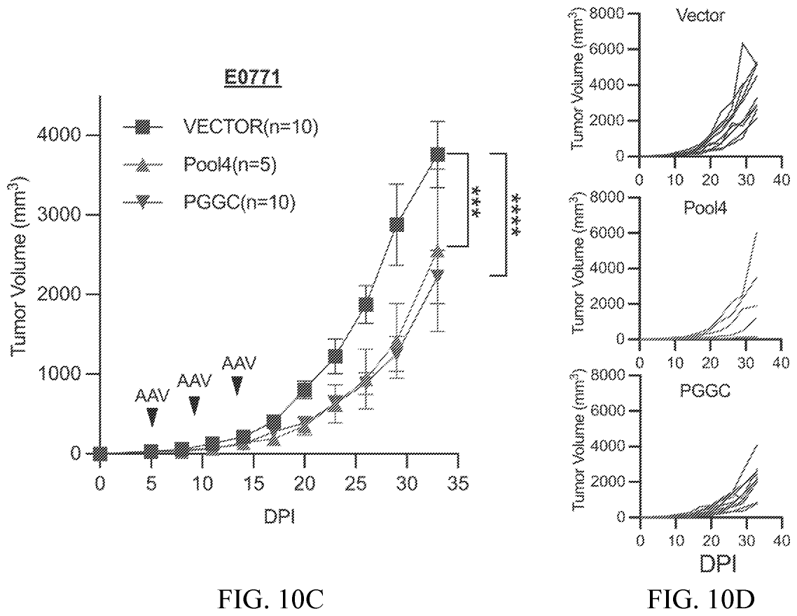

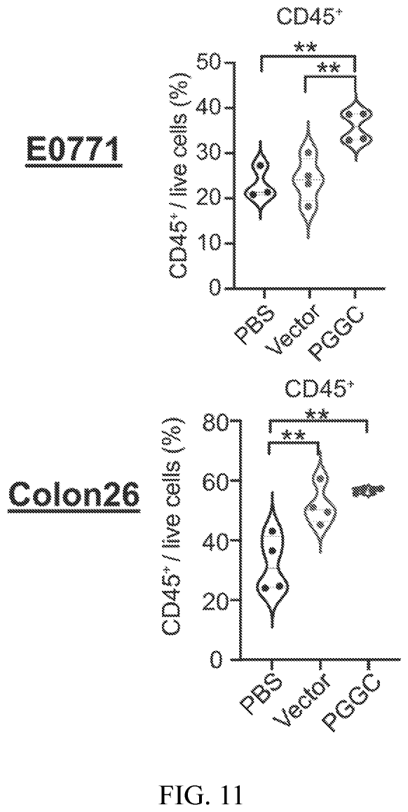

To facilitate direct delivery of these libraries into tumors, an all-in-one AAV vector was designed (AAV-U6-gRNAs-EFS-Cas13d) (FIG. 1A). Five gRNAs were designed for each target gene and produced the four AAV-MUCIG libraries accordingly. To evaluate the efficacy of these libraries, the E0771 syngeneic orthotopic tumor model of triple negative breast cancer was utilized, which is known to be moderately responsive to immunotherapy. C57BL/6Nr (B6) mice bearing E0771 tumors were treated with AAV-MUCIG libraries by intratumoral administration. All AAV-MUCIG-pools treatment led to significantly reduced tumor burden compared to the AAV-vector or PBS treatment (FIGS. 1C, 1D, 10A, 10B). Among the 4 gene pools, MUCIG-pool4 showed significantly better, and pool2 showed moderately better, therapeutic effect than pool 1 and pool3, with AAV-MUCIG-pool4 showing the strongest efficacy among the four (FIGS. 1C, 1D, 10A, 10B). These data indicated that all four compositions of AAV-MUCIG treatment had therapeutic effect in this tumor model. A trend of increase in the number of tumor-infiltrating CD8⁺ and CD4⁺ T cells was observed after MUCIG-lib2 or lib4 treatment (FIG. 8A). A trend of decreased Treg cells after AAV-MUCIG-Lib4 treatment was also observed (FIG. 8A). Myeloid cell populations including myeloid-derived suppressive cells (MDSCs) and macrophages were also reduced (FIG. 8B).

TABLE 3
MUCIG-Lib1 gRNAs

| SEQ | | | SEQ | | |
|---|---|---|---|---|---|
| ID | | | ID | | |
| NO: | Name: | Sequence: | NO: | Name: | Sequence: |
| 93 | Abl1-g1 | AATACAAAATATAGTGGCACCCA | 876 | Krt17-g4 | AGCAAGCTTTAATGGTCTCAAGC |
| 94 | Abl1-g2 | GCCACAAAATCATAGAGTGCCAC | 877 | Krt17-g5 | CCAGCCTTGTCTTCACATCCAGC |
| 95 | Abl1-g3 | CAATGAAAACACAGTGAGCACA | 878 | Krt7-g1 | TATTCTTGAAATCTTCCACCACA |
| | | G | | | |
| 96 | Abl1-g4 | AGAAGAAATGCATGACCAGCAA | 879 | Krt7-g2 | ACAAACTCATTCTCAGCAGCCGT |
| | | C | | | |
| 97 | Abl1-g5 | TCACACATGTAAACAGGAGCCTG | 880 | Krt7-g3 | TCTAACAAGAGCTGGGAAGCACA |
| 98 | Acat1-g1 | AGGTATATAAGCAAGCCCAACC | 881 | Krt7-g4 | CTGTTCCTGCAGCAGCGCCCACT |
| | | A | | | |
| 99 | Acat1-g2 | CGTTGCAAATACTAGCCAGACCG | 882 | Krt7-g5 | ATCTCATCCTGCAGACTGTCCGC |
| 100 | Acat1-g3 | ACACATAAGACTTTGAGAGGCCA | 883 | Kynu-g1 | TCCAGTATAAAAGTGCAGCCCAC |
| 101 | Acat1-g4 | AAAACAAAACATAAACAGCCAG | 884 | Kynu-g2 | CAGATATTAAAGCATGCACCAGG |
| | | T | | | |
| 102 | Acat1-g5 | GATTAAAGCCATGTACCACCCAA | 885 | Kynu-g3 | AATACTTATAGGAACACCAGCAG |
| 103 | Adam10- | ATAAAAGTTTATCGAGAGCCAAG | 886 | Kynu-g4 | AAAGATAGATCAATTGAAGGCAG |
| | g1 | | | | |
| 104 | Adam10- | TCAATGTAAAACGTGCCACCACG | 887 | Kynu-g5 | AAATGCATACTAGTAGTGGCATC |
| | g2 | | | | |
| 105 | Adam 10- | GACAAGTATTTCTTTCAGCCAGA | 888 | L1cam- | CGTTACTCAAGATCAGAGACCCC |
| | g3 | | | g1 | |
| 106 | Adam10- | AATACACAAAGTAATAAGCAGG | 889 | L1cam- | CCACAGTAACATAGTAGGCATGC |
| | g4 | C | | g2 | |
| 107 | Adam10- | CAGAATTAACACTGTCGGCAACA | 890 | L1cam- | ACCTGTAGTAGAAACTCGCACAG |
| | g5 | | | g3 | |
| 108 | Adam17- | CAGAACATCTTGAAGCACCAGA | 891 | L1cam- | CAGACCAAGCAAAAGCATACAGG |
| | g1 | G | | g4 | |
| 109 | Adam17- | CATTCATACATATACCCACACAC | 892 | L1cam- | TAAGAAAGAGACTCGAAGCCACA |
| | g2 | | | g5 | |
| 110 | Adam17- | AGTTACAGAGTTGAGAGCCACCA | 893 | Lag3-g1 | AGAAGCAAAAAGCCAAGGAGCAG |
| | g3 | | | | |
| 111 | Adam17- | GAAAACCAGAACAGACCCAACG | 894 | Lag3-g2 | CAAAAGGACCCAATCAGACAGCT |
| | g4 | A | | | |
| 112 | Adam17- | TATCTTCAGACTTATACACCAGC | 895 | Lag3-g3 | CTTCGTAGAAAGTTAGGATCCAG |
| | g5 | | | | |
| 113 | Adar-g1 | TTCACCATAAGAGAGCTGCAGTA | 896 | Lag3-g4 | AGTCACTGTGATGACCGCCAACG |
| 114 | Adar-g2 | TCAAGGAATGCAAGACAGCCAC | 897 | Lag3-g5 | CAGACAGACAGACAGACACACAC |
| | | G | | | |
| 115 | Adar-g3 | CTTTTCATAATAATGGCAGCCAG | 898 | Layn-g1 | AGGTACAGAGCAAAGAGCATCCC |
| 116 | Adar-g4 | AGACCAGAAGAATCCCAGTGCA | 899 | Layn-g2 | TAAATGACTTTATAGCAAGGCCT |
| | | C | | | |
| 117 | Adar-g5 | CTGAGCATACTCTAACAACCCGC | 900 | Layn-g3 | CAGAAGTCACCATCAGATGCCAG |
| 118 | Adipoq- | ATGCGAATATTGTGAAGCCCCCA | 901 | Layn-g4 | AAGACTGTTAGATGCTGTCAGCA |
| | g1 | | | | |
| 119 | Adipoq- | TTCACATCTTTCATGTACACCGT | 902 | Layn-g5 | CATACAGCTGAAGTGACCACAAG |
| | g2 | | | | |
| 120 | Adipoq- | AGCGATACACATAAGCGGCTTCT | 903 | Lef1-g1 | AGAGTAAACAATAAAGAGCCACC |
| | g3 | | | | |
| 121 | Adipoq- | AATATTTGTGTACAGTGAGCAGA | 904 | Lef1-g2 | AACATGAAAGCATTCAGAGGCTT |
| | g4 | | | | |
| 122 | Adipoq- | CTTTAGACATTCATACACTCAGC | 905 | Lef1-g3 | AGAAAAAGAGAAGTTTGCCAAGA |
| | g5 | | | | |
| 123 | Adora2a- | ACAAACAAACAAACAAGCCCCA | 906 | Lef1-g4 | AAATCAGAAACTAAGTGAGACGG |
| | g1 | C | | | |
| 124 | Adora2a- | TTAATGAGATTGGTCCAGCCAAC | 907 | Lef1-g5 | ACCAAAGATGACTTGATGTCGGC |
| | g2 | | | | |
| 125 | Adora2a- | AAAATCCTTAGGTAGATGGCCAG | 908 | Lgals1- | GAAAGCACAAGAGAGGTCACTGA |
| | g3 | | | g1 | |
| 126 | Adora2a- | CAGCAAATCGCAATGATGCCCTT | 909 | Lgals1- | AGACCAAGAACACATGGAGGCAT |
| | g4 | | | g2 | |
| 127 | Adora2a- | ATGATGTACACCGAGGAGCCCAT | 910 | Lgals1- | TTTATTAAGACAAATGCGGTCCG |
| | g5 | | | g3 | |
| 128 | Ager-g1 | CCATAGAGCAAGAACCAGCACC | 911 | Lgals1- | CAGTCAGAAGACTCCACCCGAGA |
| | | C | | g4 | |
| 129 | Ager-g2 | CGTTTTCGCCACAGGATAGCCCC | 912 | Lgals1- | CGAACTTTGAGACATTCCCCAGG |
| | | | | g5 | |
| 130 | Ager-g3 | TGTCAAATGTTTACTCAGCATGG | 913 | Lgals3- | AAAGGCATTCTAACTAGGGCAGC |
| | | | | g1 | |
| 131 | Ager-g4 | GATCACTGTCAGCTCTGACCGCA | 914 | Lgals3- | TTAAGCGAAAAGCTGTCTGCCAT |
| | | | | g2 | |
| 132 | Ager-g5 | CCACGCAGCTATAGGTGCCCTCA | 915 | Lgals3- | ACACAATAATAAATACATCTGCT |
| | | | | g3 | |
| 133 | Ago2-g1 | GTAAAAGTTAAGATGCCACAAC | 916 | Lgals3- | ACAGCTTGTCCTCTGACCTCCAC |
| | | A | | g4 | |
| 134 | Ago2-g2 | AGACTTAGTTAATAGCACCCAAC | 917 | Lgals3- | TCCACTTCCAGGCAGTGACGCGT |
| | | | | g5 | |
| 135 | Ago2-g3 | ATTGTCATTAGTAAGACACCCAC | 918 | Lgals9- | CCTTCACATATGATCCACACCGA |
| | | | | g1 | |
| 136 | Ago2-g4 | AAAAATAAAGCATTAGCAAGCC | 919 | Lgals9- | ATATCATGATGGACTTGGACGGG |
| | | T | | g2 | |
| 137 | Ago2-g5 | CAGCAACTATGTTACAGACCTCC | 920 | Lgals9- | GGGTACACCACAGGAGGGATTCC |
| | | | | g3 | |
| 138 | Aire-g1 | AGGTTCACATATGTGACAGCAGC | 921 | Lgals9- | AGAATTTCTTGTTCACCATCACC |
| | | | | g4 | |
| 139 | Aire-g2 | GTCACAACAGATGAGCTCACCTC | 922 | Lgals9- | GGAAAGATAAGACACAGGCAGAG |
| | | | | g5 | |
| 140 | Aire-g3 | AGTCCTTAAAGAGAATCCTCCAG | 923 | Lif-g1 | GACATAGTAATAAATAGACAGCT |
| 141 | Aire-g4 | ATCTCTACAAAGATCAGGGCCAT | 924 | Lif-g2 | TTTAAATAATAAATAAAGGCCCC |
| 142 | Aire-g5 | CGTAGCATTCCATCCCCACCTGC | 925 | Lif-g3 | GACACCCTAAAAGTGAGTCACAG |
| 143 | Alcam- | ATAGCAATCAGAATCAGAACCGT | 926 | Lif-g4 | GCATTTAACAATGTCCCAAACCC |
| | g1 | | | | |
| 144 | Alcam- | TATACATCCAATTAACAGCCACT | 927 | Lif-g5 | GTAATAGGAAATGAAGAGAGCAT |
| | g2 | | | | |
| 145 | Alcam- | CCTTAAAAAGTACCTCAGGCAGA | 928 | Lilrb4a- | AAGACCAAGGTATAGCAGCACTG |
| | g3 | | | g1 | |
| 146 | Alcam- | AGATTATAGTTTTAGACAGTCCA | 929 | Lilrb4a- | CCAAATACTATGGATGAGCTGCA |
| | g4 | | | g2 | |
| 147 | Alcam- | CAATTTCAAAAGCTTGAACCACC | 930 | Lilrb4a- | CAAAAGTAGCATAGGATGGCTGA |
| | g5 | | | g3 | |
| 148 | Aoc3-g1 | AGGCCAAAAGAAAAAGCCCACA | 931 | Lilrb4a- | ATGAGAAAAAGCAGGAGGAAGAA |
| | | A | | g4 | |
| 149 | Aoc3-g2 | AGATTGTAATACAAGCCAAACCA | 932 | Lilrb4a- | ATTAATTAAATAGAGTCCACTGG |
| | | | | g5 | |
| 150 | Aoc3-g3 | TCGTCATAATCATAGCACCTCCA | 933 | Lipa-g1 | AGTTACTAGAATCTGCCAGCAAG |
| 151 | Aoc3-g4 | AGACAAGCAGCAAAGAGAAACC | 934 | Lipa-g2 | AAAATGTAGTTAATTGAAGCAGG |
| | | C | | | |
| 152 | Aoc3-g5 | CCCAAACTTCTCACCAGCACCGA | 935 | Lipa-g3 | CAATGAAGCATAAGACAGCTAGC |
| 153 | Areg-g1 | CAGAGACAAAGATAGTGACAGC | 936 | Lipa-g4 | TCATCAAAACTGAAGGCCCAGAA |
| | | T | | | |
| 154 | Areg-g2 | AATCATCTATAATATAGCCGGAT | 937 | Lipa-g5 | CAAAGCGAAATTCCTGAGCCGAG |
| 155 | Areg-g3 | ACAGAAAGCTCAAGTCCACCGG | 938 | Lipt2-g1 | TTGAACAGTTCAGAGCCAAGCCG |
| | | C | | | |
| 156 | Areg-g4 | ACAATTGCATGTCACCACCTCCA | 939 | Lipt2-g2 | CAAACTTGAGACATTCAACCCAG |
| 157 | Areg-g5 | CTATCATAAAAAGTGACAACTGG | 940 | Lipt2-g3 | ATCGACAACTAGAAGCAACCAAG |
| 158 | Arg2-g1 | AGGCAATATTGATCCAGACAGCC | 941 | Lipt2-g4 | GCAATCACAATGGAGTGAGCCAT |
| 159 | Arg2-g2 | CAACAAGATCCAGAGCTGACAG | 942 | Lipt2-g5 | CAGTGACAAGTCTTTGAAGCGCC |
| | | C | | | |
| 160 | Arg2-g3 | AATAAAATGTTCAGGAGGCTCCA | 943 | Lpar5-g1 | ATGAGCATCAGAAAGAGACAGCT |
| 161 | Arg2-g4 | TACAGTAATAGTGTTGGTGACCA | 944 | Lpar5-g2 | AGACGAATAGACGACAGCCGCCA |
| 162 | Arg2-g5 | ACCAGATTATTGTAGGGATCATC | 945 | Lpar5-g3 | CAGCACCATTATCATCAGCACCC |
| 163 | Astl-g1 | AGAACATCTTTAGAGCCAGGCAT | 946 | Lpar5-g4 | TTGCACATGTACACGCTCACCAC |
| 164 | Astl-g2 | GGAACTAACATATTGGTACTCCG | 947 | Lpar5-g5 | GGAACAACAAGGTCAGAGCATGA |
| 165 | Astl-g3 | GAAGAACTTCCAGAGGCAACCA | 948 | Lrrc32- | CGAAGCGCTGTATAGAAGCCCAG |
| | | A | | g1 | |
| 166 | Astl-g4 | CCAGAAGCCAAGTACGTGCATG | 949 | Lrrc32- | ACAAGGTACTTAGCCTCCTCAGA |
| | | A | | g2 | |
| 167 | Astl-g5 | TGATCTAGACAGAATACACCCTC | 950 | Lrrc32- | CTTGGATGTCCAGTGAGAGCACC |
| | | | | g3 | |
| 168 | Atg10- | CCTGCAGTAATTCAACAGAGCAG | 951 | Lrrc32- | GTTCACCGTCCTACAGGGCACTT |
| | g1 | | | g4 | |
| 169 | Atg10- | CCCTAAAGTAAAGAACCGGCACT | 952 | Lrrc32- | CAGCATGGCCAGGAGTAGCAGGA |
| | g2 | | | g5 | |
| 170 | Atg10- | CAGAGGTAAATTCAGACCAACC | 953 | Ly6k-g1 | CAAAAATTCGTGTGACGGCCAAC |
| | g3 | A | | | |
| 171 | Atg10- | CATCGTTCACTAAAGCGAGCACA | 954 | Ly6k-g2 | AAATTTAATTAGAAGGATCCAGC |
| | g4 | | | | |
| 172 | Atg10- | TACATTAATTTTCAGAAACAGGC | 955 | Ly6k-g3 | AGAACTACGAGCAAGGCCACTAG |
| | g5 | | | | |
| 173 | Atg14- | TAAGACCATGTAAAGCAGCCCAT | 956 | Ly6k-g4 | CACTTATAAAATAGGAAGGGCAT |
| | g1 | | | | |
| 174 | Atg14- | ATATGAAATAAAACAAGGCCAC | 957 | Ly6k-g5 | TTTAATTAGAAGGATCCAGCAGG |
| | g2 | C | | | |
| 175 | Atg14- | ACCAAGGAAGAAACCGGACAGC | 958 | Ly75-g1 | AAAACCCATTATTTGGCAGCCAG |
| | g3 | A | | | |
| 176 | Atg14- | TAATAACTGCCAAAGCGCCACAG | 959 | Ly75-g2 | TTCCAGTAGACATAGCCACGCAC |
| | g4 | | | | |
| 177 | Atg14- | AGACACAATGTTGACGAGCTGCG | 960 | Ly75-g3 | CCATCATGACTTGAGAGCCCAAC |
| | g5 | | | | |
| 178 | Atg9a- | ACTCATTGAGAAACAGAGAGCC | 961 | Ly75-g4 | TGAGATTAAGAATCAAGGCGCAG |
| | g1 | G | | | |
| 179 | Atg9a- | TAGGACTACATAGAAGCAGCCA | 962 | Ly75-g5 | AAAACAAAACCAAAAACCCCAGC |
| | g2 | G | | | |
| 180 | Atg9a- | ATAGATAAACTTGATAAGCCGGT | 963 | Lyn-g1 | TCAGATTTGATAGTGAAGCAGCC |
| | g3 | | | | |
| 181 | Atg9a- | CAAAACATTCTAGCTGCGCGCCC | 964 | Lyn-g2 | CCATTTAAGAAACAGCATCCAGG |
| | g4 | | | | |
| 182 | Atg9a- | GTCCACCTTGTTAACCAGCTCCA | 965 | Lyn-g3 | TGTATAGAAGTCATCCAGGACAC |
| | g5 | | | | |
| 183 | Atp13a1- | TTTATAAAATTGCAGACGCCGAT | 966 | Lyn-g4 | GTTGTTATAGTAACCCATCCAGA |
| | g1 | | | | |
| 184 | Atp13a1- | CTGGAATTCAAATGACAGCACCT | 967 | Lyn-g5 | AGTGCTTAATGACATCACCATGC |
| | g2 | | | | |
| 185 | Atp13a1- | GACAGAGAATTGCAGCATCACC | 968 | Maf-g1 | ATTAACATATTTAATCCAAGCGC |
| | g3 | G | | | |
| 186 | Atp13a1- | CACTGAGAAAACATAGAGTGCA | 969 | Maf-g2 | TCCCCTAAATCATTTGAGCAGAG |
| | g4 | G | | | |
| 187 | Atp13a1- | CCCAAAGATGACATGCAGCCGA | 970 | Maf-g3 | CTAAAAGCTAGAGTGTGTGACCC |
| | g5 | G | | | |
| 188 | Atp2a3- | CCAAACTAAGCAATAGGTCAGA | 971 | Maf-g4 | ATGTTAATTCTAACTCCGACCAA |
| | g1 | G | | | |
| 189 | Atp2a3- | AGTGACAAAGTATCACACCAGG | 972 | Maf-g5 | ATTAACATATTCCATGGCCAGGG |
| | g2 | C | | | |
| 190 | Atp2a3- | GCACATATATAAACAGACACAG | 973 | Mageh1- | TCATTTAATAAGGTTAGCACAGC |
| | g3 | G | | g1 | |
| 191 | Atp2a3- | ACAAAGGAAACCAGAGCTGCCA | 974 | Mageh1- | CAAAGCGATGAAAGCCTGCAGCA |
| | g4 | G | | g2 | |
| 192 | Atp2a3- | CACCTGAAAAATGAGAGGCAGA | 975 | Mageh1- | GCAGTTTAACAATTAGCACTGGT |
| | g5 | G | | g3 | |
| 193 | Aurka- | ACATGCAGAAAAAGAAACCCCC | 976 | Mageh1- | TGTTAATATCAAATGCCAACGAC |
| | g1 | G | | g4 | |
| 194 | Aurka- | TTAAACAGCACCTTCAGAGCCAG | 977 | Mageh1- | ACAACCTTTTGAATTCAGGACCC |
| | g2 | | | g5 | |
| 195 | Aurka- | AGGAACTCATAGCAGAGAACGC | 978 | Map2k7- | TAAAAATAAAACCATCAGGCCCA |
| | g3 | C | | g1 | |
| 196 | Aurka- | ACTAAATCAGGAAAAGCAGCAT | 979 | Map2k7- | CCAGAAATGACAAGGAGCAGCAA |
| | g4 | G | | g2 | |
| 197 | Aurka- | TGAATGACAGTAAGACAGAGCG | 980 | Map2k7- | AACAGGACAGTTAAGAGCCACAG |
| | g5 | T | | g3 | |
| 198 | B3gnt3- | AAAATTAAAACGTGGCAGAGCC | 981 | Map2k7- | CAGTGCTTTCACAATCGCCACAG |
| | g1 | C | | g4 | |
| 199 | B3gnt3- | ACAAAGTGAGTTCCAAGACAGC | 982 | Map2k7- | ACATCCTTAAACCAGGACGCGAC |
| | g2 | C | | g5 | |
| 200 | B3gnt3- | AGAACCCACAAAGTAGCTACAG | 983 | Mapk7- | TCACTTAACAAGAACCGCGGCCA |
| | g3 | G | | g1 | |
| 201 | B3gnt3- | GATTAACAGTAACAGCAGGTCTA | 984 | Mapk7- | GCAGAGTAACTCAAGCCAAGCCT |
| | g4 | | | g2 | |
| 202 | B3gnt3- | TAGAACAAGAAGTCGCGCACGT | 985 | Mapk7- | CTCACCAAAGATGCAGCCCACAG |
| | g5 | G | | g3 | |
| 203 | Bach2- | ACAGAGAATAAGCATCAACAGC | 986 | Mapk7- | AAAACCTTGTATTTCACAGGCCT |
| | g1 | C | | g4 | |
| 204 | Bach2- | TCAAGACACAGATGAACAGCGC | 987 | Mapk7- | TCCTCCAGATCAAAGCCAACTCC |
| | g2 | C | | g5 | |
| 205 | Bach2- | CCTTTAAGAATTACCAACCCCAC | 988 | Mcl1-g1 | CGAAGCGCTGTATAGAAGCCCAG |
| | g3 | | | | |
| 206 | Bach2- | CAAACCATATAATTTCCCCAGGT | 989 | Mcl1-g2 | ACAAGGTACTTAGCCTCCTCAGA |
| | g4 | | | | |
| 207 | Bach2- | CAAACTGTAGCAGTGGCCCAAA | 990 | Mcl1-g3 | CTTGGATGTCCAGTGAGAGCACC |
| | g5 | G | | | |
| 208 | Batf-g1 | TATTTAGAAAACTATCCACCCCC | 991 | Mcl1-g4 | GTTCACCGTCCTACAGGGCACTT |
| 209 | Batf-g2 | CCTCAGTTTACATGCCCCTCCAA | 992 | Mcl1-g5 | CAGCATGGCCAGGAGTAGCAGGA |
| 210 | Batf-g3 | CAGCAACCTCTGACAGACCCCTG | 993 | Mdm2- | GTTTTCACTTACATACCACCAGA |
| | | | | g1 | |
| 211 | Batf-g4 | GTGTTCAATACTTGTCCAGGCCT | 994 | Mdm2- | TAAGGAAAATATAAACAGCCAAT |
| | | | | g2 | |
| 212 | Batf-g5 | CTCACATCATCAGATGAGTCCTG | 995 | Mdm2- | CAAAGCAGAGTTCTGTGACGAGC |
| | | | | g3 | |
| 213 | Bcl11b- | CAGTACAAAAGCAAACCGCAGA | 996 | Mdm2- | TTGAACAATACACAATGTGCTGC |
| | g1 | G | | g4 | |
| 214 | Bcl11b- | AGCAGATTTTATAGACCCACCAT | 997 | Mdm2- | CTTAGTCATAATATACTGGCCAA |
| | g2 | | | g5 | |
| 215 | Bc111b- | CTCCAGTTAAAAATACAGCGAGA | 998 | Med23- | AAACCTAGCATATTGCAGACCAT |
| | g3 | | | g1 | |
| 216 | Bcl11b- | CAAAACACAGAAACGGAAGCAG | 999 | Med23- | TCCAGAAATGAACTGCAGCAGCA |
| | g4 | C | | g2 | |
| 217 | Bcl11b- | AACTTGAAGGTCTTGCCGCAGAA | 1000 | Med23- | CTTGAGTATAGAAACGCCCACAT |
| | g5 | | | g3 | |
| 218 | Bcl2-g1 | AAACAAATACATAAGGCAACCA | 1001 | Med23- | CCTCCAATGATTTTCCGAACCAG |
| | | C | | g4 | |
| 219 | Bcl2-g2 | TGTATGAATAAAGGCCACACCCA | 1002 | Med23- | CATGTCATAAAATGCCACGCCAA |
| | | | | g5 | |
| 220 | Bcl2-g3 | CTTTTAGAGCAAATGCAGCCACA | 1003 | Mertk- | AACAAAGTATCTAAGACCACCAG |
| | | | | g1 | |
| 221 | Bcl2-g4 | TATCAACCTTAAAAGCAGCCCAT | 1004 | Mertk- | AAAATGAATCCACAGAAGCAGCC |
| | | | | g2 | |
| 222 | Bcl2-g5 | CCGAACTCAAAGAAGGCCACAA | 1005 | Mertk- | CATCTTACAGAAGTACGACCCAT |
| | | T | | g3 | |
| 223 | Bcor-g1 | AAACATAAAAGTATACCACCACC | 1006 | Mertk- | TTAAAGATATAAGCACTCAGCTG |
| | | | | g4 | |
| 224 | Bcor-g2 | TTGTATGAAATTACGCCAAGCAC | 1007 | Mertk- | CTCATACAGATGTGGCGAAGCAG |
| | | | | g5 | |
| 225 | Bcor-g3 | CTGAAACATTAGTGACCACGGCA | 1008 | Met-g1 | CTACTGATATTGAGACCGCACCA |
| 226 | Bcor-g4 | CCCTGTTAATTCAATGCACACCT | 1009 | Met-g2 | TACATCATCTGTATGCAGCCAAG |
| 227 | Bcor-g5 | ACCACTTTAGAAGACAGGTCAAG | 1010 | Met-g3 | AGTAAGCACAAAATTCCACAGAG |
| 228 | Birc3-g1 | ACCAGTGTAGTAAAAGCCAGCA | 1011 | Met-g4 | ACACCTTATAAACCGCCGGCAGA |
| | | C | | | |
| 229 | Birc3-g2 | TGACAGAAAAGACAATGGCCAT | 1012 | Met-g5 | CACAAAGAAATTGATGAACCGGT |
| | | G | | | |
| 230 | Birc3-g3 | GTCCTGTATAATAAAAGCCCGCA | 1013 | Mex3b- | AAAATAAAAAGATTTAACCCAGC |
| | | | | g1 | |
| 231 | Birc3-g4 | CAAGTCTTTTAAGAACGGACAGC | 1014 | Mex3b- | ACACTAAAACTATAGCGGTGCAA |
| | | | | g2 | |
| 232 | Birc3-g5 | AGAATTTCAGGAGTGCAGGCAAT | 1015 | Mex3b- | ATGAACATTCAAAAAGGATCCAC |
| | | | | g3 | |
| 233 | Braf-g1 | AGACAGTTCCAAATGACCCAGAT | 1016 | Mex3b- | TCTGTCAGCTCGATGATGCCACC |
| | | | | g4 | |
| 234 | Braf-g2 | GAATTCTGTAAACAGCACAGCAT | 1017 | Mex3b- | CGTCACAACAAAGACAGGCTCCT |
| | | | | g5 | |
| 235 | Braf-g3 | CATTCAACATTTTCACTGCCACA | 1018 | Mfge8- | AGTACAATCAGAAGGGAAGGCCA |
| | | | | g1 | |
| 236 | Braf-g4 | ATAACCACATGTTTGACAACGGA | 1019 | Mfge8- | GAAACCCATATACACAGACGAGG |
| | | | | g2 | |
| 237 | Braf-g5 | TCCACAAAATAGATCCAGACAAC | 1020 | Mfge8- | AGTCACAGAAGTCACCAGACGCG |
| | | | | g3 | |
| 238 | Bst2-g1 | AACAGTGACACTTTGAGCACCAG | 1021 | Mfge8- | TCCTCATATACAGTCCACTGCAC |
| | | | | g4 | |
| 239 | Bst2-g2 | GCAAAAGCAATAAAGCAGAACT | 1022 | Mfge8- | AATTGTGTTATTCTTCAGGCCCA |
| | | C | | g5 | |
| 240 | Bst2-g3 | AGACACCAGAAATATGTGACCCC | 1023 | Mgat1- | ACAAAGATGATAGCACCCCAAAG |
| | | | | g1 | |
| 241 | Bst2-g4 | TCAAACAACTGTGACCTGCCAAC | 1024 | Mgat1- | AACAACTTATCCAAGCAGCGCCG |
| | | | | g2 | |
| 242 | Bst2-g5 | ATAGTGATAGAAAGAGGGCGCC | 1025 | Mgat1- | CCATGAAACTAAGATCAGGCAAC |
| | | A | | g3 | |
| 243 | Btla-g1 | ATATGTATATTAATCCAGCAGCA | 1026 | Mgat1- | TCCCTGAAGATCATCAACCCCGC |
| | | | | g4 | |
| 244 | Btla-g2 | GACCTTTAAGACGCAGCACCAGC | 1027 | Mgat1- | CACGCATAGACTTTCCATCTCCC |
| | | | | g5 | |
| 245 | Btla-g3 | ATCCTTTTCAGAAAGCAGAGCAG | 1028 | Mmp12- | AATATGTAGTCTACATCCTCACG |
| | | | | g1 | |
| 246 | Btla-g4 | TAACAGAATAAAGTGGAGTGCA | 1029 | Mmp12- | AGGCAGATACAAAATACACAGAC |
| | | A | | g2 | |
| 247 | Btla-g5 | TGTAGAACAGCTATACGACCCAT | 1030 | Mmp12- | TAGATCCTGTAAGTGAGGTACCG |
| | | | | g3 | |
| 248 | C10orf54- | TCCAGAGATAGATAAAGCACCC | 1031 | Mmp12- | TTGCCCAGTTGCTTCTAGCCCAA |
| | g1 | G | | g4 | |
| 249 | C10orf54- | CCATACAGGTAATGAGAGCCCA | 1032 | Mmp12- | ATTGCCAGAGTTGAGTTGTCCAG |
| | g2 | G | | g5 | |
| 250 | C10orf54- | CAGACAAAGCTAGATCCCCAGA | 1033 | Mn1-g1 | AGAGCCTTCTAGAACACACGCCG |
| | g3 | G | | | |
| 251 | C10orf54- | CAAGACACTAATGAGCTCACAGT | 1034 | Mn1-g2 | AGATATCAGAGTGCAGAGACCGC |
| | g4 | | | | |
| 252 | C10orf54- | CCAGGAAAATAGCAAGGAGCAG | 1035 | Mn1-g3 | TAGATTTATCTACATCAGCTCAG |
| | g5 | G | | | |
| 253 | C3ar1- | ATTCAGAACAATAAGAGCAGCCT | 1036 | Mn1-g4 | AAAACATGTCAAAATGTCCTGAG |
| | g1 | | | | |
| 254 | C3ar1- | ACCATGATGAAAAACGGCACCA | 1037 | Mn1-g5 | GCAATCATGTTCTGGCAAGCAGT |
| | g2 | G | | | |
| 255 | C3ar1- | CCCAATAGACAAGTGAGACCAA | 1038 | Mrc1-g1 | CCATAGAAAGGAATCCACGCAGT |
| | g3 | G | | | |
| 256 | C3ar1- | ACAGAATGAGTTTCAGGACAGCC | 1039 | Mrc1-g2 | AAGGACAAACCAATGCAACCCAG |
| | g4 | | | | |
| 257 | C3ar1- | ACACACATCACAAAGGCTACCAC | 1040 | Mrc1-g3 | GTCTTTGTAAATAACCCACCCAT |
| | g5 | | | | |
| 258 | C5ar1- | AACGCATTATAAGACAGGACAC | 1041 | Mrc1-g4 | CCTTGCCTTTCATAACCACGCAG |
| | g1 | C | | | |
| 259 | C5ar1- | ATAAAGAAACAGATGACCACAG | 1042 | Mrc1-g5 | ACAGAGATAAAAGCCAGAAGCAG |
| | g2 | C | | | |
| 260 | C5ar1- | TATACCATGACATTTGCCCAGCA | 1043 | Msr1-g1 | ATGAAGTACAAGTGACCCCAGCA |
| | g3 | | | | |
| 261 | C5ar1- | CCTCAAGAAGAGATGCAGGCAA | 1044 | Msr1-g2 | ATCATCACAGATTGTGCCCCACT |
| | g4 | C | | | |
| 262 | C5ar1- | AATACCACATACAGTGTGCTCTG | 1045 | Msr1-g3 | CATTCAGCCATATTGGACCAGTA |
| | g5 | | | | |
| 263 | Cad-g1 | AAATACATCGAAGAGCAGTTCCA | 1046 | Msr1-g4 | ATGCTGTCATTGAACGTGCGTCA |
| 264 | Cad-g2 | CAAAGTGATTTTCAGCCAGCTGA | 1047 | Msr1-g5 | TTCCCAATTCAAAAGCTGAGCTG |
| 265 | Cad-g3 | ATCTGATTATACTTGAGGCCACA | 1048 | Muc16- | TCTGAAAAGCTGATTGAGCGCAT |
| | | | | g1 | |
| 266 | Cad-g4 | AGCAAAGCCAGAACCGAGACCA | 1049 | Muc16- | TGAGTACCAGTAGTACCGCCAAG |
| | | C | | g2 | |
| 267 | Cad-g5 | CAGTATCTGAGACTGCATCCCCA | 1050 | Muc16- | GAGACAAATAATAAGCTAGACGA |
| | | | | g3 | |
| 268 | Casp1- | GACTCAATGAAAAGTGAGCCCCT | 1051 | Muc16- | CTGTTTTGAGAATACCCATCCAC |
| | g1 | | | g4 | |
| 269 | Casp1- | AGTCTGAAAAGGATTCAACCGCG | 1052 | Muc16- | GTATGGTTATATTCAGTGGCACA |
| | g2 | | | g5 | |
| 270 | Casp1- | TAAGTGATAAAGATTTGGCTTGC | 1053 | Muc5ac- | CAGTAGTCAAATAAGCAGCCTTG |
| | g3 | | | g1 | |
| 271 | Casp1- | AGACATGATCACATAGGTCCCGT | 1054 | Muc5ac- | AGAACACATAGTTGCAGAGACCA |
| | g4 | | | g2 | |
| 272 | Casp1- | ACCACAATTGCTGTGTGCGCATG | 1055 | Muc5ac- | GCCAATGTCAGTTTCCACCACCA |
| | g5 | | | g3 | |
| 273 | Casp6- | TGACCAAGTCAAATAGGCCCACT | 1056 | Muc5ac- | GCATAGTAACAGTGGCCATCAAG |
| | g1 | | | g4 | |
| 274 | Casp6- | GTGAAATATATGTAGCAAGACA | 1057 | Muc5ac- | ATCAAAAGTGATGTAGTGGCCAT |
| | g2 | G | | g5 | |
| 275 | Casp6- | ACAGATGAAGCAATCGGCATCTA | 1058 | Myc-g1 | ATACTATTTAAGTTTGAGGCAGT |
| | g3 | | | | |
| 276 | Casp6- | ACACAAATCCTGAATGTACCAGG | 1059 | Myc-g2 | CATGCATTTTAATTCCAGCGCAT |
| | g4 | | | | |
| 277 | Casp6- | AGATCTGAAAACCTGCGAGTCAG | 1060 | Myc-g3 | AAGTTATTTACATTTCAAGGCCC |
| | g5 | | | | |
| 278 | Cblb-g1 | AAAAACCTGAAATTGCCACAGA | 1061 | Myc-g4 | CTGGAATTACTACAGCGAGTCAG |
| | | G | | | |
| 279 | Cblb-g2 | AATTCCGTAAAATAGAGCCCCAG | 1062 | Myc-g5 | CCGCAACATAGGATGGAGAGCAG |
| 280 | Cblb-g3 | GAACTGAAAAAGTAGCAGCAAG | 1063 | Mycn-g1 | TCATACTAAAGTATACAGGCCGT |
| | | G | | | |
| 281 | Cblb-g4 | GCAAGCTACATGAAGCCCAACA | 1064 | Mycn-g2 | AAACGTTTAGCAAGTCCGAGCGT |
| | | G | | | |
| 282 | Cblb-g5 | TGACCATTATCACAAGACCGAAC | 1065 | Mycn-g3 | TCAAAATGTGCAAAGTGGCAGTG |
| 283 | Cbx8-g1 | CTAGAGATAATTTTCCAGCCAGA | 1066 | Mycn-g4 | ACAGACACACTAGTGACCGCAGC |
| 284 | Cbx8-g2 | TAGTTTTACAAATATGAGCAGAT | 1067 | Mycn-g5 | AAACAAGGAAGAAACAGGCTAGG |
| 285 | Cbx8-g3 | GCAAAAAGAAAAAGTCCACCCG | 1068 | N6amt1- | CTCTGAAAACAAAATGACCCAGC |
| | | A | | g1 | |
| 286 | Cbx8-g4 | CACACCTCCATAAAGTGCACCCT | 1069 | N6amt1- | ACGGTAACTAAGTAGAACAGCCC |
| | | | | g2 | |
| 287 | Cbx8-g5 | CACGAGATATTCCATGCGTCCTT | 1070 | N6amt1- | TAAGGCTAAATCAAACGTGCCTT |
| | | | | g3 | |
| 288 | Ccl11-g1 | TTCCAGAGAAAACTGCCTGCACA | 1071 | N6amt1- | TTATTTATTGTATATGAGCACAC |
| | | | | g4 | |
| 289 | Ccl11-g2 | TACACAGTGAGTTACAGGTCAGT | 1072 | N6amt1- | AATAAAATTTCTTGGCCAGGCAA |
| | | | | g5 | |
| 290 | Ccl11-g3 | TACTGGTCATGATAAAGCAGCAG | 1073 | Nanog- | ACCAAGTTGTAAATAGAGCTCAG |
| | | | | g1 | |
| 291 | Ccl11-g4 | AAACATAATGACTTCCAGTCCCA | 1074 | Nanog- | ACAGTGTATACCAAGACCCACGC |
| | | | | g2 | |
| 292 | Ccl11-g5 | AGCACAGATCTCTTTGCCCAACC | 1075 | Nanog- | CATACGTAACAAGATCTGACGCC |
| | | | | g3 | |
| 293 | Ccl17-g1 | CCTTTGAAGTAATCCAGGCAGCA | 1076 | Nanog- | CAAAGACAATTAGAGCTATGCAG |
| | | | | g4 | |
| 294 | Ccl17-g2 | ATGTTGAAACCATGGACAGCAGC | 1077 | Nanog- | AGAGCATCTCAGTAGCAGACCCT |
| | | | | g5 | |
| 295 | Ccl17-g3 | CATGCTGCAGAAAAGTCCCCAGA | 1078 | Ncf1-g1 | CACTTTGAAGAAGTCCAGCAGGT |
| 296 | Ccl17-g4 | GTCAGAAACACGATGGCATCCCT | 1079 | Ncf1-g2 | GACATGAAAGCAAAGTCTCCGCA |
| 297 | Ccl17-g5 | GTCTGCACAGATGAGCTTGCCCT | 1080 | Ncf1-g3 | TCAAGTTGACGTAGACCAGCCAG |
| 298 | Ccnb1- | AACCGATCAATAATGGACACAGT | 1081 | Ncf1-g4 | GAACATGTACACATAGTGCTGGC |
| | g1 | | | | |
| 299 | Ccnb1- | AGAATGCACTTGAATCCCACCGA | 1082 | Ncf1-g5 | TGAAATTACAGATGTGAGCCTCG |
| | g2 | | | | |
| 300 | Ccnb1- | TTACATCAGAGAAAGCCTGACAC | 1083 | Nfatc1- | GCTAATTACACAGAGCAGACACC |
| | g3 | | | g1 | |
| 301 | Ccnb1- | AGCTACTATGAATGAAGCCAAGC | 1084 | Nfatc1- | GTAACTTAAAACTTCCAGCCACA |
| | g4 | | | g2 | |
| 302 | Ccnb1- | GTATATAGACATATAGGCTACAG | 1085 | Nfatc1- | TGCAGAAAGTTATGGCCAGACAG |
| | g5 | | | g3 | |
| 303 | Ccnq-g1 | CATCACTAAATACCTGCCACCAA | 1086 | Nfatc1- | TAACTACAAAGATAGCTGACACA |
| | | | | g4 | |
| 304 | Ccnq-g2 | GGAAATCAAGTAGTGCAGCAGG | 1087 | Nfatc1- | CCCAATGAACAGCTGTAGCGTGA |
| | | T | | g5 | |
| 305 | Ccnq-g3 | AACTCGTAGCATAAGAAGCTCAC | 1088 | Nfkb2- | TCATCCTCATAGAACCGAACCTC |
| | | | | g1 | |
| 306 | Ccnq-g4 | TACACATTCATACTTCCCCTAAG | 1089 | Nfkb2 | CCAGATTATTAAATTGAGCAGTC |
| | | | | g2 | |
| 307 | Ccnq-g5 | AACTTATGGTAAATGGTGCAAGC | 1090 | Nfkb2- | CATATCGAAATCTGAAGCCTCGC |
| | | | | g3 | |
| 308 | Ccr3-g1 | ATAATGATAGAAACACAGGCAG | 1091 | Nfkb2- | AGTCAACTAACAGATCCAGGCAC |
| | | T | | g4 | |
| 309 | Ccr3-g2 | CCAGGTAATAAAACCCAGACAG | 1092 | Nfkb2- | AATAAGATTTCGATTAGACAGGG |
| | | C | | g5 | |
| 310 | Ccr3-g3 | AAGAATTAACGACAGCACCACA | 1093 | Nfkbia- | ACATTTACAAGAAGGCGACACAG |
| | | C | | g1 | |
| 311 | Ccr3-g4 | TATCAAACATCTAAAGAGCCCAT | 1094 | Nfkbia- | GATTTCACAAAGACAACAGCCGA |
| | | | | g2 | |
| 312 | Ccr3-g5 | ATAAATTCAGGCAATGCTGCCAG | 1095 | Nfkbia- | CAACAAGAGCGAAACCAGGTCAG |
| | | | | g3 | |
| 313 | Ccr8-g1 | AACCAATGTAATAAAGGCCAGA | 1096 | Nfkbia- | TTTCCACTTATAATGTCAGACGC |
| | | G | | g4 | |
| 314 | Ccr8-g2 | AGACCAATTATCTGAGCGAGCCT | 1097 | Nfkbia- | CAAAGTCACCAAGTGCTCCACGA |
| | | | | g5 | |
| 315 | Ccr8-g3 | CCCAGCACAAACAAGACGCAGT | 1098 | Nos1-g1 | ATCAGATCTGAGATGATCACCGG |
| | | A | | | |
| 316 | Ccr8-g4 | ACTGGAAACATTGTAGCATGCCG | 1099 | Nos1-g2 | GACGCACAAACATGGGAACTAGG |
| 317 | Ccr8-g5 | ACAATGACTACGGTGAGCACCA | 1100 | Nos1-g3 | AGAAGACCTCCAAGCCAAGACGA |
| | | G | | | |
| 318 | Ccr9-g1 | CATAATGAAGACAGTGAGGACA | 1101 | Nos1-g4 | CCACAGGAACCTGAGCTACTCCG |
| | | G | | | |
| 319 | Ccr9-g2 | CACATGATGAGAAGCACACAGC | 1102 | Nos1-g5 | TCTAAGGAAGTCAAGGTTGACCA |
| | | T | | | |
| 320 | Ccr9-g3 | GCCCACAATGAACACAAGCCAG | 1103 | Nos3-g1 | CCGAGCATCAAATACCTGCAGCT |
| | | T | | | |
| 321 | Ccr9-g4 | TACCAGTAGACAAGGATGACCA | 1104 | Nos3-g2 | GAACCTTCAAGATTTAGGCCGAC |
| | | G | | | |
| 322 | Ccr9-g5 | TAGCATAGAAGAAAGCTTCAAG | 1105 | Nos3-g3 | CAAAGCATATGAAGAGGGCAGCA |
| | | C | | | |
| 323 | Cd160- | ACCAGAAGAGTATTAGCCCCTCA | 1106 | Nos3-g4 | AAGAAAGATCTTAAATTGGCAGC |
| | g1 | | | | |
| 324 | Cd160- | ACACTGATGTAACTGCAGTTCCA | 1107 | Nos3-g5 | GAACTCATGTACCAGCCGCTGAA |
| | g2 | | | | |
| 325 | Cd160- | AATGTCTTGTTAAACGAGCAGAG | 1108 | Nr4a2- | TGCCTTCAATTCATGCCACCCAC |
| | g3 | | | g1 | |
| 326 | Cd160- | ATACACGAAAGTTCACGGGCTTA | 1109 | Nr4a2 | TCTTCATTCATTCAACTCCGCCG |
| | g4 | | | g2 | |
| 327 | Cd160- | GAAGTTCACAATTGCCAGCAGGA | 1110 | Nr4a2- | GGAAACAGTCAAAAGCTGCTGCA |
| | g5 | | | g3 | |
| 328 | Cd200r1- | AAAATTAACAGGTAGCAGCAGA | 1111 | Nr4a2- | GAACCACTTCTTTAACCATCCCA |
| | g1 | G | | g4 | |
| 329 | Cd200r1- | TTATCAGTACAACTTGACCCAGC | 1112 | Nr4a2- | AGCTGATTCAAAAAGCAGGTCCT |
| | g2 | | | g5 | |
| 330 | Cd200r1- | AATAAACATGTAATTGGACACAC | 1113 | Nras-g1 | CTTTTAAATAGAAACCACCCAGT |
| | g3 | | | | |
| 331 | Cd200r1- | GAATTTTAAAGAAAGCAAAGCA | 1114 | Nras-g2 | ACCGAGATAACTGTTCAAGCCCC |
| | g4 | G | | | |
| 332 | Cd200r1- | GGAATAGAAAAGCAGCAGAGCA | 1115 | Nras-g3 | TTGCATTTATGAATACAGAGCAG |
| | g5 | G | | | |
| 333 | Cd274- | CGTAGCAAGTGACAGCAGGCTGT | 1116 | Nras-g4 | TACGTAATCACTAGGCGCCCAAG |
| | g1 | | | | |
| 334 | Cd274- | CCATCGTGACGTTGCTGCCATAC | 1117 | Nras-g5 | GTACTCAGTCATTTCACACCAGC |
| | g2 | | | | |
| 335 | Cd274- | CGCTTGTAGTCCGCACCACCGTA | 1118 | Nrp1-g1 | TTTCCGAGAAGAATCCACCACAG |
| | g3 | | | | |
| 336 | Cd274- | TAGAAAACATCATTCGCTGTGGC | 1119 | Nrp1-g2 | AGTTTCAGAGATTTGTGCAGCAA |
| | g4 | | | | |
| 337 | Cd274- | ATTTCTCCACATCTAGCATTCTC | 1120 | Nrp1-g3 | AAAGCAGAGTAACAGAGTCCCCA |
| | g5 | | | | |
| 338 | Cd276- | ATAATAGCAGTTACACAGTCTGC | 1121 | Nrp1-g4 | CATAGATATACCAGTTTCCCAGG |
| | g1 | | | | |
| 339 | Cd276- | GTGAACATCGAACAAGCCCCGCT | 1122 | Nrp1-g5 | CAAAGATGATGTAGGTGCACTCC |
| | g2 | | | | |
| 340 | Cd276- | AGCAAGAACTAAGAGGTCACTG | 1123 | Nt5e-g1 | TACAATTACAAGATAGTCCAAGG |
| | g3 | T | | | |
| 341 | Cd276- | GACAACAAAAGCCAGGGCCAGA | 1124 | Nt5e-g2 | AGATGTATTCAGAAACCACGCTG |
| | g4 | T | | | |
| 342 | Cd276- | TTTAATGAAGAGCTGACGGCCAA | 1125 | Nt5e-g3 | CTTTCGGTTAATATCGTACACCA |
| | g5 | | | | |
| 343 | Cd300lf- | CACCTTTCGTAATTCCACACCAG | 1126 | Nt5e-g4 | ATCTCAAAACCAGAGTGCCCCAG |
| | g1 | | | | |
| 344 | Cd300lf- | TGATACGATTTACACAGCATCCC | 1127 | Nt5e-g5 | CTGAGAGACAACAAGAGCCCAAA |
| | g2 | | | | |
| 345 | Cd300lf- | AAGCAAAGAGCGAGGCCACCAA | 1128 | Ntf3-g1 | CAGAAGTAACCATGGCATCCGTG |
| | g3 | C | | | |
| 346 | Cd300lf- | ACAATCAATTCAACTCCACCCAA | 1129 | Ntf3-g2 | AATAATTTATATGTGGGGACAGA |
| | g4 | | | | |
| 347 | Cd300lf- | CAGATGTAAAGATCCATGCTCCA | 1130 | Ntf3-g3 | CATAAAACAAGATGGACATCACC |
| | g5 | | | | |
| 348 | Cd38-g1 | AGATCATCAGCAATGTAGCCCAG | 1131 | Ntf3-g4 | ATAATTTATATGTGGGGACAGAT |
| 349 | Cd38-g2 | ATAAACAATACAGAAGCACCAC | 1132 | Ntf3-g5 | CCAGTGTTTGTCATCAATCCCCC |
| | | A | | | |
| 350 | Cd38-g3 | AAAACATGAATACAGAAGCACC | 1133 | Otulin- | AGCACAGAGAAGAACGGCACTTC |
| | | T | | g1 | |
| 351 | Cd38-g4 | CCAATTTAACAAGTGGGGCGTAG | 1134 | Otulin- | TCAGCAGTTTTCATTGCAGCCAG |
| | | | | g2 | |
| 352 | Cd38-g5 | CAGAGCAAACTGACCAGAACCT | 1135 | Otulin- | TATAATTCCAGCTTTGGCAGCAA |
| | | C | | g3 | |
| 353 | Cd47-g1 | CAAGCAAGACAGAAGCGCCAAG | 1136 | Otulin- | ACATCAGGAACTTCACAGCTTCG |
| | | T | | g4 | |
| 354 | Cd47-g2 | TAGAGATTACAATGAGGCCAAGT | 1137 | Otulin- | TGCTCCATAAGCGTCCGCCACCT |
| | | | | g5 | |
| 355 | Cd47-g3 | ACCAAAGCAAGGACGTAGCCCA | 1138 | Pced1b- | GTGGAAATTTAAGTCCAGCACAT |
| | | G | | g1 | |
| 356 | Cd47-g4 | CCACGATGACTGTGAGCACCAGC | 1139 | Pced1b- | AAACAAAGAGAAGTCCAAGACAG |
| | | | | g2 | |
| 357 | Cd47-g5 | TAAACAGTAGTTGAGCTGAACCT | 1140 | Pced1b- | TCCATGTACTCAGAGTAGACCCG |
| | | | | g3 | |
| 358 | Cd5-g1 | ACAAAGGACAAATGTCCAAGCG | 1141 | Pced1b- | CTACTCAAGGAATTCCGCCCATA |
| | | T | | g4 | |
| 359 | Cd5-g2 | AGAGTCCAAGGAGAAAGCCAAC | 1142 | Pced1b- | GCCTCTCATTACCTGCAGCCGAG |
| | | C | | g5 | |
| 360 | Cd5-g3 | AATTATTTAGACTCTAGGACCAT | 1143 | Pcsk1-g1 | CAGAGCAAAGATACCAGCAGCCA |
| 361 | Cd5-g4 | TCCCACTGTGATCTCTGGCGCAC | 1144 | Pcsk1-g2 | ACAGGATAAACTGTAGCAGCCAA |
| 362 | Cd5-g5 | ATAAGTCCTTGTAAGTACCCCAC | 1145 | Pcsk1-g3 | AAGCTCAATTATTAGTGGCAGAG |
| 363 | Cd55-g1 | AAATGCTAGCATTTCCAACCAGG | 1146 | Pcsk1-g4 | AGCAAGATCAGAGACACACCAGC |
| 364 | Cd55-g2 | CATATATATAACGGTCACCACCT | 1147 | Pcsk1-g5 | TCCAGCATTCTTATGCCTCCAAC |
| 365 | Cd55-g3 | TCTTGAAGACAATGACAGCATGC | 1148 | Pdcd1- | AGTTCAGCATAAGATCCTCCGAC |
| | | | | g1 | |
| 366 | Cd55-g4 | CAAAACTGAGCAACTGGAGACC | 1149 | Pdcd1- | CAAACCATTACAGAAGGCGGCCT |
| | | A | | g2 | |
| 367 | Cd55-g5 | GTTAAATTAGAATGTGCCACCTC | 1150 | Pdcd1- | AAGTCCCTAGAAGTGCCCAACAG |
| | | | | g3 | |
| 368 | Cd51-g1 | ATCAAGTTGAACAATGGAGCCAT | 1151 | Pdcd1- | AGCAGCAATACAGGGATACCCAC |
| | | | | g4 | |
| 369 | Cd51-g2 | AAAAATGCTCAAGATGGCCAGC | 1152 | Pdcd1- | CCAGTCTACGAATTTCCCACCTG |
| | | A | | g5 | |
| 370 | Cd51-g3 | AAACGACTCAAAAGGCAAGACC | 1153 | Pdcd11g2- | ATGAAAACATGAAGTGGCCACGT |
| | | G | | g1 | |
| 371 | Cd51-g4 | AGATATGCAAGTGACCCATCTAC | 1154 | Pdcd1lg2- | AAGTAGAAACAAATACCACAGTG |
| | | | | g2 | |
| 372 | Cd51-g5 | CTAGAATAAGGAAGAGAGGCAG | 1155 | Pdcd11g2- | CAAAAGTGCAAATGGCAGGTCCT |
| | | G | | g3 | |
| 373 | Cd96-g1 | AAGCCCAAACAATAGTGTGCAG | 1156 | Pdcdllg2- | GCATTCCAGAACATGCAGCTGAA |
| | | A | | g4 | |
| 374 | Cd96-g2 | ATCCACTTTTAGAAGAAGCAGCA | 1157 | Pdcdllg2- | TACAACAATTACCTTGTGACTCA |
| | | | | g5 | |
| 375 | Cd96-g3 | CATTCATTTGAGCTGAAGCAGAG | 1158 | Peg10- | TCCAAAATCAAAATGACGACCAC |
| | | | | g1 | |
| 376 | Cd96-g4 | ACAGAGTCCAATTTGTTACACCC | 1159 | Peg10- | GTCCACGAAATTTCGCAGAGCAT |
| | | | | g2 | |
| 377 | Cd96-g5 | ATGCACTCATACTTTCCACCCAG | 1160 | Peg10- | ATTTCCTAAATTACCCACCCACC |
| | | | | g3 | |
| 378 | Cda-g1 | GCGTTTTCTATGTTGCACCCAGA | 1161 | Peg10- | ACTGATACATGAAAGGACCCAGC |
| | | | | g4 | |
| 379 | Cda-g2 | AATCTGAAAAGTCACACGACAG | 1162 | Peg10- | AGGAATTCAAATTTGGGCCCACA |
| | | A | | g5 | |
| 380 | Cda-g3 | ATTATGAAGTCTTCCAAGAGCCG | 1163 | Pik3ca- | CCAAGATAAAGGTTGCCACGCAG |
| | | | | g1 | |
| 381 | Cda-g4 | ACTAGAGATGGCAATAGCCCTGA | 1164 | Pik3ca- | AGCGCACTATTTATGACCCAGAG |
| | | | | g2 | |
| 382 | Cda-g5 | ATGACTTGTCGGCAGGCTCCACA | 1165 | Pik3ca- | AGTGTTTCAATTATAGAGCACGT |
| | | | | g3 | |
| 383 | Cdh2-g1 | TAAAACATTATGTACACACACCA | 1166 | Pik3ca- | AGTAGAAATCTAGAGCGACCACT |
| | | | | g4 | |
| 384 | Cdh2-g2 | AACGGCAATAGTAGTGATCTGCC | 1167 | Pik3ca- | AAGCAGATATTGATCACCCCAGC |
| | | | | g5 | |
| 385 | Cdh2-g3 | AGCAAAATCACCATTAAGCCGGT | 1168 | Pik3cg- | CCAAGAACAAATGTGGCCACACA |
| | | | | g1 | |
| 386 | Cdh2-g4 | CAAACATGAGAACAAGGATCAG | 1169 | Pik3cg- | ACCCTTTAAAGAATGCAGCAGAG |
| | | C | | g2 | |
| 387 | Cdh2-g5 | TGTAAAATTCAAGTGTAGCTGAG | 1170 | Pik3cg- | AGCAATTCAAATGAAAGGCAGAG |
| | | | | g3 | |
| 388 | Cdk1-g1 | TTATAGTAATTAACAGCCACCAC | 1171 | Pik3cg- | CAAGAACAAGATGTAGGAACCAG |
| | | | | g4 | |
| 389 | Cdk1-g2 | AAGAATTACACTAAGCAGCACA | 1172 | Pik3cg- | TCATATCGAAAATGCCAGAGCAA |
| | | G | | g5 | |
| 390 | Cdk1-g3 | GTAGACAAACTTAAGCATCCCAG | 1173 | Pik3r5- | CACAGCAAGGAATTCAGCCTGTA |
| | | | | gl | |
| 391 | Cdk1-g4 | AGATTAAAGATTGCGCGCCACTA | 1174 | Pik3r5- | ACAAACCAAAGATGGACCTCAGC |
| | | | | g2 | |
| 392 | Cdk1-g5 | TGAAGAATCCATGAACTGCCCAG | 1175 | Pik3r5- | GTAGAACAATATGCTCACACAGC |
| | | | | g3 | |
| 393 | Cdk20- | AGCATAAATTCGAAAGCCAGCA | 1176 | Pik3r5- | TGTTCAAGAACAAAGGAGGGCAT |
| | g1 | C | | g4 | |
| 394 | Cdk20- | CTTGAAAAGATCAAGCCAGACA | 1177 | Pik3r5- | GCATCAATGAAGGTGAGCATCTC |
| | g2 | G | | g5 | |
| 395 | Cdk20- | CCCACTTTATTAAACCCACCAAG | 1178 | Pim1-g1 | CTTTACTCAGATAAAACCAGCGG |
| | g3 | | | | |
| 396 | Cdk20- | CAGTAAATCTGAATGGCCAACAC | 1179 | Pim1-g2 | TTTTATGTACAGTCAGCAGACCC |
| | g4 | | | | |
| 397 | Cdk20- | TGCAATTAGACAGAAACACCCA | 1180 | Pim1-g3 | AAAAACCAAACCAAACCCCCAAC |
| | g5 | G | | | |
| 398 | Cdk5-g1 | ACAATCAAAAGAACAGGGACCA | 1181 | Pim1-g4 | CCATTTAATAAGGTGCTGACACT |
| | | T | | | |
| 399 | Cdk5-g2 | AGTAGGTATTCATTGCACCAACG | 1182 | Pim1-g5 | CAGAAGTCTAATGACGCCCGAGA |
| 400 | Cdk5-g3 | GAAATTAAATAAAGTCCACGGA | 1183 | Pkn2-g1 | AGAGTACAATAACAACAGGCCAA |
| | | G | | | |
| 401 | Cdk5-g4 | TCAGCCAATTTCAACTCCCCATT | 1184 | Pkn2-g2 | ACGAGTAATAGCATCCAAAGCCC |
| 402 | Cdk5-g5 | GAACTCAAAAACCAGTGTCAGCT | 1185 | Pkn2-g3 | GTTCATGTAAGTATTGCAACCCA |
| 403 | Cebpb- | AACCAAAAACATCAACAACCCC | 1186 | Pkn2-g4 | CATATATAAGTACACCAAGGCCC |
| | g1 | G | | | |
| 404 | Cebpb- | GTTCTCAAAATATACATACGCCT | 1187 | Pkn2-g5 | TTTTGCATTATCAAAAGCCAGCT |
| | g2 | | | | |
| 405 | Cebpb- | CTCGTCGCTCAGCTTGTCCACCG | 1188 | Pla2g2d- | ACTCAGACAGAAAAACAGCAGCA |
| | g3 | | | g1 | |
| 406 | Cebpb- | GATTGCATCAAGTCCCGAAACCC | 1189 | Pla2g2d- | TTTACAGTAGAAAGGCACCAAGA |
| | g4 | | | g2 | |
| 407 | Cebpb- | CTCTCGCGACAGCTGCTCCACCT | 1190 | Pla2g2d- | ATTATGCAGCAAGAGATCCCAGC |
| | g5 | | | g3 | |
| 408 | Cep55- | ATATTGCTAAATAGTAGCCCAAG | 1191 | Pla2g2d- | TTCCAGAGAAAGAAACCAGGCCT |
| | g1 | | | g4 | |
| 409 | Cep55- | CCTGCAAATCAAATGAGGCAAG | 1192 | Pla2g2d- | ACAATCATGCTTCTGACAGCACC |
| | g2 | A | | g5 | |
| 410 | Cep55- | AGGAGTAAAAATATACAGCCAC | 1193 | Plac1-g1 | AGAAACAGAATTTTCAGAAGCCC |
| | g3 | T | | | |
| 411 | Cep55- | AAATGCTAGTCATTACAACAGCG | 1194 | Plac1-g2 | TGACTACACAAGAAGGACCTCCA |
| | g4 | | | | |
| 412 | Cep55- | AAGTCTAGAGTACATGCCTGCAT | 1195 | Plac1-g3 | TGAACCAATCTGTCGAGCACAGC |
| | g5 | | | | |
| 413 | Cflar-g1 | AGAAAAGCTGGATATGATAGCC | 1196 | Plac1-g4 | CCCAGAAACATTTGCACAGTCAG |
| | | C | | | |
| 414 | Cflar-g2 | CTCTGTAGAGCAATTCAGCCAAG | 1197 | Plac1-g5 | CACATATTTCGTTGATGAGCCCT |
| 415 | Cflar-g3 | ATGATATACCAAGAACACCAAC | 1198 | Plau-g1 | GTTAGATACAGAAATACACCAGC |
| | | G | | | |
| 416 | Cflar-g4 | CATACTTGCATATCGGCGAACAA | 1199 | Plau-g2 | TTTATAATTAATTTCAGAGCCAT |
| 417 | Cflar-g5 | GAAGATATTTTGTGTCGTTGCCA | 1200 | Plau-g3 | CAATATCATTATGGTAGGCCAGG |
| 418 | Chic2-g1 | TTAGACATATAATTCCCAGCACA | 1201 | Plau-g4 | GAAATCATTTATTTCCCAACAGC |
| 419 | Chic2-g2 | GTCCGATTATGTACAGAGCCACA | 1202 | Plau-g5 | AGAAGTACTTGTAGGACACGCAT |
| 420 | Chic2-g3 | AAACACTGCATTTTGGAACCGCA | 1203 | Pole-g1 | ATCCAGAGATAGTACCTTGCACA |
| 421 | Chic2-g4 | AGAACCAAGAAGAAGCAACCCC | 1204 | Pole-g2 | CAGAAATTACAGCTGTGGCAGAT |
| | | T | | | |
| 422 | Chic2-g5 | AATAAACAATAATTGCCAGGCGT | 1205 | Pole-g3 | CTTGACAATTGGAGCAGAGCCAC |
| 423 | Cish-g1 | GCACAACATAGAGAAGCCAGCT | 1206 | Pole-g4 | AAAACCGTGTTTACTTAGCAGGC |
| | | C | | | |
| 424 | Cish-g2 | TAAACAGAGATAGTCAGCTCCCA | 1207 | Pole-g5 | TCCAGAGAAGGAATTCCCAGCAT |
| 425 | Cish-g3 | TTGACAAGCAGTTAGAGTCCAGC | 1208 | Porcn-g1 | CACAAAGTAGAGGTAGCCCATGA |
| 426 | Cish-g4 | CTGAAGAAAGGACAGCAGAACC | 1209 | Porcn-g2 | CCAGCATCCAAAAGTGACCCAGT |
| | | C | | | |
| 427 | Cish-g5 | CACTACAGCTAAAAGAGTTCAGG | 1210 | Porcn-g3 | CAAAGAGCAAGTTTAAGGCTCGT |
| 428 | Cks1b- | TCCAGAAGCAAAGCAGGTACCA | 1211 | Porcn-g4 | GCCTCAGACAGAAAGCCCACAAA |
| | g1 | G | | | |
| 429 | Cks1b- | GCCAAATGACTAATATGGCCTGC | 1212 | Porcn-g5 | AAGCAGATGGTAAGAAGCAGCCA |
| | g2 | | | | |
| 430 | Cks1b- | CCTCGCTTTCAAACACACCGCCC | 1213 | Postn-g1 | AACAAAATTTAGCAGGAAACCCA |
| | g3 | | | | |
| 431 | Cks1b- | AAGAAAGCAACATGGTCACGCG | 1214 | Postn-g2 | ATGGTTAATAAAAAGCCCCAGAT |
| | g4 | A | | | |
| 432 | Cks1b- | ACACCATCCTTGTAACAGCCATG | 1215 | Postn-g3 | AGCACAGTTCACAGTGACAACCC |
| | g5 | | | | |
| 433 | Clec4e- | GTTTAAAACAAAGAGGAGCACA | 1216 | Postn-g4 | GCAGAATTAGCTTAAGAAGGCGT |
| | g1 | T | | | |
| 434 | Clec4e- | CATGCAATAAATGCAGTCCTGAG | 1217 | Postn-g5 | GGCAGCATTCATATAGCACAGTG |
| | g2 | | | | |
| 435 | Clec4e- | TAGATAGATAAGCTAGACCCAA | 1218 | Pou5f1- | ACCTTTCCAAAGAGAACGCCCAG |
| | g3 | G | | g1 | |
| 436 | Clec4e- | CCCAGTAATACATACATGTGCAG | 1219 | Pou5f1- | ACAAAATGATGAGTGACAGACAG |
| | g4 | | | g2 | |
| 437 | Clec4e- | ATTCTTTAAACTTGATGACCAGG | 1220 | Pou5f1- | TTACAGAACCATACTCGAACCAC |
| | g5 | | | g3 | |
| 438 | Clec4g- | AGCAACAGTTACGATGCCTACCG | 1221 | Pou5f1- | CACTTCAGAAACATGGTCTCCAG |
| | g1 | | | g4 | |
| 439 | Clec4g- | CAGAGAAATAGTAGCAGGAACC | 1222 | Pou5f1- | AGTAAAAGAATTTAACCCCAAAG |
| | g2 | T | | g5 | |
| 440 | Clec4g- | TATTTATATCACAATCACTCCAC | 1223 | Prc1-g1 | GCATTAATCAAATGTCCGCGAAC |
| | g3 | | | | |
| 441 | Clec4g- | TAAATATGAGAAAAGAACCCCC | 1224 | Prc1-g2 | CAGTTAATAGAACCTGCCCAAGG |
| | g4 | G | | | |
| 442 | Clec4g- | ACCAAGAGCTTGAGGAGAGACG | 1225 | Prc1-g3 | GCAACAGACTTAATGCTGCAGTT |
| | g5 | C | | | |
| 443 | Cmtm6- | CATAGAATGCAAAAGCCAACCA | 1226 | Prc1-g4 | ATCCACTTCTAATTTCAGCGCAT |
| | g1 | G | | | |
| 444 | Cmtm6- | ACAAAAATGATAGATGCCAGCA | 1227 | Prc1-g5 | ACATATATGTGTGTGAAGAGCAG |
| | g2 | G | | | |
| 445 | Cmtm6- | AAAATTCTAAGAGTGGCCACAG | 1228 | Prkci-g1 | CCAATAAGAAATATGGCCACAAG |
| | g3 | A | | | |
| 446 | Cmtm6- | CATAACATTAAGGAAAGACGCCT | 1229 | Prkci-g2 | AGATTTAAAAACAAAGACCACCA |
| | g4 | | | | |
| 447 | Cmtm6- | ACATGCAATTCAAAGGACAGCA | 1230 | Prkci-g3 | AAGCAAGAATGCAGCCCGACAAG |
| | g5 | G | | | |
| 448 | Cnot8- | AAGAAGATCAACATTGCACCGC | 1231 | Prkci-g4 | GACACAGATAACTAGACACCCAT |
| | g1 | A | | | |
| 449 | Cnot8- | TTTACAAATTCAGAGCAAACCCC | 1232 | Prkci-g5 | CCAACGATATCAAACGGAGACCT |
| | g2 | | | | |
| 450 | Cnot8- | ACAAACAAGGCAAAACAGCCCA | 1233 | Prlr-g1 | AAACAAACATTTATTGAGCCAGG |
| | g3 | C | | | |
| 451 | Cnot8- | TTTCAAATTCAGTTTCCCACAAG | 1234 | Prlr-g2 | TAATCAAACAGATGACAGCAGAG |
| | g4 | | | | |
| 452 | Cnot8- | AACCACTGTGAAATGAAAGCCA | 1235 | Prlr-g3 | CATTTTACAAATGATGAGCAGCT |
| | g5 | C | | | |
| 453 | Cop1-g1 | ATAAGAGACCATATGGCCAGCA | 1236 | Prlr-g4 | GCAAAACAGAACTAGAACCCAGG |
| | | A | | | |
| 454 | Cop1-g2 | AGCATAAGACAATGTGGCCAAC | 1237 | Prlr-g5 | TTCAGCAGAATAACCCAGGCTTA |
| | | G | | | |
| 455 | Cop1-g3 | GACACATGATTATATCCCAGCAG | 1238 | Prosl-g1 | CATCACAGTAACAAGAGTAGCCT |
| 456 | Cop1-g4 | CCTATCACAAAAATTAGCCACAC | 1239 | Pros1-g2 | GTCACATTCATTTGCCAACCCTC |
| 457 | Cop1-g5 | CTTCAAAATGAGCAGTGAGTCGC | 1240 | Pros1-g3 | CAGAAGCAAGTAAAAGCAGCTTG |
| 458 | Creb1- | TCATTTAGTTACCAACACTCCGC | 1241 | Pros1-g4 | AAAGAGATATTAGAAGAACAGGA |
| | g1 | | | | |
| 459 | Creb1- | AATTAATCTGATTTGTGGCAGTA | 1242 | Pros1-g5 | AGCCTCGTATACATCCATCCAGA |
| | g2 | | | | |
| 460 | Creb1- | AATCAGTTACACTATCCACAGAC | 1243 | Psmg1- | TGCAATTATAAACTTGGAGCAAG |
| | g3 | | | g1 | |
| 461 | Crebl- | TCATTTTCCTCATTTCCCCCAAC | 1244 | Psmg1- | GTAGCACAGATACAGAACCGCAG |
| | g4 | | | g2 | |
| 462 | Creb1- | CTAAGGTTACAGTGGGAGCAGAT | 1245 | Psmg1- | CATTCCAGAGCTTAGCACAACCG |
| | g5 | | | g3 | |
| 463 | Crp-g1 | ATCATGATCAGAAGGCACCAGA | 1246 | Psmg1- | ACAAAACATCAGTGACTGCCCCC |
| | | G | | g4 | |
| 464 | Crp-g2 | AAAAGAACTGATTCTGAGCACA | 1247 | Psmg1- | CAGAAAACATCTGTAGGGGACAG |
| | | G | | g5 | |
| 465 | Crp-g3 | GAAATTTCACAAAGACAGAACC | 1248 | Ptafr-g1 | AACAGATGATGAATACCGCCAAG |
| | | C | | | |
| 466 | Crp-g4 | CCATGAATCGTACTTCAGCACCA | 1249 | Ptafr-g2 | TACAAGTTAGCAAAGACCCATAG |
| 467 | Crp-g5 | AGACACACAGTAAAGGTGTTCA | 1250 | Ptafr-g3 | TACAAAGTTGAGTTCCAGGACAG |
| | | G | | | |
| 468 | Csf1-g1 | TCAGCAGCATAAAGAGACCAAG | 1251 | Ptafr-g4 | AAACCTTAAGTTGATGCCCCAGT |
| | | G | | | |
| 469 | Csf1-g2 | TGCACACATATTTTCAAGACCCA | 1252 | Ptafr-g5 | TCATTGAGAGCACTAGACACCCA |
| 470 | Csf1-g3 | GCCCACAATAAATAGTGGCAGTA | 1253 | Ptar1-g1 | CGCACTTATTCAGATCCACCTCG |
| 471 | Csf1-g4 | TCAGCAAGACTAGGATGATGCCC | 1254 | Ptar1-g2 | CTTGATTAAAGCATCCAACACCA |
| 472 | Csf1-g5 | CATCTATTATGTCTTGTACCAGA | 1255 | Ptar1-g3 | CACCAGAGTTGACAGGCACACTC |
| 473 | Csf1r-g1 | CACACAAGAATATATGCCAGCGT | 1256 | Ptar1-g4 | TCATCTTCTTGATTTCCGCAATC |
| 474 | Csf1r-g2 | ATAGTAAATATAGAGGCTAGCAC | 1257 | Ptar1-g5 | ACCAGAGTTGACAGGCACACTCA |
| 475 | Csf1r-g3 | CATGACAGACATACAGGCCACC | 1258 | Ptger1- | TTAAAGAGCTTTGTAGCCCCACA |
| | | A | | g1 | |
| 476 | Csf1r-g4 | CAGCAGTATTCAGTGATGACCAG | 1259 | Ptger1- | TAGAGAACATGCATGTGCCTCAG |
| | | | | g2 | |
| 477 | Csf1r-g5 | CACTTGAAGAAGTCGAGACAGG | 1260 | Ptger1- | AGCTTCAGAATAGTTCACCAGCT |
| | | C | | g3 | |
| 478 | Csf3-g1 | ACCACACTTTATTATCCGCAAGC | 1261 | Ptger1- | AATCGCTCAAAATTGCACAGCGG |
| | | | | g4 | |
| 479 | Csf3-g2 | CCTTTATACATAAAGCCATCAAG | 1262 | Ptger1- | TATTGCACACTAATGCCGCAAGG |
| | | | | g5 | |
| 480 | Csf3-g3 | CCATAGTGCACTTTGCCACAGCA | 1263 | Ptger2- | TCTTGAATATGAAGCCAGCACCA |
| | | | | g1 | |
| 481 | Csf3-g4 | GATCTAGAAGCTTAGAGCTCCAT | 1264 | Ptger2- | TCCATTAAGCAATCACGAGACAG |
| | | | | g2 | |
| 482 | Csf3-g5 | GAAATACCCGATAGAGCCTGCA | 1265 | Ptger2- | ATCCAAGAAATGGAACCGTGCAC |
| | | G | | g3 | |
| 483 | Cspg4- | CACGTAGATAAAGTTGCCACGCT | 1266 | Ptger2- | GCACCAATTCCGTTACCAGCACG |
| | g1 | | | g4 | |
| 484 | Cspg4- | TTGCCTTAAATTAACCAACCCCA | 1267 | Ptger2- | AAGCGAAATAGGTACACGCGTGA |
| | g2 | | | g5 | |
| 485 | Cspg4- | AGAAAGTTTCATATGGGCCATAG | 1268 | Ptger4- | GCACAATACTACGATGGCCACCA |
| | g3 | | | g1 | |
| 486 | Cspg4- | CTCATACAGAATATTCCCAGCAT | 1269 | Ptger4- | AGTCACATCAGAATGACAGCCAA |
| | g4 | | | g2 | |
| 487 | Cspg4- | CACAGTGATGACAAAGGCCTCA | 1270 | Ptger4- | GTTAATGAACACTCGCACCACGA |
| | g5 | G | | g3 | |
| 488 | Ctla4-g1 | GTGTTTATATTCAAACCACCAGC | 1271 | Ptger4- | CACAGATGATGCTGAGACCCGAC |
| | | | | g4 | |
| 489 | Ctla4-g2 | ATAAAATGAGTGTAAAGACCCA | 1272 | Ptger4- | CCAAGCAATTCACAAGGACACGT |
| | | G | | g5 | |
| 490 | Ctla4-g3 | TCAAAGAAACAGCAGTGACCAG | 1273 | Ptges-g1 | CCATTCCTATTCAAGCCACCACA |
| | | G | | | |
| 491 | Ctla4-g4 | TGACACAACAGAAATATCCCAGC | 1274 | Ptges-g2 | CCACAGAAAATTAAAACGCAGGA |
| 492 | Ctla4-g5 | CAATGACATAAATCTGCGTCCCG | 1275 | Ptges-g3 | CCACCTCTAAAAATAGCAAGCAC |
| 493 | Cxcl1-g1 | CAAGACATACAAACACAGCCTCC | 1276 | Ptges-g4 | GCGTACATCTTGATGACCAGCAG |
| 494 | Cxcl1-g2 | AATGTAAAATAAAAACCACACA | 1277 | Ptges-g5 | CCTATTCTAGAAGAGACACCAAG |
| | | C | | | |
| 495 | Cxcl1-g3 | TTGTATAGTGTTGTCAGAAGCCA | 1278 | Ptgs1-g1 | CTTCATGAGAAACAGCTGCCACA |
| 496 | Cxcl1-g4 | ATGACTTCGGTTTGGGTGCAGTG | 1279 | Ptgs1-g2 | AAGTTTCAAATGTGAGCATCAGC |
| 497 | Cxcl1-g5 | AATACATAAATAAATAGGACCCT | 1280 | Ptgs1-g3 | CAACATTCTGAAGAGGCCTCCAA |
| 498 | Cxcl5-g1 | ATAAAAGTTATATGCCAGCCCAG | 1281 | Ptgs1-g4 | CATTCACAAATTCCCAGAGCCAG |
| 499 | Cxcl5-g2 | AAATATATAGTTAGTGGCCCAAA | 1282 | Ptgs1-g5 | AGAACTCACTATATAGGCCAAAC |
| 500 | Cxcl5-g3 | ACAACAGTAAAAGAGGTCCCCA | 1283 | Ptgs2-g1 | TCATAGTTAAGACAGAGCAGCAC |
| | | T | | | |
| 501 | Cxcl5-g4 | AACAGCAACAGAAATGCCAGCG | 1284 | Ptgs2-g2 | ATATATTTCTTCATTAGACACCC |
| | | G | | | |
| 502 | Cxc15-g5 | GTAATATAAAGAAGTGAGACAC | 1285 | Ptgs2-g3 | TATATTTCTTCATTAGACACCCT |
| | | T | | | |
| 503 | Cybb-g1 | AAGTCAAAACAAGATGAGCGCA | 1286 | Ptgs2-g4 | GCAAACATCATATTTGAGCCTTG |
| | | T | | | |
| 504 | Cybb-g2 | GAACTAGAAGTGTTAGCCACCAT | 1287 | Ptgs2-g5 | TTTATGCGTAAATTCCAACAGCC |
| 505 | Cybb-g3 | AAGTTCAATAACAAAGACACAG | 1288 | Ptk2-g1 | CATATAATATCAAAGATGCCAGG |
| | | G | | | |
| 506 | Cybb-g4 | CCACAAGCATTGAATAGCCCCTC | 1289 | Ptk2-g2 | TTCTACAGATAGTTCGCAACCCA |
| 507 | Cybb-g5 | AGTATAATTATACTTAGGCCCAT | 1290 | Ptk2-g3 | CACAATCATTTGAAGACACCAGA |
| 508 | Cyp19a1- | ATTACGGATAAGTAATGCCCCAG | 1291 | Ptk2-g4 | GCAATAACTCAGAAGGCAGCAGT |
| | g1 | | | | |
| 509 | Cyp19a1- | TATATTTCACTTTTGCCCCCAAA | 1292 | Ptk2-g5 | TGTCATATTCTTTAGCCCAACAC |
| | g2 | | | | |
| 510 | Cyp19a1- | CATTAATGAAGTTTTCCACCACT | 1293 | Ptk7-g1 | ACAACAATAAAGTATGAGGCCAG |
| | g3 | | | | |
| 511 | Cyp19a1- | CATGAAGTACAGAGTGACCGAC | 1294 | Ptk7-g2 | TGCAATAATACAAAGGCCGCCCA |
| | g4 | A | | | |
| 512 | Cyp19a1- | TCATACTTTCTGTAGAGCCAAGA | 1295 | Ptk7-g3 | ACCATTCTGCAAAGGCCCACCAT |
| | g5 | | | | |
| 513 | Dclk1- | CAGGATATAAGTGATCACGCCAG | 1296 | Ptk7-g4 | ACCTCCACACTATTGATGCGCAA |
| | g1 | | | | |
| 514 | Dclk1- | CAAACAAAAATGCCTGAGCCCA | 1297 | Ptk7-g5 | AATGCAAGTGTAGTTGCCAGCAT |
| | g2 | G | | | |
| 515 | Dclk1- | GACTATGTATAAACACAGCAGCT | 1298 | Ptpn6-g1 | ATACAGATTCAGATGACCACAGT |
| | g3 | | | | |
| 516 | Dclk1- | CAGAAAGCAATAAATCCACACC | 1299 | Ptpn6-g2 | TGATCCAGAAAGCTGAGGACACC |
| | g4 | C | | | |
| 517 | Dclk1- | AGAAATTCAAAGAACACCACCA | 1300 | Ptpn6-g3 | ACAAACTCTAGAGATGAGCCTCA |
| | g5 | C | | | |
| 518 | Dcpla- | CCCACAAGAAGATCCAGGCCAC | 1301 | Ptpn6-g4 | CAGAGAGCACAAAATCACCAGGT |
| | g1 | A | | | |
| 519 | Dcp1a- | GCTCAGAAAAGTAAGCAGTGCA | 1302 | Ptpn6-g5 | TACAGGTCATAGAAGTCCCCTGA |
| | g2 | T | | | |
| 520 | Dcp1a- | TAGAGAATAAGACCACTCAGCA | 1303 | Ptprb-g1 | CCCCTTTATCCATTAGCACCAGC |
| | g3 | C | | | |
| 521 | Dcp1a- | AGCATAAATAGAAACCATGGCA | 1304 | Ptprb-g2 | ATGCCAATTAGAAACAGGCCAGC |
| | g4 | G | | | |
| 522 | Dcp1a- | TCAGATGTTTATGACCAGACGGA | 1305 | Ptprb-g3 | AGCACGATACGATAATGCTCCCA |
| | g5 | | | | |
| 523 | Ddit3-g1 | TCAACATGATATAATCGACGTGT | 1306 | Ptprb-g4 | TATCGTATCTTTCTGCCACACCA |
| 524 | Ddit3-g2 | TGTACCGTCTATGTGCAAGCCGA | 1307 | Ptprb-g5 | CCATCCTCAGTTGACAGCCACTG |
| 525 | Ddit3-g3 | ATGAGATATAGGTGCCCCCAATT | 1308 | Ptx3-g1 | GCAGACATTAATCTGAAAGCACC |
| 526 | Ddit3-g4 | ACTCAGCTGCCATGACTGCACGT | 1309 | Ptx3-g2 | TCATTCGTCTATTACGCACCGAA |
| 527 | Ddit3-g5 | CTCTTCAGCTAGCTGTGCCACTT | 1310 | Ptx3-g3 | AAAGAATGAACAATGGGCAACAG |
| 528 | Ddr1-g1 | CCATCCTAATATTTACTCCACAG | 1311 | Ptx3-g4 | AATTCACATACATGAGCTCGTAG |
| 529 | Ddr1-g2 | GTCACCAAGAGTAGCAGCAGCA | 1312 | Ptx3-g5 | ATGCTAATGATTCGTCAAAGCCC |
| | | G | | | |
| 530 | Ddr1-g3 | ATGCACAAAGTTCAGCGTGGCGA | 1313 | Rag2-g1 | AGAATATATGATGTAGCACACCC |
| 531 | Ddr1-g4 | CACTGAAGAGTAACCAAGGACC | 1314 | Rag2-g2 | CATAAGTATAAACCAGAGGCCAC |
| | | T | | | |
| 532 | Ddrl-g5 | CCCATGAGAATGCACTCCCAAGC | 1315 | Rag2-g3 | AGAAACATGAAAAGACAGCCCAT |
| 533 | Dgka-g1 | AGGACCAAAACAGAGCCCAGCA | 1316 | Rag2-g4 | CAAAATTCATAAGTGAGAAGCCT |
| | | T | | | |
| 534 | Dgka-g2 | TACTTACAGAAGTTACAGCTCAG | 1317 | Rag2-g5 | TATGAGATAAAATCTAGCCCAGG |
| 535 | Dgka-g3 | TCATCTTTCATAGTCACGTCCAT | 1318 | Rara-g1 | CCACAGTCAGAAGAGCAGGCAAA |
| 536 | Dgka-g4 | AAGGATGTCTGTATGGGAGCAGT | 1319 | Rara-g2 | CTCCATCTTCAATGTGATCACCC |
| 537 | Dgka-g5 | AAGTTGATCACAGAGACAGCCTG | 1320 | Rara-g3 | CACGTCGAAACATTTCTGCAGCC |
| 538 | Diaph1- | ATTCATATAGTTTCCGACCAGCA | 1321 | Rara-g4 | TCTTGACAAACAAAGCAAGGCTT |
| | g1 | | | | |
| 539 | Diaph1- | CTCAGCCAAGAAATGCAACAGA | 1322 | Rara-g5 | TATCAAAGAGGATGCCACTCCCA |
| | g2 | G | | | |
| 540 | Diaph1- | AGAAAAGCACAAATGCAGCAAG | 1323 | Rel-g1 | TACAGAGTAAGTGTCCAGCCCAA |
| | g3 | A | | | |
| 541 | Diaph1- | TGCAAAACAATTTCTCAGCCAGG | 1324 | Rel-g2 | ATGTGAAATACAAACGCAGCCAT |
| | g4 | | | | |
| 542 | Diaph1- | AGCACCAAATGTCTGCACCCAAC | 1325 | Rel-g3 | TAGCTACTATTTATACAGCACCA |
| | g5 | | | | |
| 543 | Dkk2-g1 | GTCTATAAGAGCTTAAGCAGCAG | 1326 | Rel-g4 | CATGAAGAATAGTAAGGTTCAGC |
| 544 | Dkk2-g2 | ATTATTTACATAGATGAGGCACA | 1327 | Rel-g5 | AAAACTTGAAAACACAGCCTCAC |
| 545 | Dkk2-g3 | TGCACAAATGACAAGGTTCCACC | 1328 | Ret-g1 | TGTATATAGCAAAGGCAACACCA |
| 546 | Dkk2-g4 | AGAGCAAATTTCATGGAGAGCTT | 1329 | Ret-g2 | GATTAAAACAAGACAGACCCACC |
| 547 | Dkk2-g5 | GAAGCAATCAAAGGCCAGACGC | 1330 | Ret-g3 | TCTTTCAGCATTTTCACAGCCAC |
| | | C | | | |
| 548 | Dner-g1 | CACAAGTGTTAAACGCAAAGCC | 1331 | Ret-g4 | CCAAGTCATGAATGGCAGACCCC |
| | | A | | | |
| 549 | Dner-g2 | ACAAACTGACAATAGGTGCCAG | 1332 | Ret-g5 | CAGACACAGAAGATGGACAGCAG |
| | | A | | | |
| 550 | Dner-g3 | CATATCAATCAAATACAGCCACA | 1333 | Rgr-g1 | AATCAAGACTGAATTACGCAAGC |
| 551 | Dner-g4 | AGATCAGCAAAGAATTGGCATCC | 1334 | Rgr-g2 | CATAGAATGTCATAGAACCCCTG |
| 552 | Dner-g5 | CACAGTTAAGACCTTCATAGCCG | 1335 | Rgr-g3 | GAAAGAAATCTAGCAAGCCCCGA |
| 553 | Egf17-g1 | AGTGATATAGAGACTGCAGGCAT | 1336 | Rgr-g4 | TCTCAAGCATTATTGAGCCACAT |
| 554 | Egf17-g2 | ACACTAGAAACCATGCTACAAGC | 1337 | Rgr-g5 | ACATCCACACAAACAGCACCAGA |
| 555 | Egf17-g3 | CATGAATGCTTACAGACACAGTC | 1338 | Rgs1-g1 | GTAGCACTTAAAATAGGAGCACA |
| 556 | Egf17-g4 | ACATGCTCAGTAGTACCATCTGC | 1339 | Rgs1-g2 | CTACTTAGACAAATACCAGCAGC |
| 557 | Egf17-g5 | GAACCTCCGGAAATCCCCACAGT | 1340 | Rgs1-g3 | AATAGCAATAAAATAGGAGACAT |
| 558 | Entpd1- | TAGAAAGCAGAAAACGCCCCAA | 1341 | Rgs1-g4 | TATCAAGTGATGTTACCACAGGC |
| | g1 | A | | | |
| 559 | Entpd1- | ATAGTTAATAGTAATCCACCCAT | 1342 | Rgs1-g5 | CCCCTTTCAAAGATGCCACCATT |
| | g2 | | | | |
| 560 | Entpd1- | ACACAGTATAGTCCTCGCCATAG | 1343 | Ric1-g1 | GTACACATACAGAAGCAGCAGCA |
| | g3 | | | | |
| 561 | Entpd1- | CAAGTTCAGCATGTAGCCCAAAG | 1344 | Ric1-g2 | GATTGTATAAACCTGCACACAGC |
| | g4 | | | | |
| 562 | Entpd1- | AGTCACATTAGCTGCACGAGCAC | 1345 | Ric1-g3 | TGCAAAAAGGTAATTCCACAGAG |
| | g5 | | | | |
| 563 | Entpd2- | AAATTCAGCATGTAGCCCAGCGC | 1346 | Ric1-g4 | CAGCATAATTGATAAGAACCCCC |
| | g1 | | | | |
| 564 | Entpd2- | TCAATCAGAACAGAGGCTCCACT | 1347 | Ric1-g5 | ACTTAAATAAAAATAGCACCAGA |
| | g2 | | | | |
| 565 | Entpd2- | ATGTATGAACATGGCTACAGCGC | 1348 | Ric8a-g1 | CAAAAAGTTAAGAAGAGGCCACT |
| | g3 | | | | |
| 566 | Entpd2- | AGTGACACCAACTTTCCAGCCAT | 1349 | Ric8a-g2 | GAAACACAGCGTATGCCCCACAT |
| | g4 | | | | |
| 567 | Entpd2- | ATAGAGCCGCAAATGGACCTCAT | 1350 | Ric8a-g3 | CTGACAGTATGTAGCCGACCCAA |
| | g5 | | | | |
| 568 | Epcam- | ACCATATTCATTCAGAGAGCAAC | 1351 | Ric8a-g4 | ACATCCAAACACTTGAGGGGCAA |
| | g1 | | | | |
| 569 | Epcam- | TCTAGTGAAACATGCAGCTGCAG | 1352 | Ric8a-g5 | TGAGCACAAGATTACACAGGCAT |
| | g2 | | | | |
| 570 | Epcam- | CTCACGTGCAGAATCAGTCCATC | 1353 | Rock1- | AATAAATTTAAAAGGCAGCACCT |
| | g3 | | | g1 | |
| 571 | Epcam- | CAGCTTGTAGTTGTCACAGACAC | 1354 | Rock1- | AGGTCCAAAAGTTTTGCCCGCAA |
| | g4 | | | g2 | |
| 572 | Epcam- | CACGCCCCTCCCCGCCCTCACCT | 1355 | Rock1- | TTTCATATAGAAATACCCCAACT |
| | g5 | | | g3 | |
| 573 | Epha10- | AAAAAGAGTCAAATTGGAGACA | 1356 | Rock1- | ATGTCCAGACTTATCCAGCAGCA |
| | g1 | G | | g4 | |
| 574 | Epha10- | AGAATGACGAGAAATGCAGCAC | 1357 | Rock1- | TATTTCTCATTAAATGAGCACAG |
| | g2 | C | | g5 | |
| 575 | Epha10- | ACGACATTTCACTATGTGCCCAA | 1358 | Rorc-g1 | TCCAGATCACTTTGACAGCCCCT |
| | g3 | | | | |
| 576 | Epha10- | CTTGTAGTAGACACGCACCGAGA | 1359 | Rorc-g2 | CCAAGAGTAAGTTGGCCGTCAGT |
| | g4 | | | | |
| 577 | Epha10- | ACACAAGCAGCATACCCCACAA | 1360 | Rorc-g3 | CCCAGATGACTTGTCCCCACAGA |
| | g5 | C | | | |
| 578 | Erbb2- | CGACTTTCATATAACACCCACTC | 1361 | Rorc-g4 | ACCACATACTGAATGGCCTCAGT |
| | g1 | | | | |
| 579 | Erbb2- | AGACCATAGCATACTCCAGCACA | 1362 | Rorc-g5 | ATCCTCAGAAAAACACAGGGCGC |
| | g2 | | | | |
| 580 | Erbb2- | ACACAGTGAGTTACAGACCAAG | 1363 | Ros1-g1 | GTCCAATAGAGATAGCCACCAAC |
| | g3 | C | | | |
| 581 | Erbb2- | GCAAAAACGTCTTTGACAACCCC | 1364 | Ros1-g2 | GAAATCCATATGATGCACCCAAG |
| | g4 | | | | |
| 582 | Erbb2- | ACCATCAAACACATCGGAGCCA | 1365 | Ros1-g3 | ACATTGAAAATGGCTGCAGACCT |
| | g5 | G | | | |
| 583 | Ern1-g1 | CACAGCATTTTCATGACACACCC | 1366 | Ros1-g4 | AGTCCAATTTCATTTGCAGCAAC |
| 584 | Ern1-g2 | ATGACATAGTAAAAGACACAGC | 1367 | Ros1-g5 | ACTTCCCAACAAAAGACGCAGGC |
| | | C | | | |
| 585 | Ern1-g3 | AAATCCAATTTTACAGCAGCAGG | 1368 | S100a8- | ACCCACTTTTATCACCATCGCAA |
| | | | | g1 | |
| 586 | Ern1-g4 | AGACAAAAGAATCCTGGCACAG | 1369 | S100a8- | GAAGTCATTCTTGTAGAGGGCAT |
| | | C | | g2 | |
| 587 | Ern1-g5 | AGCAACGTTGATGTGCACCACCT | 1370 | S100a8- | TCTTTGTGAGATGCCACACCCAC |
| | | | | g3 | |
| 588 | Eya3-g1 | TTCACAAACAGACGGCTGCAGAC | 1371 | S100a8- | GTAGACATCAATGAGGTTGCTCA |
| | | | | g4 | |
| 589 | Eya3-g2 | TAAGAATAATGTGCTGAGCTTGG | 1372 | S100a8- | TTTATAGAGGAAAGCTTGGCCAG |
| | | | | g5 | |
| 590 | Eya3-g3 | ACATGCTGAGATTTGACGCAAGG | 1373 | S100a9- | ATTTCCCAGAACAAAGGCCATTG |
| | | | | g1 | |
| 591 | Eya3-g4 | TTATTTGGTGTAGTCTGGGACAA | 1374 | S100a9- | CACAGATGTTGGTAAGAGCAGTG |
| | | | | g2 | |
| 592 | Eya3-g5 | CATACCTGGCCACAGTGCACCGA | 1375 | S100a9- | CATGATGTCATTTATGAGGGCTT |
| | | | | g3 | |
| 593 | F11r-g1 | ACGCCAAAAATCAAGAGTCCAA | 1376 | S100a9- | ACCTCTTAATTACTTCCCACAGC |
| | | G | | g4 | |
| 594 | F11r-g2 | CCCATTACAGTTACAATCCCGAC | 1377 | S100a9- | GCCATCAGCATCATACACTCCTC |
| | | | | g5 | |
| 595 | F11r-g3 | AATGAAGAATTCATGAAGGCCC | 1378 | S100pbp- | TACAAGACTTAGAGGCCAAACCC |
| | | G | | g1 | |
| 596 | F11r-g4 | CCTAGAAGACATTGAAGGCATCC | 1379 | S100pbp- | CATTAAGACAGTACACAGAGCCT |
| | | | | g2 | |
| 597 | F11r-g5 | AGTTTAATTAAACTGTCCCCATG | 1380 | S100pbp- | GCCACATATAAAATGAGACAGAG |
| | | | | g3 | |
| 598 | F8a-g1 | AGCCAAATTAACATGGCAGCAAT | 1381 | S100pbp- | GAAAAATCAGAAGTGCAAGACCA |
| | | | | g4 | |
| 599 | F8a-g2 | AACAGAAATTTGCTGCAACCCAA | 1382 | S100pbp- | GTCTATACTCATTATGCCCACCA |
| | | | | g5 | |
| 600 | F8a-g3 | AGCAAAGATGCATTGATCCAGG | 1383 | S1prl-gl | CATCAATACCTAGTGACAGCCGA |
| | | A | | | |
| 601 | F8a-g4 | ACTGCAATAACAGAAACAGCTCC | 1384 | S1pr1-g2 | CAGTGCAAAATCAAAGCTCCAGG |
| 602 | F8a-g5 | AGCAGCAGTACACGGGACACCT | 1385 | S1pr1-g3 | CATTTGCAACAAGATACGATCCG |
| | | C | | | |
| 603 | Fabp1- | TCATGCACGATTTCTGACACCCC | 1386 | Slpr1-g4 | TCTGATGAACAAAAGTCAGGCAG |
| | g1 | | | | |
| 604 | Fabp1- | TCTTGTAGACAATGTCGCCCAAT | 1387 | S1pr1-g5 | AGCCTTCAGTTACAGCAAAGCCA |
| | g2 | | | | |
| 605 | Fabp1- | ACCATTTTATTGTCACCTTCCAG | 1388 | S1pr4-g1 | AGAACCAAAGATGTCAGCCAGGA |
| | g3 | | | | |
| 606 | Fabp1- | GAGAAGTTCATGGTGGCAACGA | 1389 | S1pr4-g2 | CTATAAAGTGAGTTCCAGGACAG |
| | g4 | G | | | |
| 607 | Fabp1- | TCACCTTCCAGCTTGACGACTGC | 1390 | S1pr4-g3 | ACTGTACACAGAAAGCGTGCCAT |
| | g5 | | | | |
| 608 | Fabp4- | CTTTCATAACACATTCCACCACC | 1391 | S1pr4-g4 | ACATGTGACTAAGACAGAACCAC |
| | g1 | | | | |
| 609 | Fabp4- | TCATATTATTTGTACCAGAACCA | 1392 | S1pr4-g5 | ACAAAAGAGCACATAGCCCTTGG |
| | g2 | | | | |
| 610 | Fabp4- | GATTTCATCGAATTCCACGCCCA | 1393 | Scarb1- | AGGCCATTTAGAAGAGCACACCT |
| | g3 | | | g1 | |
| 611 | Fabp4- | ACAAGTACAAAATTAGACACAC | 1394 | Scarb1- | CATGCCTGAAAAATAAGGACCCT |
| | g4 | A | | g2 | |
| 612 | Fabp4- | TTTGATGCAAATTTCCATCCAGG | 1395 | Scarb1- | CCACAGAGATGACAGAAGCCGAG |
| | g5 | | | g3 | |
| 613 | Fcgr2b- | AAAAACAAAGAGCAGTGTCCAG | 1396 | Scarb1- | AGACGATAGAAAAAGCGCCAGAT |
| | g1 | G | | g4 | |
| 614 | Fcgr2b- | TAATAACAATGGCTGCGACAGCA | 1397 | Scarb1- | TCATAAAAGCACGCTGGCCCATG |
| | g2 | | | g5 | |
| 615 | Fcgr2b- | GTGAATTATAAGCAGTTCCCACG | 1398 | Selplg- | AATCAGCAGACATTGCTTCACCG |
| | g3 | | | g1 | |
| 616 | Fcgr2b- | GCTAACATCCAGAAAGGCCCAGT | 1399 | Selplg- | CCACTGGAATCAGAGAAGCAGAC |
| | g4 | | | g2 | |
| 617 | Fcgr2b- | CAGCAGCAAGATTTAGCACGGCT | 1400 | Selplg- | ATAATATGTTTATTGGATGCCTG |
| | g5 | | | g3 | |
| 618 | Fcr15-g1 | ACAACCAGTAGAGTCAGCCACAT | 1401 | Selplg- | AGAACGCAAGGACAGGTATCCAG |
| | | | | g4 | |
| 619 | Fcr15-g2 | GTAAACTTCTTAGTGGCAGCAAG | 1402 | Selplg- | AAGTGAGTCACGGATGGCCCAAG |
| | | | | g5 | |
| 620 | Fcr15-g3 | ATAACAAGTCAGTGTGACGACCT | 1403 | Sema4d- | GAAAACAGTTTAATACGGCACCT |
| | | | | g1 | |
| 621 | Fcr15-g4 | TAAAACAGAAACACTCCAACAG | 1404 | Sema4d- | AGACACAATAGCTTGGTGCAGTA |
| | | C | | g2 | |
| 622 | Fcr15-g5 | ATACAGGATACATGGAGAGCCC | 1405 | Sema4d- | AAAAGATTCTCACATGGACCCCA |
| | | C | | g3 | |
| 623 | Fgf1-g1 | CAAGCTATATAAGAAGCAACGA | 1406 | Sema4d- | ACGTAGCAAGTTCCTGGCTCCAC |
| | | G | | g4 | |
| 624 | Fgf1-g2 | TCTTGACAAATATATGCCCAGAG | 1407 | Sema4d- | TGCATAGGTACACACGTCTCCAG |
| | | | | g5 | |
| 625 | Fgf1-g3 | ATACCCATTCTTAAAGCACAGTG | 1408 | Serpinb9- | TATCAAGATAGCAAAGAGGCAGT |
| | | | | g1 | |
| 626 | Fgf1-g4 | CCTTTATATACACTTCGCCCGCA | 1409 | Serpinb9- | AAGCAATTACAAGTACAGCGACA |
| | | | | g2 | |
| 627 | Fgf1-g5 | CCTTTCAAGACACAGACCTCCCC | 1410 | Serpinb9- | CAGCAAAATTCTATGATGGCAGA |
| | | | | g3 | |
| 628 | Fitm2-g1 | GTGCAATTTCATATGACAAGCCA | 1411 | Serpinb9- | CATTATAAGATCAGGCTGACAAG |
| | | | | g4 | |
| 629 | Fitm2-g2 | CTTCCACAATCATGAGCGCACAG | 1412 | Serpinb9- | CCAAGCGCTGAAACAGAGACTCC |
| | | | | g5 | |
| 630 | Fitm2-g3 | ATATATACCTTTAATCCCAGCAC | 1413 | Sh3bp2- | AGTCACATGTAGTAATCCCGAAG |
| | | | | g1 | |
| 631 | Fitm2-g4 | AAACCAAACATGGTGCCGAACA | 1414 | Sh3bp2- | GTCAGCAAATTCTGGGCACCAAT |
| | | C | | g2 | |
| 632 | Fitm2-g5 | AACTCTAAAGAGAAGCAGAGCC | 1415 | Sh3bp2- | CCAGCAGGTGATATTCAGAGCCC |
| | | G | | g3 | |
| 633 | Fos-g1 | AATGTCAGAACATTCAGACCACC | 1416 | Sh3bp2- | ACTGCATCTCCTCAGCCGCCATG |
| | | | | g4 | |
| 634 | Fos-g2 | GCACTAGATACAATCCAGCACCA | 1417 | Sh3bp2- | CTTGCTCCCAGCCCAGCGCCTAG |
| | | | | g5 | |
| 535 | Fos-g3 | TCCACATGTCGAAAGACCTCAGG | 1418 | Shc1-g1 | ATTTTCCATTATAAGAACCCACC |
| 636 | Fos-g4 | TCTCCTCTCTGTAATGCACCAGC | 1419 | Shc1-g2 | CAAACCAAAAATTTGGCGACCAT |
| 637 | Fos-g5 | AAGACGTGTAAGTAGTGCAGCCC | 1420 | Shc1-g3 | CATTGACTGTAAGACCTCCACAC |
| 638 | Fosl1-g1 | TCTACAGACAGAAAGCGACCGA | 421 | Shc1-g4 | AAGAAGTCACCATTGAGCTGCAG |
| | | C | | | |
| 639 | Fosl1-g2 | TGTGCAAATTATGATAGGCCAGC | 1422 | Shc1-g5 | GAAGCCTCATATCTACCACCCCA |
| 640 | Fosl1-g3 | AGGAAAATAAATAGCAGCTGCC | 1423 | Shh-g1 | AGAAAAATAGACTTTCAGCAGGT |
| | | C | | | |
| 641 | Fosl1-g4 | ATAACATGAAGAAAGTGACGTG | 1424 | Shh-g2 | ACGAAACAAATAAATAGCCAGGA |
| | | G | | | |
| 642 | Fosl1-g5 | ATGAATGAAAAGTTCTTGGGCTG | 1425 | Shh-g3 | AAATATAATTTGTGGACCCCCAT |
| 643 | Foxm1- | CAAGCAGAAGCAAAGTGCAACC | 1426 | Shh-g4 | GATTCATAGTAGACCCAGTCGAA |
| | g1 | C | | | |
| 644 | Foxm1- | ATAGATATGAATCATCGCAACAG | 1427 | Shh-g5 | TTAAAAGACAAAAAGAGCCTGAT |
| | g2 | | | | |
| 645 | Foxm1- | ACCAGCATTTCTGAGACACAGCG | 1428 | Sirpa-g1 | CACACAGTAGTAGATGCCAGCAT |
| | g3 | | | | |
| 646 | Foxm1- | TGATAGTATCAAGCTAGCCCGAG | 1429 | Sirpa-g2 | AAACTGTAGATCAACAGCCGGCT |
| | g4 | | | | |
| 647 | Foxm1- | CAGAACTCATCTTTCCAAGCCAC | 1430 | Sirpa-g3 | AACATTTCTAATTCGAGGAACGT |
| | g5 | | | | |
| 648 | Fut4-g1 | TATGTCTAAACTTTAGCAGCCAC | 1431 | Sirpa-g4 | CAGTTCAGAACGGTCGAATCCCC |
| 649 | Fut4-g2 | CCAGGTTAAATTTCAGCCCCAAC | 1432 | Sirpa-g5 | AGTCACCTTCAGTTCCTTCCCCG |
| 650 | Fut4-g3 | CACACCTTTAATTCCCACCCAAG | 1433 | Skap1- | AGAAACTTATTTGATCCACCCAG |
| | | | | g1 | |
| 651 | Fut4-g4 | CGATTCGAAGTTCATCCACACCC | 1434 | Skap1- | TTTATTTCTGCTTTGCAAGCCAC |
| | | | | g2 | |
| 652 | Fut4-g5 | GGTATTTAGAAAACGCAGCCAA | 1435 | Skap1- | TAATAATCGGCATAGTCCACTCG |
| | | G | | g3 | |
| 653 | Fyb-g1 | CATGTTTATTGGAAAGAGCCGAT | 1436 | Skap1- | ACCAAAAGGATGCTTCTGACGTG |
| | | | | g4 | |
| 654 | Fyb-g2 | TGATTGTATTCCAGAAGGCGAGC | 1437 | Skap1- | CTTAAAGTCACAGGAAGCAGCAG |
| | | | | g5 | |
| 655 | Fyb-g3 | TGGTCCCAAATTTAGGCATGCTG | 1438 | Snip1-g1 | TACAAAGAGAAGAAGCCACCATC |
| 656 | Fyb-g4 | TCCAGTAGGTTTTAAAAACGGGG | 1439 | Snip1-g2 | ACTGACATATGAAACCCAGCACA |
| 657 | Fyb-g5 | TCTAGAAACTGAGTTTGGCTGTG | 1440 | Snip1-g3 | GTTCAGAAAACAAGGGCCCACAA |
| 658 | Gale-g1 | TTCTGCTTCTGCCAGCGCCACAG | 1441 | Snip1-g4 | CACTGTATTTAATAACCACACCC |
| 659 | Gale-g2 | GTTAACTCTATAGTAGTCCAGAG | 1442 | Snip1-g5 | GTCTGTTTATCGAAGACAGCCAA |
| 660 | Gale-g3 | CACGTGAATGTAATCCCTCACAC | 1443 | Socs2-g1 | GCCATTTGATCTTGAGCAGCCAT |
| 661 | Gale-g4 | AAAGCTTAATCATCAGTGCCTGG | 1444 | Socs2-g2 | ATAAATACAAGCATGGTCAGCTT |
| 662 | Gale-g5 | ATTGAAGTACCGAAGCAGCACG | 1445 | Socs2-g3 | AACACTTTGAAAAACAGGCAGGT |
| | | G | | | |
| 663 | Gch1-g1 | TAAAGAGCACTATGTCAGCCAGA | 1446 | Socs2-g4 | ACAGATCGCCATTTAACCCCGAG |
| 664 | Gch1-g2 | ATACATACAGTAAATCCACACAG | 1447 | Socs2-g5 | GTAGTCAATCAGATGAACCACAC |
| 665 | Gch1-g3 | ATTAAAGGCAGATATCCCACCAC | 1448 | Sox2-g1 | CTGCAGAATCAAAACCCAGCAAG |
| 666 | Gch1-g4 | ACTGAGACAGATAACAGCCGCA | 1449 | Sox2-g2 | GCCTGATTCCAATAACAGAGCCG |
| | | G | | | |
| 667 | Gch1-g5 | AAGATAGCCAATATGGACCCTTC | 1450 | Sox2-g3 | ATTACCAACGATATCAACCTGCA |
| 668 | Gcnt1- | AGCAAAAATGAAGACAGAGCCA | 1451 | Sox2-g4 | CTGTACAAAAATAGTCCCCCAAA |
| | g1 | T | | | |
| 669 | Gcnt1- | TAACATGAATACTCAAGGCCAGA | 1452 | Sox2-g5 | TCGGACAAAAGTTTCCACTCCGC |
| | g2 | | | | |
| 670 | Gcnt1- | CAAAAGAAAGAAATCAGAAGCC | 1453 | Sox9-g1 | GAAAACATTGCAAAGGACTCAGT |
| | g3 | A | | | |
| 671 | Gcnt1- | AATATTTTCATTTTCCAGCACGT | 1454 | Sox9-g2 | AACAGCATATACTTTCCCAGCAG |
| | g4 | | | | |
| 672 | Gcnt1- | ACATTATCAAAGCAGGATGCGAT | 1455 | Sox9-g3 | TTAACAACAGATGACCATACCCT |
| | g5 | | | | |
| 673 | Gdf15- | GTAGACATTACAGCCGTGCCCAA | 1456 | Sox9-g4 | TGCAAAGAAAAGTTCCTGGACTG |
| | g1 | | | | |
| 674 | Gdf15- | AAATACACAATCCATCCACCCAG | 1457 | Sox9-g5 | TCGCTCTCGTTCAGCAGCCTCCA |
| | g2 | | | | |
| 675 | Gdf15- | ACAGAACATGTGATGGAGTCCA | 1458 | Spn-g1 | CACAACAGCAGTAGCGCCACGAG |
| | g3 | G | | | |
| 676 | Gdf15- | AGAAAAGGCAAGTTCAGGCAAG | 1459 | Spn-g2 | AACCACATGAATACCTGGTGCCC |
| | g4 | T | | | |
| 677 | Gdf15- | CATAAGTCTGCAGTGACACACCA | 1460 | Spn-g3 | ACAGCTTCATCCTCACTGCCCAC |
| | g5 | | | | |
| 678 | Gls-g1 | TGCAGACCATTATAGCAACCCGT | 1461 | Spn-g4 | TCTCCAGTACCTCAGAGCCCTTG |
| 679 | Gls-g2 | TATTGAATGATACAGCCCACCAA | 1462 | Spn-g5 | TCTTCTAGTACTAAAGAGCCCTG |
| 680 | Gls-g3 | ACTGAATCACAAAAAGCCAGCC | 1463 | Sqstm1- | ACTTATAGCGAGTTCCCACCACA |
| | | G | | g1 | |
| 681 | Gls-g4 | CCAACATATTCATTACCAGCCAT | 1464 | Sqstm1- | ACAAACACTAAAGAGTGGCCATT |
| | | | | g2 | |
| 682 | Gls-g5 | CAACTAAAAGAATACCCCCAGC | 1465 | Sqstm1- | CAAACCAAGTCAGAGGAAGCTAC |
| | | A | | g3 | |
| 683 | Gpc2-g1 | ATCAGCAGTTGAAGTAAGCCGA | 1466 | Sqstm1- | GTTACACAAGTTAATGCCCCATC |
| | | G | | g4 | |
| 684 | Gpc2-g2 | CCTAAAACTGAACAACCCCAGCA | 1467 | Sqstm1- | TTTTGAAGACAAATGTGTCCAGT |
| | | | | g5 | |
| 685 | Gpc2-g3 | AGATGATTTTAAAAGAGGCAAC | 1468 | Srrt-g1 | TCCAACTACAAAACAAGACCCCC |
| | | A | | | |
| 686 | Gpc2-g4 | AAAGATTTAAAAGAGCTGAACC | 1469 | Srrt-g2 | AAAGTTATTGAAGAACGCCACCT |
| | | A | | | |
| 687 | Gpc2-g5 | ACTTTCAAGTTCCTTCAACCCCG | 1470 | Srrt-g3 | AGAAGGTTATCAAACCAGCCACT |
| 688 | Gpi1-g1 | CACCACCAAGTAAAGAGCCAAC | 1471 | Srrt-g4 | CGAAAAGCATCATAGTTCCCACG |
| | | C | | | |
| 689 | Gpi1-g2 | AATGTTAGAGACAAACCAGACA | 1472 | Srrt-g5 | TGCCATTTATGTTGCGGACACGA |
| | | C | | | |
| 690 | Gpi1-g3 | CTCATAACGATCAATCCTCCGAG | 1473 | Susd2- | CCACTTTAAATGTTGCACCACGA |
| | | | | g1 | |
| 691 | Gpi1-g4 | AACAACATGACACGTCAAAGCC | 1474 | Susd2- | ACCAATAGACAAAGGAAGTGCAG |
| | | C | | g2 | |
| 692 | Gpi1-g5 | CGACAAAGTGCTTTGCAACTGCA | 1475 | Susd2- | CATAGAGCCAATGAGACATGCCG |
| | | | | g3 | |
| 693 | Gpnmb- | CACAAAAGTGATATTGGAACCCA | 1476 | Susd2- | AGCAGCATCAAAATCCAAGGCAG |
| | g1 | | | g4 | |
| 694 | Gpnmb- | AGTAGAATATGTACACACACGCA | 1477 | Susd2- | AAGATGATACTCAGTAGCACCGT |
| | g2 | | | g5 | |
| 695 | Gpnmb- | ATTTTCATCCGAAGACCAGCCAC | 1478 | Tacstd2- | CAAGCAGAAAAATAGATGCAGTC |
| | g3 | | | g1 | |
| 696 | Gpnmb- | CTTAGAGAACAACAGTTCCCAGC | 1479 | Tacstd2- | CTGAGAATTAACAGGCCAACCCA |
| | g4 | | | g2 | |
| 697 | Gpnmb- | GCAGACATTTATAAAAGCCCTAG | 1480 | Tacstd2- | AGGAATTTCAGAAATGCGTCCTT |
| | g5 | | | g3 | |
| 698 | Gpr68- | ACTGACAAAGCAGTACAGCACC | 148 | Tacstd2- | CCCACCGAGTTTACGCACCAGCA |
| | g1 | G | | g4 | |
| 699 | Gpr68- | AACAGATATAGAACAGGTCTGC | 1482 | Tacstd2- | CCCCCAGCTCCTTAAGCTCCACC |
| | g2 | A | | g5 | |
| 700 | Gpr68- | GCACAGAACACTTAGCTATCACC | 1483 | Tdo2-g1 | CAAATAAATCAATAGAGGCCAAG |
| | g3 | | | | |
| 701 | Gpr68- | GAAGTCACTACATAAACCCACCT | 1484 | Tdo2-g2 | GAACAAAATGCTTTACGACAGCC |
| | g4 | | | | |
| 702 | Gpr68- | AGACTAGTTTAGAGGCAAGCTCA | 1485 | Tdo2-g3 | ATCAAACAAGCAGAGCAGCACCT |
| | g5 | | | | |
| 703 | Havcr2- | CCAAAGTCAGAAATGAAGGCGA | 1486 | Tdo2-g4 | ACAAGCAATGAACAGCCAACCAC |
| | g1 | G | | | |
| 704 | Havcr2- | AGACACCAATGATAAGTGCCAG | 1487 | Tdo2-g5 | CATGCGTATTACAGTGCAGCGAA |
| | g2 | G | | | |
| 705 | Havcr2- | CCCACCTAAGAAAGCCAGGACCT | 1488 | Tek-g1 | ACATAATCAGAAACGCCAACAGC |
| | g3 | | | | |
| 706 | Havcr2- | AGTCCTTAATTTCATCAGCCCAT | 1489 | Tek-g2 | CCACAGAGAACTAAGCCGGCTAA |
| | g4 | | | | |
| 707 | Havcr2- | TATAGTGTTAAGCATATGCCACC | 1490 | Tek-g3 | ACTAAAATGTTTCTGGCAGCCAG |
| | g5 | | | | |
| 708 | Hbegf- | TTACAGAGCAAATAGGACCCAG | 1491 | Tek-g4 | GAAGAAATCGAATAGCCATCCAC |
| | g1 | C | | | |
| 709 | Hbegf- | ACAAAGTATAAATATGAACCAG | 1492 | Tek-g5 | CCCCAAAGTAAGGCTCAGAGCTG |
| | g2 | G | | | |
| 710 | Hbegf- | ACGACAGTACTACAGCCACCACA | 1493 | Tgfb2- | CAATACATAAAATACAGGCAGAG |
| | g3 | | | g1 | |
| 711 | Hbegf- | CCAGAAAGAGCTTCAGCATCACC | 1494 | Tgfb2- | ATTTCTAAAGCAGTAGGCAGCAT |
| | g4 | | | g2 | |
| 712 | Hbegf- | ACAGAGCAATTACAGGAGGCCC | 1495 | Tgfb2- | TGTATTGTAGATCAACAGCCACT |
| | g5 | A | | g3 | |
| 713 | Hcar1- | TCCAAACAACGTTGACCGAGCAG | 1496 | Tgfb2- | AGAACCCTTAAAATAGCAGTCAG |
| | g1 | | | g4 | |
| 714 | Hcar1- | AGCAAAATATCGTGGCGCCAGC | 1497 | Tgfb2- | AAAGAAAATGCAACGCGTTCCCA |
| | g2 | A | | g5 | |
| 715 | Hcar1- | CAGAAGATACACAGTCCCCAAG | 1498 | Tgfbr1- | AATAAGACATTAACAGAGCCCAG |
| | g3 | A | | g1 | |
| 716 | Hcar1- | TCATGTGAAAGCAGAAGCCGCA | 1499 | Tgfbr1- | CCACCAATAGAACAGCGTCGAGC |
| | g4 | C | | g2 | |
| 717 | Hcar1- | GCACCAAAGACAAAACAGACGA | 1500 | Tgfbr1- | AGCATAAGTGCAATGCAGACGAA |
| | g5 | G | | g3 | |
| 718 | Hcst-g1 | GCATACATACAAACACCACCCCT | 1501 | Tgfbr1- | AAGAAGTATCCATAGTGCACAGA |
| | | | | g4 | |
| 719 | Hcst-g2 | ACAATTAGGAGTGACATGACCGC | 1502 | Tgfbr1- | GCTTCATTTAGTGCCACACCCCA |
| | | | | g5 | |
| 720 | Hcst-g3 | GCATGTTGATGTAGACTCTACCA | 1503 | Ticam2- | CTACATAGCAAATTTCAGGCCAG |
| | | | | g1 | |
| 721 | Hcst-g4 | GCAGAGACAGAGTCCCACATCC | 1504 | Ticam2- | AAAATGCTATGAAAGCAGTCACC |
| | | G | | g2 | |
| 722 | Hcst-g5 | AGAGTCCCACATCCGGAGCAGG | 1505 | Ticam2- | TAAAATTCATGAAAGAAGCAGGA |
| | | A | | g3 | |
| 723 | Hdac11- | TCAAACAGGAACTTGATAGCCAG | 1506 | Ticam2- | CCCAGATCCAAGATGACACCCAT |
| | g1 | | | g4 | |
| 724 | Hdac11- | CCTCTTTAGCAAAGCGATCCCCA | 1507 | Ticam2- | AATGAACTGTTTCTGCGACACAC |
| | g2 | | | g5 | |
| 725 | Hdac11- | ACCACCAACATTGATGGCCCAGC | 1508 | Tigit-g1 | TTAAGCAAATGAGTCCCAGCACA |
| | g3 | | | | |
| 726 | Hdac11- | CCACAATGACAAACTGTGCTGTC | 1509 | Tigit-g2 | GTCAACACTATAAATGGCCAGAA |
| | g4 | | | | |
| 727 | Hdac11- | CATGGTAATGAATATGAGTCCTG | 1510 | Tigit-g3 | ACACTGTAAGATGACAGAGCCAC |
| | g5 | | | | |
| 728 | Hdac4- | AAGAATCAAATGTTAAGCACCA | 1511 | Tigit-g4 | CACTGAAGACTGAAGCGACATGC |
| | g1 | G | | | |
| 729 | Hdac4- | CCACATTCACTAATGCCAACCAC | 1512 | Tigit-g5 | GATACAGCAATGAAGCTCTCTAG |
| | g2 | | | | |
| 730 | Hdac4- | AAAACCAAGCATGATAGCAGCG | 1513 | Tm4sf5- | AGGTACATATAGAAGCCCCAAGT |
| | g3 | C | | g1 | |
| 731 | Hdac4- | CATGTTGACATTGAAACCCACGC | 1514 | Tm4sf5- | AGACACATATCTCTGAAGGCCCA |
| | g4 | | | g2 | |
| 732 | Hdac4- | ACACACTCCAAATTTCCACCAGC | 1515 | Tm4sf5- | TTCCAGACTTGATGCGACCACCA |
| | g5 | | | g3 | |
| 733 | Hgf-g1 | CTGCATAAATAAGTAGCCCAGAT | 1516 | Tm4sf5- | TCACCAGCTGAATTCCACACAGC |
| | | | | g4 | |
| 734 | Hgf-g2 | ACTAGAAGCCAATTGCAGCAGC | 1517 | Tm4sf5- | CCCCCTCCAATGAAGCCACCCAT |
| | | A | | g5 | |
| 735 | Hgf-g3 | AATTTGAGAGCAGTAGCCAACTC | 1518 | Tnc-g1 | ATGACAAAGTACTCATAGGCCAG |
| 736 | Hgf-g4 | CTGATCCAATCTTTTCAGCCCCA | 1519 | Tnc-g2 | ACGACAAAGTGCTTATAGGCCAG |
| 737 | Hgf-g5 | CCTTTATCAATGATCCTCCACAG | 1520 | Tnc-g3 | CCTTAGAGACGATACCAGCAACT |
| 738 | Hmox1- | AACAAGACAGAAATACGAGACA | 1521 | Tnc-g4 | AAGCAATGTCTAGAGGATCCCAC |
| | g1 | G | | | |
| 739 | Hmox1- | TCACACAGAAGTTAGAGACCAA | 1522 | Tnc-g5 | CAATGCTATCAATTTCAGCCAAG |
| | g2 | G | | | |
| 740 | Hmox1- | CCCAAGAGAAGAGAGCCAGGCA | 1523 | Tnfrsf11b- | CCATCTTGAAGAAACAGCCCAGT |
| | g3 | A | | g1 | |
| 741 | Hmox1- | TACATGGCATAAATTCCCACTGC | 1524 | Tnfrsf11b- | GACACATAAAATAGTAGAAGCCA |
| | g4 | | | g2 | |
| 742 | Hmox1- | GTCAGCATCACCTGCAGCTCCTC | 1525 | Tnfrsf11b- | TGCTATGAAACCAAGCCAGCCAT |
| | g5 | | | g3 | |
| 743 | Hspa13- | ATACAAGCAGAAAATGACGCCC | 1526 | Tnfrsf11b- | TTCAAGCAGAATTCGATCTCCAG |
| | g1 | T | | g4 | |
| 744 | Hspa13- | TTTATATAAATGCTGAAGCAGCC | 1527 | Tnfrsf11b- | ACAGCAAACCTGAAGAAGGCCTC |
| | g2 | | | g5 | |
| 745 | Hspa13- | AGAAATGACAGCATTGGCAACC | 1528 | Tnfrsf17- | CCCAAGAAGATCCAGAGCACCGT |
| | g3 | G | | g1 | |
| 746 | Hspa13- | AAATAAAGAAATTATCAGCCCCT | 1529 | Tnfrsf17- | TAACGACATCTAAAACACCAGCT |
| | g4 | | | g2 | |
| 747 | Hspa13- | TCACAGAAAACTCAGCCATCCCA | 1530 | Tnfrsf17- | AGAAAATCGAGGAAGAACAGCAG |
| | g5 | | | g3 | |
| 748 | Id1-g1 | GTAAAACAATATTTTCAGCCAGT | 1531 | Tnfrsf17- | AAAAGTGCCAAAGAGAGGACCAA |
| | | | | g4 | |
| 749 | Id1-g2 | TTCAATAAAACAGAAACACGCG | 1532 | Tnfrsf17- | ACACTTTGCAAAGCAGTTGGCAC |
| | | G | | g5 | |
| 750 | Id1-g3 | CGACAGACCAAGTACCACCTCGC | 1533 | Tnfrsf1a- | ATGAAGTAAGATGATCGGACCAG |
| | | | | g1 | |
| 751 | Id1-g4 | CAGCGACACAAGATGCGATCGTC | 1534 | Tnfrsf1a- | GCAGCAATTGACAACGCTCGTGA |
| | | | | g2 | |
| 752 | Id1-g5 | GTAGTCGATTACATGCTGCAGGA | 1535 | Tnfrsf1a- | CAATTTCACGGAAGGAAGCCAGC |
| | | | | g3 | |
| 753 | Id3-g1 | CCCCCTTATTCAAAACAGACCGC | 1536 | Tnfrsf1a- | ACATACTTTCCTTGGGGACACAA |
| | | | | g4 | |
| 754 | Id3-g2 | ACCTAAAGCAGCAAACAGTGCG | 1537 | Tnfrsf1a- | ATCAGCAGAGCCAGGAGCACCAG |
| | | C | | g5 | |
| 755 | Id3-g3 | AGTTCATAATCAGGGCAGCAGA | 1538 | Tnfrsf1b- | ACAGCAAGTACAGTACCAAGCCG |
| | | G | | g1 | |
| 756 | Id3-g4 | CATGATTACAGAAAGTCACCTTC | 1539 | Tnfrsf1b- | CAGAGTAAAAGTCAAAGGCAGAG |
| | | | | g2 | |
| 757 | Id3-g5 | GATGTAGTCTATGACACGCTGCA | 1540 | Tnfrsf1b- | CAGTCCTAACATCAGCAGACCCA |
| | | | | g3 | |
| 758 | Ido1-g1 | ATTTCCACCAATAGAGAGACGAG | 1541 | Tnfrsf1b- | CTCAGAAGCAAGAATCAGGCAAG |
| | | | | g4 | |
| 759 | Ido1-g2 | AGACAGATATATGCGGAGAACG | 1542 | Tnfrsf1b- | GTACAGGAAGAACTGCAGCTCAA |
| | | T | | g5 | |
| 760 | Ido1-g3 | AATCTACATAATATACAACAGGC | 1543 | Tnfrsf8- | CAAAAATTGTGTGAAGAGCCACT |
| | | | | g1 | |
| 761 | Ido 1-g4 | GCATAAGACAGAATAGGAGGCA | 1544 | Tnfrsf8- | TACAAGAGTATGCAGCTGCCAGT |
| | | G | | g2 | |
| 762 | Ido 1-g5 | AACCTCAAAACCAGGCACGCCA | 1545 | Tnfrsf8- | GGAGAAATTTAAAGGGCACACAG |
| | | G | | g3 | |
| 763 | Igflr-g1 | CTAACATAGAACTGAGAGACCC | 1546 | Tnfrsf8- | GCAAAGCATAGTCTTGAGCAGTG |
| | | A | | g4 | |
| 764 | Igflr-g2 | TAATTTAAATATTTCCACCCAGA | 1547 | Tnfrsf8- | AGAACATGACCTCAGTGCAGCTG |
| | | | | g5 | |
| 765 | Igf1r-g3 | ACATACAGCATGATAACCAGCCC | 1548 | Tnfsf11- | TGGAATTCAGAATTGCCCGACCA |
| | | | | g1 | |
| 766 | Igflr-g4 | CAATGTAGTTATTGGACACCGCA | 1549 | Tnfsf11- | AAGAACTTATTTGCAGGTCCCAG |
| | | | | g2 | |
| 767 | Igflr-g5 | TTAATAAGCAGATTGCCCTTCAG | 1550 | Tnfsf11- | CGAAAGCAAATGTTGGCGTACAG |
| | | | | g3 | |
| 768 | Igfbp2- | AATCTTAAGTAAAAGAGACACA | 1551 | Tnfsf11- | AATAAACTACATGTGGTCACCAG |
| | g1 | G | | g4 | |
| 769 | Igfbp2- | CCCAACATGTTCATGGTCCCATC | 1552 | Tnfsf11- | TCGAAAGTACAGGAACAGAGCGA |
| | g2 | | | g5 | |
| 770 | Igfbp2- | CGTCATCACTGTCTGCAACCTGC | 1553 | Trem1- | GTCAATAATAAAATGCACACAGC |
| | g3 | | | g1 | |
| 771 | Igfbp2- | AACAGAAGCAAGGGAGGTTCAG | 1554 | Treml- | CTGAGACAAAGAACACGCACAGC |
| | g4 | C | | g2 | |
| 772 | Igfbp2- | AGAGCAGCAGCAAGAGCAACGA | 1555 | Trem1- | TAACTAGTCACTAAGCCCCAGAG |
| | g5 | C | | g3 | |
| 773 | Ikbkg-g1 | CAGAGAAGATTCTTCACCCAGCA | 1556 | Trem1- | CCTAGAGAAAGCACAGGAGCCAC |
| | | | | g4 | |
| 774 | Ikbkg-g2 | AGCAGCTCCTCACAGCGTTCCCT | 1557 | Trem1- | ATATAAGATACGTGGTTCAGCCA |
| | | | | g5 | |
| 775 | Ikbkg-g3 | GATATACATGTACTTGTGTCACA | 1558 | Trex1-g1 | CACAGAGAGAGCTTGTCCACCAC |
| 776 | Ikbkg-g4 | GAATTTGCACATAAGGAACTCCT | 1559 | Trex1-g2 | CTGTCCCTCCCTCTCAGCCACAC |
| 777 | Ikbkg-g5 | ATTTCATCTTGAAGCAGTGACAC | 1560 | Trex1-g3 | CCACACAGAAGGTACCATCTAGG |
| 778 | IL10ra- | GATTCCACAGAATAGCAGCATAG | 1561 | Trex1-g4 | CAGAGGAAAGTCATAGCGGTCAC |
| | g1 | | | | |
| 779 | IL10ra- | CAGGACCTAAACTATCACCCCAG | 1562 | Trex1-g5 | TTCCACTGACAGATGCTGAGCAG |
| | g2 | | | | |
| 780 | IL10ra- | AAGCAGACATAAGTCCTAAGCCT | 1563 | Trpm4- | CCCATAAGCTACAAGCCACACGC |
| | g3 | | | g1 | |
| 781 | IL10ra- | TATTAGGATGAAAACACACACCA | 1564 | Trpm4- | AAGCAGAATAGTTATAGTCCAGG |
| | g4 | | | g2 | |
| 782 | IL10ra- | GTTTCAAATAACCTGCGGCCAGA | 1565 | Trpm4- | CAAGAAGACGATAAGGAGCAACA |
| | g5 | | | g3 | |
| 783 | Il11-g1 | AACAGATTTCATAGAGACCCCAG | 1566 | Trpm4- | CCCAGAAGTACAGCAGCAGCTCG |
| | | | | g4 | |
| 784 | Il11-g2 | ATAAGTATAAATAAGCCACAGGT | 1567 | Trpm4- | AAGATCATGAAGTCAAGGCAGAG |
| | | | | g5 | |
| 785 | Il11-g3 | AGTCTTTAACAACAGCAGGCCCC | 1568 | Trpm7- | AATGTAGAACATATTGGCCACCA |
| | | | | g1 | |
| 786 | Il11-g4 | CACAAACAGTAACAGTCACCCAC | 1569 | Trpm7- | AAAAACTCAATTTTGGCACAGAG |
| | | | | g2 | |
| 787 | Il11-g5 | CACATTTTGGTATAAAACCCCAG | 1570 | Trpm7- | CATCAATAAGATTCTGAGCCAAG |
| | | | | g3 | |
| 788 | Il16-g1 | ACAACCATTTTATTACGGCACCA | 1571 | Trpm7- | TACAAGAGCATCAAGCATAGCCT |
| | | | | g4 | |
| 789 | Il16-g2 | GAACATTTAAAAGATGCACCCGC | 1572 | Trpm7- | CCTTCAAATATCAAAGCCACCAC |
| | | | | g5 | |
| 790 | Il16-g3 | ATCAAAGCTATAGTCCATCCGTG | 1573 | Txk-g1 | CTATAAACATTTATACAGCCCCA |
| 791 | Il16-g4 | CCAAAGTTTTCAAAGGAGCTGAT | 1574 | Txk-g2 | AAAGAGAAATGTCAGCGCTCAAG |
| 792 | Il16-g5 | AACTATCATCTAATACCAAGCAG | 1575 | Txk-g3 | CCCACATTAAAACTCCGAACGAC |
| 793 | Il18bp- | TGTAAAGATTGAAGTCAGTGCAG | 1576 | Txk-g4 | GAGATCTTTACTACGCAGGCAGA |
| | g1 | | | | |
| 794 | Il18bp- | ACATACAAAAGCAGGACCCACC | 1577 | Txk-g5 | ACGAGATATGAGACCAGCTGCAT |
| | g2 | A | | | |
| 795 | Il18bp- | CACGTTAAGTGTAAGCAGAGCTA | 1578 | Tymp-g1 | AAAACCGAAGTACATCAGCACCC |
| | g3 | | | | |
| 796 | Il18bp- | ATATACAGTTGTGACCTGACGCA | 1579 | Tymp-g2 | CAAACTTAACATCTACCACCAGA |
| | g4 | | | | |
| 797 | Il18bp- | TCAATGAAGGAACCATTGCCCAG | 1580 | Tymp-g3 | CGCTAATCCAAAGAATGGCGCCT |
| | g5 | | | | |
| 798 | Il1r1-g1 | CTGAAATCAAAAAGTCAGAGCA | 1581 | Tymp-g4 | CACAGTGATGAAGACAGGCTGCA |
| | | C | | | |
| 799 | Il1r1-g2 | GAAAAGTAAGTTTTCCAGACACC | 1582 | Tymp-g5 | TCCATCTATCACCGCGTGCACGA |
| 800 | Il1r1-g3 | TTACTCAATAAAATGAAGGCCAC | 1583 | Typo3- | CAGTATACAAGTIGTCAGCCAAG |
| | | | | g1 | |
| 801 | Il1r1-g4 | TGACATAAAACTAACCAGCACA | 1584 | Typo3- | CATGCACTAAAGTAGGAGCAGCT |
| | | G | | g2 | |
| 802 | Il1r1-g5 | CCATGAGACAAATGAGCCCCAGT | 1585 | Typo3- | AGAGTCCAAAATCAGCCACACAC |
| | | | | g3 | |
| 803 | Il23r-g1 | GTAAGAATTAATAGATGGCAAGT | 1586 | Typo3- | TACAGTACAGGAATGCAGCAGAG |
| | | | | g4 | |
| 804 | 1l23r-g2 | AAACAAGCAAACAAGCACAGCC | 1587 | Typo3- | TCTTAATGATAACAGCAGTGCTG |
| | | T | | g5 | |
| 805 | 1l23r-g3 | AAGTAAGAATTAATAGATGGCA | 1588 | Usp15- | CAAAACAAATGCCAGGTGCCTGA |
| | | A | | g1 | |
| 806 | Il23r-g4 | AAGAGCACATAAAGGGCTATCA | 1589 | Usp15- | CCACTAGAATGTCAGCATCGCAC |
| | | C | | g2 | |
| 807 | Il23r-g5 | ATCTTAACATAGCTTGAGGCAAG | 1590 | Usp15- | CCCATGAAATTCATGCAGACAGA |
| | | | | g3 | |
| 808 | Il34-g1 | CCTTTCATCATCAGAAGCTCCCA | 1591 | Usp15- | CCGAACACAGACTCACAGAGCGG |
| | | | | g4 | |
| 809 | Il34-g2 | CCCAAGACAGTATAGCCAGGCG | 1592 | Usp15- | CAGAAGAAAGTCTGCTTGACGCG |
| | | A | | g5 | |
| 810 | 1l34-g3 | CATTTAATAGAAAAAGGAACCA | 1593 | Vangl1- | TATTAAAGTCAGATGCCAACAGT |
| | | G | | g1 | |
| 811 | Il34-g4 | GCAAGAACAGTACAGCAGTTCC | 1594 | Vangl1- | CCCCATTCATTAGAAAGCCCCAC |
| | | A | | g2 | |
| 812 | Il34-g5 | CAGCACAACTGAAAAGCCCCAG | 1595 | Vangl1- | AGCCAAGACAGTTTTGACCCCAC |
| | | T | | g3 | |
| 813 | Il4r-g1 | CAGCAAAATCAGACAGCCCACA | 1596 | Vangl1- | GAAAAGAAAAGAAAAGCCCACAC |
| | | G | | g4 | |
| 814 | Il4r-g2 | AGATCCAAAATCAGAAGCCAGG | 1597 | Vangl1- | CAAAAAGAAAGATAAGGACCAGT |
| | | T | | g5 | |
| 815 | Il4r-g3 | TTCAACAACACTTAGCAGCCAGT | 1598 | Vdac2- | TCAAAGTCAACATCACAGCCGAG |
| | | | | g1 | |
| 816 | Il4r-g4 | ACTGTTAAAGACAGGCACCACCA | 1599 | Vdac2- | ACATAAAACAAACATGCACACCA |
| | | | | g2 | |
| 817 | Il4r-g5 | GACAACATCAGCTAGGAAAGCC | 1600 | Vdac2- | CGTAACCAAAGACAGCTGACCCA |
| | | C | | g3 | |
| 818 | Ippk-g1 | CCCAATGATTTCATAGCAGCCAG | 1601 | Vdac2- | CAGATGTTGAAAATTCCACACCG |
| | | | | g4 | |
| 819 | Ippk-g2 | AGCAACCAAAGTTTAAAGCAGC | 1602 | Vdac2- | TTACCTCATCTTAAAACAGCCAC |
| | | C | | g5 | |
| 820 | Ippk-g3 | AAGCAAAGACATTCACAGCCAA | 1603 | Vps13a- | TATGTATAGCATAAGCCCACCAC |
| | | G | | g1 | |
| 821 | Ippk-g4 | CATTCAAGTTTCAAAGACACCGC | 1604 | Vps13a- | TTTAAAAGCACATAAGCGCACAG |
| | | | | g2 | |
| 822 | Ippk-g5 | CAATACAGACACAGAGAAGGCC | 1605 | Vps13a- | GCCTCTTTCCAATTATCACCCAC |
| | | A | | g3 | |
| 823 | Irf9-g1 | CCAACTATGTAATACCTCACCCA | 1606 | Vps13a- | AATAACTGTAGAGTGCTCAGCCA |
| | | | | g4 | |
| 824 | Irf9-g2 | ACTTATAAGCATGAGACACAGA | 1607 | Vps13a- | CACTACAACGTTTAACAGCTCCG |
| | | G | | g5 | |
| 825 | Irf9-g3 | AAGCCTTGAATATGGCAGCATCC | 1608 | Vps35- | AAGTAAATTGTTTGCCAAGGCCC |
| | | | | g1 | |
| 826 | Irf9-g4 | CCATAGATGAAGGTGAGCAGCA | 1609 | Vps35- | AAAGAGAAAGTACAGAGACAGGA |
| | | G | | g2 | |
| 827 | Irf9-g5 | GCAAAATTTAATTTGGAGCTCAC | 1610 | Vps35- | TTCCACTAAGTTCATGAACTCCG |
| | | | | g3 | |
| 828 | Isx-g1 | GAAAACAAAGAGAAAACAGGCA | 1611 | Vps35- | CCTTTTGCCAAAACTCCAGCCAC |
| | | G | | g4 | |
| 829 | Isx-g2 | CAGTACAGTCTTTAATGGCAAGC | 1612 | Vps35- | CATATTGATGTTTCAGGTTCCAG |
| | | | | g5 | |
| 830 | Isx-g3 | GACATCATGAACAAGTACACGG | 1613 | Vps4b- | ACATTAGATTACAGAGTCCAAGC |
| | | A | | g1 | |
| 831 | Isx-g4 | AAAGAGAAAAATCAGTCCCTAT | 1614 | Vps4b- | GCACAAACAAGGTTAACCCCGAC |
| | | G | | g2 | |
| 832 | Isx-g5 | GAACATGAAGGTAGGGACACAA | 1615 | Vps4b- | AGAACTAGTTATGCACGGAGCAA |
| | | G | | g3 | |
| 833 | Itk-g1 | AATGTAAGACAGAAACCAGCAG | 1616 | Vps4b- | AACTTAGTAGACCTGTAGCAGCA |
| | | A | | g4 | |
| 834 | Itk-g2 | TAATTACAGGAAACAGTCTGCAG | 1617 | Vps4b- | CGTAATCACTGAGAGCCACACAA |
| | | | | g5 | |
| 835 | Itk-g3 | AGCCTTGAAGTAGTAGAACACCC | 1618 | Vton1- | GATGTCACACAATTGCAGAGCCC |
| | | | | g1 | |
| 836 | Itk-g4 | ATCAGTACACCAAACGACCACAC | 1619 | Vtcn1- | TCCAAAAGATGATCTGCCCCAAG |
| | | | | g2 | |
| 837 | Itk-g5 | CAAACACATACTTCTCAGCCACG | 1620 | Vtcn1- | AGAGTGACATCATAACAGCCCAT |
| | | | | g3 | |
| 838 | Kdm6b- | AGAAAATGAAAATAAAGCCCCA | 1621 | Vtcn1- | CATTTCAAAGAGCATGGCCGTAT |
| | g1 | G | | g4 | |
| 839 | Kdm6b- | ACGTCTATGTACACAACGAGCCG | 1622 | Vtcn1- | TGTGTAAATTCAGTGAGACACGT |
| | g2 | | | g5 | |
| 840 | Kdm6b- | GAACCAGTCAAGTAGTCCACACC | 1623 | Wdr4-g1 | AAATCATAGCATGAACTGCCCAG |
| | g3 | | | | |
| 841 | Kdm6b- | CTTCATGATGTTTGCCAGCCCAT | 1624 | Wdr4-g2 | GTTAAAGCAAAATAGCGGCCAGA |
| | g4 | | | | |
| 842 | Kdm6b- | CAGCACCAAAGAAGAGCTCCCCT | 1625 | Wdr4-g3 | AAGATACAAGTTTAATGAGGCAG |
| | g5 | | | | |
| 843 | Kir3dl1- | ACCATGGTTACTAAGAGCCCAGT | 1626 | Wdr4-g4 | AACACGTGACATTAGCTCCTAGA |
| | g1 | | | | |
| 844 | Kir3dl1- | CATAGCATGTGTAAGTCCCAACA | 1627 | Wdr4-g5 | AAAAACTGCTGTGTGGCATCAGA |
| | g2 | | | | |
| 845 | Kir3dl1- | GAAACCAGAACACACGAGGCTG | 1628 | Wdr7-g1 | AAATCATACCAGATGCCGCTACA |
| | g3 | A | | | |
| 846 | Kir3dl1- | ACTAAGAAGGAATTTTGCCTCGA | 1629 | Wdr7-g2 | ATACAAACAGATCGGAGCCCGCT |
| | g4 | | | | |
| 847 | Kir3dl1- | AAATTCGGGAAATGGGAGGCAT | 1630 | Wdr7-g3 | AGTTTACAAACAGAGAGCACAAG |
| | g5 | C | | | |
| 848 | Kit-g1 | CAAATATTTGTAGGTGAGCACCA | 1631 | Wdr7-g4 | ACTACAGAGCAGTTAGCCAGCTA |
| 849 | Kit-g2 | TGAATTTGTCAGAATGCAGCCAT | 1632 | Wdr7-g5 | ATGCCATCAACATAGTCCCACAG |
| 850 | Kit-g3 | GACATGTTTAAACTTGCACAGCG | 1633 | Wdr83- | TATTTATTACTTTACATGCCAGC |
| | | | | g1 | |
| 851 | Kit-g4 | TAAATTCTAGACAGTGAGCGACA | 1634 | Wdr83- | GTTATCAAAGGAGCCAGCCGCAT |
| | | | | g2 | |
| 852 | Kit-g5 | ACTTTCAAATGTGTACACGCAGC | 1635 | Wdr83- | TGTACCACATTAGAACCCACAGG |
| | | | | g3 | |
| 853 | Klf16-g1 | TCTCTCACACATATGCACACCCA | 1636 | Wdr83- | AACATTATTATTGTCCCCCAGGA |
| | | | | g4 | |
| 854 | Klf16-g2 | AGACAAAAAGAATTGGCCCCAG | 1637 | Wdr83- | TACTTTTAAATGCATCAGTCCGC |
| | | C | | g5 | |
| 855 | Klf16-g3 | CAAAGTAATCCACACACGCCACG | 1638 | Wfdc2- | ACCAGAGAGAAAGGAGGCCACAG |
| | | | | g1 | |
| 856 | Klf16-g4 | AGAAAAGAAATCCAGTCTGCAG | 1639 | Wfdc2- | CTCAGAATTTGGGTGTGGTGCAG |
| | | T | | g2 | |
| 857 | Klf16-g5 | GACACCTGAGATTTGAGTACCCA | 1640 | Wfdc2- | CCGCTGATTGAGTAGTAGTCCCA |
| | | | | g3 | |
| 858 | Klrc1-g1 | GTTACAAATAAAACAGCCCACAC | 1641 | Wfdc2- | GTCCACCTGACACTGGTCCTCAC |
| | | | | g4 | |
| 859 | Klrc1-g2 | TAAGACAAAACAGATGAGGCCC | 1642 | Wfdc2- | GTCCGTAATTGGTTCAAGCTGGG |
| | | A | | g5 | |
| 860 | Klrc1-g3 | AAATTCATCTAAAGGGAGCCAG | 1643 | Wnt2-g1 | TAAAGTTTCAAAAGACGAGCCCA |
| | | A | | | |
| 861 | Klrc1-g4 | TACACAATCTGATGAGGCCAAAG | 1644 | Wnt2-g2 | TTTCCAAAAAGAAATCAGCAGGA |
| 862 | Klrc1-g5 | CGAATAGATGATTTCCTGCTCGA | 1645 | Wnt2-g3 | GTCAATATTGTCACTGCAGCCAC |
| 863 | Klrd1-g1 | AACAATTGCACTGATGCCCAACC | 1646 | Wnt2-g4 | CACATTTATGATATTCCACTCAC |
| 864 | Klrd1-g2 | GCCTGATAACTTTCAGCACCAAC | 1647 | Wnt2-g5 | CACAACACATAACTTCGCAGCTG |
| 865 | Klrd1-g3 | CACTATAATGCATTCCAATCCAG | 1648 | Xbp1-g1 | ACTAGCAAGAAGATCCATCAAGC |
| 866 | Klrd1-g4 | GACAGACATCAGTCTCCACCGAG | 1649 | Xbp1-g2 | TCAGAATCTGAAGAGGCAACAGT |
| 867 | Klrd1-g5 | AAACAATGCAGTGCTCTGGCCTG | 1650 | Xbp1-g3 | TTCCTCAATTTTCACTACCACGT |
| 868 | Kmt2a- | AGTTACTATAAAGAGCAGACCCA | 1651 | Xbp1-g4 | CTTTTAACGAAAGAGACAGGCCT |
| | g1 | | | | |
| 869 | Kmt2a- | AAACCAGAAGCAAAGCCGACAT | 1652 | Xbp1-g5 | CACATAAGGGAAAACAAGCCCCC |
| | g2 | C | | | |
| 870 | Kmt2a- | CCACAGGATACAAAGCAGAGCT | 1653 | Zeb1-g1 | AATACATGTTAGATGGCAACACG |
| | g3 | A | | | |
| 871 | Kmt2a- | TCCAACACAGATACGTAGCTGCC | 1654 | Zeb1-g2 | CTGCTAGTTAATATAGGCCACAC |
| | g4 | | | | |
| 872 | Kmt2a- | CAGGATACAAAGCAGAGCTACT | 1655 | Zeb1-g3 | TAAGACTCAAACAAGACCACCGG |
| | g5 | C | | | |
| 873 | Krt17-g1 | TGTTCAGAACAAAGGCCACAGTT | 1656 | Zeb1-g4 | CAGAAATGACAGAATGGCCACCC |
| 874 | Krt17-g2 | TCCACGTTGATTTCGCCGCCCAC | 1657 | Zeb1-g5 | TATATGTGAGCTATAGGAGCCAG |
| 875 | Krt17-g3 | TCGGATCTTCACCTCCAGCTCAG | | | |

TABLE 4
MUCIG-Lib2 gRNAs

| SEQ | | |
|---|---|---|
| ID | | |
| NO: | Name: | Sequence: |
| 103 | Adam10-g1 | ATAAAAGTTTATCGAGAGCCAAG |
| 104 | Adam10-g2 | TCAATGTAAAACGTGCCACCACG |
| 105 | Adam10-g3 | GACAAGTATTTCTTTCAGCCAGA |
| 106 | Adam10-g4 | AATACACAAAGTAATAAGCAGGC |
| 107 | Adam10-g5 | CAGAATTAACACTGTCGGCAACA |
| 108 | Adam17-g1 | CAGAACATCTTGAAGCACCAGAG |
| 109 | Adam17-g2 | CATTCATACATATACCCACACAC |
| 110 | Adam17-g3 | AGTTACAGAGTTGAGAGCCACCA |
| 111 | Adam17-g4 | GAAAACCAGAACAGACCCAACGA |
| 112 | Adam17-g5 | TATCTTCAGACTTATACACCAGC |
| 113 | Adar-g1 | TTCACCATAAGAGAGCTGCAGTA |
| 114 | Adar-g2 | TCAAGGAATGCAAGACAGCCACG |
| 115 | Adar-g3 | CTTTTCATAATAATGGCAGCCAG |
| 116 | Adar-g4 | AGACCAGAAGAATCCCAGTGCAC |
| 117 | Adar-g5 | CTGAGCATACTCTAACAACCCGC |
| 123 | Adora2a-g1 | ACAAACAAACAAACAAGCCCCAC |
| 124 | Adora2a-g2 | TTAATGAGATTGGTCCAGCCAAC |
| 125 | Adora2a-g3 | AAAATCCTTAGGTAGATGGCCAG |
| 126 | Adora2a-g4 | CAGCAAATCGCAATGATGCCCTT |
| 127 | Adora2a-g5 | ATGATGTACACCGAGGAGCCCAT |
| 133 | Ago2-g1 | GTAAAAGTTAAGATGCCACAACA |
| 134 | Ago2-g2 | AGACTTAGTTAATAGCACCCAAC |
| 135 | Ago2-g3 | ATTGTCATTAGTAAGACACCCAC |
| 136 | Ago2-g4 | AAAAATAAAGCATTAGCAAGCCT |
| 137 | Ago2-g5 | CAGCAACTATGTTACAGACCTCC |
| 143 | Alcam-g1 | ATAGCAATCAGAATCAGAACCGT |
| 144 | Alcam-g2 | TATACATCCAATTAACAGCCACT |
| 145 | Alcam-g3 | CCTTAAAAAGTACCTCAGGCAGA |
| 146 | Alcam-g4 | AGATTATAGTTTTAGACAGTCCA |
| 147 | Alcam-g5 | CAATTTCAAAAGCTTGAACCACC |
| 168 | Atg10-g1 | CCTGCAGTAATTCAACAGAGCAG |
| 169 | Atg10-g2 | CCCTAAAGTAAAGAACCGGCACT |
| 170 | Atg10-g3 | CAGAGGTAAATTCAGACCAACCA |
| 171 | Atg10-g4 | CATCGTTCACTAAAGCGAGCACA |
| 172 | Atg10-g5 | TACATTAATTTTCAGAAACAGGC |
| 173 | Atg14-g1 | TAAGACCATGTAAAGCAGCCCAT |
| 174 | Atg14-g2 | ATATGAAATAAAACAAGGCCACC |
| 175 | Atg14-g3 | ACCAAGGAAGAAACCGGACAGCA |
| 176 | Atg14-g4 | TAATAACTGCCAAAGCGCCACAG |
| 177 | Atg14-g5 | AGACACAATGTTGACGAGCTGCG |
| 178 | Atg9a-g1 | ACTCATTGAGAAACAGAGAGCCG |
| 179 | Atg9a-g2 | TAGGACTACATAGAAGCAGCCAG |
| 180 | Atg9a-g3 | ATAGATAAACTTGATAAGCCGGT |
| 181 | Atg9a-g4 | CAAAACATTCTAGCTGCGCGCCC |
| 182 | Atg9a-g5 | GTCCACCTTGTTAACCAGCTCCA |
| 183 | Atp13a1-g1 | TTTATAAAATTGCAGACGCCGAT |
| 184 | Atp13a1-g2 | CTGGAATTCAAATGACAGCACCT |
| 185 | Atp13a1-g3 | GACAGAGAATTGCAGCATCACCG |
| 186 | Atp13a1-g4 | CACTGAGAAAACATAGAGTGCAG |
| 187 | Atp13a1-g5 | CCCAAAGATGACATGCAGCCGAG |
| 193 | Aurka-g1 | ACATGCAGAAAAAGAAACCCCCG |
| 194 | Aurka-g2 | TTAAACAGCACCTTCAGAGCCAG |
| 195 | Aurka-g3 | AGGAACTCATAGCAGAGAACGCC |
| 196 | Aurka-g4 | ACTAAATCAGGAAAAGCAGCATG |
| 197 | Aurka-g5 | TGAATGACAGTAAGACAGAGCGT |
| 218 | Bcl2-g1 | AAACAAATACATAAGGCAACCAC |
| 219 | Bcl2-g2 | TGTATGAATAAAGGCCACACCCA |
| 220 | Bcl2-g3 | CTTTTAGAGCAAATGCAGCCACA |
| 221 | Bcl2-g4 | TATCAACCTTAAAAGCAGCCCAT |
| 222 | Bcl2-g5 | CCGAACTCAAAGAAGGCCACAAT |
| 233 | Braf-g1 | AGACAGTTCCAAATGACCCAGAT |
| 234 | Braf-g2 | GAATTCTGTAAACAGCACAGCAT |
| 235 | Braf-g3 | CATTCAACATTTTCACTGCCACA |
| 236 | Braf-g4 | ATAACCACATGTTTGACAACGGA |
| 237 | Braf-g5 | TCCACAAAATAGATCCAGACAAC |
| 243 | Btla-g1 | ATATGTATATTAATCCAGCAGCA |
| 244 | Btla-g2 | GACCTTTAAGACGCAGCACCAGC |
| 245 | Btla-g3 | ATCCTTTTCAGAAAGCAGAGCAG |
| 246 | Btla-g4 | TAACAGAATAAAGTGGAGTGCAA |
| 247 | Btla-g5 | TGTAGAACAGCTATACGACCCAT |
| 248 | C10orf54-g1 | TCCAGAGATAGATAAAGCACCCG |
| 249 | C10orf54-g2 | CCATACAGGTAATGAGAGCCCAG |
| 250 | C10orf54-g3 | CAGACAAAGCTAGATCCCCAGAG |
| 251 | C10orf54-g4 | CAAGACACTAATGAGCTCACAGT |
| 252 | C10orf54-g5 | CCAGGAAAATAGCAAGGAGCAGG |
| 258 | C5ar1-g1 | AACGCATTATAAGACAGGACACC |
| 259 | C5ar1-g2 | ATAAAGAAACAGATGACCACAGC |
| 260 | C5ar1-g3 | TATACCATGACATTTGCCCAGCA |
| 261 | C5ar1-g4 | CCTCAAGAAGAGATGCAGGCAAC |
| 262 | C5ar1-g5 | AATACCACATACAGTGTGCTCTG |
| 263 | Cad-g1 | AAATACATCGAAGAGCAGTTCCA |
| 264 | Cad-g2 | CAAAGTGATTTTCAGCCAGCTGA |
| 265 | Cad-g3 | ATCTGATTATACTTGAGGCCACA |
| 266 | Cad-g4 | AGCAAAGCCAGAACCGAGACCAC |
| 267 | Cad-g5 | CAGTATCTGAGACTGCATCCCCA |
| 268 | Casp1-g1 | GACTCAATGAAAAGTGAGCCCCT |
| 269 | Casp1-g2 | AGTCTGAAAAGGATTCAACCGCG |
| 270 | Casp1-g3 | TAAGTGATAAAGATTTGGCTTGC |
| 271 | Casp1-g4 | AGACATGATCACATAGGTCCCGT |
| 272 | Casp1-g5 | ACCACAATTGCTGTGTGCGCATG |
| 278 | Cblb-g1 | AAAAACCTGAAATTGCCACAGAG |
| 279 | Cblb-g2 | AATTCCGTAAAATAGAGCCCCAG |
| 280 | Cblb-g3 | GAACTGAAAAAGTAGCAGCAAGG |
| 281 | Cblb-g4 | GCAAGCTACATGAAGCCCAACAG |
| 282 | Cblb-g5 | TGACCATTATCACAAGACCGAAC |
| 303 | Ccnq-g1 | CATCACTAAATACCTGCCACCAA |
| 304 | Ccnq-g2 | GGAAATCAAGTAGTGCAGCAGGT |
| 305 | Ccnq-g3 | AACTCGTAGCATAAGAAGCTCAC |
| 306 | Ccnq-g4 | TACACATTCATACTTCCCCTAAG |
| 307 | Ccnq-g5 | AACTTATGGTAAATGGTGCAAGC |
| 333 | Cd274-g1 | CGTAGCAAGTGACAGCAGGCTGT |
| 334 | Cd274-g2 | CCATCGTGACGTTGCTGCCATAC |
| 335 | Cd274-g3 | CGCTTGTAGTCCGCACCACCGTA |
| 336 | Cd274-g4 | TAGAAAACATCATTCGCTGTGGC |
| 337 | Cd274-g5 | ATTTCTCCACATCTAGCATTCTC |
| 338 | Cd276-g1 | ATAATAGCAGTTACACAGTCTGC |
| 339 | Cd276-g2 | GTGAACATCGAACAAGCCCCGCT |
| 340 | Cd276-g3 | AGCAAGAACTAAGAGGTCACTGT |
| 341 | Cd276-g4 | GACAACAAAAGCCAGGGCCAGAT |
| 342 | Cd276-g5 | TTTAATGAAGAGCTGACGGCCAA |
| 348 | Cd38-g1 | AGATCATCAGCAATGTAGCCCAG |
| 349 | Cd38-g2 | ATAAACAATACAGAAGCACCACA |
| 350 | Cd38-g3 | AAAACATGAATACAGAAGCACCT |
| 351 | Cd38-g4 | CCAATTTAACAAGTGGGGCGTAG |
| 352 | Cd38-g5 | CAGAGCAAACTGACCAGAACCTC |
| 353 | Cd47-g1 | CAAGCAAGACAGAAGCGCCAAGT |
| 354 | Cd47-g2 | TAGAGATTACAATGAGGCCAAGT |
| 355 | Cd47-g3 | ACCAAAGCAAGGACGTAGCCCAG |
| 356 | Cd47-g4 | CCACGATGACTGTGAGCACCAGC |
| 357 | Cd47-g5 | TAAACAGTAGTTGAGCTGAACCT |
| 358 | Cd5-g1 | ACAAAGGACAAATGTCCAAGCGT |
| 359 | Cd5-g2 | AGAGTCCAAGGAGAAAGCCAACC |
| 360 | Cd5-g3 | AATTATTTAGACTCTAGGACCAT |
| 361 | Cd5-g4 | TCCCACTGTGATCTCTGGCGCAC |
| 362 | Cd5-g5 | ATAAGTCCTTGTAAGTACCCCAC |
| 363 | Cd55-g1 | AAATGCTAGCATTTCCAACCAGG |
| 364 | Cd55-g2 | CATATATATAACGGTCACCACCT |
| 365 | Cd55-g3 | TCTTGAAGACAATGACAGCATGC |
| 366 | Cd55-g4 | CAAAACTGAGCAACTGGAGACCA |
| 367 | Cd55-g5 | GTTAAATTAGAATGTGCCACCTC |
| 408 | Cep55-g1 | ATATTGCTAAATAGTAGCCCAAG |
| 409 | Cep55-g2 | CCTGCAAATCAAATGAGGCAAGA |
| 410 | Cep55-g3 | AGGAGTAAAAATATACAGCCACT |
| 411 | Cep55-g4 | AAATGCTAGTCATTACAACAGCG |
| 412 | Cep55-g5 | AAGTCTAGAGTACATGCCTGCAT |
| 413 | Cflar-g1 | AGAAAAGCTGGATATGATAGCCC |
| 414 | Cflar-g2 | CTCTGTAGAGCAATTCAGCCAAG |
| 415 | Cflar-g3 | ATGATATACCAAGAACACCAACG |
| 416 | Cflar-g4 | CATACTTGCATATCGGCGAACAA |
| 417 | Cflar-g5 | GAAGATATTTTGTGTCGTTGCCA |
| 418 | Chic2-g1 | TTAGACATATAATTCCCAGCACA |
| 419 | Chic2-g2 | GTCCGATTATGTACAGAGCCACA |
| 420 | Chic2-g3 | AAACACTGCATTTTGGAACCGCA |
| 421 | Chic2-g4 | AGAACCAAGAAGAAGCAACCCCT |
| 422 | Chic2-g5 | AATAAACAATAATTGCCAGGCGT |
| 423 | Cish-g1 | GCACAACATAGAGAAGCCAGCTC |
| 424 | Cish-g2 | TAAACAGAGATAGTCAGCTCCCA |
| 425 | Cish-g3 | TTGACAAGCAGTTAGAGTCCAGC |
| 426 | Cish-g4 | CTGAAGAAAGGACAGCAGAACCC |
| 427 | Cish-g5 | CACTACAGCTAAAAGAGTTCAGG |
| 453 | Cop1-g1 | ATAAGAGACCATATGGCCAGCAA |
| 454 | Cop1-g2 | AGCATAAGACAATGTGGCCAACG |
| 455 | Cop1-g3 | GACACATGATTATATCCCAGCAG |
| 456 | Cop1-g4 | CCTATCACAAAAATTAGCCACAC |
| 457 | Cop1-g5 | CTTCAAAATGAGCAGTGAGTCGC |
| 458 | Creb1-g1 | TCATTTAGTTACCAACACTCCGC |
| 459 | Creb1-g2 | AATTAATCTGATTTGTGGCAGTA |
| 460 | Creb1-g3 | AATCAGTTACACTATCCACAGAC |
| 461 | Creb1-g4 | TCATTTTCCTCATTTCCCCCAAC |
| 462 | Creb1-g5 | CTAAGGTTACAGTGGGAGCAGAT |
| 468 | Csf1-g1 | TCAGCAGCATAAAGAGACCAAGG |
| 469 | Csf1-g2 | TGCACACATATTTTCAAGACCCA |
| 470 | Csf1-g3 | GCCCACAATAAATAGTGGCAGTA |
| 471 | Csf1-g4 | TCAGCAAGACTAGGATGATGCCC |
| 472 | Csf1-g5 | CATCTATTATGTCTTGTACCAGA |
| 473 | Csf1r-g1 | CACACAAGAATATATGCCAGCGT |
| 474 | Csf1r-g2 | ATAGTAAATATAGAGGCTAGCAC |
| 475 | Csf1r-g3 | CATGACAGACATACAGGCCACCA |
| 476 | Csf1r-g4 | CAGCAGTATTCAGTGATGACCAG |
| 477 | Csf1r-g5 | CACTTGAAGAAGTCGAGACAGGC |
| 478 | Csf3-g1 | ACCACACTTTATTATCCGCAAGC |
| 479 | Csf3-g2 | CCTTTATACATAAAGCCATCAAG |
| 480 | Csf3-g3 | CCATAGTGCACTTTGCCACAGCA |
| 481 | Csf3-g4 | GATCTAGAAGCTTAGAGCTCCAT |
| 482 | Csf3-g5 | GAAATACCCGATAGAGCCTGCAG |
| 483 | Cspg4-g1 | CACGTAGATAAAGTTGCCACGCT |
| 484 | Cspg4-g2 | TTGCCTTAAATTAACCAACCCCA |
| 485 | Cspg4-g3 | AGAAAGTTTCATATGGGCCATAG |
| 486 | Cspg4-g4 | CTCATACAGAATATTCCCAGCAT |
| 487 | Cspg4-g5 | CACAGTGATGACAAAGGCCTCAG |
| 488 | Ctla4-g1 | GTGTTTATATTCAAACCACCAGC |
| 489 | Ctla4-g2 | ATAAAATGAGTGTAAAGACCCAG |
| 490 | Ctla4-g3 | TCAAAGAAACAGCAGTGACCAGG |
| 491 | Ctla4-g4 | TGACACAACAGAAATATCCCAGC |
| 492 | Ctla4-g5 | CAATGACATAAATCTGCGTCCCG |
| 493 | Cxcl1-g1 | CAAGACATACAAACACAGCCTCC |
| 494 | Cxcl1-g2 | AATGTAAAATAAAAACCACACAC |
| 495 | Cxcl1-g3 | TTGTATAGTGTTGTCAGAAGCCA |
| 496 | Cxcl1-g4 | ATGACTTCGGTTTGGGTGCAGTG |
| 497 | Cxcl1-g5 | AATACATAAATAAATAGGACCCT |
| 498 | Cxcl5-g1 | ATAAAAGTTATATGCCAGCCCAG |
| 499 | Cxcl5-g2 | AAATATATAGTTAGTGGCCCAAA |
| 500 | Cxcl5-g3 | ACAACAGTAAAAGAGGTCCCCAT |
| 501 | Cxcl5-g4 | AACAGCAACAGAAATGCCAGCGG |
| 502 | Cxcl5-g5 | GTAATATAAAGAAGTGAGACACT |
| 503 | Cybb-g1 | AAGTCAAAACAAGATGAGCGCAT |
| 504 | Cybb-g2 | GAACTAGAAGTGTTAGCCACCAT |
| 505 | Cybb-g3 | AAGTTCAATAACAAAGACACAGG |
| 506 | Cybb-g4 | CCACAAGCATTGAATAGCCCCTC |
| 507 | Cybb-g5 | AGTATAATTATACTTAGGCCCAT |
| 518 | Dcp1a-g1 | CCCACAAGAAGATCCAGGCCACA |
| 519 | Dcp1a-g2 | GCTCAGAAAAGTAAGCAGTGCAT |
| 520 | Dcp1a-g3 | TAGAGAATAAGACCACTCAGCAC |
| 521 | Dcp1a-g4 | AGCATAAATAGAAACCATGGCAG |
| 522 | Dcp1a-g5 | TCAGATGTTTATGACCAGACGGA |
| 533 | Dgka-g1 | AGGACCAAAACAGAGCCCAGCAT |
| 534 | Dgka-g2 | TACTTACAGAAGTTACAGCTCAG |
| 535 | Dgka-g3 | TCATCTTTCATAGTCACGTCCAT |
| 536 | Dgka-g4 | AAGGATGTCTGTATGGGAGCAGT |
| 537 | Dgka-g5 | AAGTTGATCACAGAGACAGCCTG |
| 548 | Dner-g1 | CACAAGTGTTAAACGCAAAGCCA |
| 549 | Dner-g2 | ACAAACTGACAATAGGTGCCAGA |
| 550 | Dner-g3 | CATATCAATCAAATACAGCCACA |
| 551 | Dner-g4 | AGATCAGCAAAGAATTGGCATCC |
| 552 | Dner-g5 | CACAGTTAAGACCTTCATAGCCG |
| 558 | Entpd1-g1 | TAGAAAGCAGAAAACGCCCCAAA |
| 559 | Entpd1-g2 | ATAGTTAATAGTAATCCACCCAT |
| 560 | Entpd1-g3 | ACACAGTATAGTCCTCGCCATAG |
| 561 | Entpd1-g4 | CAAGTTCAGCATGTAGCCCAAAG |
| 562 | Entpd1-g5 | AGTCACATTAGCTGCACGAGCAC |
| 568 | Epcam-g1 | ACCATATTCATTCAGAGAGCAAC |
| 569 | Epcam-g2 | TCTAGTGAAACATGCAGCTGCAG |
| 570 | Epcam-g3 | CTCACGTGCAGAATCAGTCCATC |
| 571 | Epcam-g4 | CAGCTTGTAGTTGTCACAGACAC |
| 572 | Epcam-g5 | CACGCCCCTCCCCGCCCTCACCT |
| 578 | Erbb2-g1 | CGACTTTCATATAACACCCACTC |
| 579 | Erbb2-g2 | AGACCATAGCATACTCCAGCACA |
| 580 | Erbb2-g3 | ACACAGTGAGTTACAGACCAAGC |
| 581 | Erbb2-g4 | GCAAAAACGTCTTTGACAACCCC |
| 582 | Erbb2-g5 | ACCATCAAACACATCGGAGCCAG |
| 588 | Eya3-g1 | TTCACAAACAGACGGCTGCAGAC |
| 589 | Eya3-g2 | TAAGAATAATGTGCTGAGCTTGG |
| 590 | Eya3-g3 | ACATGCTGAGATTTGACGCAAGG |
| 591 | Eya3-g4 | TTATTTGGTGTAGTCTGGGACAA |
| 592 | Eya3-g5 | CATACCTGGCCACAGTGCACCGA |
| 598 | F8a-g1 | AGCCAAATTAACATGGCAGCAAT |
| 599 | F8a-g2 | AACAGAAATTTGCTGCAACCCAA |
| 600 | F8a-g3 | AGCAAAGATGCATTGATCCAGGA |
| 601 | F8a-g4 | ACTGCAATAACAGAAACAGCTCC |
| 602 | F8a-g5 | AGCAGCAGTACACGGGACACCTC |
| 628 | Fitm2-g1 | GTGCAATTTCATATGACAAGCCA |
| 629 | Fitm2-g2 | CTTCCACAATCATGAGCGCACAG |
| 630 | Fitm2-g3 | ATATATACCTTTAATCCCAGCAC |
| 631 | Fitm2-g4 | AAACCAAACATGGTGCCGAACAC |
| 632 | Fitm2-g5 | AACTCTAAAGAGAAGCAGAGCCG |
| 648 | Fut4-g1 | TATGTCTAAACTTTAGCAGCCAC |
| 649 | Fut4-g2 | CCAGGTTAAATTTCAGCCCCAAC |
| 650 | Fut4-g3 | CACACCTTTAATTCCCACCCAAG |
| 651 | Fut4-g4 | CGATTCGAAGTTCATCCACACCC |
| 652 | Fut4-g5 | GGTATTTAGAAAACGCAGCCAAG |
| 658 | Gale-g1 | TTCTGCTTCTGCCAGCGCCACAG |
| 659 | Gale-g2 | GTTAACTCTATAGTAGTCCAGAG |
| 660 | Gale-g3 | CACGTGAATGTAATCCCTCACAC |
| 661 | Gale-g4 | AAAGCTTAATCATCAGTGCCTGG |
| 662 | Gale-g5 | ATTGAAGTACCGAAGCAGCACGG |
| 673 | Gdf15-g1 | GTAGACATTACAGCCGTGCCCAA |
| 674 | Gdf15-g2 | AAATACACAATCCATCCACCCAG |
| 675 | Gdf15-g3 | ACAGAACATGTGATGGAGTCCAG |
| 676 | Gdf15-g4 | AGAAAAGGCAAGTTCAGGCAAGT |
| 677 | Gdf15-g5 | CATAAGTCTGCAGTGACACACCA |
| 678 | Gls-g1 | TGCAGACCATTATAGCAACCCGT |
| 679 | Gls-g2 | TATTGAATGATACAGCCCACCAA |
| 680 | Gls-g3 | ACTGAATCACAAAAAGCCAGCCG |
| 681 | Gls-g4 | CCAACATATTCATTACCAGCCAT |
| 682 | Gls-g5 | CAACTAAAAGAATACCCCCAGCA |
| 688 | Gpi1-g1 | CACCACCAAGTAAAGAGCCAACC |
| 689 | Gpi1-g2 | AATGTTAGAGACAAACCAGACAC |
| 690 | Gpi1-g3 | CTCATAACGATCAATCCTCCGAG |
| 691 | Gpi1-g4 | AACAACATGACACGTCAAAGCCC |
| 692 | Gpi1-g5 | CGACAAAGTGCTTTGCAACTGCA |
| 703 | Havcr2-g1 | CCAAAGTCAGAAATGAAGGCGAG |
| 704 | Havcr2-g2 | AGACACCAATGATAAGTGCCAGG |
| 705 | Havcr2-g3 | CCCACCTAAGAAAGCCAGGACCT |
| 706 | Havcr2-g4 | AGTCCTTAATTTCATCAGCCCAT |
| 707 | Havcr2-g5 | TATAGTGTTAAGCATATGCCACC |
| 733 | Hgf-g1 | CTGCATAAATAAGTAGCCCAGAT |
| 734 | Hgf-g2 | ACTAGAAGCCAATTGCAGCAGCA |
| 735 | Hgf-g3 | AATTTGAGAGCAGTAGCCAACTC |
| 736 | Hgf-g4 | CTGATCCAATCTTTTCAGCCCCA |
| 737 | Hgf-g5 | CCTTTATCAATGATCCTCCACAG |
| 738 | Hmox1-g1 | AACAAGACAGAAATACGAGACAG |
| 739 | Hmox1-g2 | TCACACAGAAGTTAGAGACCAAG |
| 740 | Hmox1-g3 | CCCAAGAGAAGAGAGCCAGGCAA |
| 741 | Hmox1-g4 | TACATGGCATAAATTCCCACTGC |
| 742 | Hmox1-g5 | GTCAGCATCACCTGCAGCTCCTC |
| 743 | Hspa13-g1 | ATACAAGCAGAAAATGACGCCCT |
| 744 | Hspa13-g2 | TTTATATAAATGCTGAAGCAGCC |
| 745 | Hspa13-g3 | AGAAATGACAGCATTGGCAACCG |
| 746 | Hspa13-g4 | AAATAAAGAAATTATCAGCCCCT |
| 747 | Hspa13-g5 | TCACAGAAAACTCAGCCATCCCA |
| 758 | Ido1-g1 | ATTTCCACCAATAGAGAGACGAG |
| 759 | Ido1-g2 | AGACAGATATATGCGGAGAACGT |
| 760 | Ido1-g3 | AATCTACATAATATACAACAGGC |
| 761 | Ido1-g4 | GCATAAGACAGAATAGGAGGCAG |
| 762 | Ido1-g5 | AACCTCAAAACCAGGCACGCCAG |
| 763 | Igf1r-g1 | CTAACATAGAACTGAGAGACCCA |
| 764 | Igf1r-g2 | TAATTTAAATATTTCCACCCAGA |
| 765 | Igf1r-g3 | ACATACAGCATGATAACCAGCCC |
| 766 | Igf1r-g4 | CAATGTAGTTATTGGACACCGCA |
| 767 | Igf1r-g5 | TTAATAAGCAGATTGCCCTTCAG |
| 773 | Ikbkg-g1 | CAGAGAAGATTCTTCACCCAGCA |
| 774 | Ikbkg-g2 | AGCAGCTCCTCACAGCGTTCCCT |
| 775 | Ikbkg-g3 | GATATACATGTACTTGTGTCACA |
| 776 | Ikbkg-g4 | GAATTTGCACATAAGGAACTCCT |
| 777 | Ikbkg-g5 | ATTTCATCTTGAAGCAGTGACAC |
| 778 | IL10ra-g1 | GATTCCACAGAATAGCAGCATAG |
| 779 | IL10ra-g2 | CAGGACCTAAACTATCACCCCAG |
| 780 | IL10ra-g3 | AAGCAGACATAAGTCCTAAGCCT |
| 781 | IL10ra-g4 | TATTAGGATGAAAACACACACCA |
| 782 | IL10ra-g5 | GTTTCAAATAACCTGCGGCCAGA |
| 793 | Il18bp-g1 | TGTAAAGATTGAAGTCAGTGCAG |
| 794 | Il18bp-g2 | ACATACAAAAGCAGGACCCACCA |
| 795 | Il18bp-g3 | CACGTTAAGTGTAAGCAGAGCTA |
| 796 | Il18bp-g4 | ATATACAGTTGTGACCTGACGCA |
| 797 | Il18bp-g5 | TCAATGAAGGAACCATTGCCCAG |
| 813 | Il4r-g1 | CAGCAAAATCAGACAGCCCACAG |
| 814 | Il4r-g2 | AGATCCAAAATCAGAAGCCAGGT |
| 815 | Il4r-g3 | TTCAACAACACTTAGCAGCCAGT |
| 816 | Il4r-g4 | ACTGTTAAAGACAGGCACCACCA |
| 817 | Il4r-g5 | GACAACATCAGCTAGGAAAGCCC |
| 818 | Ippk-g1 | CCCAATGATTTCATAGCAGCCAG |
| 819 | Ippk-g2 | AGCAACCAAAGTTTAAAGCAGCC |
| 820 | Ippk-g3 | AAGCAAAGACATTCACAGCCAAG |
| 821 | Ippk-g4 | CATTCAAGTTTCAAAGACACCGC |
| 822 | Ippk-g5 | CAATACAGACACAGAGAAGGCCA |
| 848 | Kit-g1 | CAAATATTTGTAGGTGAGCACCA |
| 849 | Kit-g2 | TGAATTTGTCAGAATGCAGCCAT |
| 850 | Kit-g3 | GACATGTTTAAACTTGCACAGCG |
| 851 | Kit-g4 | TAAATTCTAGACAGTGAGCGACA |
| 852 | Kit-g5 | ACTTTCAAATGTGTACACGCAGC |
| 853 | Klf16-g1 | TCTCTCACACATATGCACACCCA |
| 854 | Klf16-g2 | AGACAAAAAGAATTGGCCCCAGC |
| 855 | Klf16-g3 | CAAAGTAATCCACACACGCCACG |
| 856 | Klf16-g4 | AGAAAAGAAATCCAGTCTGCAGT |
| 857 | Klf16-g5 | GACACCTGAGATTTGAGTACCCA |
| 858 | Klrc1-g1 | GTTACAAATAAAACAGCCCACAC |
| 859 | Klrc1-g2 | TAAGACAAAACAGATGAGGCCCA |
| 860 | Klrc1-g3 | AAATTCATCTAAAGGGAGCCAGA |
| 861 | Klrc1-g4 | TACACAATCTGATGAGGCCAAAG |
| 862 | Klrc1-g5 | CGAATAGATGATTTCCTGCTCGA |
| 863 | Klrd1-g1 | AACAATTGCACTGATGCCCAACC |
| 864 | Klrd1-g2 | GCCTGATAACTTTCAGCACCAAC |
| 865 | Klrd1-g3 | CACTATAATGCATTCCAATCCAG |
| 866 | Klrd1-g4 | GACAGACATCAGTCTCCACCGAG |
| 867 | Klrd1-g5 | AAACAATGCAGTGCTCTGGCCTG |
| 868 | Kmt2a-g1 | AGTTACTATAAAGAGCAGACCCA |
| 869 | Kmt2a-g2 | AAACCAGAAGCAAAGCCGACATC |
| 870 | Kmt2a-g3 | CCACAGGATACAAAGCAGAGCTA |
| 871 | Kmt2a-g4 | TCCAACACAGATACGTAGCTGCC |
| 872 | Kmt2a-g5 | CAGGATACAAAGCAGAGCTACTC |
| 893 | Lag3-g1 | AGAAGCAAAAAGCCAAGGAGCAG |
| 894 | Lag3-g2 | CAAAAGGACCCAATCAGACAGCT |
| 895 | Lag3-g3 | CTTCGTAGAAAGTTAGGATCCAG |
| 896 | Lag3-g4 | AGTCACTGTGATGACCGCCAACG |
| 897 | Lag3-g5 | CAGACAGACAGACAGACACACAC |
| 908 | Lgals1-g1 | GAAAGCACAAGAGAGGTCACTGA |
| 909 | Lgals1-g2 | AGACCAAGAACACATGGAGGCAT |
| 910 | Lgals1-g3 | TTTATTAAGACAAATGCGGTCCG |
| 911 | Lgals1-g4 | CAGTCAGAAGACTCCACCCGAGA |
| 912 | Lgals1-g5 | CGAACTTTGAGACATTCCCCAGG |
| 913 | Lgals3-g1 | AAAGGCATTCTAACTAGGGCAGC |
| 914 | Lgals3-g2 | TTAAGCGAAAAGCTGTCTGCCAT |
| 915 | Lgals3-g3 | ACACAATAATAAATACATCTGCT |
| 916 | Lgals3-g4 | ACAGCTTGTCCTCTGACCTCCAC |
| 917 | Lgals3-g5 | TCCACTTCCAGGCAGTGACGCGT |
| 918 | Lgals9-g1 | CCTTCACATATGATCCACACCGA |
| 919 | Lgals9-g2 | ATATCATGATGGACTTGGACGGG |
| 920 | Lgals9-g3 | GGGTACACCACAGGAGGGATTCC |
| 921 | Lgals9-g4 | AGAATTTCTTGTTCACCATCACC |
| 922 | Lgals9-g5 | GGAAAGATAAGACACAGGCAGAG |
| 923 | Lif-g1 | GACATAGTAATAAATAGACAGCT |
| 924 | Lif-g2 | TTTAAATAATAAATAAAGGCCCC |
| 925 | Lif-g3 | GACACCCTAAAAGTGAGTCACAG |
| 926 | Lif-g4 | GCATTTAACAATGTCCCAAACCC |
| 927 | Lif-g5 | GTAATAGGAAATGAAGAGAGCAT |
| 938 | Lipt2-g1 | TTGAACAGTTCAGAGCCAAGCCG |
| 939 | Lipt2-g2 | CAAACTTGAGACATTCAACCCAG |
| 940 | Lipt2-g3 | ATCGACAACTAGAAGCAACCAAG |
| 941 | Lipt2-g4 | GCAATCACAATGGAGTGAGCCAT |
| 942 | Lipt2-g5 | CAGTGACAAGTCTTTGAAGCGCC |
| 948 | Lrrc32-g1 | CGAAGCGCTGTATAGAAGCCCAG |
| 949 | Lrrc32-g2 | ACAAGGTACTTAGCCTCCTCAGA |
| 950 | Lrrc32-g3 | CTTGGATGTCCAGTGAGAGCACC |
| 951 | Lrrc32-g4 | GTTCACCGTCCTACAGGGCACTT |
| 952 | Lrrc32-g5 | CAGCATGGCCAGGAGTAGCAGGA |
| 978 | Map2k7-g1 | TAAAAATAAAACCATCAGGCCCA |
| 979 | Map2k7-g2 | CCAGAAATGACAAGGAGCAGCAA |
| 980 | Map2k7-g3 | AACAGGACAGTTAAGAGCCACAG |
| 981 | Map2k7-g4 | CAGTGCTTTCACAATCGCCACAG |
| 982 | Map2k7-g5 | ACATCCTTAAACCAGGACGCGAC |
| 988 | Mcl1-g1 | CGAAGCGCTGTATAGAAGCCCAG |
| 989 | Mcl1-g2 | ACAAGGTACTTAGCCTCCTCAGA |
| 990 | Mcl1-g3 | CTTGGATGTCCAGTGAGAGCACC |
| 991 | Mcl1-g4 | GTTCACCGTCCTACAGGGCACTT |
| 992 | Mcl1-g5 | CAGCATGGCCAGGAGTAGCAGGA |
| 993 | Mdm2-g1 | GTTTTCACTTACATACCACCAGA |
| 994 | Mdm2-g2 | TAAGGAAAATATAAACAGCCAAT |
| 995 | Mdm2-g3 | CAAAGCAGAGTTCTGTGACGAGC |
| 996 | Mdm2-g4 | TTGAACAATACACAATGTGCTGC |
| 997 | Mdm2-g5 | CTTAGTCATAATATACTGGCCAA |
| 998 | Med23-g1 | AAACCTAGCATATTGCAGACCAT |
| 999 | Med23-g2 | TCCAGAAATGAACTGCAGCAGCA |
| 1000 | Med23-g3 | CTTGAGTATAGAAACGCCCACAT |
| 1001 | Med23-g4 | CCTCCAATGATTTTCCGAACCAG |
| 1002 | Med23-g5 | CATGTCATAAAATGCCACGCCAA |
| 1003 | Mertk-g1 | AACAAAGTATCTAAGACCACCAG |
| 1004 | Mertk-g2 | AAAATGAATCCACAGAAGCAGCC |
| 1005 | Mertk-g3 | CATCTTACAGAAGTACGACCCAT |
| 1006 | Mertk-g4 | TTAAAGATATAAGCACTCAGCTG |
| 1007 | Mertk-g5 | CTCATACAGATGTGGCGAAGCAG |
| 1008 | Met-g1 | CTACTGATATTGAGACCGCACCA |
| 1009 | Met-g2 | TACATCATCTGTATGCAGCCAAG |
| 1010 | Met-g3 | AGTAAGCACAAAATTCCACAGAG |
| 1011 | Met-g4 | ACACCTTATAAACCGCCGGCAGA |
| 1012 | Met-g5 | CACAAAGAAATTGATGAACCGGT |
| 1013 | Mex3b-g1 | AAAATAAAAAGATTTAACCCAGC |
| 1014 | Mex3b-g2 | ACACTAAAACTATAGCGGTGCAA |
| 1015 | Mex3b-g3 | ATGAACATTCAAAAAGGATCCAC |
| 1016 | Mex3b-g4 | TCTGTCAGCTCGATGATGCCACC |
| 1017 | Mex3b-g5 | CGTCACAACAAAGACAGGCTCCT |
| 1018 | Mfge8-g1 | AGTACAATCAGAAGGGAAGGCCA |
| 1019 | Mfge8-g2 | GAAACCCATATACACAGACGAGG |
| 1020 | Mfge8-g3 | AGTCACAGAAGTCACCAGACGCG |
| 1021 | Mfge8-g4 | TCCTCATATACAGTCCACTGCAC |
| 1022 | Mfge8-g5 | AATTGTGTTATTCTTCAGGCCCA |
| 1023 | Mgat1-g1 | ACAAAGATGATAGCACCCCAAAG |
| 1024 | Mgat1-g2 | AACAACTTATCCAAGCAGCGCCG |
| 1025 | Mgat1-g3 | CCATGAAACTAAGATCAGGCAAC |
| 1026 | Mgat1-g4 | TCCCTGAAGATCATCAACCCCGC |
| 1027 | Mgat1-g5 | CACGCATAGACTTTCCATCTCCC |
| 1038 | Mrc1-g1 | CCATAGAAAGGAATCCACGCAGT |
| 1039 | Mrc1-g2 | AAGGACAAACCAATGCAACCCAG |
| 1040 | Mrc1-g3 | GTCTTTGTAAATAACCCACCCAT |
| 1041 | Mrc1-g4 | CCTTGCCTTTCATAACCACGCAG |
| 1042 | Mrc1-g5 | ACAGAGATAAAAGCCAGAAGCAG |
| 1043 | Msr1-g1 | ATGAAGTACAAGTGACCCCAGCA |
| 1044 | Msr1-g2 | ATCATCACAGATTGTGCCCCACT |
| 1045 | Msr1-g3 | CATTCAGCCATATTGGACCAGTA |
| 1046 | Msr1-g4 | ATGCTGTCATTGAACGTGCGTCA |
| 1047 | Msr1-g5 | TTCCCAATTCAAAAGCTGAGCTG |
| 1048 | Muc16-g1 | TCTGAAAAGCTGATTGAGCGCAT |
| 1049 | Muc16-g2 | TGAGTACCAGTAGTACCGCCAAG |
| 1050 | Muc16-g3 | GAGACAAATAATAAGCTAGACGA |
| 1051 | Muc16-g4 | CTGTTTTGAGAATACCCATCCAC |
| 1052 | Muc16-g5 | GTATGGTTATATTCAGTGGCACA |
| 1053 | Muc5ac-g1 | CAGTAGTCAAATAAGCAGCCTTG |
| 1054 | Muc5ac-g2 | AGAACACATAGTTGCAGAGACCA |
| 1055 | Muc5ac-g3 | GCCAATGTCAGTTTCCACCACCA |
| 1056 | Muc5ac-g4 | GCATAGTAACAGTGGCCATCAAG |
| 1057 | Muc5ac-g5 | ATCAAAAGTGATGTAGTGGCCAT |
| 1058 | Myc-g1 | ATACTATTTAAGTTTGAGGCAGT |
| 1059 | Myc-g2 | CATGCATTTTAATTCCAGCGCAT |
| 1060 | Myc-g3 | AAGTTATTTACATTTCAAGGCCC |
| 1061 | Myc-g4 | CTGGAATTACTACAGCGAGTCAG |
| 1062 | Myc-g5 | CCGCAACATAGGATGGAGAGCAG |
| 1063 | Mycn-g1 | TCATACTAAAGTATACAGGCCGT |
| 1064 | Mycn-g2 | AAACGTTTAGCAAGTCCGAGCGT |
| 1065 | Mycn-g3 | TCAAAATGTGCAAAGTGGCAGTG |
| 1066 | Mycn-g4 | ACAGACACACTAGTGACCGCAGC |
| 1067 | Mycn-g5 | AAACAAGGAAGAAACAGGCTAGG |
| 1068 | N6amt1-g1 | CTCTGAAAACAAAATGACCCAGC |
| 1069 | N6amt1-g2 | ACGGTAACTAAGTAGAACAGCCC |
| 1070 | N6amt1-g3 | TAAGGCTAAATCAAACGTGCCTT |
| 1071 | N6amt1-g4 | TTATTTATTGTATATGAGCACAC |
| 1072 | N6amt1-g5 | AATAAAATTTCTTGGCCAGGCAA |
| 1073 | Nanog-g1 | ACCAAGTTGTAAATAGAGCTCAG |
| 1074 | Nanog-g2 | ACAGTGTATACCAAGACCCACGC |
| 1075 | Nanog-g3 | CATACGTAACAAGATCTGACGCC |
| 1076 | Nanog-g4 | CAAAGACAATTAGAGCTATGCAG |
| 1077 | Nanog-g5 | AGAGCATCTCAGTAGCAGACCCT |
| 1113 | Nras-g1 | CTTTTAAATAGAAACCACCCAGT |
| 1114 | Nras-g2 | ACCGAGATAACTGTTCAAGCCCC |
| 1115 | Nras-g3 | TTGCATTTATGAATACAGAGCAG |
| 1116 | Nras-g4 | TACGTAATCACTAGGCGCCCAAG |
| 1117 | Nras-g5 | GTACTCAGTCATTTCACACCAGC |
| 1118 | Nrp1-g1 | TTTCCGAGAAGAATCCACCACAG |
| 1119 | Nrp1-g2 | AGTTTCAGAGATTTGTGCAGCAA |
| 1120 | Nrp1-g3 | AAAGCAGAGTAACAGAGTCCCCA |
| 1121 | Nrp1-g4 | CATAGATATACCAGTTTCCCAGG |
| 1122 | Nrp1-g5 | CAAAGATGATGTAGGTGCACTCC |
| 1123 | Nt5e-g1 | TACAATTACAAGATAGTCCAAGG |
| 1124 | Nt5e-g2 | AGATGTATTCAGAAACCACGCTG |
| 1125 | Nt5e-g3 | CTTTCGGTTAATATCGTACACCA |
| 1126 | Nt5e-g4 | ATCTCAAAACCAGAGTGCCCCAG |
| 1127 | Nt5e-g5 | CTGAGAGACAACAAGAGCCCAAA |
| 1133 | Otulin-g1 | AGCACAGAGAAGAACGGCACTTC |
| 1134 | Otulin-g2 | TCAGCAGTTTTCATTGCAGCCAG |
| 1135 | Otulin-g3 | TATAATTCCAGCTTTGGCAGCAA |
| 1136 | Otulin-g4 | ACATCAGGAACTTCACAGCTTCG |
| 1137 | Otulin-g5 | TGCTCCATAAGCGTCCGCCACCT |
| 1138 | Pced1b-g1 | GTGGAAATTTAAGTCCAGCACAT |
| 1139 | Pced1b-g2 | AAACAAAGAGAAGTCCAAGACAG |
| 1140 | Pced1b-g3 | TCCATGTACTCAGAGTAGACCCG |
| 1141 | Pced1b-g4 | CTACTCAAGGAATTCCGCCCATA |
| 1142 | Pced1b-g5 | GCCTCTCATTACCTGCAGCCGAG |
| 1148 | Pdcd1-g1 | AGTTCAGCATAAGATCCTCCGAC |
| 1149 | Pdcd1-g2 | CAAACCATTACAGAAGGCGGCCT |
| 1150 | Pdcd1-g3 | AAGTCCCTAGAAGTGCCCAACAG |
| 1151 | Pdcd1-g4 | AGCAGCAATACAGGGATACCCAC |
| 1152 | Pdcd1-g5 | CCAGTCTACGAATTTCCCACCTG |
| 1153 | Pdcd1lg2-g1 | ATGAAAACATGAAGTGGCCACGT |
| 1154 | Pdcd1lg2-g2 | AAGTAGAAACAAATACCACAGTG |
| 1155 | Pdcd1lg2-g3 | CAAAAGTGCAAATGGCAGGTCCT |
| 1156 | Pdcd1lg2-g4 | GCATTCCAGAACATGCAGCTGAA |
| 1157 | Pdcd1lg2-g5 | TACAACAATTACCTTGTGACTCA |
| 1163 | Pik3ca-g1 | CCAAGATAAAGGTTGCCACGCAG |
| 1164 | Pik3ca-g2 | AGCGCACTATTTATGACCCAGAG |
| 1165 | Pik3ca-g3 | AGTGTTTCAATTATAGAGCACGT |
| 1166 | Pik3ca-g4 | AGTAGAAATCTAGAGCGACCACT |
| 1167 | Pik3ca-g5 | AAGCAGATATTGATCACCCCAGC |
| 1178 | Pim1-g1 | CTTTACTCAGATAAAACCAGCGG |
| 1179 | Pim1-g2 | TTTTATGTACAGTCAGCAGACCC |
| 1180 | Pim1-g3 | AAAAACCAAACCAAACCCCCAAC |
| 1181 | Pim1-g4 | CCATTTAATAAGGTGCTGACACT |
| 1182 | Pim1-g5 | CAGAAGTCTAATGACGCCCGAGA |
| 1183 | Pkn2-g1 | AGAGTACAATAACAACAGGCCAA |
| 1184 | Pkn2-g2 | ACGAGTAATAGCATCCAAAGCCC |
| 1185 | Pkn2-g3 | GTTCATGTAAGTATTGCAACCCA |
| 1186 | Pkn2-g4 | CATATATAAGTACACCAAGGCCC |
| 1187 | Pkn2-g5 | TTTTGCATTATCAAAAGCCAGCT |
| 1193 | Plac1-g1 | AGAAACAGAATTTTCAGAAGCCC |
| 1194 | Plac1-g2 | TGACTACACAAGAAGGACCTCCA |
| 1195 | Plac1-g3 | TGAACCAATCTGTCGAGCACAGC |
| 1196 | Plac1-g4 | CCCAGAAACATTTGCACAGTCAG |
| 1197 | Plac1-g5 | CACATATTTCGTTGATGAGCCCT |
| 1203 | Pole-g1 | ATCCAGAGATAGTACCTTGCACA |
| 1204 | Pole-g2 | CAGAAATTACAGCTGTGGCAGAT |
| 1205 | Pole-g3 | CTTGACAATTGGAGCAGAGCCAC |
| 1206 | Pole-g4 | AAAACCGTGTTTACTTAGCAGGC |
| 1207 | Pole-g5 | TCCAGAGAAGGAATTCCCAGCAT |
| 1228 | Prkci-g1 | CCAATAAGAAATATGGCCACAAG |
| 1229 | Prkci-g2 | AGATTTAAAAACAAAGACCACCA |
| 1230 | Prkci-g3 | AAGCAAGAATGCAGCCCGACAAG |
| 1231 | Prkci-g4 | GACACAGATAACTAGACACCCAT |
| 1232 | Prkci-g5 | CCAACGATATCAAACGGAGACCT |
| 1243 | Psmg1-g1 | TGCAATTATAAACTTGGAGCAAG |
| 1244 | Psmg1-g2 | GTAGCACAGATACAGAACCGCAG |
| 1245 | Psmg1-g3 | CATTCCAGAGCTTAGCACAACCG |
| 1246 | Psmg1-g4 | ACAAAACATCAGTGACTGCCCCC |
| 1247 | Psmg1-g5 | CAGAAAACATCTGTAGGGGACAG |
| 1253 | Ptar1-g1 | CGCACTTATTCAGATCCACCTCG |
| 1254 | Ptar1-g2 | CTTGATTAAAGCATCCAACACCA |
| 1255 | Ptar1-g3 | CACCAGAGTTGACAGGCACACTC |
| 1256 | Ptar1-g4 | TCATCTTCTTGATTTCCGCAATC |
| 1257 | Ptar1-g5 | ACCAGAGTTGACAGGCACACTCA |
| 1263 | Ptger2-g1 | TCTTGAATATGAAGCCAGCACCA |
| 1264 | Ptger2-g2 | TCCATTAAGCAATCACGAGACAG |
| 1265 | Ptger2-g3 | ATCCAAGAAATGGAACCGTGCAC |
| 1266 | Ptger2-g4 | GCACCAATTCCGTTACCAGCACG |
| 1267 | Ptger2-g5 | AAGCGAAATAGGTACACGCGTGA |
| 1268 | Ptger4-g1 | GCACAATACTACGATGGCCACCA |
| 1269 | Ptger4-g2 | AGTCACATCAGAATGACAGCCAA |
| 1270 | Ptger4-g3 | GTTAATGAACACTCGCACCACGA |
| 1271 | Ptger4-g4 | CACAGATGATGCTGAGACCCGAC |
| 1272 | Ptger4-g5 | CCAAGCAATTCACAAGGACACGT |
| 1283 | Ptgs2-g1 | TCATAGTTAAGACAGAGCAGCAC |
| 1284 | Ptgs2-g2 | ATATATTTCTTCATTAGACACCC |
| 1285 | Ptgs2-g3 | TATATTTCTTCATTAGACACCCT |
| 1286 | Ptgs2-g4 | GCAAACATCATATTTGAGCCTTG |
| 1287 | Ptgs2-g5 | TTTATGCGTAAATTCCAACAGCC |
| 1288 | Ptk2-g1 | CATATAATATCAAAGATGCCAGG |
| 1289 | Ptk2-g2 | TTCTACAGATAGTTCGCAACCCA |
| 1290 | Ptk2-g3 | CACAATCATTTGAAGACACCAGA |
| 1291 | Ptk2-g4 | GCAATAACTCAGAAGGCAGCAGT |
| 1292 | Ptk2-g5 | TGTCATATTCTTTAGCCCAACAC |
| 1298 | Ptpn6-g1 | ATACAGATTCAGATGACCACAGT |
| 1299 | Ptpn6-g2 | TGATCCAGAAAGCTGAGGACACC |
| 1300 | Ptpn6-g3 | ACAAACTCTAGAGATGAGCCTCA |
| 1301 | Ptpn6-g4 | CAGAGAGCACAAAATCACCAGGT |
| 1302 | Ptpn6-g5 | TACAGGTCATAGAAGTCCCCTGA |
| 1308 | Ptx3-g1 | GCAGACATTAATCTGAAAGCACC |
| 1309 | Ptx3-g2 | TCATTCGTCTATTACGCACCGAA |
| 1310 | Ptx3-g3 | AAAGAATGAACAATGGGCAACAG |
| 1311 | Ptx3-g4 | AATTCACATACATGAGCTCGTAG |
| 1312 | Ptx3-g5 | ATGCTAATGATTCGTCAAAGCCC |
| 1328 | Ret-g1 | TGTATATAGCAAAGGCAACACCA |
| 1329 | Ret-g2 | GATTAAAACAAGACAGACCCACC |
| 1330 | Ret-g3 | TCTTTCAGCATTTTCACAGCCAC |
| 1331 | Ret-g4 | CCAAGTCATGAATGGCAGACCCC |
| 1332 | Ret-g5 | CAGACACAGAAGATGGACAGCAG |
| 1343 | Ric1-g1 | GTACACATACAGAAGCAGCAGCA |
| 1344 | Ric1-g2 | GATTGTATAAACCTGCACACAGC |
| 1345 | Ric1-g3 | TGCAAAAAGGTAATTCCACAGAG |
| 1346 | Ric1-g4 | CAGCATAATTGATAAGAACCCCC |
| 1347 | Ric1-g5 | ACTTAAATAAAAATAGCACCAGA |
| 1348 | Ric8a-g1 | CAAAAAGTTAAGAAGAGGCCACT |
| 1349 | Ric8a-g2 | GAAACACAGCGTATGCCCCACAT |
| 1350 | Ric8a-g3 | CTGACAGTATGTAGCCGACCCAA |
| 1351 | Ric8a-g4 | ACATCCAAACACTTGAGGGGCAA |
| 1352 | Ric8a-g5 | TGAGCACAAGATTACACAGGCAT |
| 1353 | Rock1-g1 | AATAAATTTAAAAGGCAGCACCT |
| 1354 | Rock1-g2 | AGGTCCAAAAGTTTTGCCCGCAA |
| 1355 | Rock1-g3 | TTTCATATAGAAATACCCCAACT |
| 1356 | Rock1-g4 | ATGTCCAGACTTATCCAGCAGCA |
| 1357 | Rock1-g5 | TATTTCTCATTAAATGAGCACAG |
| 1358 | Rorc-g1 | TCCAGATCACTTTGACAGCCCCT |
| 1359 | Rorc-g2 | CCAAGAGTAAGTTGGCCGTCAGT |
| 1360 | Rorc-g3 | CCCAGATGACTTGTCCCCACAGA |
| 1361 | Rorc-g4 | ACCACATACTGAATGGCCTCAGT |
| 1362 | Rorc-g5 | ATCCTCAGAAAAACACAGGGCGC |
| 1363 | Ros1-g1 | GTCCAATAGAGATAGCCACCAAC |
| 1364 | Ros1-g2 | GAAATCCATATGATGCACCCAAG |
| 1365 | Ros1-g3 | ACATTGAAAATGGCTGCAGACCT |
| 1366 | Ros1-g4 | AGTCCAATTTCATTTGCAGCAAC |
| 1367 | Ros1-g5 | ACTTCCCAACAAAAGACGCAGGC |
| 1368 | S100a8-g1 | ACCCACTTTTATCACCATCGCAA |
| 1369 | S100a8-g2 | GAAGTCATTCTTGTAGAGGGCAT |
| 1370 | S100a8-g3 | TCTTTGTGAGATGCCACACCCAC |
| 1371 | S100a8-g4 | GTAGACATCAATGAGGTTGCTCA |
| 1372 | S100a8-g5 | TTTATAGAGGAAAGCTTGGCCAG |
| 1373 | S100a9-g1 | ATTTCCCAGAACAAAGGCCATTG |
| 1374 | S100a9-g2 | CACAGATGTTGGTAAGAGCAGTG |
| 1375 | S100a9-g3 | CATGATGTCATTTATGAGGGCTT |
| 1376 | S100a9-g4 | ACCTCTTAATTACTTCCCACAGC |
| 1377 | S100a9-g5 | GCCATCAGCATCATACACTCCTC |
| 1378 | S100pbp-g1 | TACAAGACTTAGAGGCCAAACCC |
| 1379 | S100pbp-g2 | CATTAAGACAGTACACAGAGCCT |
| 1380 | S100pbp-g3 | GCCACATATAAAATGAGACAGAG |
| 1381 | S100pbp-g4 | GAAAAATCAGAAGTGCAAGACCA |
| 1382 | S100pbp-g5 | GTCTATACTCATTATGCCCACCA |
| 1383 | S1pr1-g1 | CATCAATACCTAGTGACAGCCGA |
| 1384 | S1pr1-g2 | CAGTGCAAAATCAAAGCTCCAGG |
| 1385 | S1pr1-g3 | CATTTGCAACAAGATACGATCCG |
| 1386 | S1pr1-g4 | TCTGATGAACAAAAGTCAGGCAG |
| 1387 | S1pr1-g5 | AGCCTTCAGTTACAGCAAAGCCA |
| 1403 | Sema4d-g1 | GAAAACAGTTTAATACGGCACCT |
| 1404 | Sema4d-g2 | AGACACAATAGCTTGGTGCAGTA |
| 1405 | Sema4d-g3 | AAAAGATTCTCACATGGACCCCA |
| 1406 | Sema4d-g4 | ACGTAGCAAGTTCCTGGCTCCAC |
| 1407 | Sema4d-g5 | TGCATAGGTACACACGTCTCCAG |
| 1408 | Serpinb9-g1 | TATCAAGATAGCAAAGAGGCAGT |
| 1409 | Serpinb9-g2 | AAGCAATTACAAGTACAGCGACA |
| 1410 | Serpinb9-g3 | CAGCAAAATTCTATGATGGCAGA |
| 1411 | Serpinb9-g4 | CATTATAAGATCAGGCTGACAAG |
| 1412 | Serpinb9-g5 | CCAAGCGCTGAAACAGAGACTCC |
| 1418 | Shc1-g1 | ATTTTCCATTATAAGAACCCACC |
| 1419 | Shc1-g2 | CAAACCAAAAATTTGGCGACCAT |
| 1420 | Shc1-g3 | CATTGACTGTAAGACCTCCACAC |
| 1421 | Shc1-g4 | AAGAAGTCACCATTGAGCTGCAG |
| 1422 | Shc1-g5 | GAAGCCTCATATCTACCACCCCA |
| 1423 | Shh-g1 | AGAAAAATAGACTTTCAGCAGGT |
| 1424 | Shh-g2 | ACGAAACAAATAAATAGCCAGGA |
| 1425 | Shh-g3 | AAATATAATTTGTGGACCCCCAT |
| 1426 | Shh-g4 | GATTCATAGTAGACCCAGTCGAA |
| 1427 | Shh-g5 | TTAAAAGACAAAAAGAGCCTGAT |
| 1428 | Sirpa-g1 | CACACAGTAGTAGATGCCAGCAT |
| 1429 | Sirpa-g2 | AAACTGTAGATCAACAGCCGGCT |
| 1430 | Sirpa-g3 | AACATTTCTAATTCGAGGAACGT |
| 1431 | Sirpa-g4 | CAGTTCAGAACGGTCGAATCCCC |
| 1432 | Sirpa-g5 | AGTCACCTTCAGTTCCTTCCCCG |
| 1448 | Sox2-g1 | CTGCAGAATCAAAACCCAGCAAG |
| 1449 | Sox2-g2 | GCCTGATTCCAATAACAGAGCCG |
| 1450 | Sox2-g3 | ATTACCAACGATATCAACCTGCA |
| 1451 | Sox2-g4 | CTGTACAAAAATAGTCCCCCAAA |
| 1452 | Sox2-g5 | TCGGACAAAAGTTTCCACTCCGC |
| 1468 | Srrt-g1 | TCCAACTACAAAACAAGACCCCC |
| 1469 | Srrt-g2 | AAAGTTATTGAAGAACGCCACCT |
| 1470 | Srrt-g3 | AGAAGGTTATCAAACCAGCCACT |
| 1471 | Srrt-g4 | CGAAAAGCATCATAGTTCCCACG |
| 1472 | Srrt-g5 | TGCCATTTATGTTGCGGACACGA |
| 1478 | Tacstd2-g1 | CAAGCAGAAAAATAGATGCAGTC |
| 1479 | Tacstd2-g2 | CTGAGAATTAACAGGCCAACCCA |
| 1480 | Tacstd2-g3 | AGGAATTTCAGAAATGCGTCCTT |
| 1481 | Tacstd2-g4 | CCCACCGAGTTTACGCACCAGCA |
| 1482 | Tacstd2-g5 | CCCCCAGCTCCTTAAGCTCCACC |
| 1483 | Tdo2-g1 | CAAATAAATCAATAGAGGCCAAG |
| 1484 | Tdo2-g2 | GAACAAAATGCTTTACGACAGCC |
| 1485 | Tdo2-g3 | ATCAAACAAGCAGAGCAGCACCT |
| 1486 | Tdo2-g4 | ACAAGCAATGAACAGCCAACCAC |
| 1487 | Tdo2-g5 | CATGCGTATTACAGTGCAGCGAA |
| 1493 | Tgfb2-g1 | CAATACATAAAATACAGGCAGAG |
| 1494 | Tgfb2-g2 | ATTTCTAAAGCAGTAGGCAGCAT |
| 1495 | Tgfb2-g3 | TGTATTGTAGATCAACAGCCACT |
| 1496 | Tgfb2-g4 | AGAACCCTTAAAATAGCAGTCAG |
| 1497 | Tgfb2-g5 | AAAGAAAATGCAACGCGTTCCCA |
| 1498 | Tgfbr1-g1 | AATAAGACATTAACAGAGCCCAG |
| 1499 | Tgfbr1-g2 | CCACCAATAGAACAGCGTCGAGC |
| 1500 | Tgfbr1-g3 | AGCATAAGTGCAATGCAGACGAA |
| 1501 | Tgfbr1-g4 | AAGAAGTATCCATAGTGCACAGA |
| 1502 | Tgfbr1-g5 | GCTTCATTTAGTGCCACACCCCA |
| 1508 | Tigit-g1 | TTAAGCAAATGAGTCCCAGCACA |
| 1509 | Tigit-g2 | GTCAACACTATAAATGGCCAGAA |
| 1510 | Tigit-g3 | ACACTGTAAGATGACAGAGCCAC |
| 1511 | Tigit-g4 | CACTGAAGACTGAAGCGACATGC |
| 1512 | Tigit-g5 | GATACAGCAATGAAGCTCTCTAG |
| 1528 | Tnfrsf17-g1 | CCCAAGAAGATCCAGAGCACCGT |
| 1529 | Tnfrsf17-g2 | TAACGACATCTAAAACACCAGCT |
| 1530 | Tnfrsf17-g3 | AGAAAATCGAGGAAGAACAGCAG |
| 1531 | Tnfrsf17-g4 | AAAAGTGCCAAAGAGAGGACCAA |
| 1532 | Tnfrsf17-g5 | ACACTTTGCAAAGCAGTTGGCAC |
| 1533 | Tnfrsf1a-g1 | ATGAAGTAAGATGATCGGACCAG |
| 1534 | Tnfrsf1a-g2 | GCAGCAATTGACAACGCTCGTGA |
| 1535 | Tnfrsf1a-g3 | CAATTTCACGGAAGGAAGCCAGC |
| 1536 | Tnfrsf1a-g4 | ACATACTTTCCTTGGGGACACAA |
| 1537 | Tnfrsf1a-g5 | ATCAGCAGAGCCAGGAGCACCAG |
| 1538 | Tnfrsf1b-g1 | ACAGCAAGTACAGTACCAAGCCG |
| 1539 | Tnfrsf1b-g2 | CAGAGTAAAAGTCAAAGGCAGAG |
| 1540 | Tnfrsf1b-g3 | CAGTCCTAACATCAGCAGACCCA |
| 1541 | Tnfrsf1b-g4 | CTCAGAAGCAAGAATCAGGCAAG |
| 1542 | Tnfrsf1b-g5 | GTACAGGAAGAACTGCAGCTCAA |
| 1543 | Tnfrsf8-g1 | CAAAAATTGTGTGAAGAGCCACT |
| 1544 | Tnfrsf8-g2 | TACAAGAGTATGCAGCTGCCAGT |
| 1545 | Tnfrsf8-g3 | GGAGAAATTTAAAGGGCACACAG |
| 1546 | Tnfrsf8-g4 | GCAAAGCATAGTCTTGAGCAGTG |
| 1547 | Tnfrsf8-g5 | AGAACATGACCTCAGTGCAGCTG |
| 1548 | Tnfsf11-g1 | TGGAATTCAGAATTGCCCGACCA |
| 1549 | Tnfsf11-g2 | AAGAACTTATTTGCAGGTCCCAG |
| 1550 | Tnfsf11-g3 | CGAAAGCAAATGTTGGCGTACAG |
| 1551 | Tnfsf11-g4 | AATAAACTACATGTGGTCACCAG |
| 1552 | Tnfsf11-g5 | TCGAAAGTACAGGAACAGAGCGA |
| 1568 | Trpm7-g1 | AATGTAGAACATATTGGCCACCA |
| 1569 | Trpm7-g2 | AAAAACTCAATTTTGGCACAGAG |
| 1570 | Trpm7-g3 | CATCAATAAGATTCTGAGCCAAG |
| 1571 | Trpm7-g4 | TACAAGAGCATCAAGCATAGCCT |
| 1572 | Trpm7-g5 | CCTTCAAATATCAAAGCCACCAC |
| 1573 | Txk-g1 | CTATAAACATTTATACAGCCCCA |
| 1574 | Txk-g2 | AAAGAGAAATGTCAGCGCTCAAG |
| 1575 | Txk-g3 | CCCACATTAAAACTCCGAACGAC |
| 1576 | Txk-g4 | GAGATCTTTACTACGCAGGCAGA |
| 1577 | Txk-g5 | ACGAGATATGAGACCAGCTGCAT |
| 1598 | Vdac2-g1 | TCAAAGTCAACATCACAGCCGAG |
| 1599 | Vdac2-g2 | ACATAAAACAAACATGCACACCA |
| 1600 | Vdac2-g3 | CGTAACCAAAGACAGCTGACCCA |
| 1601 | Vdac2-g4 | CAGATGTTGAAAATTCCACACCG |
| 1602 | Vdac2-g5 | TTACCTCATCTTAAAACAGCCAC |
| 1603 | Vps13a-g1 | TATGTATAGCATAAGCCCACCAC |
| 1604 | Vps13a-g2 | TTTAAAAGCACATAAGCGCACAG |
| 1605 | Vps13a-g3 | GCCTCTTTCCAATTATCACCCAC |
| 1606 | Vps13a-g4 | AATAACTGTAGAGTGCTCAGCCA |
| 1607 | Vps13a-g5 | CACTACAACGTTTAACAGCTCCG |
| 1608 | Vps35-g1 | AAGTAAATTGTTTGCCAAGGCCC |
| 1609 | Vps35-g2 | AAAGAGAAAGTACAGAGACAGGA |
| 1610 | Vps35-g3 | TTCCACTAAGTTCATGAACTCCG |
| 1611 | Vps35-g4 | CCTTTTGCCAAAACTCCAGCCAC |
| 1612 | Vps35-g5 | CATATTGATGTTTCAGGTTCCAG |
| 1613 | Vps4b-g1 | ACATTAGATTACAGAGTCCAAGC |
| 1614 | Vps4b-g2 | GCACAAACAAGGTTAACCCCGAC |
| 1615 | Vps4b-g3 | AGAACTAGTTATGCACGGAGCAA |
| 1616 | Vps4b-g4 | AACTTAGTAGACCTGTAGCAGCA |
| 1617 | Vps4b-g5 | CGTAATCACTGAGAGCCACACAA |
| 1618 | Vtcn1-g1 | GATGTCACACAATTGCAGAGCCC |
| 1619 | Vtcn1-g2 | TCCAAAAGATGATCTGCCCCAAG |
| 1620 | Vtcn1-g3 | AGAGTGACATCATAACAGCCCAT |
| 1621 | Vtcn1-g4 | CATTTCAAAGAGCATGGCCGTAT |
| 1622 | Vtcn1-g5 | TGTGTAAATTCAGTGAGACACGT |
| 1628 | Wdr7-g1 | AAATCATACCAGATGCCGCTACA |
| 1629 | Wdr7-g2 | ATACAAACAGATCGGAGCCCGCT |
| 1630 | Wdr7-g3 | AGTTTACAAACAGAGAGCACAAG |
| 1631 | Wdr7-g4 | ACTACAGAGCAGTTAGCCAGCTA |
| 1632 | Wdr7-g5 | ATGCCATCAACATAGTCCCACAG |
| 1633 | Wdr83-g1 | TATTTATTACTTTACATGCCAGC |
| 1634 | Wdr83-g2 | GTTATCAAAGGAGCCAGCCGCAT |
| 1635 | Wdr83-g3 | TGTACCACATTAGAACCCACAGG |
| 1636 | Wdr83-g4 | AACATTATTATTGTCCCCCAGGA |
| 1637 | Wdr83-g5 | TACTTTTAAATGCATCAGTCCGC |
| 1638 | Wfdc2-g1 | ACCAGAGAGAAAGGAGGCCACAG |
| 1639 | Wfdc2-g2 | CTCAGAATTTGGGTGTGGTGCAG |
| 1640 | Wfdc2-g3 | CCGCTGATTGAGTAGTAGTCCCA |
| 1641 | Wfdc2-g4 | GTCCACCTGACACTGGTCCTCAC |
| 1642 | Wfdc2-g5 | GTCCGTAATTGGTTCAAGCTGGG |

TABLE 5
MUCIG-Lib3 gRNAs

| SEQ | | |
|---|---|---|
| ID | | |
| NO: | Name: | Sequence: |
| 103 | Adam10-g1 | ATAAAAGTTTATCGAGAGCCAAG |
| 104 | Adam10-g2 | TCAATGTAAAACGTGCCACCACG |
| 105 | Adam10-g3 | GACAAGTATTTCTTTCAGCCAGA |
| 106 | Adam10-g4 | AATACACAAAGTAATAAGCAGGC |
| 107 | Adam10-g5 | CAGAATTAACACTGTCGGCAACA |
| 108 | Adam17-g1 | CAGAACATCTTGAAGCACCAGAG |
| 109 | Adam17-g2 | CATTCATACATATACCCACACAC |
| 110 | Adam17-g3 | AGTTACAGAGTTGAGAGCCACCA |
| 111 | Adam17-g4 | GAAAACCAGAACAGACCCAACGA |
| 112 | Adam17-g5 | TATCTTCAGACTTATACACCAGC |
| 113 | Adar-g1 | TTCACCATAAGAGAGCTGCAGTA |
| 114 | Adar-g2 | TCAAGGAATGCAAGACAGCCACG |
| 115 | Adar-g3 | CTTTTCATAATAATGGCAGCCAG |
| 116 | Adar-g4 | AGACCAGAAGAATCCCAGTGCAC |
| 117 | Adar-g5 | CTGAGCATACTCTAACAACCCGC |
| 123 | Adora2a-g1 | ACAAACAAACAAACAAGCCCCAC |
| 124 | Adora2a-g2 | TTAATGAGATTGGTCCAGCCAAC |
| 125 | Adora2a-g3 | AAAATCCTTAGGTAGATGGCCAG |
| 126 | Adora2a-g4 | CAGCAAATCGCAATGATGCCCTT |
| 127 | Adora2a-g5 | ATGATGTACACCGAGGAGCCCAT |
| 168 | Atg10-g1 | CCTGCAGTAATTCAACAGAGCAG |
| 169 | Atg10-g2 | CCCTAAAGTAAAGAACCGGCACT |
| 170 | Atg10-g3 | CAGAGGTAAATTCAGACCAACCA |
| 171 | Atg10-g4 | CATCGTTCACTAAAGCGAGCACA |
| 172 | Atg10-g5 | TACATTAATTTTCAGAAACAGGC |
| 233 | Braf-g1 | AGACAGTTCCAAATGACCCAGAT |
| 234 | Braf-g2 | GAATTCTGTAAACAGCACAGCAT |
| 235 | Braf-g3 | CATTCAACATTTTCACTGCCACA |
| 236 | Braf-g4 | ATAACCACATGTTTGACAACGGA |
| 237 | Braf-g5 | TCCACAAAATAGATCCAGACAAC |
| 243 | Btla-g1 | ATATGTATATTAATCCAGCAGCA |
| 244 | Btla-g2 | GACCTTTAAGACGCAGCACCAGC |
| 245 | Btla-g3 | ATCCTTTTCAGAAAGCAGAGCAG |
| 246 | Btla-g4 | TAACAGAATAAAGTGGAGTGCAA |
| 247 | Btla-g5 | TGTAGAACAGCTATACGACCCAT |
| 248 | C10orf54-g1 | TCCAGAGATAGATAAAGCACCCG |
| 249 | C10orf54-g2 | CCATACAGGTAATGAGAGCCCAG |
| 250 | C10orf54-g3 | CAGACAAAGCTAGATCCCCAGAG |
| 251 | C10orf54-g4 | CAAGACACTAATGAGCTCACAGT |
| 252 | C10orf54-g5 | CCAGGAAAATAGCAAGGAGCAGG |
| 278 | Cblb-g1 | AAAAACCTGAAATTGCCACAGAG |
| 279 | Cblb-g2 | AATTCCGTAAAATAGAGCCCCAG |
| 280 | Cblb-g3 | GAACTGAAAAAGTAGCAGCAAGG |
| 281 | Cblb-g4 | GCAAGCTACATGAAGCCCAACAG |
| 282 | Cblb-g5 | TGACCATTATCACAAGACCGAAC |
| 333 | Cd274-g1 | CGTAGCAAGTGACAGCAGGCTGT |
| 334 | Cd274-g2 | CCATCGTGACGTTGCTGCCATAC |
| 335 | Cd274-g3 | CGCTTGTAGTCCGCACCACCGTA |
| 336 | Cd274-g4 | TAGAAAACATCATTCGCTGTGGC |
| 337 | Cd274-g5 | ATTTCTCCACATCTAGCATTCTC |
| 338 | Cd276-g1 | ATAATAGCAGTTACACAGTCTGC |
| 339 | Cd276-g2 | GTGAACATCGAACAAGCCCCGCT |
| 340 | Cd276-g3 | AGCAAGAACTAAGAGGTCACTGT |
| 341 | Cd276-g4 | GACAACAAAAGCCAGGGCCAGAT |
| 342 | Cd276-g5 | TTTAATGAAGAGCTGACGGCCAA |
| 353 | Cd47-g1 | CAAGCAAGACAGAAGCGCCAAGT |
| 354 | Cd47-g2 | TAGAGATTACAATGAGGCCAAGT |
| 355 | Cd47-g3 | ACCAAAGCAAGGACGTAGCCCAG |
| 356 | Cd47-g4 | CCACGATGACTGTGAGCACCAGC |
| 357 | Cd47-g5 | TAAACAGTAGTTGAGCTGAACCT |
| 358 | Cd5-g1 | ACAAAGGACAAATGTCCAAGCGT |
| 359 | Cd5-g2 | AGAGTCCAAGGAGAAAGCCAACC |
| 360 | Cd5-g3 | AATTATTTAGACTCTAGGACCAT |
| 361 | Cd5-g4 | TCCCACTGTGATCTCTGGCGCAC |
| 362 | Cd5-g5 | ATAAGTCCTTGTAAGTACCCCAC |
| 363 | Cd55-g1 | AAATGCTAGCATTTCCAACCAGG |
| 364 | Cd55-g2 | CATATATATAACGGTCACCACCT |
| 365 | Cd55-g3 | TCTTGAAGACAATGACAGCATGC |
| 366 | Cd55-g4 | CAAAACTGAGCAACTGGAGACCA |
| 367 | Cd55-g5 | GTTAAATTAGAATGTGCCACCTC |
| 468 | Csf1-g1 | TCAGCAGCATAAAGAGACCAAGG |
| 469 | Csf1-g2 | TGCACACATATTTTCAAGACCCA |
| 470 | Csf1-g3 | GCCCACAATAAATAGTGGCAGTA |
| 471 | Csf1-g4 | TCAGCAAGACTAGGATGATGCCC |
| 472 | Csf1-g5 | CATCTATTATGTCTTGTACCAGA |
| 473 | Csf1r-g1 | CACACAAGAATATATGCCAGCGT |
| 474 | Csf1r-g2 | ATAGTAAATATAGAGGCTAGCAC |
| 475 | Csf1r-g3 | CATGACAGACATACAGGCCACCA |
| 476 | Csf1r-g4 | CAGCAGTATTCAGTGATGACCAG |
| 477 | Csf1r-g5 | CACTTGAAGAAGTCGAGACAGGC |
| 488 | Ctla4-g1 | GTGTTTATATTCAAACCACCAGC |
| 489 | Ctla4-g2 | ATAAAATGAGTGTAAAGACCCAG |
| 490 | Ctla4-g3 | TCAAAGAAACAGCAGTGACCAGG |
| 491 | Ctla4-g4 | TGACACAACAGAAATATCCCAGC |
| 492 | Ctla4-g5 | CAATGACATAAATCTGCGTCCCG |
| 493 | Cxcl1-g1 | CAAGACATACAAACACAGCCTCC |
| 494 | Cxcl1-g2 | AATGTAAAATAAAAACCACACAC |
| 495 | Cxcl1-g3 | TTGTATAGTGTTGTCAGAAGCCA |
| 496 | Cxcl1-g4 | ATGACTTCGGTTTGGGTGCAGTG |
| 497 | Cxcl1-g5 | AATACATAAATAAATAGGACCCT |
| 498 | Cxcl5-g1 | ATAAAAGTTATATGCCAGCCCAG |
| 499 | Cxcl5-g2 | AAATATATAGTTAGTGGCCCAAA |
| 500 | Cxcl5-g3 | ACAACAGTAAAAGAGGTCCCCAT |
| 501 | Cxcl5-g4 | AACAGCAACAGAAATGCCAGCGG |
| 502 | Cxcl5-g5 | GTAATATAAAGAAGTGAGACACT |
| 558 | Entpd1-g1 | TAGAAAGCAGAAAACGCCCCAAA |
| 559 | Entpd1-g2 | ATAGTTAATAGTAATCCACCCAT |
| 560 | Entpd1-g3 | ACACAGTATAGTCCTCGCCATAG |
| 561 | Entpd1-g4 | CAAGTTCAGCATGTAGCCCAAAG |
| 562 | Entpd1-g5 | AGTCACATTAGCTGCACGAGCAC |
| 568 | Epcam-g1 | ACCATATTCATTCAGAGAGCAAC |
| 569 | Epcam-g2 | TCTAGTGAAACATGCAGCTGCAG |
| 570 | Epcam-g3 | CTCACGTGCAGAATCAGTCCATC |
| 571 | Epcam-g4 | CAGCTTGTAGTTGTCACAGACAC |
| 572 | Epcam-g5 | CACGCCCCTCCCCGCCCTCACCT |
| 578 | Erbb2-g1 | CGACTTTCATATAACACCCACTC |
| 579 | Erbb2-g2 | AGACCATAGCATACTCCAGCACA |
| 580 | Erbb2-g3 | ACACAGTGAGTTACAGACCAAGC |
| 581 | Erbb2-g4 | GCAAAAACGTCTTTGACAACCCC |
| 582 | Erbb2-g5 | ACCATCAAACACATCGGAGCCAG |
| 628 | Fitm2-g1 | GTGCAATTTCATATGACAAGCCA |
| 629 | Fitm2-g2 | CTTCCACAATCATGAGCGCACAG |
| 630 | Fitm2-g3 | ATATATACCTTTAATCCCAGCAC |
| 631 | Fitm2-g4 | AAACCAAACATGGTGCCGAACAC |
| 632 | Fitm2-g5 | AACTCTAAAGAGAAGCAGAGCCG |
| 688 | Gpi1-g1 | CACCACCAAGTAAAGAGCCAACC |
| 689 | Gpi1-g2 | AATGTTAGAGACAAACCAGACAC |
| 690 | Gpi1-g3 | CTCATAACGATCAATCCTCCGAG |
| 691 | Gpi1-g4 | AACAACATGACACGTCAAAGCCC |
| 692 | Gpi1-g5 | CGACAAAGTGCTTTGCAACTGCA |
| 703 | Havcr2-g1 | CCAAAGTCAGAAATGAAGGCGAG |
| 704 | Havcr2-g2 | AGACACCAATGATAAGTGCCAGG |
| 705 | Havcr2-g3 | CCCACCTAAGAAAGCCAGGACCT |
| 706 | Havcr2-g4 | AGTCCTTAATTTCATCAGCCCAT |
| 707 | Havcr2-g5 | TATAGTGTTAAGCATATGCCACC |
| 758 | Ido1-g1 | ATTTCCACCAATAGAGAGACGAG |
| 759 | Ido1-g2 | AGACAGATATATGCGGAGAACGT |
| 760 | Ido1-g3 | AATCTACATAATATACAACAGGC |
| 761 | Ido1-g4 | GCATAAGACAGAATAGGAGGCAG |
| 762 | Ido1-g5 | AACCTCAAAACCAGGCACGCCAG |
| 763 | Ikbkg-g1 | CAGAGAAGATTCTTCACCCAGCA |
| 764 | Ikbkg-g2 | AGCAGCTCCTCACAGCGTTCCCT |
| 765 | Ikbkg-g3 | GATATACATGTACTTGTGTCACA |
| 766 | Ikbkg-g4 | GAATTTGCACATAAGGAACTCCT |
| 767 | Ikbkg-g5 | ATTTCATCTTGAAGCAGTGACAC |
| 848 | Kit-g1 | CAAATATTTGTAGGTGAGCACCA |
| 849 | Kit-g2 | TGAATTTGTCAGAATGCAGCCAT |
| 850 | Kit-g3 | GACATGTTTAAACTTGCACAGCG |
| 851 | Kit-g4 | TAAATTCTAGACAGTGAGCGACA |
| 852 | Kit-g5 | ACTTTCAAATGTGTACACGCAGC |
| 858 | Klrc1-g1 | GTTACAAATAAAACAGCCCACAC |
| 859 | Klrc1-g2 | TAAGACAAAACAGATGAGGCCCA |
| 860 | Klrc1-g3 | AAATTCATCTAAAGGGAGCCAGA |
| 861 | Klrc1-g4 | TACACAATCTGATGAGGCCAAAG |
| 862 | Klrc1-g5 | CGAATAGATGATTTCCTGCTCGA |
| 893 | Lag3-g1 | AGAAGCAAAAAGCCAAGGAGCAG |
| 894 | Lag3-g2 | CAAAAGGACCCAATCAGACAGCT |
| 895 | Lag3-g3 | CTTCGTAGAAAGTTAGGATCCAG |
| 896 | Lag3-g4 | AGTCACTGTGATGACCGCCAACG |
| 897 | Lag3-g5 | CAGACAGACAGACAGACACACAC |
| 908 | Lgals1-g1 | GAAAGCACAAGAGAGGTCACTGA |
| 909 | Lgals1-g2 | AGACCAAGAACACATGGAGGCAT |
| 910 | Lgals1-g3 | TTTATTAAGACAAATGCGGTCCG |
| 911 | Lgals1-g4 | CAGTCAGAAGACTCCACCCGAGA |
| 912 | Lgals1-g5 | CGAACTTTGAGACATTCCCCAGG |
| 913 | Lgals3-g1 | AAAGGCATTCTAACTAGGGCAGC |
| 914 | Lgals3-g2 | TTAAGCGAAAAGCTGTCTGCCAT |
| 915 | Lgals3-g3 | ACACAATAATAAATACATCTGCT |
| 916 | Lgals3-g4 | ACAGCTTGTCCTCTGACCTCCAC |
| 917 | Lgals3-g5 | TCCACTTCCAGGCAGTGACGCGT |
| 918 | Lgals9-g1 | CCTTCACATATGATCCACACCGA |
| 919 | Lgals9-g2 | ATATCATGATGGACTTGGACGGG |
| 920 | Lgals9-g3 | GGGTACACCACAGGAGGGATTCC |
| 921 | Lgals9-g4 | AGAATTTCTTGTTCACCATCACC |
| 922 | Lgals9-g5 | GGAAAGATAAGACACAGGCAGAG |
| 978 | Map2k7-g1 | TAAAAATAAAACCATCAGGCCCA |
| 979 | Map2k7-g2 | CCAGAAATGACAAGGAGCAGCAA |
| 980 | Map2k7-g3 | AACAGGACAGTTAAGAGCCACAG |
| 981 | Map2k7-g4 | CAGTGCTTTCACAATCGCCACAG |
| 982 | Map2k7-g5 | ACATCCTTAAACCAGGACGCGAC |
| 1043 | Msr1-g1 | ATGAAGTACAAGTGACCCCAGCA |
| 1044 | Msr1-g2 | ATCATCACAGATTGTGCCCCACT |
| 1045 | Msr1-g3 | CATTCAGCCATATTGGACCAGTA |
| 1046 | Msr1-g4 | ATGCTGTCATTGAACGTGCGTCA |
| 1047 | Msr1-g5 | TTCCCAATTCAAAAGCTGAGCTG |
| 1058 | Myc-g1 | ATACTATTTAAGTTTGAGGCAGT |
| 1059 | Myc-g2 | CATGCATTTTAATTCCAGCGCAT |
| 1060 | Myc-g3 | AAGTTATTTACATTTCAAGGCCC |
| 1061 | Myc-g4 | CTGGAATTACTACAGCGAGTCAG |
| 1062 | Myc-g5 | CCGCAACATAGGATGGAGAGCAG |
| 1118 | Nrp1-g1 | TTTCCGAGAAGAATCCACCACAG |
| 1119 | Nrp1-g2 | AGTTTCAGAGATTTGTGCAGCAA |
| 1120 | Nrp1-g3 | AAAGCAGAGTAACAGAGTCCCCA |
| 1121 | Nrp1-g4 | CATAGATATACCAGTTTCCCAGG |
| 1122 | Nrp1-g5 | CAAAGATGATGTAGGTGCACTCC |
| 1123 | Nt5e-g1 | TACAATTACAAGATAGTCCAAGG |
| 1124 | Nt5e-g2 | AGATGTATTCAGAAACCACGCTG |
| 1125 | Nt5e-g3 | CTTTCGGTTAATATCGTACACCA |
| 1126 | Nt5e-g4 | ATCTCAAAACCAGAGTGCCCCAG |
| 1127 | Nt5e-g5 | CTGAGAGACAACAAGAGCCCAAA |
| 1133 | Otulin-g1 | AGCACAGAGAAGAACGGCACTTC |
| 1134 | Otulin-g2 | TCAGCAGTTTTCATTGCAGCCAG |
| 1135 | Otulin-g3 | TATAATTCCAGCTTTGGCAGCAA |
| 1136 | Otulin-g4 | ACATCAGGAACTTCACAGCTTCG |
| 1137 | Otulin-g5 | TGCTCCATAAGCGTCCGCCACCT |
| 1148 | Pdcd1-g1 | AGTTCAGCATAAGATCCTCCGAC |
| 1149 | Pdcd1-g2 | CAAACCATTACAGAAGGCGGCCT |
| 1150 | Pdcd1-g3 | AAGTCCCTAGAAGTGCCCAACAG |
| 1151 | Pdcd1-g4 | AGCAGCAATACAGGGATACCCAC |
| 1152 | Pdcd1-g5 | CCAGTCTACGAATTTCCCACCTG |
| 1153 | Pdcd1lg2-g1 | ATGAAAACATGAAGTGGCCACGT |
| 1154 | Pdcd1lg2-g2 | AAGTAGAAACAAATACCACAGTG |
| 1155 | Pdcd1lg2-g3 | CAAAAGTGCAAATGGCAGGTCCT |
| 1156 | Pdcd1lg2-g4 | GCATTCCAGAACATGCAGCTGAA |
| 1157 | Pdcd1lg2-g5 | TACAACAATTACCTTGTGACTCA |
| 1283 | Ptgs2-g1 | TCATAGTTAAGACAGAGCAGCAC |
| 1284 | Ptgs2-g2 | ATATATTTCTTCATTAGACACCC |
| 1285 | Ptgs2-g3 | TATATTTCTTCATTAGACACCCT |
| 1286 | Ptgs2-g4 | GCAAACATCATATTTGAGCCTTG |
| 1287 | Ptgs2-g5 | TTTATGCGTAAATTCCAACAGCC |
| 1288 | Ptk2-g1 | CATATAATATCAAAGATGCCAGG |
| 1289 | Ptk2-g2 | TTCTACAGATAGTTCGCAACCCA |
| 1290 | Ptk2-g3 | CACAATCATTTGAAGACACCAGA |
| 1291 | Ptk2-g4 | GCAATAACTCAGAAGGCAGCAGT |
| 1292 | Ptk2-g5 | TGTCATATTCTTTAGCCCAACAC |
| 1348 | Ric8a-g1 | CAAAAAGTTAAGAAGAGGCCACT |
| 1349 | Ric8a-g2 | GAAACACAGCGTATGCCCCACAT |
| 1350 | Ric8a-g3 | CTGACAGTATGTAGCCGACCCAA |
| 1351 | Ric8a-g4 | ACATCCAAACACTTGAGGGGCAA |
| 1352 | Ric8a-g5 | TGAGCACAAGATTACACAGGCAT |
| 1368 | S100a8-g1 | ACCCACTTTTATCACCATCGCAA |
| 1369 | S100a8-g2 | GAAGTCATTCTTGTAGAGGGCAT |
| 1370 | S100a8-g3 | TCTTTGTGAGATGCCACACCCAC |
| 1371 | S100a8-g4 | GTAGACATCAATGAGGTTGCTCA |
| 1372 | S100a8-g5 | TTTATAGAGGAAAGCTTGGCCAG |
| 1373 | S100a9-g1 | ATTTCCCAGAACAAAGGCCATTG |
| 1374 | S100a9-g2 | CACAGATGTTGGTAAGAGCAGTG |
| 1375 | S100a9-g3 | CATGATGTCATTTATGAGGGCTT |
| 1376 | S100a9-g4 | ACCTCTTAATTACTTCCCACAGC |
| 1377 | S100a9-g5 | GCCATCAGCATCATACACTCCTC |
| 1448 | Sox2-g1 | CTGCAGAATCAAAACCCAGCAAG |
| 1449 | Sox2-g2 | GCCTGATTCCAATAACAGAGCCG |
| 1450 | Sox2-g3 | ATTACCAACGATATCAACCTGCA |
| 1451 | Sox2-g4 | CTGTACAAAAATAGTCCCCCAAA |
| 1452 | Sox2-g5 | TCGGACAAAAGTTTCCACTCCGC |
| 1508 | Tigit-g1 | TTAAGCAAATGAGTCCCAGCACA |
| 1509 | Tigit-g2 | GTCAACACTATAAATGGCCAGAA |
| 1510 | Tigit-g3 | ACACTGTAAGATGACAGAGCCAC |
| 1511 | Tigit-g4 | CACTGAAGACTGAAGCGACATGC |
| 1512 | Tigit-g5 | GATACAGCAATGAAGCTCTCTAG |
| 1538 | Tnfrsf1b-g1 | ACAGCAAGTACAGTACCAAGCCG |
| 1539 | Tnfrsf1b-g2 | CAGAGTAAAAGTCAAAGGCAGAG |
| 1540 | Tnfrsf1b-g3 | CAGTCCTAACATCAGCAGACCCA |
| 1541 | Tnfrsf1b-g4 | CTCAGAAGCAAGAATCAGGCAAG |
| 1542 | Tnfrsf1b-g5 | GTACAGGAAGAACTGCAGCTCAA |
| 1543 | Tnfrsf8-g1 | CAAAAATTGTGTGAAGAGCCACT |
| 1544 | Tnfrsf8-g2 | TACAAGAGTATGCAGCTGCCAGT |
| 1545 | Tnfrsf8-g3 | GGAGAAATTTAAAGGGCACACAG |
| 1546 | Tnfrsf8-g4 | GCAAAGCATAGTCTTGAGCAGTG |
| 1547 | Tnfrsf8-g5 | AGAACATGACCTCAGTGCAGCTG |
| 1548 | Tnfsf11-g1 | TGGAATTCAGAATTGCCCGACCA |
| 1549 | Tnfsf11-g2 | AAGAACTTATTTGCAGGTCCCAG |
| 1550 | Tnfsf11-g3 | CGAAAGCAAATGTTGGCGTACAG |
| 1551 | Tnfsf11-g4 | AATAAACTACATGTGGTCACCAG |
| 1552 | Tnfsf11-g5 | TCGAAAGTACAGGAACAGAGCGA |
| 1598 | Vdac2-g1 | TCAAAGTCAACATCACAGCCGAG |
| 1599 | Vdac2-g2 | ACATAAAACAAACATGCACACCA |
| 1600 | Vdac2-g3 | CGTAACCAAAGACAGCTGACCCA |
| 1601 | Vdac2-g4 | CAGATGTTGAAAATTCCACACCG |
| 1602 | Vdac2-g5 | TTACCTCATCTTAAAACAGCCAC |
| 1608 | Vps35-g1 | AAGTAAATTGTTTGCCAAGGCCC |
| 1609 | Vps35-g2 | AAAGAGAAAGTACAGAGACAGGA |
| 1610 | Vps35-g3 | TTCCACTAAGTTCATGAACTCCG |
| 1611 | Vps35-g4 | CCTTTTGCCAAAACTCCAGCCAC |
| 1612 | Vps35-g5 | CATATTGATGTTTCAGGTTCCAG |
| 1613 | Vps4b-g1 | ACATTAGATTACAGAGTCCAAGC |
| 1614 | Vps4b-g2 | GCACAAACAAGGTTAACCCCGAC |
| 1615 | Vps4b-g3 | AGAACTAGTTATGCACGGAGCAA |
| 1616 | Vps4b-g4 | AACTTAGTAGACCTGTAGCAGCA |
| 1617 | Vps4b-g5 | CGTAATCACTGAGAGCCACACAA |
| 1618 | Vtcn1-g1 | GATGTCACACAATTGCAGAGCCC |
| 1619 | Vtcn1-g2 | TCCAAAAGATGATCTGCCCCAAG |
| 1620 | Vtcn1-g3 | AGAGTGACATCATAACAGCCCAT |
| 1621 | Vtcn1-g4 | CATTTCAAAGAGCATGGCCGTAT |
| 1622 | Vtcn1-g5 | TGTGTAAATTCAGTGAGACACGT |

TABLE 6
MUCIG-Lib4 gRNAs

| SEQ | | |
|---|---|---|
| ID | | |
| NO: | Name: | Sequence: |
| 113 | Adar-g1 | TTCACCATAAGAGAGCTGCAGTA |
| 114 | Adar-g2 | TCAAGGAATGCAAGACAGCCACG |
| 115 | Adar-g3 | CTTTTCATAATAATGGCAGCCAG |
| 116 | Adar-g4 | AGACCAGAAGAATCCCAGTGCAC |
| 117 | Adar-g5 | CTGAGCATACTCTAACAACCCGC |
| 123 | Adora2a-g1 | ACAAACAAACAAACAAGCCCCAC |
| 124 | Adora2a-g2 | TTAATGAGATTGGTCCAGCCAAC |
| 125 | Adora2a-g3 | AAAATCCTTAGGTAGATGGCCAG |
| 126 | Adora2a-g4 | CAGCAAATCGCAATGATGCCCTT |
| 127 | Adora2a-g5 | ATGATGTACACCGAGGAGCCCAT |
| 243 | Btla-g1 | ATATGTATATTAATCCAGCAGCA |
| 244 | Btla-g2 | GACCTTTAAGACGCAGCACCAGC |
| 245 | Btla-g3 | ATCCTTTTCAGAAAGCAGAGCAG |
| 246 | Btla-g4 | TAACAGAATAAAGTGGAGTGCAA |
| 247 | Btla-g5 | TGTAGAACAGCTATACGACCCAT |
| 248 | C10orf54-g1 | TCCAGAGATAGATAAAGCACCCG |
| 249 | C10orf54-g2 | CCATACAGGTAATGAGAGCCCAG |
| 250 | C10orf54-g3 | CAGACAAAGCTAGATCCCCAGAG |
| 251 | C10orf54-g4 | CAAGACACTAATGAGCTCACAGT |
| 252 | C10orf54-g5 | CCAGGAAAATAGCAAGGAGCAGG |
| 333 | Cd274-g1 | CGTAGCAAGTGACAGCAGGCTGT |
| 334 | Cd274-g2 | CCATCGTGACGTTGCTGCCATAC |
| 335 | Cd274-g3 | CGCTTGTAGTCCGCACCACCGTA |
| 336 | Cd274-g4 | TAGAAAACATCATTCGCTGTGGC |
| 337 | Cd274-g5 | ATTTCTCCACATCTAGCATTCTC |
| 338 | Cd276-g1 | ATAATAGCAGTTACACAGTCTGC |
| 339 | Cd276-g2 | GTGAACATCGAACAAGCCCCGCT |
| 340 | Cd276-g3 | AGCAAGAACTAAGAGGTCACTGT |
| 341 | Cd276-g4 | GACAACAAAAGCCAGGGCCAGAT |
| 342 | Cd276-g5 | TTTAATGAAGAGCTGACGGCCAA |
| 353 | Cd47-g1 | CAAGCAAGACAGAAGCGCCAAGT |
| 354 | Cd47-g2 | TAGAGATTACAATGAGGCCAAGT |
| 355 | Cd47-g3 | ACCAAAGCAAGGACGTAGCCCAG |
| 356 | Cd47-g4 | CCACGATGACTGTGAGCACCAGC |
| 357 | Cd47-g5 | TAAACAGTAGTTGAGCTGAACCT |
| 488 | Ctla4-g1 | GTGTTTATATTCAAACCACCAGC |
| 489 | Ctla4-g2 | ATAAAATGAGTGTAAAGACCCAG |
| 490 | Ctla4-g3 | TCAAAGAAACAGCAGTGACCAGG |
| 491 | Ctla4-g4 | TGACACAACAGAAATATCCCAGC |
| 492 | Ctla4-g5 | CAATGACATAAATCTGCGTCCCG |
| 558 | Entpd1-g1 | TAGAAAGCAGAAAACGCCCCAAA |
| 559 | Entpd1-g2 | ATAGTTAATAGTAATCCACCCAT |
| 560 | Entpd1-g3 | ACACAGTATAGTCCTCGCCATAG |
| 561 | Entpd1-g4 | CAAGTTCAGCATGTAGCCCAAAG |
| 562 | Entpd1-g5 | AGTCACATTAGCTGCACGAGCAC |
| 703 | Havcr2-g1 | CCAAAGTCAGAAATGAAGGCGAG |
| 704 | Havcr2-g2 | AGACACCAATGATAAGTGCCAGG |
| 705 | Havcr2-g3 | CCCACCTAAGAAAGCCAGGACCT |
| 706 | Havcr2-g4 | AGTCCTTAATTTCATCAGCCCAT |
| 707 | Havcr2-g5 | TATAGTGTTAAGCATATGCCACC |
| 758 | Ido1-g1 | ATTTCCACCAATAGAGAGACGAG |
| 759 | Ido1-g2 | AGACAGATATATGCGGAGAACGT |
| 760 | Ido1-g3 | AATCTACATAATATACAACAGGC |
| 761 | Ido1-g4 | GCATAAGACAGAATAGGAGGCAG |
| 762 | Ido1-g5 | AACCTCAAAACCAGGCACGCCAG |
| 893 | Lag3-g1 | AGAAGCAAAAAGCCAAGGAGCAG |
| 894 | Lag3-g2 | CAAAAGGACCCAATCAGACAGCT |
| 895 | Lag3-g3 | CTTCGTAGAAAGTTAGGATCCAG |
| 896 | Lag3-g4 | AGTCACTGTGATGACCGCCAACG |
| 897 | Lag3-g5 | CAGACAGACAGACAGACACACAC |
| 913 | Lgals3-g1 | AAAGGCATTCTAACTAGGGCAGC |
| 914 | Lgals3-g2 | TTAAGCGAAAAGCTGTCTGCCAT |
| 915 | Lgals3-g3 | ACACAATAATAAATACATCTGCT |
| 916 | Lgals3-g4 | ACAGCTTGTCCTCTGACCTCCAC |
| 917 | Lgals3-g5 | TCCACTTCCAGGCAGTGACGCGT |
| 918 | Lgals9-g1 | CCTTCACATATGATCCACACCGA |
| 919 | Lgals9-g2 | ATATCATGATGGACTTGGACGGG |
| 920 | Lgals9-g3 | GGGTACACCACAGGAGGGATTCC |
| 921 | Lgals9-g4 | AGAATTTCTTGTTCACCATCACC |
| 922 | Lgals9-g5 | GGAAAGATAAGACACAGGCAGAG |
| 1123 | Nt5e-g1 | TACAATTACAAGATAGTCCAAGG |
| 1124 | Nt5e-g2 | AGATGTATTCAGAAACCACGCTG |
| 1125 | Nt5e-g3 | CTTTCGGTTAATATCGTACACCA |
| 1126 | Nt5e-g4 | ATCTCAAAACCAGAGTGCCCCAG |
| 1127 | Nt5e-g5 | CTGAGAGACAACAAGAGCCCAAA |
| 1148 | Pdcd1-g1 | AGTTCAGCATAAGATCCTCCGAC |
| 1149 | Pdcd1-g2 | CAAACCATTACAGAAGGCGGCCT |
| 1150 | Pdcd1-g3 | AAGTCCCTAGAAGTGCCCAACAG |
| 1151 | Pdcd1-g4 | AGCAGCAATACAGGGATACCCAC |
| 1152 | Pdcd1-g5 | CCAGTCTACGAATTTCCCACCTG |
| 1153 | Pdcd1lg2-g1 | ATGAAAACATGAAGTGGCCACGT |
| 1154 | Pdcd1lg2-g2 | AAGTAGAAACAAATACCACAGTG |
| 1155 | Pdcd1lg2-g3 | CAAAAGTGCAAATGGCAGGTCCT |
| 1156 | Pdcd1lg2-g4 | GCATTCCAGAACATGCAGCTGAA |
| 1157 | Pdcd1lg2-g5 | TACAACAATTACCTTGTGACTCA |
| 1508 | Tigit-g1 | TTAAGCAAATGAGTCCCAGCACA |
| 1509 | Tigit-g2 | GTCAACACTATAAATGGCCAGAA |
| 1510 | Tigit-g3 | ACACTGTAAGATGACAGAGCCAC |
| 1511 | Tigit-g4 | CACTGAAGACTGAAGCGACATGC |
| 1512 | Tigit-g5 | GATACAGCAATGAAGCTCTCTAG |
| 1618 | Vtcn1-g1 | GATGTCACACAATTGCAGAGCCC |
| 1619 | Vtcn1-g2 | TCCAAAAGATGATCTGCCCCAAG |
| 1620 | Vtcn1-g3 | AGAGTGACATCATAACAGCCCAT |
| 1621 | Vtcn1-g4 | CATTTCAAAGAGCATGGCCGTAT |
| 1622 | Vtcn1-g5 | TGTGTAAATTCAGTGAGACACGT |

### Example 3: A Four-Gene AAV-MUCIG Composition Elicits Potent Anti-Tumor Immunity

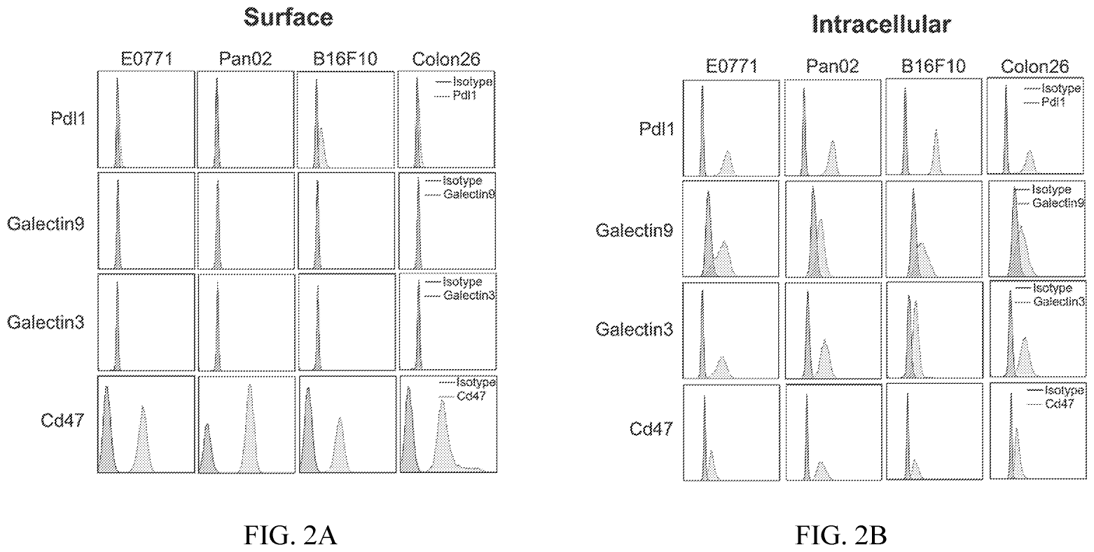

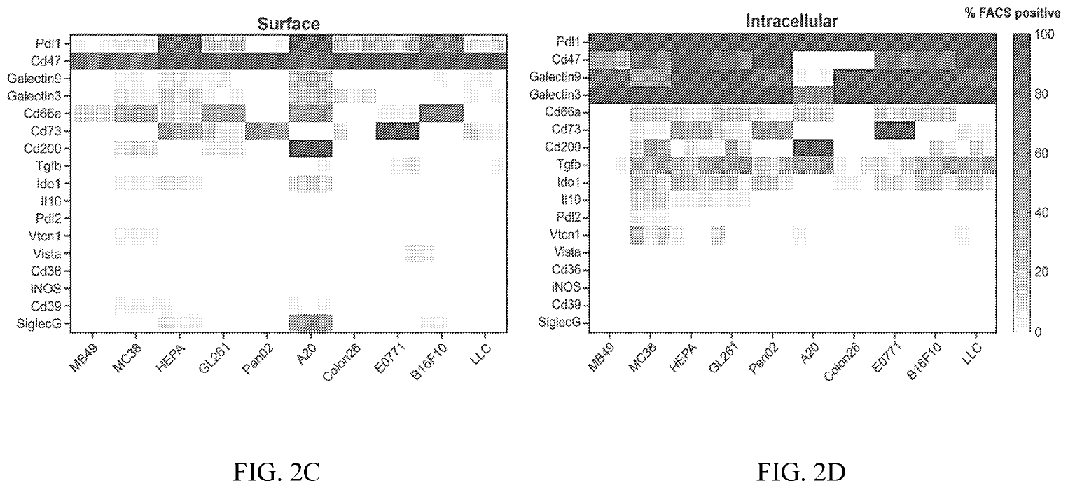

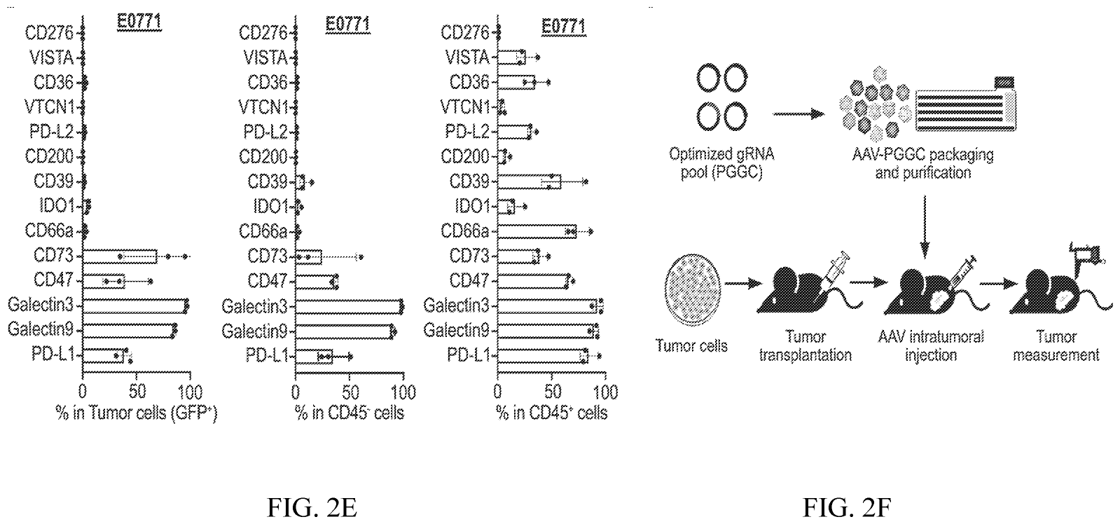

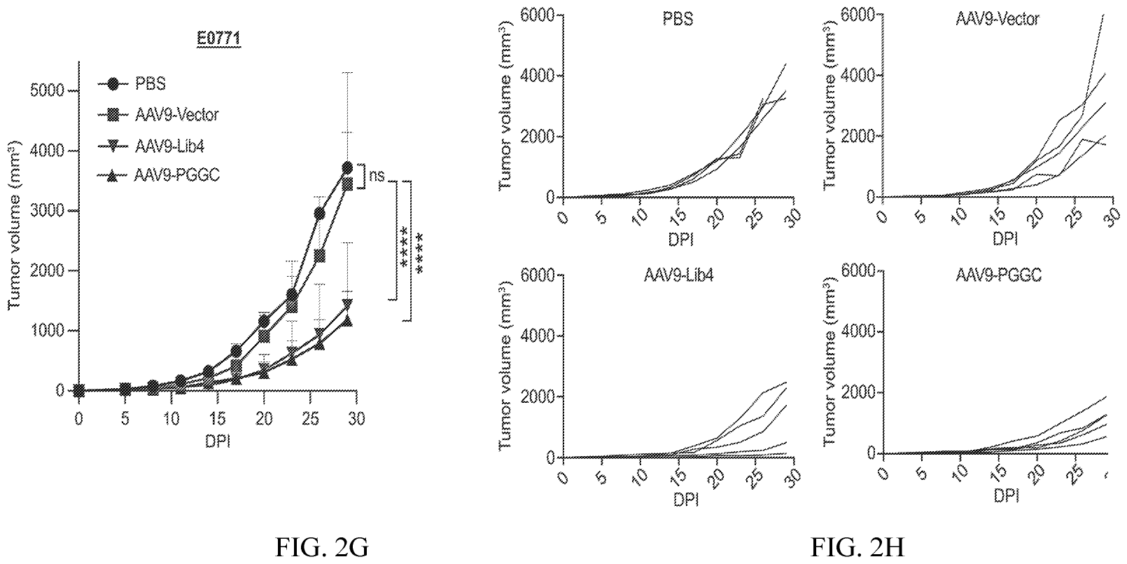

While two of the AAV-MUCIG libraries had evidence of anti-tumor responses, it was reasoned that further optimization of the library might increase treatment efficacy by reducing the proportion of therapeutically neutral or detrimental gRNAs that are delivered to the tumor. To further refine the MUCIG-lib4 library, protein-level expression of the genes targeted in MUCIG-Lib4 was assessed across a panel of syngeneic cancer cell lines that represent various tumor types. As interest was primarily in assessing tumor-derived factors, the genes that are primarily expressed in non-tumor cells were excluded, such as the T cell checkpoints Pdcd1, Lag3, and Haver2. In addition to the genes targeted in Lib4, these studies also tested other known immunosuppressive genes, such as Tgf-8. Through a combined evaluation of both surface and intracellular expression, 4 genes were pinpointed (Pd-LI Cd247, Cd47, Galectin9 Lgals9, and Galectin3 Lgals3) that were highly expressed at the protein-level across different cancer cell lines (FIGS. 2A-2D). The human cancer gene expression database was also examined and confirmed that the human orthologs of these genes are expressed across a variety of human tumors, supporting their clinical relevance. Galectin9 and Galectin3 were exclusively expressed intracellularly among all cell lines (FIGS. 2B, 2D). Of note, current standard monoclonal antibodies cannot inhibit such intracellular targets, however this is achievable by Cas13d-mediated silencing as shown elsewhere herein (FIG. 15). CD47 was highly expressed on the surface and also expressed intracellularly (FIGS. 2A, 2B). Surprisingly, PD-L1 was highly expressed intracellularly, even in cell lines with absent surface expression of PD-L1 (FIGS. 2A, 2B). Since immune checkpoints are often induced in the process of tumorigenesis, expression of these genes was tested in an in vivo E0771 tumor model, by flow cytometry analysis of these four proteins in dissociated single cells from tumor samples. The results showed that all four factors (Pd-11, Cd47, Galectin9, and Galectin3) were expressed in both tumor and immune cells (FIG. 2E).

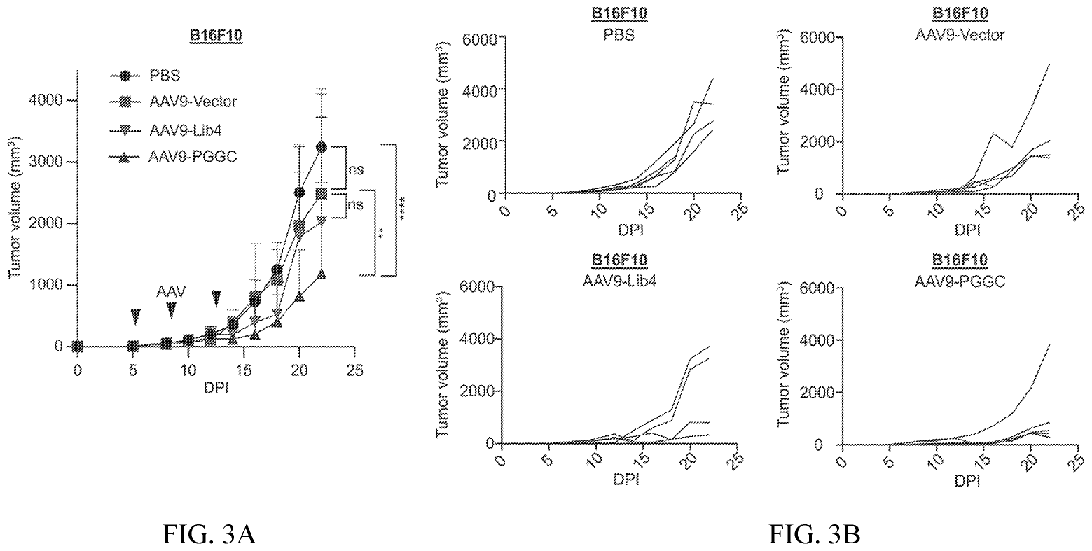

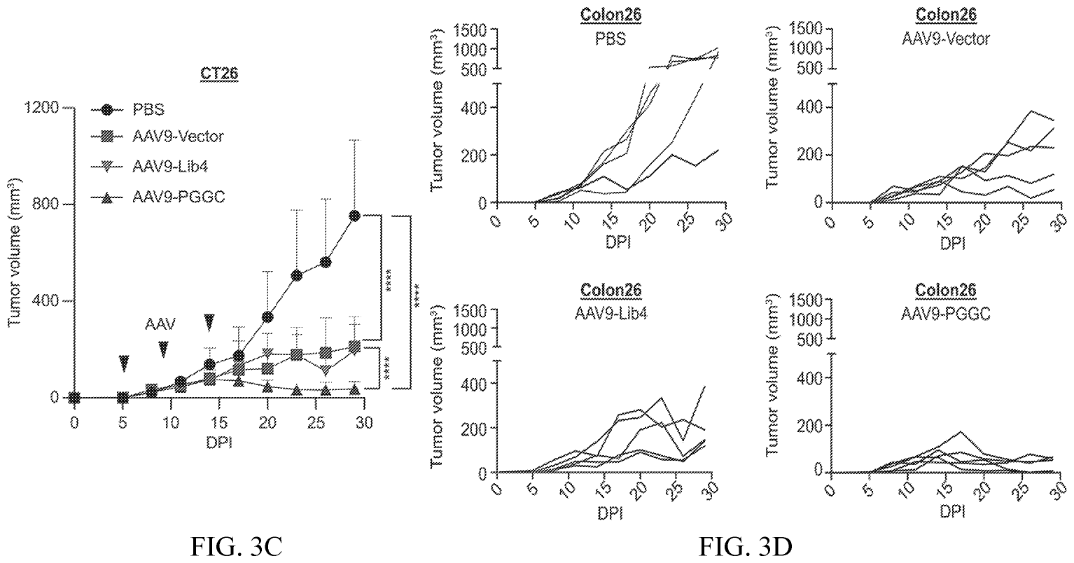

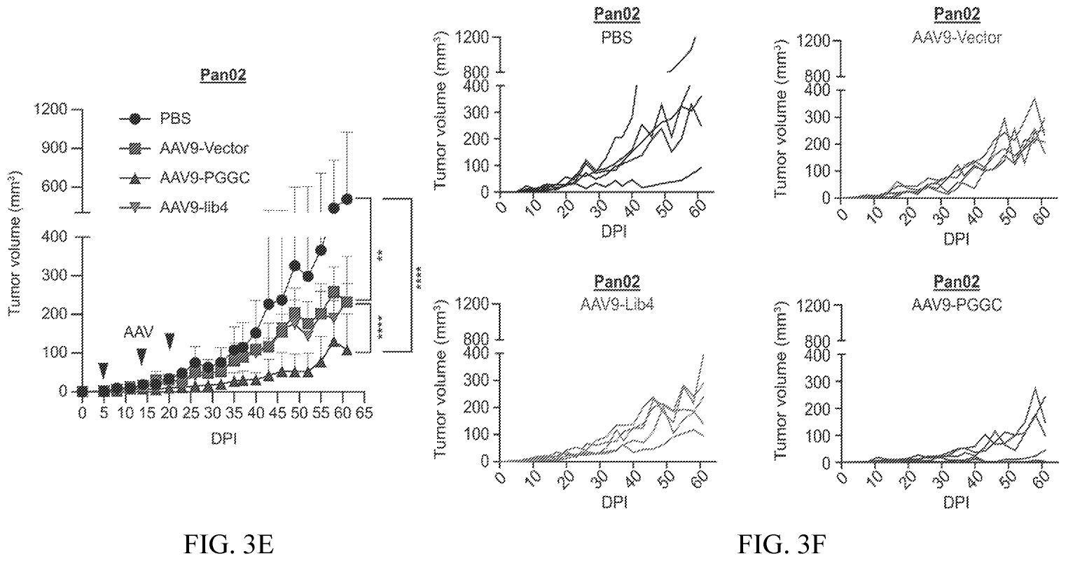

A gRNA composition was then designed which targeted these four genes as a rational and simplified version of MUCIG (named PGGC). The AAV-PGGC pool was then delivered into E0771 tumor-bearing mice by intratumoral injection (FIG. 2F). It was found that treatment with AAV-PGGC (Pdl1-g14, Galectin9-g9, Galectin3-g2, Cd47-g2) led to significant reduction of tumor growth, with an efficacy level similar to the AAV-Lib4 group (FIGS. 2G-H). To assess whether these effects were more broadly applicable to other tumor models, the antitumor effect of AAV-PGGC was similarly evaluated in three representative models based on their levels of responsiveness to immune checkpoint blockade antibody therapeutics, including B16F10 melanoma (FIGs. A,B), CT26 colon cancer (FIGS. 3C,D), and Pan02 pancreatic cancer (FIGS. 3E,F) mouse models. In all three models, AAV-PGGC showed significant antitumor efficacy when compared with the control group (FIGS. 3A-F). Importantly, even in models where AAV-Lib4 treatment failed to reduce tumor growth, AAV-PGGC demonstrated efficacy across all models (FIG. 3).

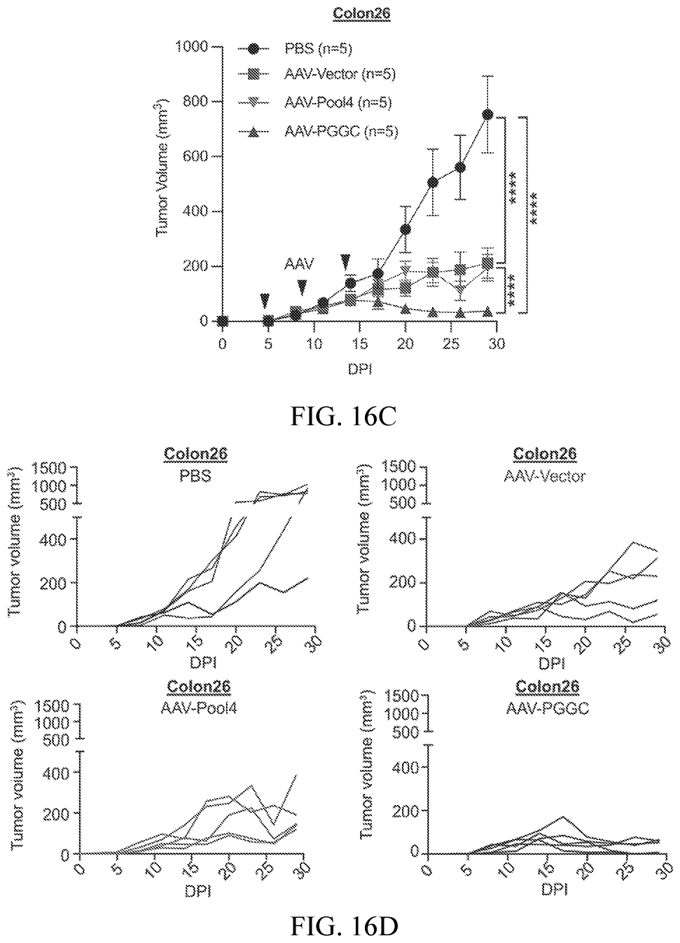

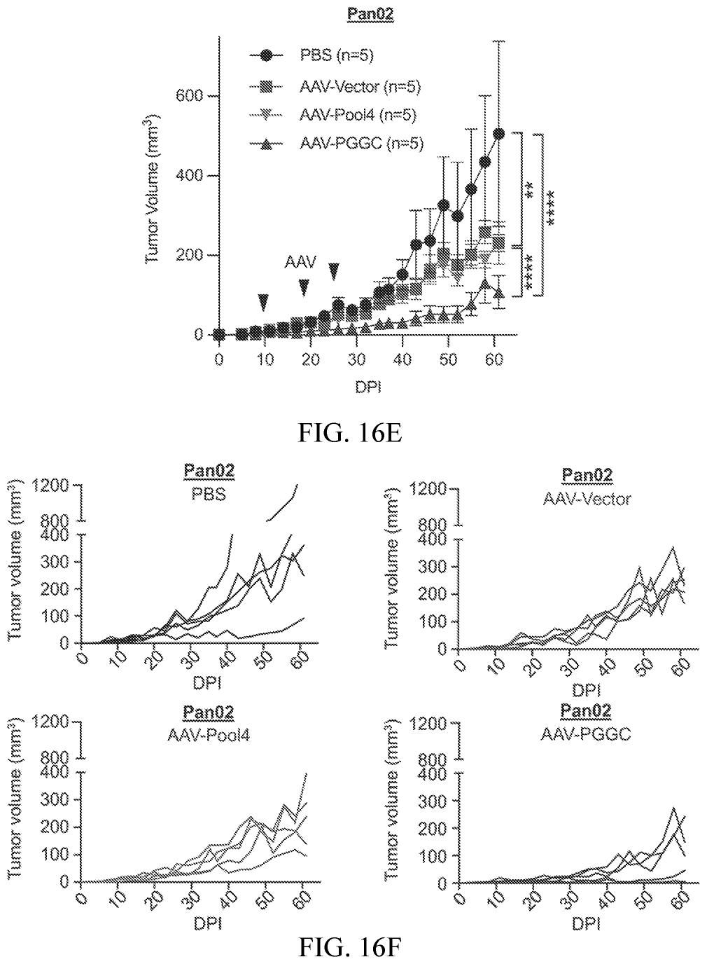

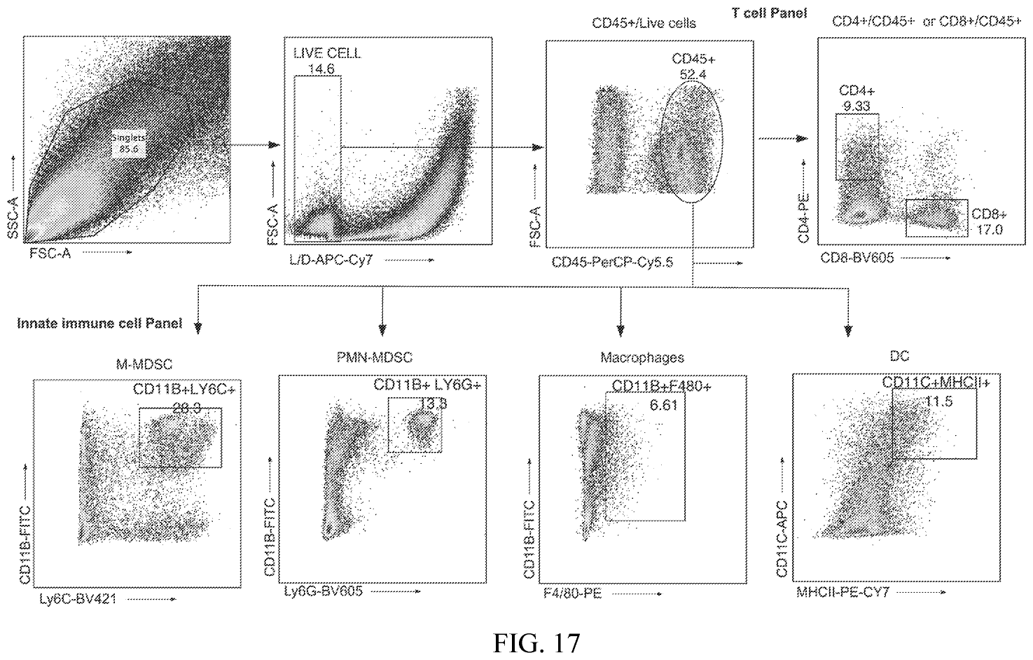

To assess whether these effects were more broadly applicable to other tumor models, the anti-tumor effect of AAV-PGGC was similarly evaluated in three representative models with different levels of responsiveness to immune checkpoint blockade antibody therapeutics, including B16F10 melanoma (resistant) (FIGS. 16A & 16B), Colon26 colon cancer (sensitive) (FIGS. 16C & 16D), and Pan02 pancreatic cancer (resistant) (FIGS. 16E & 16F) mouse models. In all three models, AAV-PGGC showed significant in vivo anti-tumor efficacy when compared with the control group (FIG. 16). Importantly, in these three models where AAV-pool4 treatment failed to reduce tumor growth, AAV-PGGC still demonstrated significant efficacy across all models (FIG. 16). These data suggest that this four gene formula of Cas13d/gRNA-pool (AAV-PGGC) is effective across different cancer types in animal models.

### Example 4: AAV-PGGC Treatment Promotes T Cell Tumor Infiltration while Hampering the Recruitment of Immunosuppressive Cells

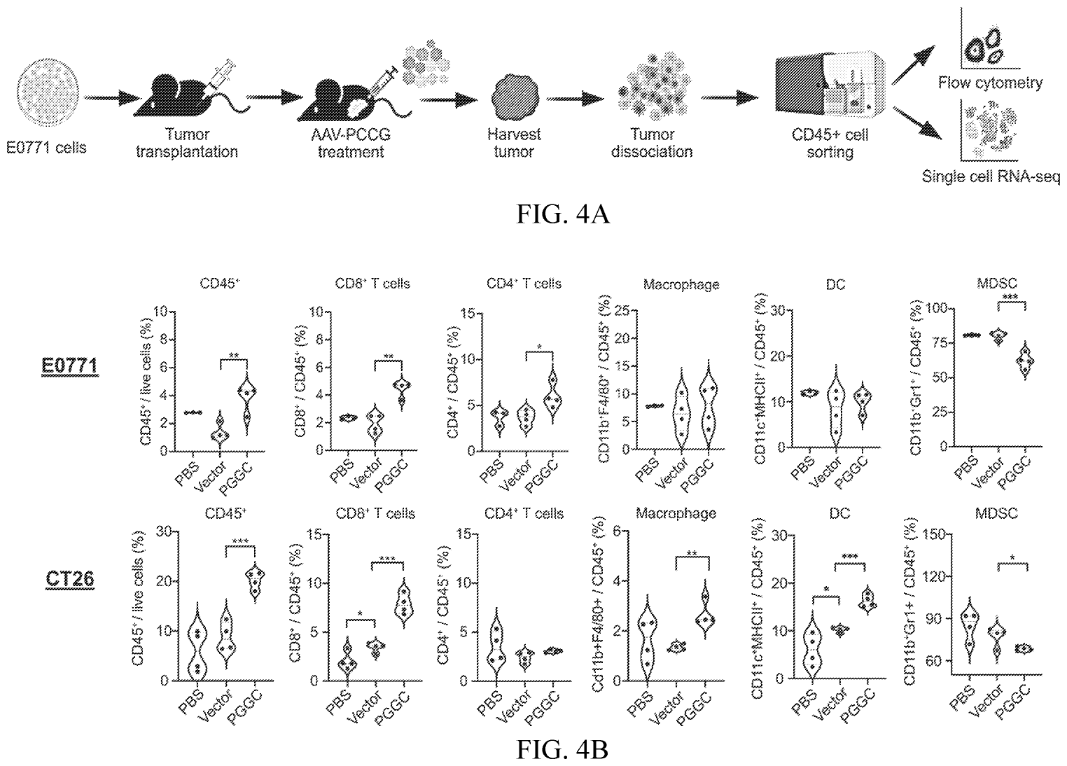

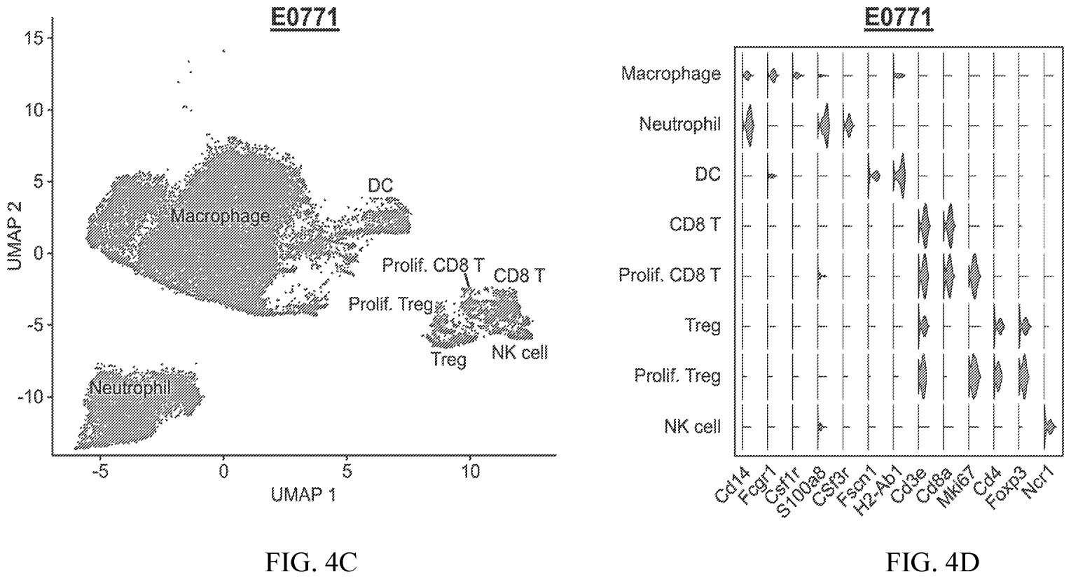

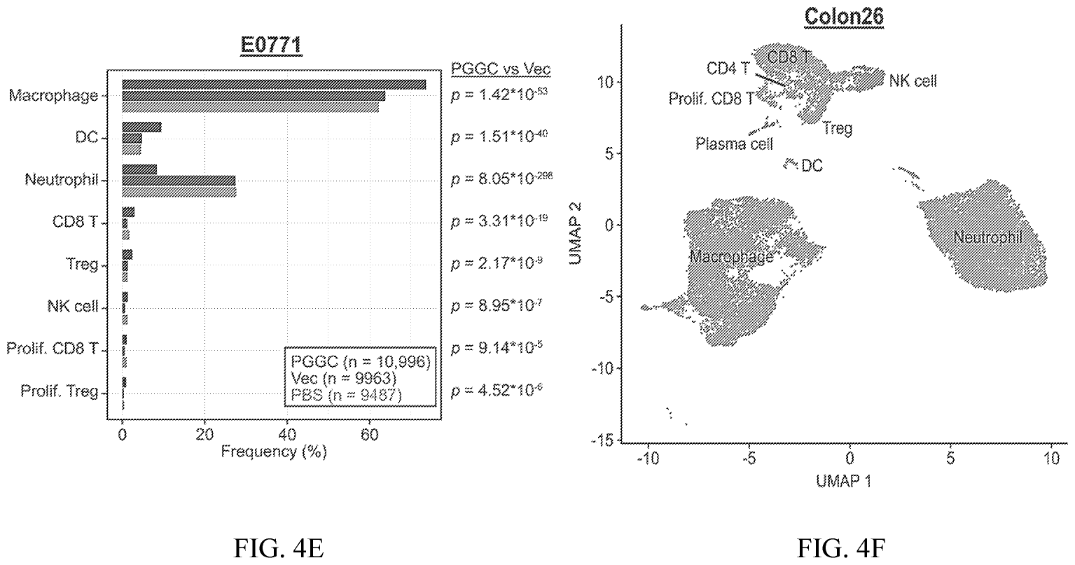

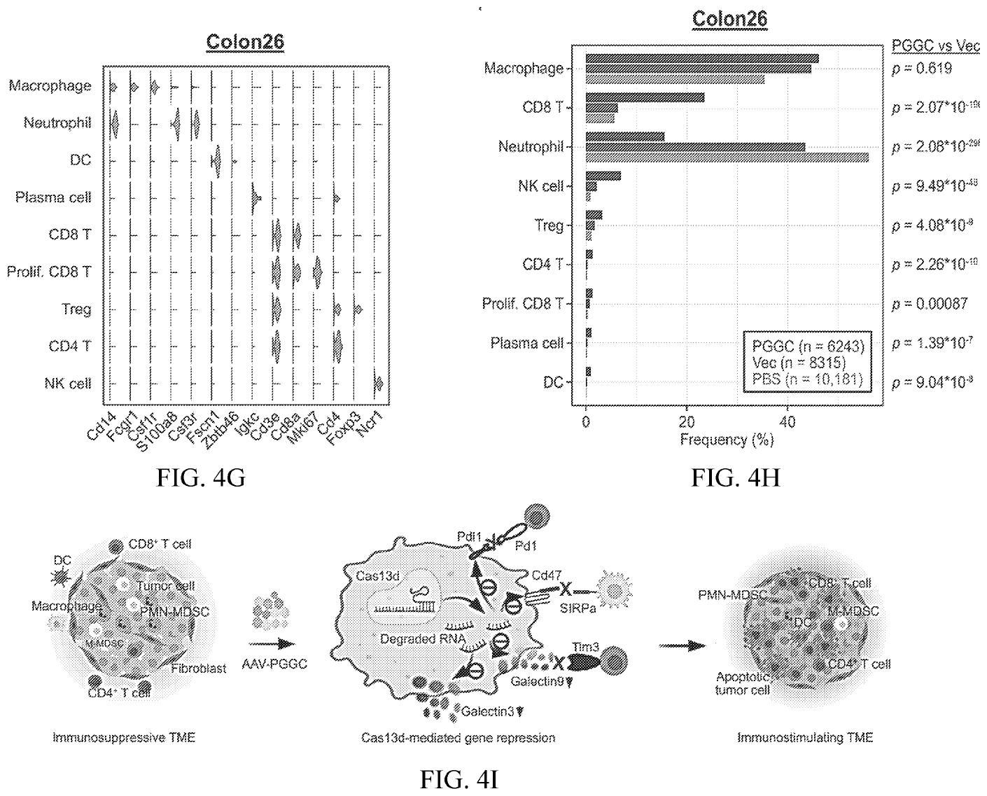

Without wishing to be bound by theory, it was hypothesized that the therapeutic efficacy of AAV-PGGC was based on its modulatory effect on the immune composition of the TME. By FACS analysis, studies then profiled tumor-infiltrating lymphoid and myeloid cell populations in mice that received either PBS, AAV-vector, or AAV-PGGC treatment in two different syngeneic tumor models (E0771 and Colon26) (FIGS. 4A & 17). In the E0771 tumor model, more tumor infiltrating lymphocytes (TILs) were observed in the AAV-PGGC treated mice than the control groups including CD45⁺ cells (FIGS. 4B & 11). These studies also found a significant increase of CD8⁺ and CD4⁺ T cells in the AAV-PGGC treated mice compared to controls (FIG. 4B). In addition, though there were no substantial changes in the macrophage or the DC population, there was a significant decrease of Cd11b⁺ Grl⁺ MDSCs, a heterogeneous cell population with the capacity to functionally suppress T cell responses (FIG. 4B). In another tumor model, CT26, a similar increase of CD8⁺ TILs was observed in the AAV-PGGC treatment group compared to control groups (FIG. 4B). While there was no change in CD4⁺ TILs in the CT26 model, there were more tumor-infiltrating macrophages and DCs in AAV-PGGC treated tumors than the controls (FIG. 4B). The latter are critical for antigen presentation and for priming adaptive immune responses

To systematically investigate the effect of AAV-PGGC treatment on the immune cell populations and their transcriptomics in the TME, single-cell RNA-seq (scRNA-seq) of tumor-infiltrating immune cells was performed in mice treated with PBS, AAV-Vector, or AAV-PGGC (FIGS. 4C-D). Consistent with the FACS analysis, scRNA-seq of the E0771 tumor model revealed significant changes in multiple immune cell populations after AAV-PGGC treatment (FIG. 4E), including an increase of CD8⁺ T cells and proliferating CD8⁺ T cells. Similarly, in the CT26 model, more CD8⁺ T cells and proliferating CD8+ T cells was observed following AAV-PGGC treatment (FIGS. 4F-H). On the other hand, there was a substantial reduction of neutrophil abundances in AAV-PGGC treatment group compared with PBS or vector control group (FIG. 4E), which was also observed in the CT26 model (FIG. 4H).

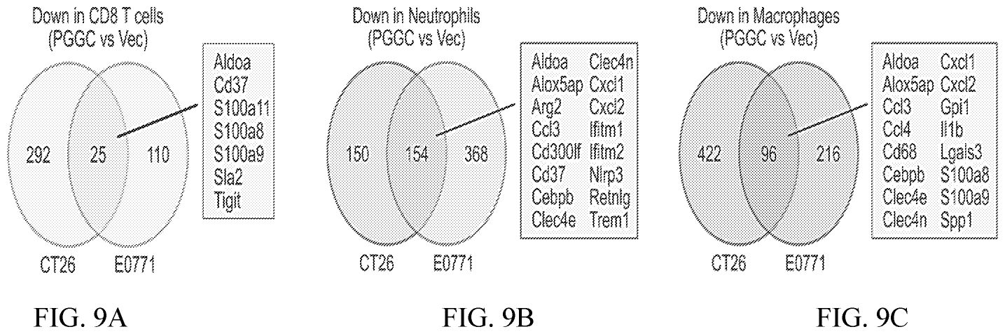

These studies subsequently identified differentially expressed genes in the cell types whose abundances were most affected by AAV-PGGC, including CD8⁺ T cells, neutrophils and macrophages. Many genes associated with key immunosuppressive functions were found to be downregulated across both E0771 and CT26 models, including Arg2, Il1b, Trem1, S100a8, S100a9, Tigit, and Cd37 (FIG. 9). Cd37 can inhibit Cd3-induced T cell proliferation, and in Cd8⁺ T cells, the studies of the present disclosure found Cd37 was downregulated in AAV-PGGC treatment group when compared with vector group (FIG. 9A). Tigit, a marker of T cell exhaustion, was also decreased in Cd8⁺ T cells of AAV-PGGC treatment group (FIG. 9A). Arg2, which has been implicated in the immunosuppressive functions of neutrophils, was downregulated in the AAV-PGGC group along with Ifitm1 and Ifitm3, two genes that play a role in suppressing interferon mediated immunity (FIG. 9B). S100a8 and S100a9, two factors that help recruit MDSCs to the TME, were downregulated in macrophages and CD8⁺ T cells from AAV-PGGC treated tumors (FIG. 9C). Consistent with the observed reductions in tumor-associated neutrophils after AAV-PGGC treatment, the neutrophil-recruiting chemokines Cxcl1 and Cxcl2 were significantly downregulated in both neutrophils and macrophages isolated from tumors treated with AAV-PGGC. These data indicate that AAV-PGGC treatment can effectively reverse the immunosuppressive TME, promoting T cell infiltration and reducing MDSC recruitment (FIG. 4I).

### Example 5: AAV-PGGC Combined with Anti-GR1 Treatments Inhibit Tumor Growth and Metastasis

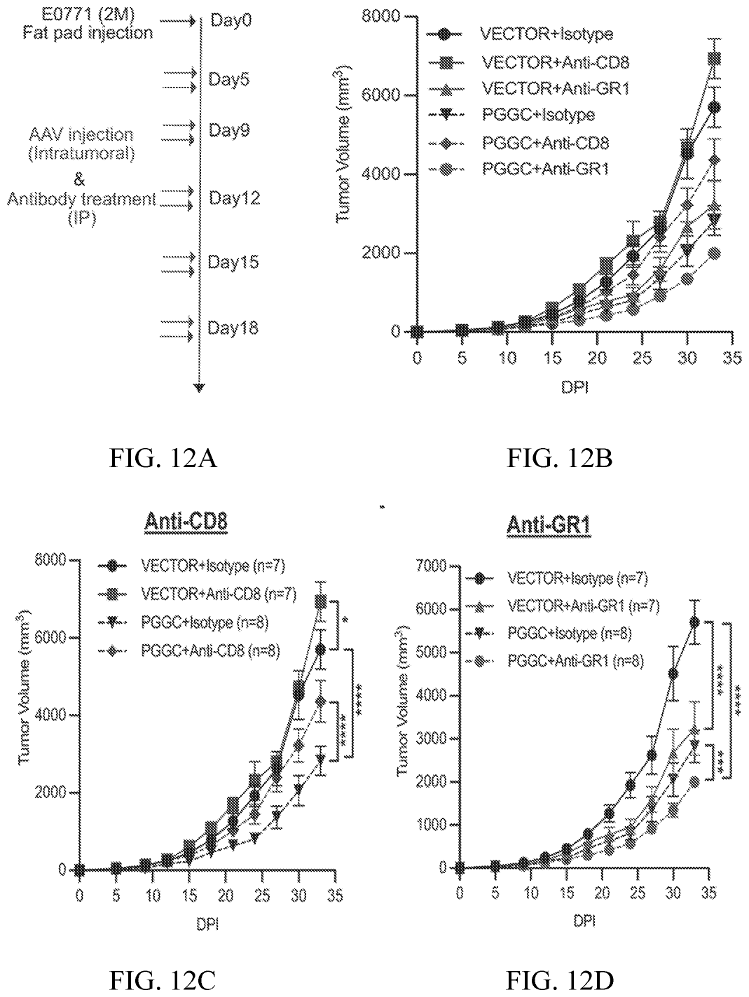

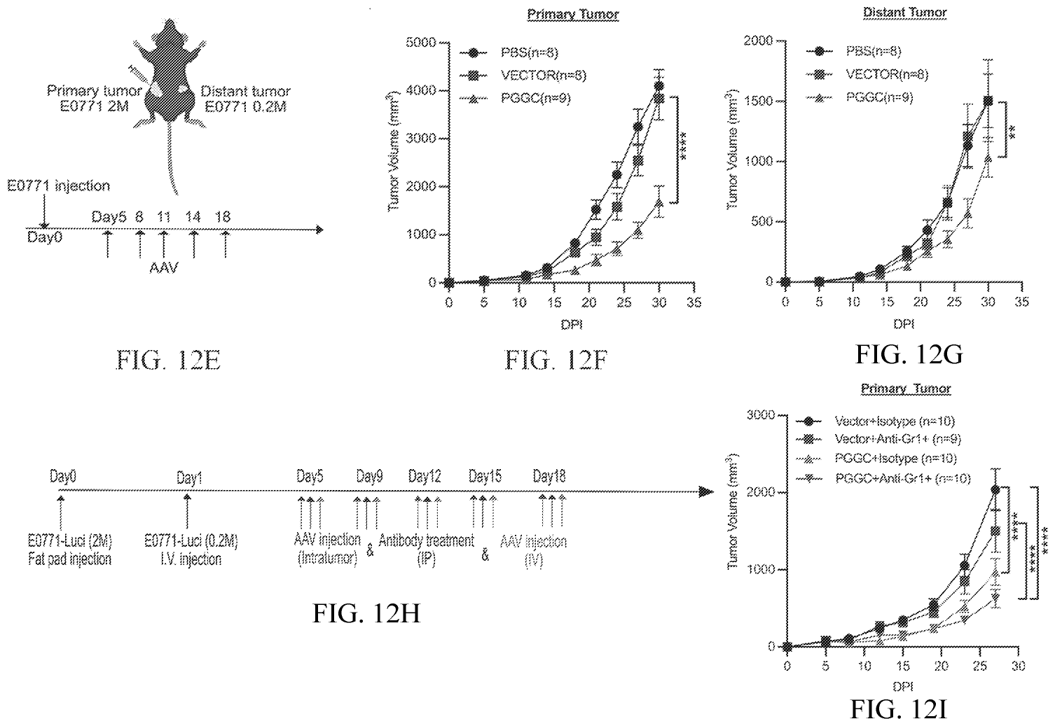

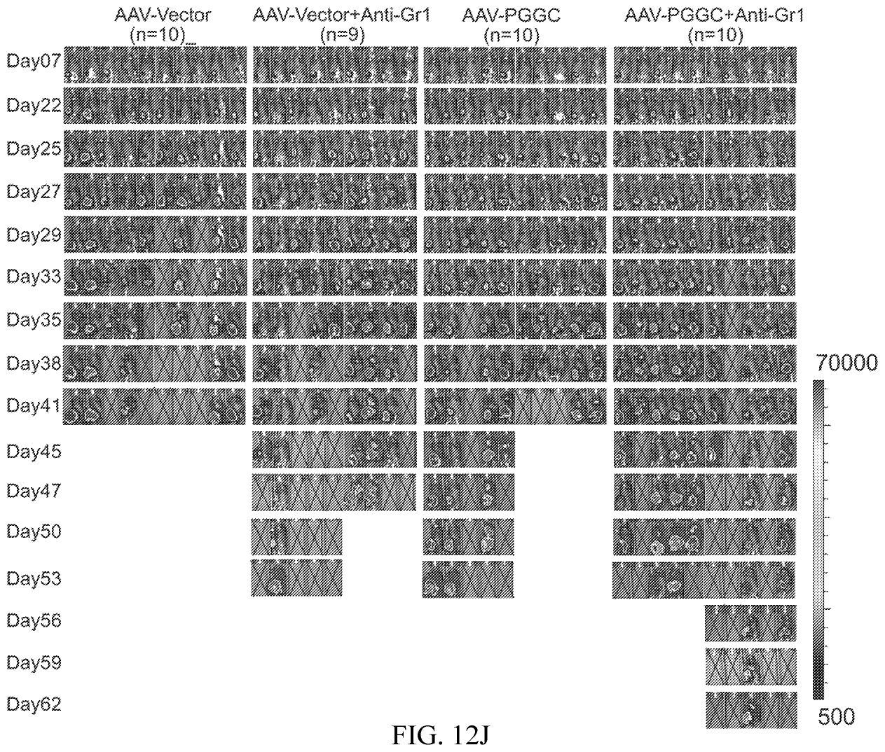

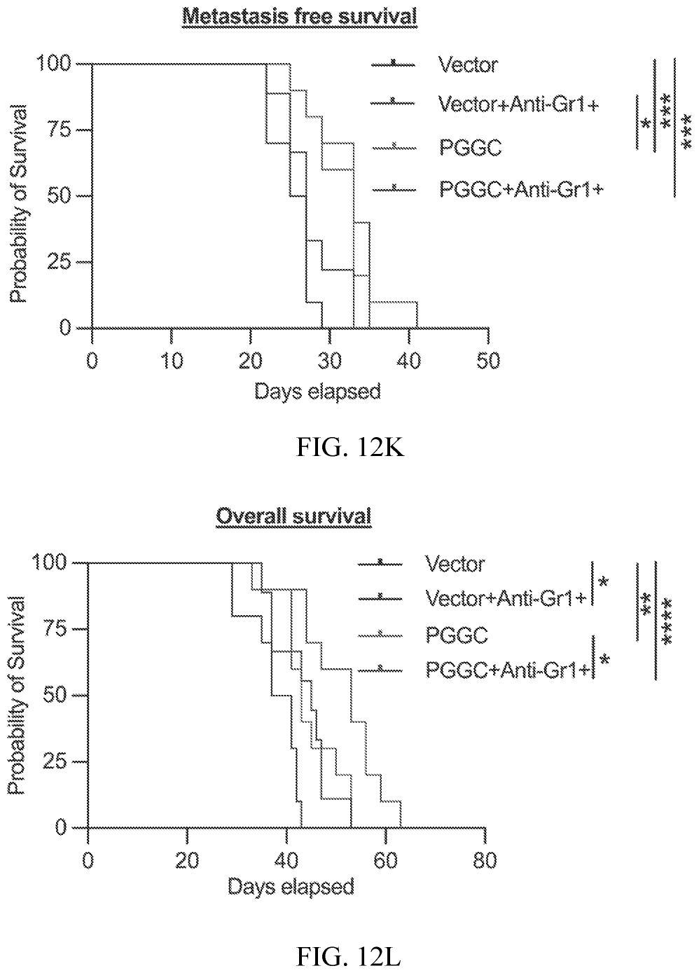

Given the increase of CD8⁺ T cells and reduction of neutrophils in the tumor microenvironment after AAV-PGGC treatment, studies next tested how these two cell populations influence the therapeutic efficacy of AAV-PGGC. CD8⁺ T cell or MDSC/neutrophils depletion was performed by in vivo injection of anti-CD8 or anti-GR1 antibody, respectively (FIG. 12A). Results demonstrated that mice with CD8⁺ T cell depletion partially impaired the anti-tumor effect of AAV-PGGC (FIGS. 12B & 12C), which indicate that AAV-PGGC treatment is partially dependent on CD8⁺ T cells. Meanwhile, depletion of MDSCs and neutrophils by anti-GR1 in combination with AAV-PGGC treatment could further reduce the tumor burden when comparing to either AAV-PGGC or anti-GR1 antibody alone (FIGS. 12B and 12D). These data suggested that CD8⁺ T cells and MDSCs/neutrophils together play critical roles in AAV-PGGC therapy, and combinatorial treatment with AAV-PGGC plus anti-GR1 have a synergistic effect.

Subsequent studies next sought to determine whether the local AAV-PGGC treatment induced anti-tumor effect could extend to distant tumor site. A dual-sites E0771 tumor model was utilized to evaluate the systemic anti-tumor effect of AAV-PGGC against both the injected and non-injected distant sites (FIG. 12E). In this E0771 dual tumor model, different numbers of cells were injected into both mammary fat pads to model a primary tumor and a distant tumor. Then AAV-PGGC was injected only to the primary tumor site. It was found that AAV-PGGC inhibited tumor growth not only at the injected primary site but also the non-injected distant site (FIGS. 12F & 12G). These data suggest that AAV-PGGC has a systemic anti-tumor activity.

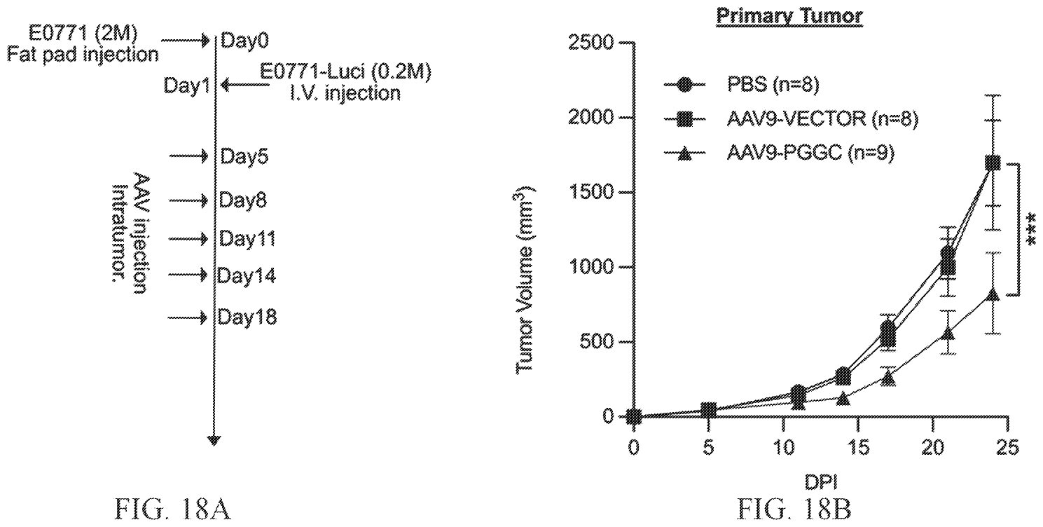

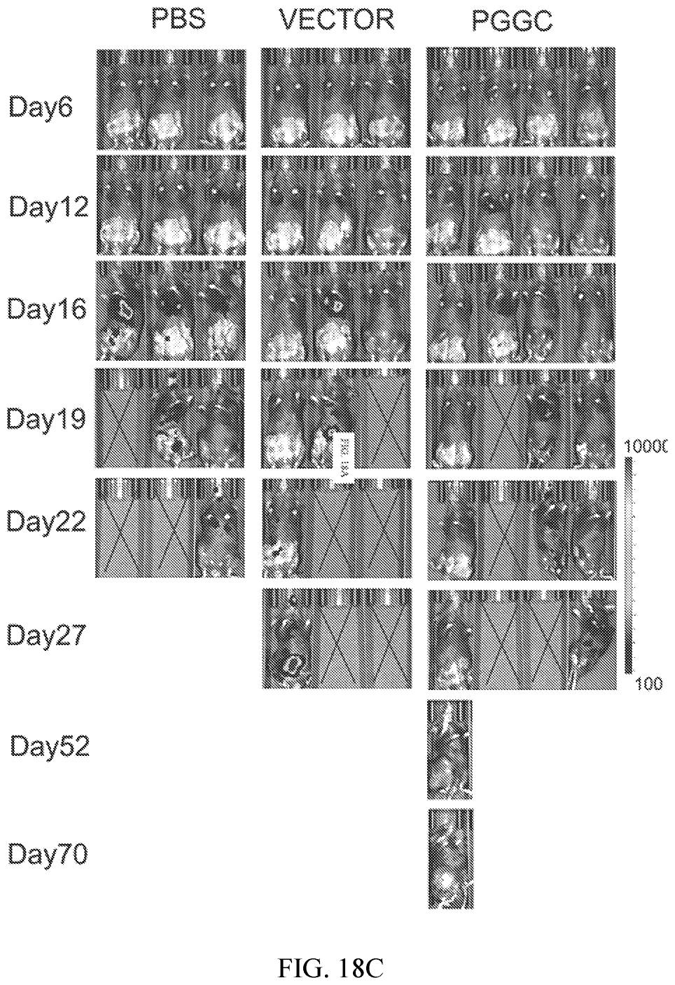

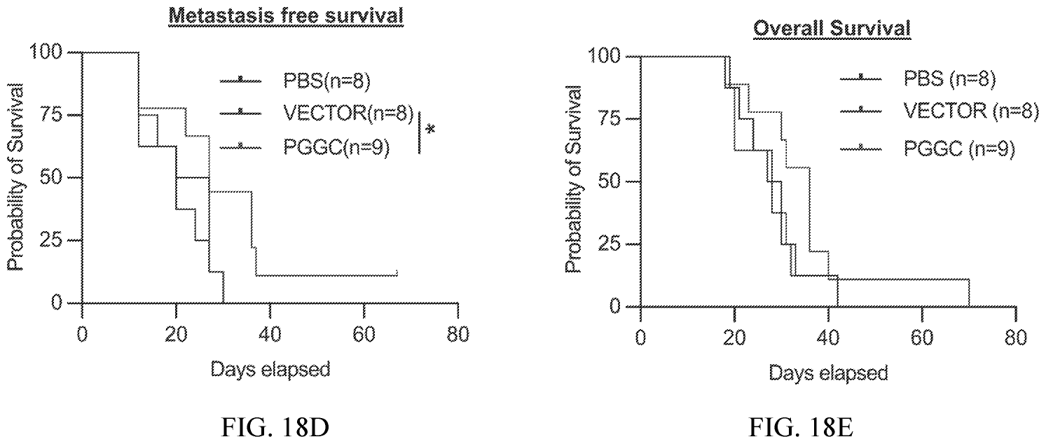

Without wishing to be bound by theory, it was hypothesized that AAV-PGGC could have a therapeutic effect on metastatic cancer in internal organs. To investigate this possibility, a tumor model was used which comprised the injection of E0771 cells into the fat pad to develop an orthotopic tumor burden, and intravenous injection of luciferase-expressing E0771 to model lung metastatic tumor burden (FIG. 18A). Tumor-bearing mice were treated with AAV-PGGC by intratumoral injection, primary tumor volume was measured for primary tumor burden, and bioluminescent signal was measured for lung metastatic burden. Mice treated with AAV-PGGC had significant reduction of primary tumor growth (FIG. 18B). Importantly, AAV-PGGC also significantly extended lung metastasis free survival when comparing to Cas13d-vector control group, with a numerical effect on overall survival (FIG. 18C-18E). These data indicate AAV-PGGC local treatment has certain moderate therapeutic effect on metastatic cancer in internal organs.

Because AAV-PGGC in combination with anti-GRI antibody had synergistic anti-tumor activity, subsequent studies examined whether the combined treatment could have stronger efficacy against metastases. Again the orthoptic injection of primary tumor and intravenous injection to model lung metastasis (FIG. 12H) was utilized. Due to the limited effect on lung metastatic tumor by local AAV-PGGC injection alone, AAV-PGGC was injected by both intratumoral and intravenous injection for the goal of better metastatic tumor targeting, and combined with anti-GR1⁺ antibody treatment given by intraperitoneal (i.p.) injection (FIG. 12H). While anti-GR1 alone has little effect, significant tumor suppression was observed by AAV-PGGC alone or AAV-PGGC plus anti-GR1 combo treatment (FIG. 12I). In this E0771 metastatic tumor model, the AAV-PGGC plus anti-GR1 combo showed the strongest therapeutic effect among all treatment groups, against both primary tumor and metastatic disease (FIGS. 12I-12L). The effect of treatments on metastatic disease were reflected by IVIS imaging of metastatic tumor burden (FIG. 12J), metastasis-free survival (FIG. 12K), and overall survival (FIG. 12L). These data indicated that AAV-PGGC in combination with anti-GR1 antibody treatment had significant efficacy against a systemic disease with internal organ metastasis in a syngeneic orthotopic tumor model.

### Example 6

The TME is enriched with immunosuppressive factors that can be derived from tumor cells, tumor-associated fibroblasts or the infiltrating immunosuppressive cells. Immunosuppressive factors produced by immunosuppressive cells can either inhibit effective anti-tumor immunity by their immune checkpoint function, or attract and recruit immature immune cells and induce their differentiation into immune suppressive cells, such as MDSCs, M2 macrophages, or regulatory T cells (Tregs). They can also influence T cell access to the tumor core or inhibit T cell activation and proliferation. Tumor immunosuppressive factors are a prime target for therapeutic intervention, as they enable tumor cells to escape elimination by the immune system. A number of preclinical studies have demonstrated that neutralization of immunosuppressive factors can in certain embodiments reverse the immunosuppressive TME and promote antitumor immunity.

Various strategies have been developed to repress such genes or their activity, including siRNAs, antisense oligos, antagonistic antibodies, and small molecule inhibitors. However, the efficacy of monotherapies targeting immunosuppressive factors is limited to only a subset of patients, prompting the present efforts to explore efficient approaches for combinatorial immunotherapy. In certain embodiments, cancer gene therapy, such as local tumor overexpression of OX40L or other combinational cytokines, has the potential to promote tumor regression. However, because the payloads for transgene overexpression are often sizable, it is difficult to multiplex a large number of transgenes expressing immunostimulatory factors as a combinatorial therapy. Herein, the studies of the present disclosure take the converse approach by simultaneously repressing multiple immunosuppressive genes directly in the TME. The present disclosure leverages the modularity of the CRISPR/Cas13d system to devise multiple combinatorial immunotherapies, demonstrating the anti-tumor efficacy of several different libraries of varying complexity. Because multiplexing gRNAs is simple, it is readily feasible to generate and pool gRNA libraries that target a large number of immunosuppressive genes.

Because the relative abundance of each gRNA will influence its silencing efficiency, optimizing the size of the library is crucial for MUCIG therapy. Thus, the studies disclosed herein rationally refined the library composition and tested five different compositions of libraries at different scales. These studies demonstrated that a simple AAV-PGGC combination therapy against four immune checkpoints, PD-L1, CD47, Galectin-3, and Galectin-9, had significant antitumor activity in several different tumor models, including breast cancer (E0771), melanoma (B16F10), pancreatic cancer (Pan02), and colon cancer (CT26). These results suggest that the concept of MUCIG is not limited to a single tumor type and can potentially be broadly applicable. To understand the mechanisms of action behind the anti-tumor efficacy, the studies disclosed herein investigated the TME change upon AAV-PGGC treatment by FACS and scRNA-seq. It was found that AAV-PGGC therapy enhanced CD8⁺ T cell infiltration and reduced MDSC abundances. On the transcriptional level, consistent down-regulation of multiple immunosuppressive genes was observed in two different cancer models, and a concordant reduction in the neutrophil chemoattractants Cxcl1 and Cxcl2. These data suggested that AAV-PGGC therapy can attenuate the immunosuppressive TME, thereby enhancing antitumor immune responses.

Key challenges with tumor gene therapy include on-target-specificity and gene delivery efficiency. Cas13d binds and cleaves single-strand RNA, thus avoiding safety concerns stemming from unintended DNA damage caused by Cas9 or Cas12a. In addition, Cas13d is more compact compared to Cas9, Cas12a, and many other Cas13 family members, conferring a key advantage for viral vector delivery. Herein, AAVs were utilized to deliver the Cas13d-gRNA payload into tumors, as AAVs can efficiently deliver foreign genetic materials in vivo with minimal toxicity. Indeed, persistent exogenous gene expression was observed herein up to two weeks after the final intratumoral injection of AAV. While the diversity of immunosuppressive pathways that are engaged across different tumors poses an important challenge for off-the-shelf usage, the MUCIG approach, with the versatility of targeting virtually any reasonable combinations of genes using CRISPR-Cas13d and gRNA pools, offers far greater flexibility and modularity compared to conventional antagonistic antibodies or small molecules. By further customizing the cocktail of immunosuppressive factors that is targeted by MUCIG, or by utilizing more specific delivery vehicles, the therapeutic window can be optimized to minimize off-tumor toxicity while maintaining anti-tumor efficacy.

In summary, the studies presented herein provide a demonstration of MUCIG, a versatile strategy for combinatorial cancer immunotherapy by multiplexed targeting of the immunosuppressive gene collections. By simultaneously unraveling multiple facets of the immunosuppressive TME, MUCIG is able to drive customized anti-tumor immune responses.

### Enumerated Embodiments

The following enumerated embodiments are provided, the numbering of which should not be construed as designating levels of importance.

Embodiment 1. A method of enhancing an immune response in a subject in need thereof, the method comprising administering to the subject an effective amount of a gene silencing system, wherein the gene silencing system decreases expression of at least one endogenous immunosuppressive gene in a target cell, thereby enhancing the immune response.

Embodiment 2. A method of enhancing an anti-tumor immune response in a subject in need thereof, the method comprising administering to the subject an effective amount of a gene silencing system, wherein the gene silencing system decreases expression of at least one endogenous immunosuppressive gene in a target cell, thereby enhancing the anti-tumor immune response.

Embodiment 3. The method of any one of Embodiments 1-2, wherein the gene silencing system is a CRISPR-based gene silencing system which comprises a plurality of AAV-CRISPR vectors, wherein the plurality of AAV-CRISPR vectors comprises a Cas nuclease and a plurality of guide RNAs (gRNAs) homologous to mRNA from a plurality of target genes associated with immune suppression.

Embodiment 4. The method of Embodiment 3, wherein the gRNA sequences comprise at least one nucleic acid sequence selected from the group consisting of SEQ ID NOs: 1-1657.

Embodiment 5. The method of Embodiment 3, wherein the plurality of gRNAs comprise the nucleotide sequences consisting of SEQ ID NOs: 1-1657.

Embodiment 6. The method of Embodiment 3, wherein the gRNA sequences comprise at least one nucleic acid sequence selected from the group consisting of SEQ ID NOs: 1-92.

Embodiment 7. The method of Embodiment 3, wherein the plurality of gRNAs comprise the nucleotide sequences consisting of SEQ ID NOs: 1-92.

Embodiment 8. The method of Embodiment 3, wherein the target genes are selected from the group consisting of Pdl1, Galectin9, Galectin3, and Cd47, or any combination thereof.

Embodiment 9. The method of Embodiment 3, wherein the CRISPR-based gene silencing system is selected from the group consisting of a type III (Cmr/Csm) system, a type VI system, and a type II system.

Embodiment 10. The method of Embodiment 9, wherein the type VI system comprises a Cas13 nuclease.

Embodiment 11. The method of Embodiment 3, wherein the Cas nuclease is a Cas13 nuclease.

Embodiment 12. The method of Embodiment 11, wherein the Cas13 nuclease is selected from the group consisting of Cas13a, Cas13b, Cas13c, and Cas13d.

Embodiment 13. The method of Embodiment 11, wherein the Cas13 nuclease is Cas13d.

Embodiment 14. The method of any one of Embodiments 1-2, wherein the target cell is an immune cell.

Embodiment 15. The method of any one of Embodiments 1-2, wherein the target cell is a T cell.

Embodiment 16. The method of Embodiment 2, wherein the target cell is a tumor cell.

Embodiment 17. The method of Embodiment 2, wherein the target cell is a immune cell and a tumor cell.

Embodiment 18. The method of any one of Embodiments 1-2, wherein the gene silencing system comprises an RNA interference (RNAi) system.

Embodiment 19. The method of Embodiment 18, wherein the RNAi system is selected from a shRNA-based system, an siRNA-based system, and a miRNA-based system.

Embodiment 20. The method of Embodiment 18, wherein the RNAi system targets an endogenous RNA sequence comprising the nucleic acid sequence set forth in SEQ ID NOs: 1658-1665.

Embodiment 21. The method of Embodiment 18, wherein the RNAi system targets a gene selected from the group consisting of CD200, CD66, Galectin 3, CD47, or any combination thereof.

Embodiment 22. The method of Embodiment 18, wherein the RNAi system is an shRNA system.

Embodiment 23. The method of Embodiment 22, wherein the shRNA system comprises at least one nucleic acid selected from the group consisting of SEQ ID NO: 1666-1681.

Embodiment 24. The method of Embodiment 3, wherein the AAV-CRISPR vector is selected from the group consisting of AAV1, AAV2, AAV3, AAV3B, AAV4, AAV5, AAV6, AAV7, AAV8, AAV9, AAV12, AAV-B1, AAV-DJ, AAV-Retro, AAVrh8, AAVrh10, AAVrh25, Anc80L65, LK03, AAVrh18, AAVrh74, AAVrh32.33, AAVrh39, AAVrh43, Oligo001, PHP-B, and Spark100.

Embodiment 25. The method of Embodiment 3, wherein the AAV-CRISPR vector is AAV9.

Embodiment 26. The method of any one of Embodiments 1-2, wherein administering the effective amount of the gene silencing system comprises a one dose, a two dose, a three dose, a four dose, or a multi-dose treatment.

Embodiment 27. The method of Embodiment 2, wherein the tumor is a cancer selected from the group consisting of breast cancer, lung cancer, pancreatic cancer, melanoma, glioma, hepatoma, colon cancer, and brain cancer.

Embodiment 28. The method of Embodiment 2, wherein the administration of the gene silencing system results in increased CD8+ T cell infiltration into the tumor.

Embodiment 29. The method of Embodiment 2, wherein the gene silencing system is administered intratumoraly.

Embodiment 30. The method of Embodiment 2, further comprising administering an additional anti-tumor treatment.

Embodiment 31. The method of Embodiment 30, wherein the additional anti-tumor treatment is selected from the group consisting of chemotherapy, radiation, surgery, an immune checkpoint inhibitor, and an immune checkpoint blockade antibody.

Embodiment 32. The method of any one of Embodiments 1-2, wherein the subject is a mammal.

Embodiment 33. The method of any one of Embodiments 1-2, wherein the subject is a human.

Embodiment 34. A vector comprising an adeno-associated virus (AAV) genome, a U6 promoter sequence, a gRNA sequence, an EFS promoter sequence, and a Cas nuclease gene.

Embodiment 35. The vector of Embodiment 34, wherein the gRNA sequence comprises at least one nucleic acid sequence selected from the group consisting of SEQ ID NOs: 1-1657.

Embodiment 36. The vector of Embodiment 34, wherein the Cas nuclease is a RNA-targeting nuclease.

Embodiment 37. The vector of Embodiment 34, wherein the Cas nuclease is a Cas13 nuclease.

Embodiment 38. The vector of Embodiment 37, wherein the Cas13 nuclease is selected from the group consisting of Cas13a, Cas13b, Cas13c, and Cas13d.

Embodiment 39. The vector of Embodiment 37, wherein the Cas13 nuclease is Cas13d.

Embodiment 40. A composition comprising a gRNA library, wherein the gRNA library comprises a plurality of gRNAs that target a plurality of immunosuppressive genes in a cell.

Embodiment 41. The composition of Embodiment 40, wherein the plurality of gRNAs comprise at least one gRNA selected from the group consisting of SEQ ID NOs: 1-1657.

Embodiment 42. The composition of Embodiment 40, wherein the plurality of gRNAs comprise the nucleic acid sequences of SEQ ID NOs: 1-1657.

Embodiment 43. The composition of Embodiment 40, wherein the plurality of gRNAs comprise the nucleic acid sequences of SEQ ID NOs: 3-92.

Embodiment 44. The composition of Embodiment 40, wherein the plurality of gRNAs comprise the nucleic acid sequences of SEQ ID NOs: 93-1657.

Embodiment 45. The composition of Embodiment 40, wherein the gRNA library is packaged into an AAV vector.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While the present disclosure has been disclosed with reference to specific embodiments, it is apparent that other embodiments and variations of the present disclosure may be devised by others skilled in the art without departing from the true spirit and scope of the present disclosure. The appended claims are intended to be construed to include all such embodiments and equivalent variations.

1. A method of enhancing an immune response in a subject in need thereof,
the method comprising administering to the subject an effective amount of a gene silencing system,
wherein the gene silencing system decreases expression of at least one endogenous immunosuppressive gene in a target cell of the subject,
thereby enhancing the immune response in the subject.

2. A method of enhancing an anti-tumor immune response in a subject in need thereof,
the method comprising administering to the subject having a tumor an effective amount of a gene silencing system,
wherein the gene silencing system decreases expression of at least one endogenous immunosuppressive gene in a target cell of the subject,
thereby enhancing the anti-tumor immune response in the subject.

3. The method of claim 1, wherein the gene silencing system is a CRISPR-based gene silencing system which comprises a plurality of AAV-CRISPR vectors, wherein the plurality of AAV-CRISPR vectors comprises a Cas nuclease and a plurality of guide RNAs (gRNAs) homologous to mRNA from a plurality of target genes associated with immune suppression.

4. The method of claim 3, wherein the gRNA sequences comprise at least one nucleic acid sequence selected from the group consisting of SEQ ID NOs: 1-1657.

5. The method of claim 3, wherein the plurality of gRNAs comprise the nucleotide sequences of SEQ ID NOs: 1-1657.

6. The method of claim 3, wherein the gRNA sequences comprise at least one nucleic acid sequence selected from the group consisting of SEQ ID NOs: 1-92.

7. The method of claim 3, wherein the plurality of gRNAs comprise the nucleotide sequences of SEQ ID NOs: 1-92.

8. The method of claim 3, wherein the target genes are selected from the group consisting of Pdl1, Galectin9, Galectin3, and Cd47, or any combination thereof.

9. The method of claim 3, wherein the CRISPR-based gene silencing system is selected from the group consisting of a type III (Cmr/Csm) system, a type VI system, and a type II system.

10. The method of claim 9, wherein the type VI system comprises a Cas13 nuclease.

11. The method of claim 3, wherein the Cas nuclease is a Cas13 nuclease.

12. The method of claim 11, wherein the Cas13 nuclease is selected from the group consisting of Cas13a, Cas13b, Cas13c, and Cas13d.

13. The method of claim 11, wherein the Cas13 nuclease is Cas13d.

14. The method of claim 1, wherein the target cell is an immune cell.

15. The method of claim 1, wherein the target cell is a T cell.

16. The method of claim 2, wherein the target cell is a tumor cell.

17. The method of claim 2, wherein the target cell is a immune cell and a tumor cell.

18. The method of claim 1, wherein the gene silencing system comprises an RNA interference (RNAi) system.

19. The method of claim 18, wherein the RNAi system is selected from a shRNA-based system, an siRNA-based system, and a miRNA-based system.

20. The method of claim 18, wherein the RNAi system targets an endogenous RNA sequence comprising the nucleic acid sequences set forth in SEQ ID NOs: 1658-1665.

21. The method of claim 18, wherein the RNAi system targets a gene selected from the group consisting of CD200, CD66, Galectin 3, CD47, or any combination thereof.

22. The method of claim 18, wherein the RNAi system is an shRNA system.

23. The method of claim 22, wherein the shRNA system comprises at least one nucleic acid selected from the group consisting of SEQ ID NO: 1666-1681.

24. The method of claim 3, wherein the AAV-CRISPR vector is selected from the group consisting of AAV1, AAV2, AAV3, AAV3B, AAV4, AAV5, AAV6, AAV7, AAV8, AAV9, AAV12, AAV-B1, AAV-DJ, AAV-Retro, AAVrh8, AAVrh10, AAVrh25, Anc80L65, LK03, AAVrh18, AAVrh74, AAVrh32.33, AAVrh39, AAVrh43, Oligo001, PHP-B, and Spark 100.

25. The method of claim 3, wherein the AAV-CRISPR vector is AAV9.

26. The method of claim 1, wherein administering the effective amount of the gene silencing system comprises a one dose, a two dose, a three dose, a four dose, or a multi-dose treatment.

27. The method of claim 2, wherein the tumor is a cancer selected from the group consisting of breast cancer, lung cancer, pancreatic cancer, melanoma, glioma, hepatoma, colon cancer, and brain cancer.

28. The method of claim 2, wherein the administration of the gene silencing system results in increased CD8+ T cell infiltration into the tumor.

29. The method of claim 2, wherein the gene silencing system is administered intratumoraly.

30. The method of claim 2, further comprising administering an additional anti-tumor treatment to the subject.

31. The method of claim 30, wherein the additional anti-tumor treatment is selected from the group consisting of chemotherapy, radiation, surgery, an immune checkpoint inhibitor, and an immune checkpoint blockade antibody.

32. The method of claim 1, wherein the subject is a mammal.

33. The method of claim 1, wherein the subject is a human.

34. A vector comprising an adeno-associated virus (AAV) genome, a U6 promoter sequence, a gRNA sequence, an EFS promoter sequence, and a Cas nuclease gene.

35. The vector of claim 34, wherein the gRNA sequence comprises at least one nucleic acid sequence selected from the group consisting of SEQ ID NOs: 1-1657.

36. The vector of claim 34, wherein the Cas nuclease is a RNA-targeting nuclease.

37. The vector of claim 34, wherein the Cas nuclease is a Cas13 nuclease.

38. The vector of claim 37, wherein the Cas13 nuclease is selected from the group consisting of Cas13a, Cas13b, Cas13c, and Cas13d.

39. The vector of claim 37, wherein the Cas13 nuclease is Cas13d.

40. A composition comprising a gRNA library, wherein the gRNA library comprises a plurality of gRNAs that target a plurality of immunosuppressive genes in a cell.

41. The composition of claim 40, wherein the plurality of gRNAs comprise at least one gRNA selected from the group consisting of SEQ ID NOs: 1-1657.

42. The composition of claim 40, wherein the plurality of gRNAs comprise the nucleic acid sequences of SEQ ID NOs: 1-1657.

43. The composition of claim 40, wherein the plurality of gRNAs comprise the nucleic acid sequences of SEQ ID NOs: 3-92.

44. The composition of claim 40, wherein the plurality of gRNAs comprise the nucleic acid sequences of SEQ ID NOs: 93-1657.

45. The composition of claim 40, wherein the gRNA library is packaged into an AAV vector.


SEQUENCE LISTING (1681 sequences)

SEQ ID NO: 1 - RNA, 23 nt, synthetic construct
  source 1..23: mol_type = other RNA
cgagggcgacttaaccttaggat

SEQ ID NO: 2 - RNA, 23 nt, synthetic construct
  source 1..23: mol_type = other RNA
gtatcccatagtccttaaattgg

SEQ ID NO: 3 - RNA, 22 nt, synthetic construct
  source 1..22: mol_type = other RNA
aatgaggtaaatgtaatgctag

SEQ ID NO: 4 - RNA, 22 nt, synthetic construct
  source 1..22: mol_type = other RNA
gttgattttgcggtatggggca

SEQ ID NO: 5 - RNA, 22 nt, synthetic construct
  source 1..22: mol_type = other RNA
aatgagatgagatgttgagtgc

SEQ ID NO: 6 - RNA, 22 nt, synthetic construct
  source 1..22: mol_type = other RNA
tttgagcttgtatcttcaacgc

SEQ ID NO: 7 - RNA, 22 nt, synthetic construct
  source 1..22: mol_type = other RNA
ttagttcatgctcagaagtggc

SEQ ID NO: 8 - RNA, 22 nt, synthetic construct
  source 1..22: mol_type = other RNA
ttatgcagcagtaaacgcctgc

SEQ ID NO: 9 - RNA, 22 nt, synthetic construct
  source 1..22: mol_type = other RNA
ctttggagccgtgatagtaaac

SEQ ID NO: 10 - RNA, 22 nt, synthetic construct
  source 1..22: mol_type = other RNA
ctcgaattgtgtatcatttcgg

SEQ ID NO: 11 - RNA, 22 nt, synthetic construct
  source 1..22: mol_type = other RNA
gaatcacttgctcatcttcctt

SEQ ID NO: 12 - RNA, 22 nt, synthetic construct
  source 1..22: mol_type = other RNA
aaggtcctcctctcctgccaca

SEQ ID NO: 13 - RNA, 23 nt, synthetic construct
  source 1..23: mol_type = other RNA
tataatgccagcaaatatcctca

SEQ ID NO: 14 - RNA, 23 nt, synthetic construct
  source 1..23: mol_type = other RNA
cgtagcaagtgacagcaggctgt

SEQ ID NO: 15 - RNA, 23 nt, synthetic construct
  source 1..23: mol_type = other RNA
gctgccatactccaccacgtaca

SEQ ID NO: 16 - RNA, 23 nt, synthetic construct
  source 1..23: mol_type = other RNA
ccatcgtgacgttgctgccatac

SEQ ID NO: 17 - RNA, 23 nt, synthetic construct
  source 1..23: mol_type = other RNA
taacgcaagcaggtccagctccc

SEQ ID NO: 18 - RNA, 23 nt, synthetic construct
  source 1..23: mol_type = other RNA
tttcccagtacaccactaacgca

SEQ ID NO: 19 - RNA, 23 nt, synthetic construct
  source 1..23: mol_type = other RNA
acttgctcatcttccttttccca

SEQ ID NO: 20 - RNA, 23 nt, synthetic construct
  source 1..23: mol_type = other RNA
ccctgaagttgctgtgctgaggc

SEQ ID NO: 21 - RNA, 23 nt, synthetic construct
  source 1..23: mol_type = other RNA
ctggtcctttggcagcgaggctc

SEQ ID NO: 22 - RNA, 23 nt, synthetic construct
  source 1..23: mol_type = other RNA
tgatctgaagggcagcatttccc

SEQ ID NO: 23 - RNA, 23 nt, synthetic construct
  source 1..23: mol_type = other RNA
ctgcgtcctgcagcttgacgtct

SEQ ID NO: 24 - RNA, 23 nt, synthetic construct
  source 1..23: mol_type = other RNA
ctgattatgcagcagtaaacgcc

SEQ ID NO: 25 - RNA, 23 nt, synthetic construct
  source 1..23: mol_type = other RNA
cgcttgtagtccgcaccaccgta

SEQ ID NO: 26 - RNA, 23 nt, synthetic construct
  source 1..23: mol_type = other RNA
tcagcgtgattcgcttgtagtcc

SEQ ID NO: 27 - RNA, 23 nt, synthetic construct
  source 1..23: mol_type = other RNA
tctggttgattttgcggtatggg

SEQ ID NO: 28 - RNA, 23 nt, synthetic construct
  source 1..23: mol_type = other RNA
gcctgacatattagttcatgctc

SEQ ID NO: 29 - RNA, 23 nt, synthetic construct
  source 1..23: mol_type = other RNA
ctcggcctgacatattagttcat

SEQ ID NO: 30 - RNA, 23 nt, synthetic construct
  source 1..23: mol_type = other RNA
ggttggtggtcactgtttgtcca

SEQ ID NO: 31 - RNA, 23 nt, synthetic construct
  source 1..23: mol_type = other RNA
tggtgacacttctcttcccactc

SEQ ID NO: 32 - RNA, 23 nt, synthetic construct
  source 1..23: mol_type = other RNA
tctgtccgggaagtggtgacact

SEQ ID NO: 33 - RNA, 23 nt, synthetic construct
  source 1..23: mol_type = other RNA
gactgctggtcacattgagaagc

SEQ ID NO: 34 - RNA, 23 nt, synthetic construct
  source 1..23: mol_type = other RNA
tagaaaacatcattcgctgtggc

SEQ ID NO: 35 - RNA, 23 nt, synthetic construct
  source 1..23: mol_type = other RNA
tgtgatctccaaaacgtacagta

SEQ ID NO: 36 - RNA, 23 nt, synthetic construct
  source 1..23: mol_type = other RNA
ctccgctgtgtggttttgccctg

SEQ ID NO: 37 - RNA, 23 nt, synthetic construct
  source 1..23: mol_type = other RNA
tgttctgtggaggatgtgttgca

SEQ ID NO: 38 - RNA, 23 nt, synthetic construct
  source 1..23: mol_type = other RNA
tggatcccagaagcacccagtga

SEQ ID NO: 39 - RNA, 23 nt, synthetic construct
  source 1..23: mol_type = other RNA
aagaggaggaccgtggacactac

SEQ ID NO: 40 - RNA, 23 nt, synthetic construct
  source 1..23: mol_type = other RNA
tctcaagaagaggaggaccgtgg

SEQ ID NO: 41 - RNA, 23 nt, synthetic construct
  source 1..23: mol_type = other RNA
atttctccacatctagcattctc

SEQ ID NO: 42 - RNA, 23 nt, synthetic construct
  source 1..23: mol_type = other RNA
ggttttttgagcttgtatcttca

SEQ ID NO: 43 - RNA, 22 nt, synthetic construct
  source 1..22: mol_type = other RNA
ttaatgtatggagactgggcac

SEQ ID NO: 44 - RNA, 22 nt, synthetic construct
  source 1..22: mol_type = other RNA
gagttttctgtttgcgcccctt

SEQ ID NO: 45 - RNA, 22 nt, synthetic construct
  source 1..22: mol_type = other RNA
atatagctagcttgggggcaat

SEQ ID NO: 46 - RNA, 23 nt, synthetic construct
  source 1..23: mol_type = other RNA
gggttaatgtatggagactgggc

SEQ ID NO: 47 - RNA, 23 nt, synthetic construct
  source 1..23: mol_type = other RNA
gtgggcaggacgaaagttctgag

SEQ ID NO: 48 - RNA, 23 nt, synthetic construct
  source 1..23: mol_type = other RNA
gaaccatatggatggtagtttga

SEQ ID NO: 49 - RNA, 23 nt, synthetic construct
  source 1..23: mol_type = other RNA
gggtacaccacaggagggattcc

SEQ ID NO: 50 - RNA, 23 nt, synthetic construct
  source 1..23: mol_type = other RNA
ccatttggaatgggggtgtagaa

SEQ ID NO: 51 - RNA, 23 nt, synthetic construct
  source 1..23: mol_type = other RNA
atatcatgatggacttggacggg

SEQ ID NO: 52 - RNA, 23 nt, synthetic construct
  source 1..23: mol_type = other RNA
ggcaagacattgcctgatatcat

SEQ ID NO: 53 - RNA, 23 nt, synthetic construct
  source 1..23: mol_type = other RNA
ccttcacatatgatccacaccga

SEQ ID NO: 54 - RNA, 23 nt, synthetic construct
  source 1..23: mol_type = other RNA
agctaggaaacagaaaccacagc

SEQ ID NO: 55 - RNA, 23 nt, synthetic construct
  source 1..23: mol_type = other RNA
ataaaggacagcttcaagcagcc

SEQ ID NO: 56 - RNA, 23 nt, synthetic construct
  source 1..23: mol_type = other RNA
agaatttcttgttcaccatcacc

SEQ ID NO: 57 - RNA, 23 nt, synthetic construct
  source 1..23: mol_type = other RNA
gaaagcaatgtcacctccacagc

SEQ ID NO: 58 - RNA, 23 nt, synthetic construct
  source 1..23: mol_type = other RNA
ggaaagataagacacaggcagag

SEQ ID NO: 59 - RNA, 23 nt, synthetic construct
  source 1..23: mol_type = other RNA
ttgaactctgacctctgcaccag

SEQ ID NO: 60 - RNA, 23 nt, synthetic construct
  source 1..23: mol_type = other RNA
cgatcagagatctgagcaaaccc

SEQ ID NO: 61 - RNA, 23 nt, synthetic construct
  source 1..23: mol_type = other RNA
tagagtgttgatatcctgcaagt

SEQ ID NO: 62 - RNA, 23 nt, synthetic construct
  source 1..23: mol_type = other RNA
acacagcctagaaaacccccttt

SEQ ID NO: 63 - RNA, 24 nt, synthetic construct
  source 1..24: mol_type = other RNA
ttactgtggacattgtgggtcagt

SEQ ID NO: 64 - RNA, 24 nt, synthetic construct
  source 1..24: mol_type = other RNA
atcctcccaagcagacttcgctct

SEQ ID NO: 65 - RNA, 23 nt, synthetic construct
  source 1..23: mol_type = other RNA
aaaggtatggtataggctggggt

SEQ ID NO: 66 - RNA, 23 nt, synthetic construct
  source 1..23: mol_type = other RNA
tgaaagttcaccacaaacctttg

SEQ ID NO: 67 - RNA, 23 nt, synthetic construct
  source 1..23: mol_type = other RNA
aactcttggtagtcccctggagg

SEQ ID NO: 68 - RNA, 23 nt, synthetic construct
  source 1..23: mol_type = other RNA
tacaattacaagatagtccaagg

SEQ ID NO: 69 - RNA, 23 nt, synthetic construct
  source 1..23: mol_type = other RNA
agatgtattcagaaaccacgctg

SEQ ID NO: 70 - RNA, 23 nt, synthetic construct
  source 1..23: mol_type = other RNA
ctttcggttaatatcgtacacca

SEQ ID NO: 71 - RNA, 23 nt, synthetic construct
  source 1..23: mol_type = other RNA
atctcaaaaccagagtgccccag

SEQ ID NO: 72 - RNA, 23 nt, synthetic construct
  source 1..23: mol_type = other RNA
ctgagagacaacaagagcccaaa

SEQ ID NO: 73 - RNA, 23 nt, synthetic construct
  source 1..23: mol_type = other RNA
acatcagattcaagtagaccagg

SEQ ID NO: 74 - RNA, 23 nt, synthetic construct
  source 1..23: mol_type = other RNA
aagagacacatgtagcagccctc

SEQ ID NO: 75 - RNA, 23 nt, synthetic construct
  source 1..23: mol_type = other RNA
tcatagaagcacatagagaacgg

SEQ ID NO: 76 - RNA, 23 nt, synthetic construct
  source 1..23: mol_type = other RNA
aaagtcacagaaaaagaagctgg

SEQ ID NO: 77 - RNA, 23 nt, synthetic construct
  source 1..23: mol_type = other RNA
ccatcagcagaacagtagcaggt

SEQ ID NO: 78 - RNA, 23 nt, synthetic construct
  source 1..23: mol_type = other RNA
caaacagacagtagcagaggcca

SEQ ID NO: 79 - RNA, 23 nt, synthetic construct
  source 1..23: mol_type = other RNA
gaagcaaaatatacacaggctgt

SEQ ID NO: 80 - RNA, 23 nt, synthetic construct
  source 1..23: mol_type = other RNA
gtacatgaaatcgcacagtcgcc

SEQ ID NO: 81 - RNA, 23 nt, synthetic construct
  source 1..23: mol_type = other RNA
ttaaaagaaacacaagaaggcag

SEQ ID NO: 82 - RNA, 23 nt, synthetic construct
  source 1..23: mol_type = other RNA
gacagaaatctagtggccctaag

SEQ ID NO: 83 - RNA, 23 nt, synthetic construct
  source 1..23: mol_type = other RNA
ccagtatcataaaaacccccaaa

SEQ ID NO: 84 - RNA, 23 nt, synthetic construct
  source 1..23: mol_type = other RNA
ttaagcgaaaagctgtctgccat

SEQ ID NO: 85 - RNA, 23 nt, synthetic construct
  source 1..23: mol_type = other RNA
cagtcctaagatttgacatccag

SEQ ID NO: 86 - RNA, 23 nt, synthetic construct
  source 1..23: mol_type = other RNA
cacacagcttacttacaaccacc

SEQ ID NO: 87 - RNA, 23 nt, synthetic construct
  source 1..23: mol_type = other RNA
agggcatataaagacaggcagct

SEQ ID NO: 88 - RNA, 23 nt, synthetic construct
  source 1..23: mol_type = other RNA
caagcaagacagaagcgccaagt

SEQ ID NO: 89 - RNA, 23 nt, synthetic construct
  source 1..23: mol_type = other RNA
tagagattacaatgaggccaagt

SEQ ID NO: 90 - RNA, 23 nt, synthetic construct
  source 1..23: mol_type = other RNA
accaaagcaaggacgtagcccag

SEQ ID NO: 91 - RNA, 23 nt, synthetic construct
  source 1..23: mol_type = other RNA
ccacgatgactgtgagcaccagc

SEQ ID NO: 92 - RNA, 23 nt, synthetic construct
  source 1..23: mol_type = other RNA
taaacagtagttgagctgaacct

SEQ ID NO: 93 - RNA, 23 nt, synthetic construct
  source 1..23: mol_type = other RNA
aatacaaaatatagtggcaccca

SEQ ID NO: 94 - RNA, 23 nt, synthetic construct
  source 1..23: mol_type = other RNA
gccacaaaatcatagagtgccac

SEQ ID NO: 95 - RNA, 23 nt, synthetic construct
  source 1..23: mol_type = other RNA
caatgaaaacacagtgagcacag

SEQ ID NO: 96 - RNA, 23 nt, synthetic construct
  source 1..23: mol_type = other RNA
agaagaaatgcatgaccagcaac

SEQ ID NO: 97 - RNA, 23 nt, synthetic construct
  source 1..23: mol_type = other RNA
tcacacatgtaaacaggagcctg

SEQ ID NO: 98 - RNA, 23 nt, synthetic construct
  source 1..23: mol_type = other RNA
aggtatataagcaagcccaacca

SEQ ID NO: 99 - RNA, 23 nt, synthetic construct
  source 1..23: mol_type = other RNA
cgttgcaaatactagccagaccg

SEQ ID NO: 100 - RNA, 23 nt, synthetic construct
  source 1..23: mol_type = other RNA
acacataagactttgagaggcca

SEQ ID NO: 101 - RNA, 23 nt, synthetic construct
  source 1..23: mol_type = other RNA
aaaacaaaacataaacagccagt

SEQ ID NO: 102 - RNA, 23 nt, synthetic construct
  source 1..23: mol_type = other RNA
gattaaagccatgtaccacccaa

SEQ ID NO: 103 - RNA, 23 nt, synthetic construct
  source 1..23: mol_type = other RNA
ataaaagtttatcgagagccaag

SEQ ID NO: 104 - RNA, 23 nt, synthetic construct
  source 1..23: mol_type = other RNA
tcaatgtaaaacgtgccaccacg

SEQ ID NO: 105 - RNA, 23 nt, synthetic construct
  source 1..23: mol_type = other RNA
gacaagtatttctttcagccaga

SEQ ID NO: 106 - RNA, 23 nt, synthetic construct
  source 1..23: mol_type = other RNA
aatacacaaagtaataagcaggc

SEQ ID NO: 107 - RNA, 23 nt, synthetic construct
  source 1..23: mol_type = other RNA
cagaattaacactgtcggcaaca

SEQ ID NO: 108 - RNA, 23 nt, synthetic construct
  source 1..23: mol_type = other RNA
cagaacatcttgaagcaccagag

SEQ ID NO: 109 - RNA, 23 nt, synthetic construct
  source 1..23: mol_type = other RNA
cattcatacatatacccacacac

SEQ ID NO: 110 - RNA, 23 nt, synthetic construct
  source 1..23: mol_type = other RNA
agttacagagttgagagccacca

SEQ ID NO: 111 - RNA, 23 nt, synthetic construct
  source 1..23: mol_type = other RNA
gaaaaccagaacagacccaacga

SEQ ID NO: 112 - RNA, 23 nt, synthetic construct
  source 1..23: mol_type = other RNA
tatcttcagacttatacaccagc

SEQ ID NO: 113 - RNA, 23 nt, synthetic construct
  source 1..23: mol_type = other RNA
ttcaccataagagagctgcagta

SEQ ID NO: 114 - RNA, 23 nt, synthetic construct
  source 1..23: mol_type = other RNA
tcaaggaatgcaagacagccacg

SEQ ID NO: 115 - RNA, 23 nt, synthetic construct
  source 1..23: mol_type = other RNA
cttttcataataatggcagccag

SEQ ID NO: 116 - RNA, 23 nt, synthetic construct
  source 1..23: mol_type = other RNA
agaccagaagaatcccagtgcac

SEQ ID NO: 117 - RNA, 23 nt, synthetic construct
  source 1..23: mol_type = other RNA
ctgagcatactctaacaacccgc

SEQ ID NO: 118 - RNA, 23 nt, synthetic construct
  source 1..23: mol_type = other RNA
atgcgaatattgtgaagccccca

SEQ ID NO: 119 - RNA, 23 nt, synthetic construct
  source 1..23: mol_type = other RNA
ttcacatctttcatgtacaccgt

SEQ ID NO: 120 - RNA, 23 nt, synthetic construct
  source 1..23: mol_type = other RNA
agcgatacacataagcggcttct

SEQ ID NO: 121 - RNA, 23 nt, synthetic construct
  source 1..23: mol_type = other RNA
aatatttgtgtacagtgagcaga

SEQ ID NO: 122 - RNA, 23 nt, synthetic construct
  source 1..23: mol_type = other RNA
ctttagacattcatacactcagc

SEQ ID NO: 123 - RNA, 23 nt, synthetic construct
  source 1..23: mol_type = other RNA
acaaacaaacaaacaagccccac

SEQ ID NO: 124 - RNA, 23 nt, synthetic construct
  source 1..23: mol_type = other RNA
ttaatgagattggtccagccaac

SEQ ID NO: 125 - RNA, 23 nt, synthetic construct
  source 1..23: mol_type = other RNA
aaaatccttaggtagatggccag

SEQ ID NO: 126 - RNA, 23 nt, synthetic construct
  source 1..23: mol_type = other RNA
cagcaaatcgcaatgatgccctt

SEQ ID NO: 127 - RNA, 23 nt, synthetic construct
  source 1..23: mol_type = other RNA
atgatgtacaccgaggagcccat

SEQ ID NO: 128 - RNA, 23 nt, synthetic construct
  source 1..23: mol_type = other RNA
ccatagagcaagaaccagcaccc

SEQ ID NO: 129 - RNA, 23 nt, synthetic construct
  source 1..23: mol_type = other RNA
cgttttcgccacaggatagcccc

SEQ ID NO: 130 - RNA, 23 nt, synthetic construct
  source 1..23: mol_type = other RNA
tgtcaaatgtttactcagcatgg

SEQ ID NO: 131 - RNA, 23 nt, synthetic construct
  source 1..23: mol_type = other RNA
gatcactgtcagctctgaccgca

SEQ ID NO: 132 - RNA, 23 nt, synthetic construct
  source 1..23: mol_type = other RNA
ccacgcagctataggtgccctca

SEQ ID NO: 133 - RNA, 23 nt, synthetic construct
  source 1..23: mol_type = other RNA
gtaaaagttaagatgccacaaca

SEQ ID NO: 134 - RNA, 23 nt, synthetic construct
  source 1..23: mol_type = other RNA
agacttagttaatagcacccaac

SEQ ID NO: 135 - RNA, 23 nt, synthetic construct
  source 1..23: mol_type = other RNA
attgtcattagtaagacacccac

SEQ ID NO: 136 - RNA, 23 nt, synthetic construct
  source 1..23: mol_type = other RNA
aaaaataaagcattagcaagcct

SEQ ID NO: 137 - RNA, 23 nt, synthetic construct
  source 1..23: mol_type = other RNA
cagcaactatgttacagacctcc

SEQ ID NO: 138 - RNA, 23 nt, synthetic construct
  source 1..23: mol_type = other RNA
aggttcacatatgtgacagcagc

SEQ ID NO: 139 - RNA, 23 nt, synthetic construct
  source 1..23: mol_type = other RNA
gtcacaacagatgagctcacctc

SEQ ID NO: 140 - RNA, 23 nt, synthetic construct
  source 1..23: mol_type = other RNA
agtccttaaagagaatcctccag

SEQ ID NO: 141 - RNA, 23 nt, synthetic construct
  source 1..23: mol_type = other RNA
atctctacaaagatcagggccat

SEQ ID NO: 142 - RNA, 23 nt, synthetic construct
  source 1..23: mol_type = other RNA
cgtagcattccatccccacctgc

SEQ ID NO: 143 - RNA, 23 nt, synthetic construct
  source 1..23: mol_type = other RNA
atagcaatcagaatcagaaccgt

SEQ ID NO: 144 - RNA, 23 nt, synthetic construct
  source 1..23: mol_type = other RNA
tatacatccaattaacagccact

SEQ ID NO: 145 - RNA, 23 nt, synthetic construct
  source 1..23: mol_type = other RNA
ccttaaaaagtacctcaggcaga

SEQ ID NO: 146 - RNA, 23 nt, synthetic construct
  source 1..23: mol_type = other RNA
agattatagttttagacagtcca

SEQ ID NO: 147 - RNA, 23 nt, synthetic construct
  source 1..23: mol_type = other RNA
caatttcaaaagcttgaaccacc

SEQ ID NO: 148 - RNA, 23 nt, synthetic construct
  source 1..23: mol_type = other RNA
aggccaaaagaaaaagcccacaa

SEQ ID NO: 149 - RNA, 23 nt, synthetic construct
  source 1..23: mol_type = other RNA
agattgtaatacaagccaaacca

SEQ ID NO: 150 - RNA, 23 nt, synthetic construct
  source 1..23: mol_type = other RNA
tcgtcataatcatagcacctcca

SEQ ID NO: 151 - RNA, 23 nt, synthetic construct
  source 1..23: mol_type = other RNA
agacaagcagcaaagagaaaccc

SEQ ID NO: 152 - RNA, 23 nt, synthetic construct
  source 1..23: mol_type = other RNA
cccaaacttctcaccagcaccga

SEQ ID NO: 153 - RNA, 23 nt, synthetic construct
  source 1..23: mol_type = other RNA
cagagacaaagatagtgacagct

SEQ ID NO: 154 - RNA, 23 nt, synthetic construct
  source 1..23: mol_type = other RNA
aatcatctataatatagccggat

SEQ ID NO: 155 - RNA, 23 nt, synthetic construct
  source 1..23: mol_type = other RNA
acagaaagctcaagtccaccggc

SEQ ID NO: 156 - RNA, 23 nt, synthetic construct
  source 1..23: mol_type = other RNA
acaattgcatgtcaccacctcca

SEQ ID NO: 157 - RNA, 23 nt, synthetic construct
  source 1..23: mol_type = other RNA
ctatcataaaaagtgacaactgg

SEQ ID NO: 158 - RNA, 23 nt, synthetic construct
  source 1..23: mol_type = other RNA
aggcaatattgatccagacagcc

SEQ ID NO: 159 - RNA, 23 nt, synthetic construct
  source 1..23: mol_type = other RNA
caacaagatccagagctgacagc

SEQ ID NO: 160 - RNA, 23 nt, synthetic construct
  source 1..23: mol_type = other RNA
aataaaatgttcaggaggctcca

SEQ ID NO: 161 - RNA, 23 nt, synthetic construct
  source 1..23: mol_type = other RNA
tacagtaatagtgttggtgacca

SEQ ID NO: 162 - RNA, 23 nt, synthetic construct
  source 1..23: mol_type = other RNA
accagattattgtagggatcatc

SEQ ID NO: 163 - RNA, 23 nt, synthetic construct
  source 1..23: mol_type = other RNA
agaacatctttagagccaggcat

SEQ ID NO: 164 - RNA, 23 nt, synthetic construct
  source 1..23: mol_type = other RNA
ggaactaacatattggtactccg

SEQ ID NO: 165 - RNA, 23 nt, synthetic construct
  source 1..23: mol_type = other RNA
gaagaacttccagaggcaaccaa

SEQ ID NO: 166 - RNA, 23 nt, synthetic construct
  source 1..23: mol_type = other RNA
ccagaagccaagtacgtgcatga

SEQ ID NO: 167 - RNA, 23 nt, synthetic construct
  source 1..23: mol_type = other RNA
tgatctagacagaatacaccctc

SEQ ID NO: 168 - RNA, 23 nt, synthetic construct
  source 1..23: mol_type = other RNA
cctgcagtaattcaacagagcag

SEQ ID NO: 169 - RNA, 23 nt, synthetic construct
  source 1..23: mol_type = other RNA
ccctaaagtaaagaaccggcact

SEQ ID NO: 170 - RNA, 23 nt, synthetic construct
  source 1..23: mol_type = other RNA
cagaggtaaattcagaccaacca

SEQ ID NO: 171 - RNA, 23 nt, synthetic construct
  source 1..23: mol_type = other RNA
catcgttcactaaagcgagcaca

SEQ ID NO: 172 - RNA, 23 nt, synthetic construct
  source 1..23: mol_type = other RNA
tacattaattttcagaaacaggc

SEQ ID NO: 173 - RNA, 23 nt, synthetic construct
  source 1..23: mol_type = other RNA
taagaccatgtaaagcagcccat

SEQ ID NO: 174 - RNA, 23 nt, synthetic construct
  source 1..23: mol_type = other RNA
atatgaaataaaacaaggccacc

SEQ ID NO: 175 - RNA, 23 nt, synthetic construct
  source 1..23: mol_type = other RNA
accaaggaagaaaccggacagca

SEQ ID NO: 176 - RNA, 23 nt, synthetic construct
  source 1..23: mol_type = other RNA
taataactgccaaagcgccacag

SEQ ID NO: 177 - RNA, 23 nt, synthetic construct
  source 1..23: mol_type = other RNA
agacacaatgttgacgagctgcg

SEQ ID NO: 178 - RNA, 23 nt, synthetic construct
  source 1..23: mol_type = other RNA
actcattgagaaacagagagccg

SEQ ID NO: 179 - RNA, 23 nt, synthetic construct
  source 1..23: mol_type = other RNA
taggactacatagaagcagccag

SEQ ID NO: 180 - RNA, 23 nt, synthetic construct
  source 1..23: mol_type = other RNA
atagataaacttgataagccggt

SEQ ID NO: 181 - RNA, 23 nt, synthetic construct
  source 1..23: mol_type = other RNA
caaaacattctagctgcgcgccc

SEQ ID NO: 182 - RNA, 23 nt, synthetic construct
  source 1..23: mol_type = other RNA
gtccaccttgttaaccagctcca

SEQ ID NO: 183 - RNA, 23 nt, synthetic construct
  source 1..23: mol_type = other RNA
tttataaaattgcagacgccgat

SEQ ID NO: 184 - RNA, 23 nt, synthetic construct
  source 1..23: mol_type = other RNA
ctggaattcaaatgacagcacct

SEQ ID NO: 185 - RNA, 23 nt, synthetic construct
  source 1..23: mol_type = other RNA
gacagagaattgcagcatcaccg

SEQ ID NO: 186 - RNA, 23 nt, synthetic construct
  source 1..23: mol_type = other RNA
cactgagaaaacatagagtgcag

SEQ ID NO: 187 - RNA, 23 nt, synthetic construct
  source 1..23: mol_type = other RNA
cccaaagatgacatgcagccgag

SEQ ID NO: 188 - RNA, 23 nt, synthetic construct
  source 1..23: mol_type = other RNA
ccaaactaagcaataggtcagag

SEQ ID NO: 189 - RNA, 23 nt, synthetic construct
  source 1..23: mol_type = other RNA
agtgacaaagtatcacaccaggc

SEQ ID NO: 190 - RNA, 23 nt, synthetic construct
  source 1..23: mol_type = other RNA
gcacatatataaacagacacagg

SEQ ID NO: 191 - RNA, 23 nt, synthetic construct
  source 1..23: mol_type = other RNA
acaaaggaaaccagagctgccag

SEQ ID NO: 192 - RNA, 23 nt, synthetic construct
  source 1..23: mol_type = other RNA
cacctgaaaaatgagaggcagag

SEQ ID NO: 193 - RNA, 23 nt, synthetic construct
  source 1..23: mol_type = other RNA
acatgcagaaaaagaaacccccg

SEQ ID NO: 194 - RNA, 23 nt, synthetic construct
  source 1..23: mol_type = other RNA
ttaaacagcaccttcagagccag

SEQ ID NO: 195 - RNA, 23 nt, synthetic construct
  source 1..23: mol_type = other RNA
aggaactcatagcagagaacgcc

SEQ ID NO: 196 - RNA, 23 nt, synthetic construct
  source 1..23: mol_type = other RNA
actaaatcaggaaaagcagcatg

SEQ ID NO: 197 - RNA, 23 nt, synthetic construct
  source 1..23: mol_type = other RNA
tgaatgacagtaagacagagcgt

SEQ ID NO: 198 - RNA, 23 nt, synthetic construct
  source 1..23: mol_type = other RNA
aaaattaaaacgtggcagagccc

SEQ ID NO: 199 - RNA, 23 nt, synthetic construct
  source 1..23: mol_type = other RNA
acaaagtgagttccaagacagcc

SEQ ID NO: 200 - RNA, 23 nt, synthetic construct
  source 1..23: mol_type = other RNA
agaacccacaaagtagctacagg

SEQ ID NO: 201 - RNA, 23 nt, synthetic construct
  source 1..23: mol_type = other RNA
gattaacagtaacagcaggtcta

SEQ ID NO: 202 - RNA, 23 nt, synthetic construct
  source 1..23: mol_type = other RNA
tagaacaagaagtcgcgcacgtg

SEQ ID NO: 203 - RNA, 23 nt, synthetic construct
  source 1..23: mol_type = other RNA
acagagaataagcatcaacagcc

SEQ ID NO: 204 - RNA, 23 nt, synthetic construct
  source 1..23: mol_type = other RNA
tcaagacacagatgaacagcgcc

SEQ ID NO: 205 - RNA, 23 nt, synthetic construct
  source 1..23: mol_type = other RNA
cctttaagaattaccaaccccac

SEQ ID NO: 206 - RNA, 23 nt, synthetic construct
  source 1..23: mol_type = other RNA
caaaccatataatttccccaggt

SEQ ID NO: 207 - RNA, 23 nt, synthetic construct
  source 1..23: mol_type = other RNA
caaactgtagcagtggcccaaag

SEQ ID NO: 208 - RNA, 23 nt, synthetic construct
  source 1..23: mol_type = other RNA
tatttagaaaactatccaccccc

SEQ ID NO: 209 - RNA, 23 nt, synthetic construct
  source 1..23: mol_type = other RNA
cctcagtttacatgcccctccaa

SEQ ID NO: 210 - RNA, 23 nt, synthetic construct
  source 1..23: mol_type = other RNA
cagcaacctctgacagacccctg

SEQ ID NO: 211 - RNA, 23 nt, synthetic construct
  source 1..23: mol_type = other RNA
gtgttcaatacttgtccaggcct

SEQ ID NO: 212 - RNA, 23 nt, synthetic construct
  source 1..23: mol_type = other RNA
ctcacatcatcagatgagtcctg

SEQ ID NO: 213 - RNA, 23 nt, synthetic construct
  source 1..23: mol_type = other RNA
cagtacaaaagcaaaccgcagag

SEQ ID NO: 214 - RNA, 23 nt, synthetic construct
  source 1..23: mol_type = other RNA
agcagattttatagacccaccat

SEQ ID NO: 215 - RNA, 23 nt, synthetic construct
  source 1..23: mol_type = other RNA
ctccagttaaaaatacagcgaga

SEQ ID NO: 216 - RNA, 23 nt, synthetic construct
  source 1..23: mol_type = other RNA
caaaacacagaaacggaagcagc

SEQ ID NO: 217 - RNA, 23 nt, synthetic construct
  source 1..23: mol_type = other RNA
aacttgaaggtcttgccgcagaa

SEQ ID NO: 218 - RNA, 23 nt, synthetic construct
  source 1..23: mol_type = other RNA
aaacaaatacataaggcaaccac

SEQ ID NO: 219 - RNA, 23 nt, synthetic construct
  source 1..23: mol_type = other RNA
tgtatgaataaaggccacaccca

SEQ ID NO: 220 - RNA, 23 nt, synthetic construct
  source 1..23: mol_type = other RNA
cttttagagcaaatgcagccaca

SEQ ID NO: 221 - RNA, 23 nt, synthetic construct
  source 1..23: mol_type = other RNA
tatcaaccttaaaagcagcccat

SEQ ID NO: 222 - RNA, 23 nt, synthetic construct
  source 1..23: mol_type = other RNA
ccgaactcaaagaaggccacaat

SEQ ID NO: 223 - RNA, 23 nt, synthetic construct
  source 1..23: mol_type = other RNA
aaacataaaagtataccaccacc

SEQ ID NO: 224 - RNA, 23 nt, synthetic construct
  source 1..23: mol_type = other RNA
ttgtatgaaattacgccaagcac

SEQ ID NO: 225 - RNA, 23 nt, synthetic construct
  source 1..23: mol_type = other RNA
ctgaaacattagtgaccacggca

SEQ ID NO: 226 - RNA, 23 nt, synthetic construct
  source 1..23: mol_type = other RNA
ccctgttaattcaatgcacacct

SEQ ID NO: 227 - RNA, 23 nt, synthetic construct
  source 1..23: mol_type = other RNA
accactttagaagacaggtcaag

SEQ ID NO: 228 - RNA, 23 nt, synthetic construct
  source 1..23: mol_type = other RNA
accagtgtagtaaaagccagcac

SEQ ID NO: 229 - RNA, 23 nt, synthetic construct
  source 1..23: mol_type = other RNA
tgacagaaaagacaatggccatg

SEQ ID NO: 230 - RNA, 23 nt, synthetic construct
  source 1..23: mol_type = other RNA
gtcctgtataataaaagcccgca

SEQ ID NO: 231 - RNA, 23 nt, synthetic construct
  source 1..23: mol_type = other RNA
caagtcttttaagaacggacagc

SEQ ID NO: 232 - RNA, 23 nt, synthetic construct
  source 1..23: mol_type = other RNA
agaatttcaggagtgcaggcaat

SEQ ID NO: 233 - RNA, 23 nt, synthetic construct
  source 1..23: mol_type = other RNA
agacagttccaaatgacccagat

SEQ ID NO: 234 - RNA, 23 nt, synthetic construct
  source 1..23: mol_type = other RNA
gaattctgtaaacagcacagcat

SEQ ID NO: 235 - RNA, 23 nt, synthetic construct
  source 1..23: mol_type = other RNA
cattcaacattttcactgccaca

SEQ ID NO: 236 - RNA, 23 nt, synthetic construct
  source 1..23: mol_type = other RNA
ataaccacatgtttgacaacgga

SEQ ID NO: 237 - RNA, 23 nt, synthetic construct
  source 1..23: mol_type = other RNA
tccacaaaatagatccagacaac

SEQ ID NO: 238 - RNA, 23 nt, synthetic construct
  source 1..23: mol_type = other RNA
aacagtgacactttgagcaccag

SEQ ID NO: 239 - RNA, 23 nt, synthetic construct
  source 1..23: mol_type = other RNA
gcaaaagcaataaagcagaactc

SEQ ID NO: 240 - RNA, 23 nt, synthetic construct
  source 1..23: mol_type = other RNA
agacaccagaaatatgtgacccc

SEQ ID NO: 241 - RNA, 23 nt, synthetic construct
  source 1..23: mol_type = other RNA
tcaaacaactgtgacctgccaac

SEQ ID NO: 242 - RNA, 23 nt, synthetic construct
  source 1..23: mol_type = other RNA
atagtgatagaaagagggcgcca

SEQ ID NO: 243 - RNA, 23 nt, synthetic construct
  source 1..23: mol_type = other RNA
atatgtatattaatccagcagca

SEQ ID NO: 244 - RNA, 23 nt, synthetic construct
  source 1..23: mol_type = other RNA
gacctttaagacgcagcaccagc

SEQ ID NO: 245 - RNA, 23 nt, synthetic construct
  source 1..23: mol_type = other RNA
atccttttcagaaagcagagcag

SEQ ID NO: 246 - RNA, 23 nt, synthetic construct
  source 1..23: mol_type = other RNA
taacagaataaagtggagtgcaa

SEQ ID NO: 247 - RNA, 23 nt, synthetic construct
  source 1..23: mol_type = other RNA
tgtagaacagctatacgacccat

SEQ ID NO: 248 - RNA, 23 nt, synthetic construct
  source 1..23: mol_type = other RNA
tccagagatagataaagcacccg

SEQ ID NO: 249 - RNA, 23 nt, synthetic construct
  source 1..23: mol_type = other RNA
ccatacaggtaatgagagcccag

SEQ ID NO: 250 - RNA, 23 nt, synthetic construct
  source 1..23: mol_type = other RNA
cagacaaagctagatccccagag

SEQ ID NO: 251 - RNA, 23 nt, synthetic construct
  source 1..23: mol_type = other RNA
caagacactaatgagctcacagt

SEQ ID NO: 252 - RNA, 23 nt, synthetic construct
  source 1..23: mol_type = other RNA
ccaggaaaatagcaaggagcagg

SEQ ID NO: 253 - RNA, 23 nt, synthetic construct
  source 1..23: mol_type = other RNA
attcagaacaataagagcagcct

SEQ ID NO: 254 - RNA, 23 nt, synthetic construct
  source 1..23: mol_type = other RNA
accatgatgaaaaacggcaccag

SEQ ID NO: 255 - RNA, 23 nt, synthetic construct
  source 1..23: mol_type = other RNA
cccaatagacaagtgagaccaag

SEQ ID NO: 256 - RNA, 23 nt, synthetic construct
  source 1..23: mol_type = other RNA
acagaatgagtttcaggacagcc

SEQ ID NO: 257 - RNA, 23 nt, synthetic construct
  source 1..23: mol_type = other RNA
acacacatcacaaaggctaccac

SEQ ID NO: 258 - RNA, 23 nt, synthetic construct
  source 1..23: mol_type = other RNA
aacgcattataagacaggacacc

SEQ ID NO: 259 - RNA, 23 nt, synthetic construct
  source 1..23: mol_type = other RNA
ataaagaaacagatgaccacagc

SEQ ID NO: 260 - RNA, 23 nt, synthetic construct
  source 1..23: mol_type = other RNA
tataccatgacatttgcccagca

SEQ ID NO: 261 - RNA, 23 nt, synthetic construct
  source 1..23: mol_type = other RNA
cctcaagaagagatgcaggcaac

SEQ ID NO: 262 - RNA, 23 nt, synthetic construct
  source 1..23: mol_type = other RNA
aataccacatacagtgtgctctg

SEQ ID NO: 263 - RNA, 23 nt, synthetic construct
  source 1..23: mol_type = other RNA
aaatacatcgaagagcagttcca

SEQ ID NO: 264 - RNA, 23 nt, synthetic construct
  source 1..23: mol_type = other RNA
caaagtgattttcagccagctga

SEQ ID NO: 265 - RNA, 23 nt, synthetic construct
  source 1..23: mol_type = other RNA
atctgattatacttgaggccaca

SEQ ID NO: 266 - RNA, 23 nt, synthetic construct
  source 1..23: mol_type = other RNA
agcaaagccagaaccgagaccac

SEQ ID NO: 267 - RNA, 23 nt, synthetic construct
  source 1..23: mol_type = other RNA
cagtatctgagactgcatcccca

SEQ ID NO: 268 - RNA, 23 nt, synthetic construct
  source 1..23: mol_type = other RNA
gactcaatgaaaagtgagcccct

SEQ ID NO: 269 - RNA, 23 nt, synthetic construct
  source 1..23: mol_type = other RNA
agtctgaaaaggattcaaccgcg

SEQ ID NO: 270 - RNA, 23 nt, synthetic construct
  source 1..23: mol_type = other RNA
taagtgataaagatttggcttgc

SEQ ID NO: 271 - RNA, 23 nt, synthetic construct
  source 1..23: mol_type = other RNA
agacatgatcacataggtcccgt

SEQ ID NO: 272 - RNA, 23 nt, synthetic construct
  source 1..23: mol_type = other RNA
accacaattgctgtgtgcgcatg

SEQ ID NO: 273 - RNA, 23 nt, synthetic construct
  source 1..23: mol_type = other RNA
tgaccaagtcaaataggcccact

SEQ ID NO: 274 - RNA, 23 nt, synthetic construct
  source 1..23: mol_type = other RNA
gtgaaatatatgtagcaagacag

SEQ ID NO: 275 - RNA, 23 nt, synthetic construct
  source 1..23: mol_type = other RNA
acagatgaagcaatcggcatcta

SEQ ID NO: 276 - RNA, 23 nt, synthetic construct
  source 1..23: mol_type = other RNA
acacaaatcctgaatgtaccagg

SEQ ID NO: 277 - RNA, 23 nt, synthetic construct
  source 1..23: mol_type = other RNA
agatctgaaaacctgcgagtcag

SEQ ID NO: 278 - RNA, 23 nt, synthetic construct
  source 1..23: mol_type = other RNA
aaaaacctgaaattgccacagag

SEQ ID NO: 279 - RNA, 23 nt, synthetic construct
  source 1..23: mol_type = other RNA
aattccgtaaaatagagccccag

SEQ ID NO: 280 - RNA, 23 nt, synthetic construct
  source 1..23: mol_type = other RNA
gaactgaaaaagtagcagcaagg

SEQ ID NO: 281 - RNA, 23 nt, synthetic construct
  source 1..23: mol_type = other RNA
gcaagctacatgaagcccaacag

SEQ ID NO: 282 - RNA, 23 nt, synthetic construct
  source 1..23: mol_type = other RNA
tgaccattatcacaagaccgaac

SEQ ID NO: 283 - RNA, 23 nt, synthetic construct
  source 1..23: mol_type = other RNA
ctagagataattttccagccaga

SEQ ID NO: 284 - RNA, 23 nt, synthetic construct
  source 1..23: mol_type = other RNA
tagttttacaaatatgagcagat

SEQ ID NO: 285 - RNA, 23 nt, synthetic construct
  source 1..23: mol_type = other RNA
gcaaaaagaaaaagtccacccga

SEQ ID NO: 286 - RNA, 23 nt, synthetic construct
  source 1..23: mol_type = other RNA
cacacctccataaagtgcaccct

SEQ ID NO: 287 - RNA, 23 nt, synthetic construct
  source 1..23: mol_type = other RNA
cacgagatattccatgcgtcctt

SEQ ID NO: 288 - RNA, 23 nt, synthetic construct
  source 1..23: mol_type = other RNA
ttccagagaaaactgcctgcaca

SEQ ID NO: 289 - RNA, 23 nt, synthetic construct
  source 1..23: mol_type = other RNA
tacacagtgagttacaggtcagt

SEQ ID NO: 290 - RNA, 23 nt, synthetic construct
  source 1..23: mol_type = other RNA
tactggtcatgataaagcagcag

SEQ ID NO: 291 - RNA, 23 nt, synthetic construct
  source 1..23: mol_type = other RNA
aaacataatgacttccagtccca

SEQ ID NO: 292 - RNA, 23 nt, synthetic construct
  source 1..23: mol_type = other RNA
agcacagatctctttgcccaacc

SEQ ID NO: 293 - RNA, 23 nt, synthetic construct
  source 1..23: mol_type = other RNA
cctttgaagtaatccaggcagca

SEQ ID NO: 294 - RNA, 23 nt, synthetic construct
  source 1..23: mol_type = other RNA
atgttgaaaccatggacagcagc

SEQ ID NO: 295 - RNA, 23 nt, synthetic construct
  source 1..23: mol_type = other RNA
catgctgcagaaaagtccccaga

SEQ ID NO: 296 - RNA, 23 nt, synthetic construct
  source 1..23: mol_type = other RNA
gtcagaaacacgatggcatccct

SEQ ID NO: 297 - RNA, 23 nt, synthetic construct
  source 1..23: mol_type = other RNA
gtctgcacagatgagcttgccct

SEQ ID NO: 298 - RNA, 23 nt, synthetic construct
  source 1..23: mol_type = other RNA
aaccgatcaataatggacacagt

SEQ ID NO: 299 - RNA, 23 nt, synthetic construct
  source 1..23: mol_type = other RNA
agaatgcacttgaatcccaccga

SEQ ID NO: 300 - RNA, 23 nt, synthetic construct
  source 1..23: mol_type = other RNA
ttacatcagagaaagcctgacac

SEQ ID NO: 301 - RNA, 23 nt, synthetic construct
  source 1..23: mol_type = other RNA
agctactatgaatgaagccaagc

SEQ ID NO: 302 - RNA, 23 nt, synthetic construct
  source 1..23: mol_type = other RNA
gtatatagacatataggctacag

SEQ ID NO: 303 - RNA, 23 nt, synthetic construct
  source 1..23: mol_type = other RNA
catcactaaatacctgccaccaa

SEQ ID NO: 304 - RNA, 23 nt, synthetic construct
  source 1..23: mol_type = other RNA
ggaaatcaagtagtgcagcaggt

SEQ ID NO: 305 - RNA, 23 nt, synthetic construct
  source 1..23: mol_type = other RNA
aactcgtagcataagaagctcac

SEQ ID NO: 306 - RNA, 23 nt, synthetic construct
  source 1..23: mol_type = other RNA
tacacattcatacttcccctaag

SEQ ID NO: 307 - RNA, 23 nt, synthetic construct
  source 1..23: mol_type = other RNA
aacttatggtaaatggtgcaagc

SEQ ID NO: 308 - RNA, 23 nt, synthetic construct
  source 1..23: mol_type = other RNA
ataatgatagaaacacaggcagt

SEQ ID NO: 309 - RNA, 23 nt, synthetic construct
  source 1..23: mol_type = other RNA
ccaggtaataaaacccagacagc

SEQ ID NO: 310 - RNA, 23 nt, synthetic construct
  source 1..23: mol_type = other RNA
aagaattaacgacagcaccacac

SEQ ID NO: 311 - RNA, 23 nt, synthetic construct
  source 1..23: mol_type = other RNA
tatcaaacatctaaagagcccat

SEQ ID NO: 312 - RNA, 23 nt, synthetic construct
  source 1..23: mol_type = other RNA
ataaattcaggcaatgctgccag

SEQ ID NO: 313 - RNA, 23 nt, synthetic construct
  source 1..23: mol_type = other RNA
aaccaatgtaataaaggccagag

SEQ ID NO: 314 - RNA, 23 nt, synthetic construct
  source 1..23: mol_type = other RNA
agaccaattatctgagcgagcct

SEQ ID NO: 315 - RNA, 23 nt, synthetic construct
  source 1..23: mol_type = other RNA
cccagcacaaacaagacgcagta

SEQ ID NO: 316 - RNA, 23 nt, synthetic construct
  source 1..23: mol_type = other RNA
actggaaacattgtagcatgccg

SEQ ID NO: 317 - RNA, 23 nt, synthetic construct
  source 1..23: mol_type = other RNA
acaatgactacggtgagcaccag

SEQ ID NO: 318 - RNA, 23 nt, synthetic construct
  source 1..23: mol_type = other RNA
cataatgaagacagtgaggacag

SEQ ID NO: 319 - RNA, 23 nt, synthetic construct
  source 1..23: mol_type = other RNA
cacatgatgagaagcacacagct

SEQ ID NO: 320 - RNA, 23 nt, synthetic construct
  source 1..23: mol_type = other RNA
gcccacaatgaacacaagccagt

SEQ ID NO: 321 - RNA, 23 nt, synthetic construct
  source 1..23: mol_type = other RNA
taccagtagacaaggatgaccag

SEQ ID NO: 322 - RNA, 23 nt, synthetic construct
  source 1..23: mol_type = other RNA
tagcatagaagaaagcttcaagc

SEQ ID NO: 323 - RNA, 23 nt, synthetic construct
  source 1..23: mol_type = other RNA
accagaagagtattagcccctca

SEQ ID NO: 324 - RNA, 23 nt, synthetic construct
  source 1..23: mol_type = other RNA
acactgatgtaactgcagttcca

SEQ ID NO: 325 - RNA, 23 nt, synthetic construct
  source 1..23: mol_type = other RNA
aatgtcttgttaaacgagcagag

SEQ ID NO: 326 - RNA, 23 nt, synthetic construct
  source 1..23: mol_type = other RNA
atacacgaaagttcacgggctta

SEQ ID NO: 327 - RNA, 23 nt, synthetic construct
  source 1..23: mol_type = other RNA
gaagttcacaattgccagcagga

SEQ ID NO: 328 - RNA, 23 nt, synthetic construct
  source 1..23: mol_type = other RNA
aaaattaacaggtagcagcagag

SEQ ID NO: 329 - RNA, 23 nt, synthetic construct
  source 1..23: mol_type = other RNA
ttatcagtacaacttgacccagc

SEQ ID NO: 330 - RNA, 23 nt, synthetic construct
  source 1..23: mol_type = other RNA
aataaacatgtaattggacacac

SEQ ID NO: 331 - RNA, 23 nt, synthetic construct
  source 1..23: mol_type = other RNA
gaattttaaagaaagcaaagcag

SEQ ID NO: 332 - RNA, 23 nt, synthetic construct
  source 1..23: mol_type = other RNA
ggaatagaaaagcagcagagcag

SEQ ID NO: 333 - RNA, 23 nt, synthetic construct
  source 1..23: mol_type = other RNA
cgtagcaagtgacagcaggctgt

SEQ ID NO: 334 - RNA, 23 nt, synthetic construct
  source 1..23: mol_type = other RNA
ccatcgtgacgttgctgccatac

SEQ ID NO: 335 - RNA, 23 nt, synthetic construct
  source 1..23: mol_type = other RNA
cgcttgtagtccgcaccaccgta

SEQ ID NO: 336 - RNA, 23 nt, synthetic construct
  source 1..23: mol_type = other RNA
tagaaaacatcattcgctgtggc

SEQ ID NO: 337 - RNA, 23 nt, synthetic construct
  source 1..23: mol_type = other RNA
atttctccacatctagcattctc

SEQ ID NO: 338 - RNA, 23 nt, synthetic construct
  source 1..23: mol_type = other RNA
ataatagcagttacacagtctgc

SEQ ID NO: 339 - RNA, 23 nt, synthetic construct
  source 1..23: mol_type = other RNA
gtgaacatcgaacaagccccgct

SEQ ID NO: 340 - RNA, 23 nt, synthetic construct
  source 1..23: mol_type = other RNA
agcaagaactaagaggtcactgt

SEQ ID NO: 341 - RNA, 23 nt, synthetic construct
  source 1..23: mol_type = other RNA
gacaacaaaagccagggccagat

SEQ ID NO: 342 - RNA, 23 nt, synthetic construct
  source 1..23: mol_type = other RNA
tttaatgaagagctgacggccaa

SEQ ID NO: 343 - RNA, 23 nt, synthetic construct
  source 1..23: mol_type = other RNA
cacctttcgtaattccacaccag

SEQ ID NO: 344 - RNA, 23 nt, synthetic construct
  source 1..23: mol_type = other RNA
tgatacgatttacacagcatccc

SEQ ID NO: 345 - RNA, 23 nt, synthetic construct
  source 1..23: mol_type = other RNA
aagcaaagagcgaggccaccaac

SEQ ID NO: 346 - RNA, 23 nt, synthetic construct
  source 1..23: mol_type = other RNA
acaatcaattcaactccacccaa

SEQ ID NO: 347 - RNA, 23 nt, synthetic construct
  source 1..23: mol_type = other RNA
cagatgtaaagatccatgctcca

SEQ ID NO: 348 - RNA, 23 nt, synthetic construct
  source 1..23: mol_type = other RNA
agatcatcagcaatgtagcccag

SEQ ID NO: 349 - RNA, 23 nt, synthetic construct
  source 1..23: mol_type = other RNA
ataaacaatacagaagcaccaca

SEQ ID NO: 350 - RNA, 23 nt, synthetic construct
  source 1..23: mol_type = other RNA
aaaacatgaatacagaagcacct

SEQ ID NO: 351 - RNA, 23 nt, synthetic construct
  source 1..23: mol_type = other RNA
ccaatttaacaagtggggcgtag

SEQ ID NO: 352 - RNA, 23 nt, synthetic construct
  source 1..23: mol_type = other RNA
cagagcaaactgaccagaacctc

SEQ ID NO: 353 - RNA, 23 nt, synthetic construct
  source 1..23: mol_type = other RNA
caagcaagacagaagcgccaagt

SEQ ID NO: 354 - RNA, 23 nt, synthetic construct
  source 1..23: mol_type = other RNA
tagagattacaatgaggccaagt

SEQ ID NO: 355 - RNA, 23 nt, synthetic construct
  source 1..23: mol_type = other RNA
accaaagcaaggacgtagcccag

SEQ ID NO: 356 - RNA, 23 nt, synthetic construct
  source 1..23: mol_type = other RNA
ccacgatgactgtgagcaccagc

SEQ ID NO: 357 - RNA, 23 nt, synthetic construct
  source 1..23: mol_type = other RNA
taaacagtagttgagctgaacct

SEQ ID NO: 358 - RNA, 23 nt, synthetic construct
  source 1..23: mol_type = other RNA
acaaaggacaaatgtccaagcgt

SEQ ID NO: 359 - RNA, 23 nt, synthetic construct
  source 1..23: mol_type = other RNA
agagtccaaggagaaagccaacc

SEQ ID NO: 360 - RNA, 23 nt, synthetic construct
  source 1..23: mol_type = other RNA
aattatttagactctaggaccat

SEQ ID NO: 361 - RNA, 23 nt, synthetic construct
  source 1..23: mol_type = other RNA
tcccactgtgatctctggcgcac

SEQ ID NO: 362 - RNA, 23 nt, synthetic construct
  source 1..23: mol_type = other RNA
ataagtccttgtaagtaccccac

SEQ ID NO: 363 - RNA, 23 nt, synthetic construct
  source 1..23: mol_type = other RNA
aaatgctagcatttccaaccagg

SEQ ID NO: 364 - RNA, 23 nt, synthetic construct
  source 1..23: mol_type = other RNA
catatatataacggtcaccacct

SEQ ID NO: 365 - RNA, 23 nt, synthetic construct
  source 1..23: mol_type = other RNA
tcttgaagacaatgacagcatgc

SEQ ID NO: 366 - RNA, 23 nt, synthetic construct
  source 1..23: mol_type = other RNA
caaaactgagcaactggagacca

SEQ ID NO: 367 - RNA, 23 nt, synthetic construct
  source 1..23: mol_type = other RNA
gttaaattagaatgtgccacctc

SEQ ID NO: 368 - RNA, 23 nt, synthetic construct
  source 1..23: mol_type = other RNA
atcaagttgaacaatggagccat

SEQ ID NO: 369 - RNA, 23 nt, synthetic construct
  source 1..23: mol_type = other RNA
aaaaatgctcaagatggccagca

SEQ ID NO: 370 - RNA, 23 nt, synthetic construct
  source 1..23: mol_type = other RNA
aaacgactcaaaaggcaagaccg

SEQ ID NO: 371 - RNA, 23 nt, synthetic construct
  source 1..23: mol_type = other RNA
agatatgcaagtgacccatctac

SEQ ID NO: 372 - RNA, 23 nt, synthetic construct
  source 1..23: mol_type = other RNA
ctagaataaggaagagaggcagg

SEQ ID NO: 373 - RNA, 23 nt, synthetic construct
  source 1..23: mol_type = other RNA
aagcccaaacaatagtgtgcaga

SEQ ID NO: 374 - RNA, 23 nt, synthetic construct
  source 1..23: mol_type = other RNA
atccacttttagaagaagcagca

SEQ ID NO: 375 - RNA, 23 nt, synthetic construct
  source 1..23: mol_type = other RNA
cattcatttgagctgaagcagag

SEQ ID NO: 376 - RNA, 23 nt, synthetic construct
  source 1..23: mol_type = other RNA
acagagtccaatttgttacaccc

SEQ ID NO: 377 - RNA, 23 nt, synthetic construct
  source 1..23: mol_type = other RNA
atgcactcatactttccacccag

SEQ ID NO: 378 - RNA, 23 nt, synthetic construct
  source 1..23: mol_type = other RNA
gcgttttctatgttgcacccaga

SEQ ID NO: 379 - RNA, 23 nt, synthetic construct
  source 1..23: mol_type = other RNA
aatctgaaaagtcacacgacaga

SEQ ID NO: 380 - RNA, 23 nt, synthetic construct
  source 1..23: mol_type = other RNA
attatgaagtcttccaagagccg

SEQ ID NO: 381 - RNA, 23 nt, synthetic construct
  source 1..23: mol_type = other RNA
actagagatggcaatagccctga

SEQ ID NO: 382 - RNA, 23 nt, synthetic construct
  source 1..23: mol_type = other RNA
atgacttgtcggcaggctccaca

SEQ ID NO: 383 - RNA, 23 nt, synthetic construct
  source 1..23: mol_type = other RNA
taaaacattatgtacacacacca

SEQ ID NO: 384 - RNA, 23 nt, synthetic construct
  source 1..23: mol_type = other RNA
aacggcaatagtagtgatctgcc

SEQ ID NO: 385 - RNA, 23 nt, synthetic construct
  source 1..23: mol_type = other RNA
agcaaaatcaccattaagccggt

SEQ ID NO: 386 - RNA, 23 nt, synthetic construct
  source 1..23: mol_type = other RNA
caaacatgagaacaaggatcagc

SEQ ID NO: 387 - RNA, 23 nt, synthetic construct
  source 1..23: mol_type = other RNA
tgtaaaattcaagtgtagctgag

SEQ ID NO: 388 - RNA, 23 nt, synthetic construct
  source 1..23: mol_type = other RNA
ttatagtaattaacagccaccac

SEQ ID NO: 389 - RNA, 23 nt, synthetic construct
  source 1..23: mol_type = other RNA
aagaattacactaagcagcacag

SEQ ID NO: 390 - RNA, 23 nt, synthetic construct
  source 1..23: mol_type = other RNA
gtagacaaacttaagcatcccag

SEQ ID NO: 391 - RNA, 23 nt, synthetic construct
  source 1..23: mol_type = other RNA
agattaaagattgcgcgccacta

SEQ ID NO: 392 - RNA, 23 nt, synthetic construct
  source 1..23: mol_type = other RNA
tgaagaatccatgaactgcccag

SEQ ID NO: 393 - RNA, 23 nt, synthetic construct
  source 1..23: mol_type = other RNA
agcataaattcgaaagccagcac

SEQ ID NO: 394 - RNA, 23 nt, synthetic construct
  source 1..23: mol_type = other RNA
cttgaaaagatcaagccagacag

SEQ ID NO: 395 - RNA, 23 nt, synthetic construct
  source 1..23: mol_type = other RNA
cccactttattaaacccaccaag

SEQ ID NO: 396 - RNA, 23 nt, synthetic construct
  source 1..23: mol_type = other RNA
cagtaaatctgaatggccaacac

SEQ ID NO: 397 - RNA, 23 nt, synthetic construct
  source 1..23: mol_type = other RNA
tgcaattagacagaaacacccag

SEQ ID NO: 398 - RNA, 23 nt, synthetic construct
  source 1..23: mol_type = other RNA
acaatcaaaagaacagggaccat

SEQ ID NO: 399 - RNA, 23 nt, synthetic construct
  source 1..23: mol_type = other RNA
agtaggtattcattgcaccaacg

SEQ ID NO: 400 - RNA, 23 nt, synthetic construct
  source 1..23: mol_type = other RNA
gaaattaaataaagtccacggag

SEQ ID NO: 401 - RNA, 23 nt, synthetic construct
  source 1..23: mol_type = other RNA
tcagccaatttcaactccccatt

SEQ ID NO: 402 - RNA, 23 nt, synthetic construct
  source 1..23: mol_type = other RNA
gaactcaaaaaccagtgtcagct

SEQ ID NO: 403 - RNA, 23 nt, synthetic construct
  source 1..23: mol_type = other RNA
aaccaaaaacatcaacaaccccg

SEQ ID NO: 404 - RNA, 23 nt, synthetic construct
  source 1..23: mol_type = other RNA
gttctcaaaatatacatacgcct

SEQ ID NO: 405 - RNA, 23 nt, synthetic construct
  source 1..23: mol_type = other RNA
ctcgtcgctcagcttgtccaccg

SEQ ID NO: 406 - RNA, 23 nt, synthetic construct
  source 1..23: mol_type = other RNA
gattgcatcaagtcccgaaaccc

SEQ ID NO: 407 - RNA, 23 nt, synthetic construct
  source 1..23: mol_type = other RNA
ctctcgcgacagctgctccacct

SEQ ID NO: 408 - RNA, 23 nt, synthetic construct
  source 1..23: mol_type = other RNA
atattgctaaatagtagcccaag

SEQ ID NO: 409 - RNA, 23 nt, synthetic construct
  source 1..23: mol_type = other RNA
cctgcaaatcaaatgaggcaaga

SEQ ID NO: 410 - RNA, 23 nt, synthetic construct
  source 1..23: mol_type = other RNA
aggagtaaaaatatacagccact

SEQ ID NO: 411 - RNA, 23 nt, synthetic construct
  source 1..23: mol_type = other RNA
aaatgctagtcattacaacagcg

SEQ ID NO: 412 - RNA, 23 nt, synthetic construct
  source 1..23: mol_type = other RNA
aagtctagagtacatgcctgcat

SEQ ID NO: 413 - RNA, 23 nt, synthetic construct
  source 1..23: mol_type = other RNA
agaaaagctggatatgatagccc

SEQ ID NO: 414 - RNA, 23 nt, synthetic construct
  source 1..23: mol_type = other RNA
ctctgtagagcaattcagccaag

SEQ ID NO: 415 - RNA, 23 nt, synthetic construct
  source 1..23: mol_type = other RNA
atgatataccaagaacaccaacg

SEQ ID NO: 416 - RNA, 23 nt, synthetic construct
  source 1..23: mol_type = other RNA
catacttgcatatcggcgaacaa

SEQ ID NO: 417 - RNA, 23 nt, synthetic construct
  source 1..23: mol_type = other RNA
gaagatattttgtgtcgttgcca

SEQ ID NO: 418 - RNA, 23 nt, synthetic construct
  source 1..23: mol_type = other RNA
ttagacatataattcccagcaca

SEQ ID NO: 419 - RNA, 23 nt, synthetic construct
  source 1..23: mol_type = other RNA
gtccgattatgtacagagccaca

SEQ ID NO: 420 - RNA, 23 nt, synthetic construct
  source 1..23: mol_type = other RNA
aaacactgcattttggaaccgca

SEQ ID NO: 421 - RNA, 23 nt, synthetic construct
  source 1..23: mol_type = other RNA
agaaccaagaagaagcaacccct

SEQ ID NO: 422 - RNA, 23 nt, synthetic construct
  source 1..23: mol_type = other RNA
aataaacaataattgccaggcgt

SEQ ID NO: 423 - RNA, 23 nt, synthetic construct
  source 1..23: mol_type = other RNA
gcacaacatagagaagccagctc

SEQ ID NO: 424 - RNA, 23 nt, synthetic construct
  source 1..23: mol_type = other RNA
taaacagagatagtcagctccca

SEQ ID NO: 425 - RNA, 23 nt, synthetic construct
  source 1..23: mol_type = other RNA
ttgacaagcagttagagtccagc

SEQ ID NO: 426 - RNA, 23 nt, synthetic construct
  source 1..23: mol_type = other RNA
ctgaagaaaggacagcagaaccc

SEQ ID NO: 427 - RNA, 23 nt, synthetic construct
  source 1..23: mol_type = other RNA
cactacagctaaaagagttcagg

SEQ ID NO: 428 - RNA, 23 nt, synthetic construct
  source 1..23: mol_type = other RNA
tccagaagcaaagcaggtaccag

SEQ ID NO: 429 - RNA, 23 nt, synthetic construct
  source 1..23: mol_type = other RNA
gccaaatgactaatatggcctgc

SEQ ID NO: 430 - RNA, 23 nt, synthetic construct
  source 1..23: mol_type = other RNA
cctcgctttcaaacacaccgccc

SEQ ID NO: 431 - RNA, 23 nt, synthetic construct
  source 1..23: mol_type = other RNA
aagaaagcaacatggtcacgcga

SEQ ID NO: 432 - RNA, 23 nt, synthetic construct
  source 1..23: mol_type = other RNA
acaccatccttgtaacagccatg

SEQ ID NO: 433 - RNA, 23 nt, synthetic construct
  source 1..23: mol_type = other RNA
gtttaaaacaaagaggagcacat

SEQ ID NO: 434 - RNA, 23 nt, synthetic construct
  source 1..23: mol_type = other RNA
catgcaataaatgcagtcctgag

SEQ ID NO: 435 - RNA, 23 nt, synthetic construct
  source 1..23: mol_type = other RNA
tagatagataagctagacccaag

SEQ ID NO: 436 - RNA, 23 nt, synthetic construct
  source 1..23: mol_type = other RNA
cccagtaatacatacatgtgcag

SEQ ID NO: 437 - RNA, 23 nt, synthetic construct
  source 1..23: mol_type = other RNA
attctttaaacttgatgaccagg

SEQ ID NO: 438 - RNA, 23 nt, synthetic construct
  source 1..23: mol_type = other RNA
agcaacagttacgatgcctaccg

SEQ ID NO: 439 - RNA, 23 nt, synthetic construct
  source 1..23: mol_type = other RNA
cagagaaatagtagcaggaacct

SEQ ID NO: 440 - RNA, 23 nt, synthetic construct
  source 1..23: mol_type = other RNA
tatttatatcacaatcactccac

SEQ ID NO: 441 - RNA, 23 nt, synthetic construct
  source 1..23: mol_type = other RNA
taaatatgagaaaagaacccccg

SEQ ID NO: 442 - RNA, 23 nt, synthetic construct
  source 1..23: mol_type = other RNA
accaagagcttgaggagagacgc

SEQ ID NO: 443 - RNA, 23 nt, synthetic construct
  source 1..23: mol_type = other RNA
catagaatgcaaaagccaaccag

SEQ ID NO: 444 - RNA, 23 nt, synthetic construct
  source 1..23: mol_type = other RNA
acaaaaatgatagatgccagcag

SEQ ID NO: 445 - RNA, 23 nt, synthetic construct
  source 1..23: mol_type = other RNA
aaaattctaagagtggccacaga

SEQ ID NO: 446 - RNA, 23 nt, synthetic construct
  source 1..23: mol_type = other RNA
cataacattaaggaaagacgcct

SEQ ID NO: 447 - RNA, 23 nt, synthetic construct
  source 1..23: mol_type = other RNA
acatgcaattcaaaggacagcag

SEQ ID NO: 448 - RNA, 23 nt, synthetic construct
  source 1..23: mol_type = other RNA
aagaagatcaacattgcaccgca

SEQ ID NO: 449 - RNA, 23 nt, synthetic construct
  source 1..23: mol_type = other RNA
tttacaaattcagagcaaacccc

SEQ ID NO: 450 - RNA, 23 nt, synthetic construct
  source 1..23: mol_type = other RNA
acaaacaaggcaaaacagcccac

SEQ ID NO: 451 - RNA, 23 nt, synthetic construct
  source 1..23: mol_type = other RNA
tttcaaattcagtttcccacaag

SEQ ID NO: 452 - RNA, 23 nt, synthetic construct
  source 1..23: mol_type = other RNA
aaccactgtgaaatgaaagccac

SEQ ID NO: 453 - RNA, 23 nt, synthetic construct
  source 1..23: mol_type = other RNA
ataagagaccatatggccagcaa

SEQ ID NO: 454 - RNA, 23 nt, synthetic construct
  source 1..23: mol_type = other RNA
agcataagacaatgtggccaacg

SEQ ID NO: 455 - RNA, 23 nt, synthetic construct
  source 1..23: mol_type = other RNA
gacacatgattatatcccagcag

SEQ ID NO: 456 - RNA, 23 nt, synthetic construct
  source 1..23: mol_type = other RNA
cctatcacaaaaattagccacac

SEQ ID NO: 457 - RNA, 23 nt, synthetic construct
  source 1..23: mol_type = other RNA
cttcaaaatgagcagtgagtcgc

SEQ ID NO: 458 - RNA, 23 nt, synthetic construct
  source 1..23: mol_type = other RNA
tcatttagttaccaacactccgc

SEQ ID NO: 459 - RNA, 23 nt, synthetic construct
  source 1..23: mol_type = other RNA
aattaatctgatttgtggcagta

SEQ ID NO: 460 - RNA, 23 nt, synthetic construct
  source 1..23: mol_type = other RNA
aatcagttacactatccacagac

SEQ ID NO: 461 - RNA, 23 nt, synthetic construct
  source 1..23: mol_type = other RNA
tcattttcctcatttcccccaac

SEQ ID NO: 462 - RNA, 23 nt, synthetic construct
  source 1..23: mol_type = other RNA
ctaaggttacagtgggagcagat

SEQ ID NO: 463 - RNA, 23 nt, synthetic construct
  source 1..23: mol_type = other RNA
atcatgatcagaaggcaccagag

SEQ ID NO: 464 - RNA, 23 nt, synthetic construct
  source 1..23: mol_type = other RNA
aaaagaactgattctgagcacag

SEQ ID NO: 465 - RNA, 23 nt, synthetic construct
  source 1..23: mol_type = other RNA
gaaatttcacaaagacagaaccc

SEQ ID NO: 466 - RNA, 23 nt, synthetic construct
  source 1..23: mol_type = other RNA
ccatgaatcgtacttcagcacca

SEQ ID NO: 467 - RNA, 23 nt, synthetic construct
  source 1..23: mol_type = other RNA
agacacacagtaaaggtgttcag

SEQ ID NO: 468 - RNA, 23 nt, synthetic construct
  source 1..23: mol_type = other RNA
tcagcagcataaagagaccaagg

SEQ ID NO: 469 - RNA, 23 nt, synthetic construct
  source 1..23: mol_type = other RNA
tgcacacatattttcaagaccca

SEQ ID NO: 470 - RNA, 23 nt, synthetic construct
  source 1..23: mol_type = other RNA
gcccacaataaatagtggcagta

SEQ ID NO: 471 - RNA, 23 nt, synthetic construct
  source 1..23: mol_type = other RNA
tcagcaagactaggatgatgccc

SEQ ID NO: 472 - RNA, 23 nt, synthetic construct
  source 1..23: mol_type = other RNA
catctattatgtcttgtaccaga

SEQ ID NO: 473 - RNA, 23 nt, synthetic construct
  source 1..23: mol_type = other RNA
cacacaagaatatatgccagcgt

SEQ ID NO: 474 - RNA, 23 nt, synthetic construct
  source 1..23: mol_type = other RNA
atagtaaatatagaggctagcac

SEQ ID NO: 475 - RNA, 23 nt, synthetic construct
  source 1..23: mol_type = other RNA
catgacagacatacaggccacca

SEQ ID NO: 476 - RNA, 23 nt, synthetic construct
  source 1..23: mol_type = other RNA
cagcagtattcagtgatgaccag

SEQ ID NO: 477 - RNA, 23 nt, synthetic construct
  source 1..23: mol_type = other RNA
cacttgaagaagtcgagacaggc

SEQ ID NO: 478 - RNA, 23 nt, synthetic construct
  source 1..23: mol_type = other RNA
accacactttattatccgcaagc

SEQ ID NO: 479 - RNA, 23 nt, synthetic construct
  source 1..23: mol_type = other RNA
cctttatacataaagccatcaag

SEQ ID NO: 480 - RNA, 23 nt, synthetic construct
  source 1..23: mol_type = other RNA
ccatagtgcactttgccacagca

SEQ ID NO: 481 - RNA, 23 nt, synthetic construct
  source 1..23: mol_type = other RNA
gatctagaagcttagagctccat

SEQ ID NO: 482 - RNA, 23 nt, synthetic construct
  source 1..23: mol_type = other RNA
gaaatacccgatagagcctgcag

SEQ ID NO: 483 - RNA, 23 nt, synthetic construct
  source 1..23: mol_type = other RNA
cacgtagataaagttgccacgct

SEQ ID NO: 484 - RNA, 23 nt, synthetic construct
  source 1..23: mol_type = other RNA
ttgccttaaattaaccaacccca

SEQ ID NO: 485 - RNA, 23 nt, synthetic construct
  source 1..23: mol_type = other RNA
agaaagtttcatatgggccatag

SEQ ID NO: 486 - RNA, 23 nt, synthetic construct
  source 1..23: mol_type = other RNA
ctcatacagaatattcccagcat

SEQ ID NO: 487 - RNA, 23 nt, synthetic construct
  source 1..23: mol_type = other RNA
cacagtgatgacaaaggcctcag

SEQ ID NO: 488 - RNA, 23 nt, synthetic construct
  source 1..23: mol_type = other RNA
gtgtttatattcaaaccaccagc

SEQ ID NO: 489 - RNA, 23 nt, synthetic construct
  source 1..23: mol_type = other RNA
ataaaatgagtgtaaagacccag

SEQ ID NO: 490 - RNA, 23 nt, synthetic construct
  source 1..23: mol_type = other RNA
tcaaagaaacagcagtgaccagg

SEQ ID NO: 491 - RNA, 23 nt, synthetic construct
  source 1..23: mol_type = other RNA
tgacacaacagaaatatcccagc

SEQ ID NO: 492 - RNA, 23 nt, synthetic construct
  source 1..23: mol_type = other RNA
caatgacataaatctgcgtcccg

SEQ ID NO: 493 - RNA, 23 nt, synthetic construct
  source 1..23: mol_type = other RNA
caagacatacaaacacagcctcc

SEQ ID NO: 494 - RNA, 23 nt, synthetic construct
  source 1..23: mol_type = other RNA
aatgtaaaataaaaaccacacac

SEQ ID NO: 495 - RNA, 23 nt, synthetic construct
  source 1..23: mol_type = other RNA
ttgtatagtgttgtcagaagcca

SEQ ID NO: 496 - RNA, 23 nt, synthetic construct
  source 1..23: mol_type = other RNA
atgacttcggtttgggtgcagtg

SEQ ID NO: 497 - RNA, 23 nt, synthetic construct
  source 1..23: mol_type = other RNA
aatacataaataaataggaccct

SEQ ID NO: 498 - RNA, 23 nt, synthetic construct
  source 1..23: mol_type = other RNA
ataaaagttatatgccagcccag

SEQ ID NO: 499 - RNA, 23 nt, synthetic construct
  source 1..23: mol_type = other RNA
aaatatatagttagtggcccaaa

SEQ ID NO: 500 - RNA, 23 nt, synthetic construct
  source 1..23: mol_type = other RNA
acaacagtaaaagaggtccccat

SEQ ID NO: 501 - RNA, 23 nt, synthetic construct
  source 1..23: mol_type = other RNA
aacagcaacagaaatgccagcgg

SEQ ID NO: 502 - RNA, 23 nt, synthetic construct
  source 1..23: mol_type = other RNA
gtaatataaagaagtgagacact

SEQ ID NO: 503 - RNA, 23 nt, synthetic construct
  source 1..23: mol_type = other RNA
aagtcaaaacaagatgagcgcat

SEQ ID NO: 504 - RNA, 23 nt, synthetic construct
  source 1..23: mol_type = other RNA
gaactagaagtgttagccaccat

SEQ ID NO: 505 - RNA, 23 nt, synthetic construct
  source 1..23: mol_type = other RNA
aagttcaataacaaagacacagg

SEQ ID NO: 506 - RNA, 23 nt, synthetic construct
  source 1..23: mol_type = other RNA
ccacaagcattgaatagcccctc

SEQ ID NO: 507 - RNA, 23 nt, synthetic construct
  source 1..23: mol_type = other RNA
agtataattatacttaggcccat

SEQ ID NO: 508 - RNA, 23 nt, synthetic construct
  source 1..23: mol_type = other RNA
attacggataagtaatgccccag

SEQ ID NO: 509 - RNA, 23 nt, synthetic construct
  source 1..23: mol_type = other RNA
tatatttcacttttgcccccaaa

SEQ ID NO: 510 - RNA, 23 nt, synthetic construct
  source 1..23: mol_type = other RNA
cattaatgaagttttccaccact

SEQ ID NO: 511 - RNA, 23 nt, synthetic construct
  source 1..23: mol_type = other RNA
catgaagtacagagtgaccgaca

SEQ ID NO: 512 - RNA, 23 nt, synthetic construct
  source 1..23: mol_type = other RNA
tcatactttctgtagagccaaga

SEQ ID NO: 513 - RNA, 23 nt, synthetic construct
  source 1..23: mol_type = other RNA
caggatataagtgatcacgccag

SEQ ID NO: 514 - RNA, 23 nt, synthetic construct
  source 1..23: mol_type = other RNA
caaacaaaaatgcctgagcccag

SEQ ID NO: 515 - RNA, 23 nt, synthetic construct
  source 1..23: mol_type = other RNA
gactatgtataaacacagcagct

SEQ ID NO: 516 - RNA, 23 nt, synthetic construct
  source 1..23: mol_type = other RNA
cagaaagcaataaatccacaccc

SEQ ID NO: 517 - RNA, 23 nt, synthetic construct
  source 1..23: mol_type = other RNA
agaaattcaaagaacaccaccac

SEQ ID NO: 518 - RNA, 23 nt, synthetic construct
  source 1..23: mol_type = other RNA
cccacaagaagatccaggccaca

SEQ ID NO: 519 - RNA, 23 nt, synthetic construct
  source 1..23: mol_type = other RNA
gctcagaaaagtaagcagtgcat

SEQ ID NO: 520 - RNA, 23 nt, synthetic construct
  source 1..23: mol_type = other RNA
tagagaataagaccactcagcac

SEQ ID NO: 521 - RNA, 23 nt, synthetic construct
  source 1..23: mol_type = other RNA
agcataaatagaaaccatggcag

SEQ ID NO: 522 - RNA, 23 nt, synthetic construct
  source 1..23: mol_type = other RNA
tcagatgtttatgaccagacgga

SEQ ID NO: 523 - RNA, 23 nt, synthetic construct
  source 1..23: mol_type = other RNA
tcaacatgatataatcgacgtgt

SEQ ID NO: 524 - RNA, 23 nt, synthetic construct
  source 1..23: mol_type = other RNA
tgtaccgtctatgtgcaagccga

SEQ ID NO: 525 - RNA, 23 nt, synthetic construct
  source 1..23: mol_type = other RNA
atgagatataggtgcccccaatt

SEQ ID NO: 526 - RNA, 23 nt, synthetic construct
  source 1..23: mol_type = other RNA
actcagctgccatgactgcacgt

SEQ ID NO: 527 - RNA, 23 nt, synthetic construct
  source 1..23: mol_type = other RNA
ctcttcagctagctgtgccactt

SEQ ID NO: 528 - RNA, 23 nt, synthetic construct
  source 1..23: mol_type = other RNA
ccatcctaatatttactccacag

SEQ ID NO: 529 - RNA, 23 nt, synthetic construct
  source 1..23: mol_type = other RNA
gtcaccaagagtagcagcagcag

SEQ ID NO: 530 - RNA, 23 nt, synthetic construct
  source 1..23: mol_type = other RNA
atgcacaaagttcagcgtggcga

SEQ ID NO: 531 - RNA, 23 nt, synthetic construct
  source 1..23: mol_type = other RNA
cactgaagagtaaccaaggacct

SEQ ID NO: 532 - RNA, 23 nt, synthetic construct
  source 1..23: mol_type = other RNA
cccatgagaatgcactcccaagc

SEQ ID NO: 533 - RNA, 23 nt, synthetic construct
  source 1..23: mol_type = other RNA
aggaccaaaacagagcccagcat

SEQ ID NO: 534 - RNA, 23 nt, synthetic construct
  source 1..23: mol_type = other RNA
tacttacagaagttacagctcag

SEQ ID NO: 535 - RNA, 23 nt, synthetic construct
  source 1..23: mol_type = other RNA
tcatctttcatagtcacgtccat

SEQ ID NO: 536 - RNA, 23 nt, synthetic construct
  source 1..23: mol_type = other RNA
aaggatgtctgtatgggagcagt

SEQ ID NO: 537 - RNA, 23 nt, synthetic construct
  source 1..23: mol_type = other RNA
aagttgatcacagagacagcctg

SEQ ID NO: 538 - RNA, 23 nt, synthetic construct
  source 1..23: mol_type = other RNA
attcatatagtttccgaccagca

SEQ ID NO: 539 - RNA, 23 nt, synthetic construct
  source 1..23: mol_type = other RNA
ctcagccaagaaatgcaacagag

SEQ ID NO: 540 - RNA, 23 nt, synthetic construct
  source 1..23: mol_type = other RNA
agaaaagcacaaatgcagcaaga

SEQ ID NO: 541 - RNA, 23 nt, synthetic construct
  source 1..23: mol_type = other RNA
tgcaaaacaatttctcagccagg

SEQ ID NO: 542 - RNA, 23 nt, synthetic construct
  source 1..23: mol_type = other RNA
agcaccaaatgtctgcacccaac

SEQ ID NO: 543 - RNA, 23 nt, synthetic construct
  source 1..23: mol_type = other RNA
gtctataagagcttaagcagcag

SEQ ID NO: 544 - RNA, 23 nt, synthetic construct
  source 1..23: mol_type = other RNA
attatttacatagatgaggcaca

SEQ ID NO: 545 - RNA, 23 nt, synthetic construct
  source 1..23: mol_type = other RNA
tgcacaaatgacaaggttccacc

SEQ ID NO: 546 - RNA, 23 nt, synthetic construct
  source 1..23: mol_type = other RNA
agagcaaatttcatggagagctt

SEQ ID NO: 547 - RNA, 23 nt, synthetic construct
  source 1..23: mol_type = other RNA
gaagcaatcaaaggccagacgcc

SEQ ID NO: 548 - RNA, 23 nt, synthetic construct
  source 1..23: mol_type = other RNA
cacaagtgttaaacgcaaagcca

SEQ ID NO: 549 - RNA, 23 nt, synthetic construct
  source 1..23: mol_type = other RNA
acaaactgacaataggtgccaga

SEQ ID NO: 550 - RNA, 23 nt, synthetic construct
  source 1..23: mol_type = other RNA
catatcaatcaaatacagccaca

SEQ ID NO: 551 - RNA, 23 nt, synthetic construct
  source 1..23: mol_type = other RNA
agatcagcaaagaattggcatcc

SEQ ID NO: 552 - RNA, 23 nt, synthetic construct
  source 1..23: mol_type = other RNA
cacagttaagaccttcatagccg

SEQ ID NO: 553 - RNA, 23 nt, synthetic construct
  source 1..23: mol_type = other RNA
agtgatatagagactgcaggcat

SEQ ID NO: 554 - RNA, 23 nt, synthetic construct
  source 1..23: mol_type = other RNA
acactagaaaccatgctacaagc

SEQ ID NO: 555 - RNA, 23 nt, synthetic construct
  source 1..23: mol_type = other RNA
catgaatgcttacagacacagtc

SEQ ID NO: 556 - RNA, 23 nt, synthetic construct
  source 1..23: mol_type = other RNA
acatgctcagtagtaccatctgc

SEQ ID NO: 557 - RNA, 23 nt, synthetic construct
  source 1..23: mol_type = other RNA
gaacctccggaaatccccacagt

SEQ ID NO: 558 - RNA, 23 nt, synthetic construct
  source 1..23: mol_type = other RNA
tagaaagcagaaaacgccccaaa

SEQ ID NO: 559 - RNA, 23 nt, synthetic construct
  source 1..23: mol_type = other RNA
atagttaatagtaatccacccat

SEQ ID NO: 560 - RNA, 23 nt, synthetic construct
  source 1..23: mol_type = other RNA
acacagtatagtcctcgccatag

SEQ ID NO: 561 - RNA, 23 nt, synthetic construct
  source 1..23: mol_type = other RNA
caagttcagcatgtagcccaaag

SEQ ID NO: 562 - RNA, 23 nt, synthetic construct
  source 1..23: mol_type = other RNA
agtcacattagctgcacgagcac

SEQ ID NO: 563 - RNA, 23 nt, synthetic construct
  source 1..23: mol_type = other RNA
aaattcagcatgtagcccagcgc

SEQ ID NO: 564 - RNA, 23 nt, synthetic construct
  source 1..23: mol_type = other RNA
tcaatcagaacagaggctccact

SEQ ID NO: 565 - RNA, 23 nt, synthetic construct
  source 1..23: mol_type = other RNA
atgtatgaacatggctacagcgc

SEQ ID NO: 566 - RNA, 23 nt, synthetic construct
  source 1..23: mol_type = other RNA
agtgacaccaactttccagccat

SEQ ID NO: 567 - RNA, 23 nt, synthetic construct
  source 1..23: mol_type = other RNA
atagagccgcaaatggacctcat

SEQ ID NO: 568 - RNA, 23 nt, synthetic construct
  source 1..23: mol_type = other RNA
accatattcattcagagagcaac

SEQ ID NO: 569 - RNA, 23 nt, synthetic construct
  source 1..23: mol_type = other RNA
tctagtgaaacatgcagctgcag

SEQ ID NO: 570 - RNA, 23 nt, synthetic construct
  source 1..23: mol_type = other RNA
ctcacgtgcagaatcagtccatc

SEQ ID NO: 571 - RNA, 23 nt, synthetic construct
  source 1..23: mol_type = other RNA
cagcttgtagttgtcacagacac

SEQ ID NO: 572 - RNA, 23 nt, synthetic construct
  source 1..23: mol_type = other RNA
cacgcccctccccgccctcacct

SEQ ID NO: 573 - RNA, 23 nt, synthetic construct
  source 1..23: mol_type = other RNA
aaaaagagtcaaattggagacag

SEQ ID NO: 574 - RNA, 23 nt, synthetic construct
  source 1..23: mol_type = other RNA
agaatgacgagaaatgcagcacc

SEQ ID NO: 575 - RNA, 23 nt, synthetic construct
  source 1..23: mol_type = other RNA
acgacatttcactatgtgcccaa

SEQ ID NO: 576 - RNA, 23 nt, synthetic construct
  source 1..23: mol_type = other RNA
cttgtagtagacacgcaccgaga

SEQ ID NO: 577 - RNA, 23 nt, synthetic construct
  source 1..23: mol_type = other RNA
acacaagcagcataccccacaac

SEQ ID NO: 578 - RNA, 23 nt, synthetic construct
  source 1..23: mol_type = other RNA
cgactttcatataacacccactc

SEQ ID NO: 579 - RNA, 23 nt, synthetic construct
  source 1..23: mol_type = other RNA
agaccatagcatactccagcaca

SEQ ID NO: 580 - RNA, 23 nt, synthetic construct
  source 1..23: mol_type = other RNA
acacagtgagttacagaccaagc

SEQ ID NO: 581 - RNA, 23 nt, synthetic construct
  source 1..23: mol_type = other RNA
gcaaaaacgtctttgacaacccc

SEQ ID NO: 582 - RNA, 23 nt, synthetic construct
  source 1..23: mol_type = other RNA
accatcaaacacatcggagccag

SEQ ID NO: 583 - RNA, 23 nt, synthetic construct
  source 1..23: mol_type = other RNA
cacagcattttcatgacacaccc

SEQ ID NO: 584 - RNA, 23 nt, synthetic construct
  source 1..23: mol_type = other RNA
atgacatagtaaaagacacagcc

SEQ ID NO: 585 - RNA, 23 nt, synthetic construct
  source 1..23: mol_type = other RNA
aaatccaattttacagcagcagg

SEQ ID NO: 586 - RNA, 23 nt, synthetic construct
  source 1..23: mol_type = other RNA
agacaaaagaatcctggcacagc

SEQ ID NO: 587 - RNA, 23 nt, synthetic construct
  source 1..23: mol_type = other RNA
agcaacgttgatgtgcaccacct

SEQ ID NO: 588 - RNA, 23 nt, synthetic construct
  source 1..23: mol_type = other RNA
ttcacaaacagacggctgcagac

SEQ ID NO: 589 - RNA, 23 nt, synthetic construct
  source 1..23: mol_type = other RNA
taagaataatgtgctgagcttgg

SEQ ID NO: 590 - RNA, 23 nt, synthetic construct
  source 1..23: mol_type = other RNA
acatgctgagatttgacgcaagg

SEQ ID NO: 591 - RNA, 23 nt, synthetic construct
  source 1..23: mol_type = other RNA
ttatttggtgtagtctgggacaa

SEQ ID NO: 592 - RNA, 23 nt, synthetic construct
  source 1..23: mol_type = other RNA
catacctggccacagtgcaccga

SEQ ID NO: 593 - RNA, 23 nt, synthetic construct
  source 1..23: mol_type = other RNA
acgccaaaaatcaagagtccaag

SEQ ID NO: 594 - RNA, 23 nt, synthetic construct
  source 1..23: mol_type = other RNA
cccattacagttacaatcccgac

SEQ ID NO: 595 - RNA, 23 nt, synthetic construct
  source 1..23: mol_type = other RNA
aatgaagaattcatgaaggcccg

SEQ ID NO: 596 - RNA, 23 nt, synthetic construct
  source 1..23: mol_type = other RNA
cctagaagacattgaaggcatcc

SEQ ID NO: 597 - RNA, 23 nt, synthetic construct
  source 1..23: mol_type = other RNA
agtttaattaaactgtccccatg

SEQ ID NO: 598 - RNA, 23 nt, synthetic construct
  source 1..23: mol_type = other RNA
agccaaattaacatggcagcaat

SEQ ID NO: 599 - RNA, 23 nt, synthetic construct
  source 1..23: mol_type = other RNA
aacagaaatttgctgcaacccaa

SEQ ID NO: 600 - RNA, 23 nt, synthetic construct
  source 1..23: mol_type = other RNA
agcaaagatgcattgatccagga

SEQ ID NO: 601 - RNA, 23 nt, synthetic construct
  source 1..23: mol_type = other RNA
actgcaataacagaaacagctcc

SEQ ID NO: 602 - RNA, 23 nt, synthetic construct
  source 1..23: mol_type = other RNA
agcagcagtacacgggacacctc

SEQ ID NO: 603 - RNA, 23 nt, synthetic construct
  source 1..23: mol_type = other RNA
tcatgcacgatttctgacacccc

SEQ ID NO: 604 - RNA, 23 nt, synthetic construct
  source 1..23: mol_type = other RNA
tcttgtagacaatgtcgcccaat

SEQ ID NO: 605 - RNA, 23 nt, synthetic construct
  source 1..23: mol_type = other RNA
accattttattgtcaccttccag

SEQ ID NO: 606 - RNA, 23 nt, synthetic construct
  source 1..23: mol_type = other RNA
gagaagttcatggtggcaacgag

SEQ ID NO: 607 - RNA, 23 nt, synthetic construct
  source 1..23: mol_type = other RNA
tcaccttccagcttgacgactgc

SEQ ID NO: 608 - RNA, 23 nt, synthetic construct
  source 1..23: mol_type = other RNA
ctttcataacacattccaccacc

SEQ ID NO: 609 - RNA, 23 nt, synthetic construct
  source 1..23: mol_type = other RNA
tcatattatttgtaccagaacca

SEQ ID NO: 610 - RNA, 23 nt, synthetic construct
  source 1..23: mol_type = other RNA
gatttcatcgaattccacgccca

SEQ ID NO: 611 - RNA, 23 nt, synthetic construct
  source 1..23: mol_type = other RNA
acaagtacaaaattagacacaca

SEQ ID NO: 612 - RNA, 23 nt, synthetic construct
  source 1..23: mol_type = other RNA
tttgatgcaaatttccatccagg

SEQ ID NO: 613 - RNA, 23 nt, synthetic construct
  source 1..23: mol_type = other RNA
aaaaacaaagagcagtgtccagg

SEQ ID NO: 614 - RNA, 23 nt, synthetic construct
  source 1..23: mol_type = other RNA
taataacaatggctgcgacagca

SEQ ID NO: 615 - RNA, 23 nt, synthetic construct
  source 1..23: mol_type = other RNA
gtgaattataagcagttcccacg

SEQ ID NO: 616 - RNA, 23 nt, synthetic construct
  source 1..23: mol_type = other RNA
gctaacatccagaaaggcccagt

SEQ ID NO: 617 - RNA, 23 nt, synthetic construct
  source 1..23: mol_type = other RNA
cagcagcaagatttagcacggct

SEQ ID NO: 618 - RNA, 23 nt, synthetic construct
  source 1..23: mol_type = other RNA
acaaccagtagagtcagccacat

SEQ ID NO: 619 - RNA, 23 nt, synthetic construct
  source 1..23: mol_type = other RNA
gtaaacttcttagtggcagcaag

SEQ ID NO: 620 - RNA, 23 nt, synthetic construct
  source 1..23: mol_type = other RNA
ataacaagtcagtgtgacgacct

SEQ ID NO: 621 - RNA, 23 nt, synthetic construct
  source 1..23: mol_type = other RNA
taaaacagaaacactccaacagc

SEQ ID NO: 622 - RNA, 23 nt, synthetic construct
  source 1..23: mol_type = other RNA
atacaggatacatggagagcccc

SEQ ID NO: 623 - RNA, 23 nt, synthetic construct
  source 1..23: mol_type = other RNA
caagctatataagaagcaacgag

SEQ ID NO: 624 - RNA, 23 nt, synthetic construct
  source 1..23: mol_type = other RNA
tcttgacaaatatatgcccagag

SEQ ID NO: 625 - RNA, 23 nt, synthetic construct
  source 1..23: mol_type = other RNA
atacccattcttaaagcacagtg

SEQ ID NO: 626 - RNA, 23 nt, synthetic construct
  source 1..23: mol_type = other RNA
cctttatatacacttcgcccgca

SEQ ID NO: 627 - RNA, 23 nt, synthetic construct
  source 1..23: mol_type = other RNA
cctttcaagacacagacctcccc

SEQ ID NO: 628 - RNA, 23 nt, synthetic construct
  source 1..23: mol_type = other RNA
gtgcaatttcatatgacaagcca

SEQ ID NO: 629 - RNA, 23 nt, synthetic construct
  source 1..23: mol_type = other RNA
cttccacaatcatgagcgcacag

SEQ ID NO: 630 - RNA, 23 nt, synthetic construct
  source 1..23: mol_type = other RNA
atatatacctttaatcccagcac

SEQ ID NO: 631 - RNA, 23 nt, synthetic construct
  source 1..23: mol_type = other RNA
aaaccaaacatggtgccgaacac

SEQ ID NO: 632 - RNA, 23 nt, synthetic construct
  source 1..23: mol_type = other RNA
aactctaaagagaagcagagccg

SEQ ID NO: 633 - RNA, 23 nt, synthetic construct
  source 1..23: mol_type = other RNA
aatgtcagaacattcagaccacc

SEQ ID NO: 634 - RNA, 23 nt, synthetic construct
  source 1..23: mol_type = other RNA
gcactagatacaatccagcacca

SEQ ID NO: 635 - RNA, 23 nt, synthetic construct
  source 1..23: mol_type = other RNA
tccacatgtcgaaagacctcagg

SEQ ID NO: 636 - RNA, 23 nt, synthetic construct
  source 1..23: mol_type = other RNA
tctcctctctgtaatgcaccagc

SEQ ID NO: 637 - RNA, 23 nt, synthetic construct
  source 1..23: mol_type = other RNA
aagacgtgtaagtagtgcagccc

SEQ ID NO: 638 - RNA, 23 nt, synthetic construct
  source 1..23: mol_type = other RNA
tctacagacagaaagcgaccgac

SEQ ID NO: 639 - RNA, 23 nt, synthetic construct
  source 1..23: mol_type = other RNA
tgtgcaaattatgataggccagc

SEQ ID NO: 640 - RNA, 23 nt, synthetic construct
  source 1..23: mol_type = other RNA
aggaaaataaatagcagctgccc

SEQ ID NO: 641 - RNA, 23 nt, synthetic construct
  source 1..23: mol_type = other RNA
ataacatgaagaaagtgacgtgg

SEQ ID NO: 642 - RNA, 23 nt, synthetic construct
  source 1..23: mol_type = other RNA
atgaatgaaaagttcttgggctg

SEQ ID NO: 643 - RNA, 23 nt, synthetic construct
  source 1..23: mol_type = other RNA
caagcagaagcaaagtgcaaccc

SEQ ID NO: 644 - RNA, 23 nt, synthetic construct
  source 1..23: mol_type = other RNA
atagatatgaatcatcgcaacag

SEQ ID NO: 645 - RNA, 23 nt, synthetic construct
  source 1..23: mol_type = other RNA
accagcatttctgagacacagcg

SEQ ID NO: 646 - RNA, 23 nt, synthetic construct
  source 1..23: mol_type = other RNA
tgatagtatcaagctagcccgag

SEQ ID NO: 647 - RNA, 23 nt, synthetic construct
  source 1..23: mol_type = other RNA
cagaactcatctttccaagccac

SEQ ID NO: 648 - RNA, 23 nt, synthetic construct
  source 1..23: mol_type = other RNA
tatgtctaaactttagcagccac

SEQ ID NO: 649 - RNA, 23 nt, synthetic construct
  source 1..23: mol_type = other RNA
ccaggttaaatttcagccccaac

SEQ ID NO: 650 - RNA, 23 nt, synthetic construct
  source 1..23: mol_type = other RNA
cacacctttaattcccacccaag

SEQ ID NO: 651 - RNA, 23 nt, synthetic construct
  source 1..23: mol_type = other RNA
cgattcgaagttcatccacaccc

SEQ ID NO: 652 - RNA, 23 nt, synthetic construct
  source 1..23: mol_type = other RNA
ggtatttagaaaacgcagccaag

SEQ ID NO: 653 - RNA, 23 nt, synthetic construct
  source 1..23: mol_type = other RNA
catgtttattggaaagagccgat

SEQ ID NO: 654 - RNA, 23 nt, synthetic construct
  source 1..23: mol_type = other RNA
tgattgtattccagaaggcgagc

SEQ ID NO: 655 - RNA, 23 nt, synthetic construct
  source 1..23: mol_type = other RNA
tggtcccaaatttaggcatgctg

SEQ ID NO: 656 - RNA, 23 nt, synthetic construct
  source 1..23: mol_type = other RNA
tccagtaggttttaaaaacgggg

SEQ ID NO: 657 - RNA, 23 nt, synthetic construct
  source 1..23: mol_type = other RNA
tctagaaactgagtttggctgtg

SEQ ID NO: 658 - RNA, 23 nt, synthetic construct
  source 1..23: mol_type = other RNA
ttctgcttctgccagcgccacag

SEQ ID NO: 659 - RNA, 23 nt, synthetic construct
  source 1..23: mol_type = other RNA
gttaactctatagtagtccagag

SEQ ID NO: 660 - RNA, 23 nt, synthetic construct
  source 1..23: mol_type = other RNA
cacgtgaatgtaatccctcacac

SEQ ID NO: 661 - RNA, 23 nt, synthetic construct
  source 1..23: mol_type = other RNA
aaagcttaatcatcagtgcctgg

SEQ ID NO: 662 - RNA, 23 nt, synthetic construct
  source 1..23: mol_type = other RNA
attgaagtaccgaagcagcacgg

SEQ ID NO: 663 - RNA, 23 nt, synthetic construct
  source 1..23: mol_type = other RNA
taaagagcactatgtcagccaga

SEQ ID NO: 664 - RNA, 23 nt, synthetic construct
  source 1..23: mol_type = other RNA
atacatacagtaaatccacacag

SEQ ID NO: 665 - RNA, 23 nt, synthetic construct
  source 1..23: mol_type = other RNA
attaaaggcagatatcccaccac

SEQ ID NO: 666 - RNA, 23 nt, synthetic construct
  source 1..23: mol_type = other RNA
actgagacagataacagccgcag

SEQ ID NO: 667 - RNA, 23 nt, synthetic construct
  source 1..23: mol_type = other RNA
aagatagccaatatggacccttc

SEQ ID NO: 668 - RNA, 23 nt, synthetic construct
  source 1..23: mol_type = other RNA
agcaaaaatgaagacagagccat

SEQ ID NO: 669 - RNA, 23 nt, synthetic construct
  source 1..23: mol_type = other RNA
taacatgaatactcaaggccaga

SEQ ID NO: 670 - RNA, 23 nt, synthetic construct
  source 1..23: mol_type = other RNA
caaaagaaagaaatcagaagcca

SEQ ID NO: 671 - RNA, 23 nt, synthetic construct
  source 1..23: mol_type = other RNA
aatattttcattttccagcacgt

SEQ ID NO: 672 - RNA, 23 nt, synthetic construct
  source 1..23: mol_type = other RNA
acattatcaaagcaggatgcgat

SEQ ID NO: 673 - RNA, 23 nt, synthetic construct
  source 1..23: mol_type = other RNA
gtagacattacagccgtgcccaa

SEQ ID NO: 674 - RNA, 23 nt, synthetic construct
  source 1..23: mol_type = other RNA
aaatacacaatccatccacccag

SEQ ID NO: 675 - RNA, 23 nt, synthetic construct
  source 1..23: mol_type = other RNA
acagaacatgtgatggagtccag

SEQ ID NO: 676 - RNA, 23 nt, synthetic construct
  source 1..23: mol_type = other RNA
agaaaaggcaagttcaggcaagt

SEQ ID NO: 677 - RNA, 23 nt, synthetic construct
  source 1..23: mol_type = other RNA
cataagtctgcagtgacacacca

SEQ ID NO: 678 - RNA, 23 nt, synthetic construct
  source 1..23: mol_type = other RNA
tgcagaccattatagcaacccgt

SEQ ID NO: 679 - RNA, 23 nt, synthetic construct
  source 1..23: mol_type = other RNA
tattgaatgatacagcccaccaa

SEQ ID NO: 680 - RNA, 23 nt, synthetic construct
  source 1..23: mol_type = other RNA
actgaatcacaaaaagccagccg

SEQ ID NO: 681 - RNA, 23 nt, synthetic construct
  source 1..23: mol_type = other RNA
ccaacatattcattaccagccat

SEQ ID NO: 682 - RNA, 23 nt, synthetic construct
  source 1..23: mol_type = other RNA
caactaaaagaatacccccagca

SEQ ID NO: 683 - RNA, 23 nt, synthetic construct
  source 1..23: mol_type = other RNA
atcagcagttgaagtaagccgag

SEQ ID NO: 684 - RNA, 23 nt, synthetic construct
  source 1..23: mol_type = other RNA
cctaaaactgaacaaccccagca

SEQ ID NO: 685 - RNA, 23 nt, synthetic construct
  source 1..23: mol_type = other RNA
agatgattttaaaagaggcaaca

SEQ ID NO: 686 - RNA, 23 nt, synthetic construct
  source 1..23: mol_type = other RNA
aaagatttaaaagagctgaacca

SEQ ID NO: 687 - RNA, 23 nt, synthetic construct
  source 1..23: mol_type = other RNA
actttcaagttccttcaaccccg

SEQ ID NO: 688 - RNA, 23 nt, synthetic construct
  source 1..23: mol_type = other RNA
caccaccaagtaaagagccaacc

SEQ ID NO: 689 - RNA, 23 nt, synthetic construct
  source 1..23: mol_type = other RNA
aatgttagagacaaaccagacac

SEQ ID NO: 690 - RNA, 23 nt, synthetic construct
  source 1..23: mol_type = other RNA
ctcataacgatcaatcctccgag

SEQ ID NO: 691 - RNA, 23 nt, synthetic construct
  source 1..23: mol_type = other RNA
aacaacatgacacgtcaaagccc

SEQ ID NO: 692 - RNA, 23 nt, synthetic construct
  source 1..23: mol_type = other RNA
cgacaaagtgctttgcaactgca

SEQ ID NO: 693 - RNA, 23 nt, synthetic construct
  source 1..23: mol_type = other RNA
cacaaaagtgatattggaaccca

SEQ ID NO: 694 - RNA, 23 nt, synthetic construct
  source 1..23: mol_type = other RNA
agtagaatatgtacacacacgca

SEQ ID NO: 695 - RNA, 23 nt, synthetic construct
  source 1..23: mol_type = other RNA
attttcatccgaagaccagccac

SEQ ID NO: 696 - RNA, 23 nt, synthetic construct
  source 1..23: mol_type = other RNA
cttagagaacaacagttcccagc

SEQ ID NO: 697 - RNA, 23 nt, synthetic construct
  source 1..23: mol_type = other RNA
gcagacatttataaaagccctag

SEQ ID NO: 698 - RNA, 23 nt, synthetic construct
  source 1..23: mol_type = other RNA
actgacaaagcagtacagcaccg

SEQ ID NO: 699 - RNA, 23 nt, synthetic construct
  source 1..23: mol_type = other RNA
aacagatatagaacaggtctgca

SEQ ID NO: 700 - RNA, 23 nt, synthetic construct
  source 1..23: mol_type = other RNA
gcacagaacacttagctatcacc

SEQ ID NO: 701 - RNA, 23 nt, synthetic construct
  source 1..23: mol_type = other RNA
gaagtcactacataaacccacct

SEQ ID NO: 702 - RNA, 23 nt, synthetic construct
  source 1..23: mol_type = other RNA
agactagtttagaggcaagctca

SEQ ID NO: 703 - RNA, 23 nt, synthetic construct
  source 1..23: mol_type = other RNA
ccaaagtcagaaatgaaggcgag

SEQ ID NO: 704 - RNA, 23 nt, synthetic construct
  source 1..23: mol_type = other RNA
agacaccaatgataagtgccagg

SEQ ID NO: 705 - RNA, 23 nt, synthetic construct
  source 1..23: mol_type = other RNA
cccacctaagaaagccaggacct

SEQ ID NO: 706 - RNA, 23 nt, synthetic construct
  source 1..23: mol_type = other RNA
agtccttaatttcatcagcccat

SEQ ID NO: 707 - RNA, 23 nt, synthetic construct
  source 1..23: mol_type = other RNA
tatagtgttaagcatatgccacc

SEQ ID NO: 708 - RNA, 23 nt, synthetic construct
  source 1..23: mol_type = other RNA
ttacagagcaaataggacccagc

SEQ ID NO: 709 - RNA, 23 nt, synthetic construct
  source 1..23: mol_type = other RNA
acaaagtataaatatgaaccagg

SEQ ID NO: 710 - RNA, 23 nt, synthetic construct
  source 1..23: mol_type = other RNA
acgacagtactacagccaccaca

SEQ ID NO: 711 - RNA, 23 nt, synthetic construct
  source 1..23: mol_type = other RNA
ccagaaagagcttcagcatcacc

SEQ ID NO: 712 - RNA, 23 nt, synthetic construct
  source 1..23: mol_type = other RNA
acagagcaattacaggaggccca

SEQ ID NO: 713 - RNA, 23 nt, synthetic construct
  source 1..23: mol_type = other RNA
tccaaacaacgttgaccgagcag

SEQ ID NO: 714 - RNA, 23 nt, synthetic construct
  source 1..23: mol_type = other RNA
agcaaaatatcgtggcgccagca

SEQ ID NO: 715 - RNA, 23 nt, synthetic construct
  source 1..23: mol_type = other RNA
cagaagatacacagtccccaaga

SEQ ID NO: 716 - RNA, 23 nt, synthetic construct
  source 1..23: mol_type = other RNA
tcatgtgaaagcagaagccgcac

SEQ ID NO: 717 - RNA, 23 nt, synthetic construct
  source 1..23: mol_type = other RNA
gcaccaaagacaaaacagacgag

SEQ ID NO: 718 - RNA, 23 nt, synthetic construct
  source 1..23: mol_type = other RNA
gcatacatacaaacaccacccct

SEQ ID NO: 719 - RNA, 23 nt, synthetic construct
  source 1..23: mol_type = other RNA
acaattaggagtgacatgaccgc

SEQ ID NO: 720 - RNA, 23 nt, synthetic construct
  source 1..23: mol_type = other RNA
gcatgttgatgtagactctacca

SEQ ID NO: 721 - RNA, 23 nt, synthetic construct
  source 1..23: mol_type = other RNA
gcagagacagagtcccacatccg

SEQ ID NO: 722 - RNA, 23 nt, synthetic construct
  source 1..23: mol_type = other RNA
agagtcccacatccggagcagga

SEQ ID NO: 723 - RNA, 23 nt, synthetic construct
  source 1..23: mol_type = other RNA
tcaaacaggaacttgatagccag

SEQ ID NO: 724 - RNA, 23 nt, synthetic construct
  source 1..23: mol_type = other RNA
cctctttagcaaagcgatcccca

SEQ ID NO: 725 - RNA, 23 nt, synthetic construct
  source 1..23: mol_type = other RNA
accaccaacattgatggcccagc

SEQ ID NO: 726 - RNA, 23 nt, synthetic construct
  source 1..23: mol_type = other RNA
ccacaatgacaaactgtgctgtc

SEQ ID NO: 727 - RNA, 23 nt, synthetic construct
  source 1..23: mol_type = other RNA
catggtaatgaatatgagtcctg

SEQ ID NO: 728 - RNA, 23 nt, synthetic construct
  source 1..23: mol_type = other RNA
aagaatcaaatgttaagcaccag

SEQ ID NO: 729 - RNA, 23 nt, synthetic construct
  source 1..23: mol_type = other RNA
ccacattcactaatgccaaccac

SEQ ID NO: 730 - RNA, 23 nt, synthetic construct
  source 1..23: mol_type = other RNA
aaaaccaagcatgatagcagcgc

SEQ ID NO: 731 - RNA, 23 nt, synthetic construct
  source 1..23: mol_type = other RNA
catgttgacattgaaacccacgc

SEQ ID NO: 732 - RNA, 23 nt, synthetic construct
  source 1..23: mol_type = other RNA
acacactccaaatttccaccagc

SEQ ID NO: 733 - RNA, 23 nt, synthetic construct
  source 1..23: mol_type = other RNA
ctgcataaataagtagcccagat

SEQ ID NO: 734 - RNA, 23 nt, synthetic construct
  source 1..23: mol_type = other RNA
actagaagccaattgcagcagca

SEQ ID NO: 735 - RNA, 23 nt, synthetic construct
  source 1..23: mol_type = other RNA
aatttgagagcagtagccaactc

SEQ ID NO: 736 - RNA, 23 nt, synthetic construct
  source 1..23: mol_type = other RNA
ctgatccaatcttttcagcccca

SEQ ID NO: 737 - RNA, 23 nt, synthetic construct
  source 1..23: mol_type = other RNA
cctttatcaatgatcctccacag

SEQ ID NO: 738 - RNA, 23 nt, synthetic construct
  source 1..23: mol_type = other RNA
aacaagacagaaatacgagacag

SEQ ID NO: 739 - RNA, 23 nt, synthetic construct
  source 1..23: mol_type = other RNA
tcacacagaagttagagaccaag

SEQ ID NO: 740 - RNA, 23 nt, synthetic construct
  source 1..23: mol_type = other RNA
cccaagagaagagagccaggcaa

SEQ ID NO: 741 - RNA, 23 nt, synthetic construct
  source 1..23: mol_type = other RNA
tacatggcataaattcccactgc

SEQ ID NO: 742 - RNA, 23 nt, synthetic construct
  source 1..23: mol_type = other RNA
gtcagcatcacctgcagctcctc

SEQ ID NO: 743 - RNA, 23 nt, synthetic construct
  source 1..23: mol_type = other RNA
atacaagcagaaaatgacgccct

SEQ ID NO: 744 - RNA, 23 nt, synthetic construct
  source 1..23: mol_type = other RNA
tttatataaatgctgaagcagcc

SEQ ID NO: 745 - RNA, 23 nt, synthetic construct
  source 1..23: mol_type = other RNA
agaaatgacagcattggcaaccg

SEQ ID NO: 746 - RNA, 23 nt, synthetic construct
  source 1..23: mol_type = other RNA
aaataaagaaattatcagcccct

SEQ ID NO: 747 - RNA, 23 nt, synthetic construct
  source 1..23: mol_type = other RNA
tcacagaaaactcagccatccca

SEQ ID NO: 748 - RNA, 23 nt, synthetic construct
  source 1..23: mol_type = other RNA
gtaaaacaatattttcagccagt

SEQ ID NO: 749 - RNA, 23 nt, synthetic construct
  source 1..23: mol_type = other RNA
ttcaataaaacagaaacacgcgg

SEQ ID NO: 750 - RNA, 23 nt, synthetic construct
  source 1..23: mol_type = other RNA
cgacagaccaagtaccacctcgc

SEQ ID NO: 751 - RNA, 23 nt, synthetic construct
  source 1..23: mol_type = other RNA
cagcgacacaagatgcgatcgtc

SEQ ID NO: 752 - RNA, 23 nt, synthetic construct
  source 1..23: mol_type = other RNA
gtagtcgattacatgctgcagga

SEQ ID NO: 753 - RNA, 23 nt, synthetic construct
  source 1..23: mol_type = other RNA
cccccttattcaaaacagaccgc

SEQ ID NO: 754 - RNA, 23 nt, synthetic construct
  source 1..23: mol_type = other RNA
acctaaagcagcaaacagtgcgc

SEQ ID NO: 755 - RNA, 23 nt, synthetic construct
  source 1..23: mol_type = other RNA
agttcataatcagggcagcagag

SEQ ID NO: 756 - RNA, 23 nt, synthetic construct
  source 1..23: mol_type = other RNA
catgattacagaaagtcaccttc

SEQ ID NO: 757 - RNA, 23 nt, synthetic construct
  source 1..23: mol_type = other RNA
gatgtagtctatgacacgctgca

SEQ ID NO: 758 - RNA, 23 nt, synthetic construct
  source 1..23: mol_type = other RNA
atttccaccaatagagagacgag

SEQ ID NO: 759 - RNA, 23 nt, synthetic construct
  source 1..23: mol_type = other RNA
agacagatatatgcggagaacgt

SEQ ID NO: 760 - RNA, 23 nt, synthetic construct
  source 1..23: mol_type = other RNA
aatctacataatatacaacaggc

SEQ ID NO: 761 - RNA, 23 nt, synthetic construct
  source 1..23: mol_type = other RNA
gcataagacagaataggaggcag

SEQ ID NO: 762 - RNA, 23 nt, synthetic construct
  source 1..23: mol_type = other RNA
aacctcaaaaccaggcacgccag

SEQ ID NO: 763 - RNA, 23 nt, synthetic construct
  source 1..23: mol_type = other RNA
ctaacatagaactgagagaccca

SEQ ID NO: 764 - RNA, 23 nt, synthetic construct
  source 1..23: mol_type = other RNA
taatttaaatatttccacccaga

SEQ ID NO: 765 - RNA, 23 nt, synthetic construct
  source 1..23: mol_type = other RNA
acatacagcatgataaccagccc

SEQ ID NO: 766 - RNA, 23 nt, synthetic construct
  source 1..23: mol_type = other RNA
caatgtagttattggacaccgca

SEQ ID NO: 767 - RNA, 23 nt, synthetic construct
  source 1..23: mol_type = other RNA
ttaataagcagattgcccttcag

SEQ ID NO: 768 - RNA, 23 nt, synthetic construct
  source 1..23: mol_type = other RNA
aatcttaagtaaaagagacacag

SEQ ID NO: 769 - RNA, 23 nt, synthetic construct
  source 1..23: mol_type = other RNA
cccaacatgttcatggtcccatc

SEQ ID NO: 770 - RNA, 23 nt, synthetic construct
  source 1..23: mol_type = other RNA
cgtcatcactgtctgcaacctgc

SEQ ID NO: 771 - RNA, 23 nt, synthetic construct
  source 1..23: mol_type = other RNA
aacagaagcaagggaggttcagc

SEQ ID NO: 772 - RNA, 23 nt, synthetic construct
  source 1..23: mol_type = other RNA
agagcagcagcaagagcaacgac

SEQ ID NO: 773 - RNA, 23 nt, synthetic construct
  source 1..23: mol_type = other RNA
cagagaagattcttcacccagca

SEQ ID NO: 774 - RNA, 23 nt, synthetic construct
  source 1..23: mol_type = other RNA
agcagctcctcacagcgttccct

SEQ ID NO: 775 - RNA, 23 nt, synthetic construct
  source 1..23: mol_type = other RNA
gatatacatgtacttgtgtcaca

SEQ ID NO: 776 - RNA, 23 nt, synthetic construct
  source 1..23: mol_type = other RNA
gaatttgcacataaggaactcct

SEQ ID NO: 777 - RNA, 23 nt, synthetic construct
  source 1..23: mol_type = other RNA
atttcatcttgaagcagtgacac

SEQ ID NO: 778 - RNA, 23 nt, synthetic construct
  source 1..23: mol_type = other RNA
gattccacagaatagcagcatag

SEQ ID NO: 779 - RNA, 23 nt, synthetic construct
  source 1..23: mol_type = other RNA
caggacctaaactatcaccccag

SEQ ID NO: 780 - RNA, 23 nt, synthetic construct
  source 1..23: mol_type = other RNA
aagcagacataagtcctaagcct

SEQ ID NO: 781 - RNA, 23 nt, synthetic construct
  source 1..23: mol_type = other RNA
tattaggatgaaaacacacacca

SEQ ID NO: 782 - RNA, 23 nt, synthetic construct
  source 1..23: mol_type = other RNA
gtttcaaataacctgcggccaga

SEQ ID NO: 783 - RNA, 23 nt, synthetic construct
  source 1..23: mol_type = other RNA
aacagatttcatagagaccccag

SEQ ID NO: 784 - RNA, 23 nt, synthetic construct
  source 1..23: mol_type = other RNA
ataagtataaataagccacaggt

SEQ ID NO: 785 - RNA, 23 nt, synthetic construct
  source 1..23: mol_type = other RNA
agtctttaacaacagcaggcccc

SEQ ID NO: 786 - RNA, 23 nt, synthetic construct
  source 1..23: mol_type = other RNA
cacaaacagtaacagtcacccac

SEQ ID NO: 787 - RNA, 23 nt, synthetic construct
  source 1..23: mol_type = other RNA
cacattttggtataaaaccccag

SEQ ID NO: 788 - RNA, 23 nt, synthetic construct
  source 1..23: mol_type = other RNA
acaaccattttattacggcacca

SEQ ID NO: 789 - RNA, 23 nt, synthetic construct
  source 1..23: mol_type = other RNA
gaacatttaaaagatgcacccgc

SEQ ID NO: 790 - RNA, 23 nt, synthetic construct
  source 1..23: mol_type = other RNA
atcaaagctatagtccatccgtg

SEQ ID NO: 791 - RNA, 23 nt, synthetic construct
  source 1..23: mol_type = other RNA
ccaaagttttcaaaggagctgat

SEQ ID NO: 792 - RNA, 23 nt, synthetic construct
  source 1..23: mol_type = other RNA
aactatcatctaataccaagcag

SEQ ID NO: 793 - RNA, 23 nt, synthetic construct
  source 1..23: mol_type = other RNA
tgtaaagattgaagtcagtgcag

SEQ ID NO: 794 - RNA, 23 nt, synthetic construct
  source 1..23: mol_type = other RNA
acatacaaaagcaggacccacca

SEQ ID NO: 795 - RNA, 23 nt, synthetic construct
  source 1..23: mol_type = other RNA
cacgttaagtgtaagcagagcta

SEQ ID NO: 796 - RNA, 23 nt, synthetic construct
  source 1..23: mol_type = other RNA
atatacagttgtgacctgacgca

SEQ ID NO: 797 - RNA, 23 nt, synthetic construct
  source 1..23: mol_type = other RNA
tcaatgaaggaaccattgcccag

SEQ ID NO: 798 - RNA, 23 nt, synthetic construct
  source 1..23: mol_type = other RNA
ctgaaatcaaaaagtcagagcac

SEQ ID NO: 799 - RNA, 23 nt, synthetic construct
  source 1..23: mol_type = other RNA
gaaaagtaagttttccagacacc

SEQ ID NO: 800 - RNA, 23 nt, synthetic construct
  source 1..23: mol_type = other RNA
ttactcaataaaatgaaggccac

SEQ ID NO: 801 - RNA, 23 nt, synthetic construct
  source 1..23: mol_type = other RNA
tgacataaaactaaccagcacag

SEQ ID NO: 802 - RNA, 23 nt, synthetic construct
  source 1..23: mol_type = other RNA
ccatgagacaaatgagccccagt

SEQ ID NO: 803 - RNA, 23 nt, synthetic construct
  source 1..23: mol_type = other RNA
gtaagaattaatagatggcaagt

SEQ ID NO: 804 - RNA, 23 nt, synthetic construct
  source 1..23: mol_type = other RNA
aaacaagcaaacaagcacagcct

SEQ ID NO: 805 - RNA, 23 nt, synthetic construct
  source 1..23: mol_type = other RNA
aagtaagaattaatagatggcaa

SEQ ID NO: 806 - RNA, 23 nt, synthetic construct
  source 1..23: mol_type = other RNA
aagagcacataaagggctatcac

SEQ ID NO: 807 - RNA, 23 nt, synthetic construct
  source 1..23: mol_type = other RNA
atcttaacatagcttgaggcaag

SEQ ID NO: 808 - RNA, 23 nt, synthetic construct
  source 1..23: mol_type = other RNA
cctttcatcatcagaagctccca

SEQ ID NO: 809 - RNA, 23 nt, synthetic construct
  source 1..23: mol_type = other RNA
cccaagacagtatagccaggcga

SEQ ID NO: 810 - RNA, 23 nt, synthetic construct
  source 1..23: mol_type = other RNA
catttaatagaaaaaggaaccag

SEQ ID NO: 811 - RNA, 23 nt, synthetic construct
  source 1..23: mol_type = other RNA
gcaagaacagtacagcagttcca

SEQ ID NO: 812 - RNA, 23 nt, synthetic construct
  source 1..23: mol_type = other RNA
cagcacaactgaaaagccccagt

SEQ ID NO: 813 - RNA, 23 nt, synthetic construct
  source 1..23: mol_type = other RNA
cagcaaaatcagacagcccacag

SEQ ID NO: 814 - RNA, 23 nt, synthetic construct
  source 1..23: mol_type = other RNA
agatccaaaatcagaagccaggt

SEQ ID NO: 815 - RNA, 23 nt, synthetic construct
  source 1..23: mol_type = other RNA
ttcaacaacacttagcagccagt

SEQ ID NO: 816 - RNA, 23 nt, synthetic construct
  source 1..23: mol_type = other RNA
actgttaaagacaggcaccacca

SEQ ID NO: 817 - RNA, 23 nt, synthetic construct
  source 1..23: mol_type = other RNA
gacaacatcagctaggaaagccc

SEQ ID NO: 818 - RNA, 23 nt, synthetic construct
  source 1..23: mol_type = other RNA
cccaatgatttcatagcagccag

SEQ ID NO: 819 - RNA, 23 nt, synthetic construct
  source 1..23: mol_type = other RNA
agcaaccaaagtttaaagcagcc

SEQ ID NO: 820 - RNA, 23 nt, synthetic construct
  source 1..23: mol_type = other RNA
aagcaaagacattcacagccaag

SEQ ID NO: 821 - RNA, 23 nt, synthetic construct
  source 1..23: mol_type = other RNA
cattcaagtttcaaagacaccgc

SEQ ID NO: 822 - RNA, 23 nt, synthetic construct
  source 1..23: mol_type = other RNA
caatacagacacagagaaggcca

SEQ ID NO: 823 - RNA, 23 nt, synthetic construct
  source 1..23: mol_type = other RNA
ccaactatgtaatacctcaccca

SEQ ID NO: 824 - RNA, 23 nt, synthetic construct
  source 1..23: mol_type = other RNA
acttataagcatgagacacagag

SEQ ID NO: 825 - RNA, 23 nt, synthetic construct
  source 1..23: mol_type = other RNA
aagccttgaatatggcagcatcc

SEQ ID NO: 826 - RNA, 23 nt, synthetic construct
  source 1..23: mol_type = other RNA
ccatagatgaaggtgagcagcag

SEQ ID NO: 827 - RNA, 23 nt, synthetic construct
  source 1..23: mol_type = other RNA
gcaaaatttaatttggagctcac

SEQ ID NO: 828 - RNA, 23 nt, synthetic construct
  source 1..23: mol_type = other RNA
gaaaacaaagagaaaacaggcag

SEQ ID NO: 829 - RNA, 23 nt, synthetic construct
  source 1..23: mol_type = other RNA
cagtacagtctttaatggcaagc

SEQ ID NO: 830 - RNA, 23 nt, synthetic construct
  source 1..23: mol_type = other RNA
gacatcatgaacaagtacacgga

SEQ ID NO: 831 - RNA, 23 nt, synthetic construct
  source 1..23: mol_type = other RNA
aaagagaaaaatcagtccctatg

SEQ ID NO: 832 - RNA, 23 nt, synthetic construct
  source 1..23: mol_type = other RNA
gaacatgaaggtagggacacaag

SEQ ID NO: 833 - RNA, 23 nt, synthetic construct
  source 1..23: mol_type = other RNA
aatgtaagacagaaaccagcaga

SEQ ID NO: 834 - RNA, 23 nt, synthetic construct
  source 1..23: mol_type = other RNA
taattacaggaaacagtctgcag

SEQ ID NO: 835 - RNA, 23 nt, synthetic construct
  source 1..23: mol_type = other RNA
agccttgaagtagtagaacaccc

SEQ ID NO: 836 - RNA, 23 nt, synthetic construct
  source 1..23: mol_type = other RNA
atcagtacaccaaacgaccacac

SEQ ID NO: 837 - RNA, 23 nt, synthetic construct
  source 1..23: mol_type = other RNA
caaacacatacttctcagccacg

SEQ ID NO: 838 - RNA, 23 nt, synthetic construct
  source 1..23: mol_type = other RNA
agaaaatgaaaataaagccccag

SEQ ID NO: 839 - RNA, 23 nt, synthetic construct
  source 1..23: mol_type = other RNA
acgtctatgtacacaacgagccg

SEQ ID NO: 840 - RNA, 23 nt, synthetic construct
  source 1..23: mol_type = other RNA
gaaccagtcaagtagtccacacc

SEQ ID NO: 841 - RNA, 23 nt, synthetic construct
  source 1..23: mol_type = other RNA
cttcatgatgtttgccagcccat

SEQ ID NO: 842 - RNA, 23 nt, synthetic construct
  source 1..23: mol_type = other RNA
cagcaccaaagaagagctcccct

SEQ ID NO: 843 - RNA, 23 nt, synthetic construct
  source 1..23: mol_type = other RNA
accatggttactaagagcccagt

SEQ ID NO: 844 - RNA, 23 nt, synthetic construct
  source 1..23: mol_type = other RNA
catagcatgtgtaagtcccaaca

SEQ ID NO: 845 - RNA, 23 nt, synthetic construct
  source 1..23: mol_type = other RNA
gaaaccagaacacacgaggctga

SEQ ID NO: 846 - RNA, 23 nt, synthetic construct
  source 1..23: mol_type = other RNA
actaagaaggaattttgcctcga

SEQ ID NO: 847 - RNA, 23 nt, synthetic construct
  source 1..23: mol_type = other RNA
aaattcgggaaatgggaggcatc

SEQ ID NO: 848 - RNA, 23 nt, synthetic construct
  source 1..23: mol_type = other RNA
caaatatttgtaggtgagcacca

SEQ ID NO: 849 - RNA, 23 nt, synthetic construct
  source 1..23: mol_type = other RNA
tgaatttgtcagaatgcagccat

SEQ ID NO: 850 - RNA, 23 nt, synthetic construct
  source 1..23: mol_type = other RNA
gacatgtttaaacttgcacagcg

SEQ ID NO: 851 - RNA, 23 nt, synthetic construct
  source 1..23: mol_type = other RNA
taaattctagacagtgagcgaca

SEQ ID NO: 852 - RNA, 23 nt, synthetic construct
  source 1..23: mol_type = other RNA
actttcaaatgtgtacacgcagc

SEQ ID NO: 853 - RNA, 23 nt, synthetic construct
  source 1..23: mol_type = other RNA
tctctcacacatatgcacaccca

SEQ ID NO: 854 - RNA, 23 nt, synthetic construct
  source 1..23: mol_type = other RNA
agacaaaaagaattggccccagc

SEQ ID NO: 855 - RNA, 23 nt, synthetic construct
  source 1..23: mol_type = other RNA
caaagtaatccacacacgccacg

SEQ ID NO: 856 - RNA, 23 nt, synthetic construct
  source 1..23: mol_type = other RNA
agaaaagaaatccagtctgcagt

SEQ ID NO: 857 - RNA, 23 nt, synthetic construct
  source 1..23: mol_type = other RNA
gacacctgagatttgagtaccca

SEQ ID NO: 858 - RNA, 23 nt, synthetic construct
  source 1..23: mol_type = other RNA
gttacaaataaaacagcccacac

SEQ ID NO: 859 - RNA, 23 nt, synthetic construct
  source 1..23: mol_type = other RNA
taagacaaaacagatgaggccca

SEQ ID NO: 860 - RNA, 23 nt, synthetic construct
  source 1..23: mol_type = other RNA
aaattcatctaaagggagccaga

SEQ ID NO: 861 - RNA, 23 nt, synthetic construct
  source 1..23: mol_type = other RNA
tacacaatctgatgaggccaaag

SEQ ID NO: 862 - RNA, 23 nt, synthetic construct
  source 1..23: mol_type = other RNA
cgaatagatgatttcctgctcga

SEQ ID NO: 863 - RNA, 23 nt, synthetic construct
  source 1..23: mol_type = other RNA
aacaattgcactgatgcccaacc

SEQ ID NO: 864 - RNA, 23 nt, synthetic construct
  source 1..23: mol_type = other RNA
gcctgataactttcagcaccaac

SEQ ID NO: 865 - RNA, 23 nt, synthetic construct
  source 1..23: mol_type = other RNA
cactataatgcattccaatccag

SEQ ID NO: 866 - RNA, 23 nt, synthetic construct
  source 1..23: mol_type = other RNA
gacagacatcagtctccaccgag

SEQ ID NO: 867 - RNA, 23 nt, synthetic construct
  source 1..23: mol_type = other RNA
aaacaatgcagtgctctggcctg

SEQ ID NO: 868 - RNA, 23 nt, synthetic construct
  source 1..23: mol_type = other RNA
agttactataaagagcagaccca

SEQ ID NO: 869 - RNA, 23 nt, synthetic construct
  source 1..23: mol_type = other RNA
aaaccagaagcaaagccgacatc

SEQ ID NO: 870 - RNA, 23 nt, synthetic construct
  source 1..23: mol_type = other RNA
ccacaggatacaaagcagagcta

SEQ ID NO: 871 - RNA, 23 nt, synthetic construct
  source 1..23: mol_type = other RNA
tccaacacagatacgtagctgcc

SEQ ID NO: 872 - RNA, 23 nt, synthetic construct
  source 1..23: mol_type = other RNA
caggatacaaagcagagctactc

SEQ ID NO: 873 - RNA, 23 nt, synthetic construct
  source 1..23: mol_type = other RNA
tgttcagaacaaaggccacagtt

SEQ ID NO: 874 - RNA, 23 nt, synthetic construct
  source 1..23: mol_type = other RNA
tccacgttgatttcgccgcccac

SEQ ID NO: 875 - RNA, 23 nt, synthetic construct
  source 1..23: mol_type = other RNA
tcggatcttcacctccagctcag

SEQ ID NO: 876 - RNA, 23 nt, synthetic construct
  source 1..23: mol_type = other RNA
agcaagctttaatggtctcaagc

SEQ ID NO: 877 - RNA, 23 nt, synthetic construct
  source 1..23: mol_type = other RNA
ccagccttgtcttcacatccagc

SEQ ID NO: 878 - RNA, 23 nt, synthetic construct
  source 1..23: mol_type = other RNA
tattcttgaaatcttccaccaca

SEQ ID NO: 879 - RNA, 23 nt, synthetic construct
  source 1..23: mol_type = other RNA
acaaactcattctcagcagccgt

SEQ ID NO: 880 - RNA, 23 nt, synthetic construct
  source 1..23: mol_type = other RNA
tctaacaagagctgggaagcaca

SEQ ID NO: 881 - RNA, 23 nt, synthetic construct
  source 1..23: mol_type = other RNA
ctgttcctgcagcagcgcccact

SEQ ID NO: 882 - RNA, 23 nt, synthetic construct
  source 1..23: mol_type = other RNA
atctcatcctgcagactgtccgc

SEQ ID NO: 883 - RNA, 23 nt, synthetic construct
  source 1..23: mol_type = other RNA
tccagtataaaagtgcagcccac

SEQ ID NO: 884 - RNA, 23 nt, synthetic construct
  source 1..23: mol_type = other RNA
cagatattaaagcatgcaccagg

SEQ ID NO: 885 - RNA, 23 nt, synthetic construct
  source 1..23: mol_type = other RNA
aatacttataggaacaccagcag

SEQ ID NO: 886 - RNA, 23 nt, synthetic construct
  source 1..23: mol_type = other RNA
aaagatagatcaattgaaggcag

SEQ ID NO: 887 - RNA, 23 nt, synthetic construct
  source 1..23: mol_type = other RNA
aaatgcatactagtagtggcatc

SEQ ID NO: 888 - RNA, 23 nt, synthetic construct
  source 1..23: mol_type = other RNA
cgttactcaagatcagagacccc

SEQ ID NO: 889 - RNA, 23 nt, synthetic construct
  source 1..23: mol_type = other RNA
ccacagtaacatagtaggcatgc

SEQ ID NO: 890 - RNA, 23 nt, synthetic construct
  source 1..23: mol_type = other RNA
acctgtagtagaaactcgcacag

SEQ ID NO: 891 - RNA, 23 nt, synthetic construct
  source 1..23: mol_type = other RNA
cagaccaagcaaaagcatacagg

SEQ ID NO: 892 - RNA, 23 nt, synthetic construct
  source 1..23: mol_type = other RNA
taagaaagagactcgaagccaca

SEQ ID NO: 893 - RNA, 23 nt, synthetic construct
  source 1..23: mol_type = other RNA
agaagcaaaaagccaaggagcag

SEQ ID NO: 894 - RNA, 23 nt, synthetic construct
  source 1..23: mol_type = other RNA
caaaaggacccaatcagacagct

SEQ ID NO: 895 - RNA, 23 nt, synthetic construct
  source 1..23: mol_type = other RNA
cttcgtagaaagttaggatccag

SEQ ID NO: 896 - RNA, 23 nt, synthetic construct
  source 1..23: mol_type = other RNA
agtcactgtgatgaccgccaacg

SEQ ID NO: 897 - RNA, 23 nt, synthetic construct
  source 1..23: mol_type = other RNA
cagacagacagacagacacacac

SEQ ID NO: 898 - RNA, 23 nt, synthetic construct
  source 1..23: mol_type = other RNA
aggtacagagcaaagagcatccc

SEQ ID NO: 899 - RNA, 23 nt, synthetic construct
  source 1..23: mol_type = other RNA
taaatgactttatagcaaggcct

SEQ ID NO: 900 - RNA, 23 nt, synthetic construct
  source 1..23: mol_type = other RNA
cagaagtcaccatcagatgccag

SEQ ID NO: 901 - RNA, 23 nt, synthetic construct
  source 1..23: mol_type = other RNA
aagactgttagatgctgtcagca

SEQ ID NO: 902 - RNA, 23 nt, synthetic construct
  source 1..23: mol_type = other RNA
catacagctgaagtgaccacaag

SEQ ID NO: 903 - RNA, 23 nt, synthetic construct
  source 1..23: mol_type = other RNA
agagtaaacaataaagagccacc

SEQ ID NO: 904 - RNA, 23 nt, synthetic construct
  source 1..23: mol_type = other RNA
aacatgaaagcattcagaggctt

SEQ ID NO: 905 - RNA, 23 nt, synthetic construct
  source 1..23: mol_type = other RNA
agaaaaagagaagtttgccaaga

SEQ ID NO: 906 - RNA, 23 nt, synthetic construct
  source 1..23: mol_type = other RNA
aaatcagaaactaagtgagacgg

SEQ ID NO: 907 - RNA, 23 nt, synthetic construct
  source 1..23: mol_type = other RNA
accaaagatgacttgatgtcggc

SEQ ID NO: 908 - RNA, 23 nt, synthetic construct
  source 1..23: mol_type = other RNA
gaaagcacaagagaggtcactga

SEQ ID NO: 909 - RNA, 23 nt, synthetic construct
  source 1..23: mol_type = other RNA
agaccaagaacacatggaggcat

SEQ ID NO: 910 - RNA, 23 nt, synthetic construct
  source 1..23: mol_type = other RNA
tttattaagacaaatgcggtccg

SEQ ID NO: 911 - RNA, 23 nt, synthetic construct
  source 1..23: mol_type = other RNA
cagtcagaagactccacccgaga

SEQ ID NO: 912 - RNA, 23 nt, synthetic construct
  source 1..23: mol_type = other RNA
cgaactttgagacattccccagg

SEQ ID NO: 913 - RNA, 23 nt, synthetic construct
  source 1..23: mol_type = other RNA
aaaggcattctaactagggcagc

SEQ ID NO: 914 - RNA, 23 nt, synthetic construct
  source 1..23: mol_type = other RNA
ttaagcgaaaagctgtctgccat

SEQ ID NO: 915 - RNA, 23 nt, synthetic construct
  source 1..23: mol_type = other RNA
acacaataataaatacatctgct

SEQ ID NO: 916 - RNA, 23 nt, synthetic construct
  source 1..23: mol_type = other RNA
acagcttgtcctctgacctccac

SEQ ID NO: 917 - RNA, 23 nt, synthetic construct
  source 1..23: mol_type = other RNA
tccacttccaggcagtgacgcgt

SEQ ID NO: 918 - RNA, 23 nt, synthetic construct
  source 1..23: mol_type = other RNA
ccttcacatatgatccacaccga

SEQ ID NO: 919 - RNA, 23 nt, synthetic construct
  source 1..23: mol_type = other RNA
atatcatgatggacttggacggg

SEQ ID NO: 920 - RNA, 23 nt, synthetic construct
  source 1..23: mol_type = other RNA
gggtacaccacaggagggattcc

SEQ ID NO: 921 - RNA, 23 nt, synthetic construct
  source 1..23: mol_type = other RNA
agaatttcttgttcaccatcacc

SEQ ID NO: 922 - RNA, 23 nt, synthetic construct
  source 1..23: mol_type = other RNA
ggaaagataagacacaggcagag

SEQ ID NO: 923 - RNA, 23 nt, synthetic construct
  source 1..23: mol_type = other RNA
gacatagtaataaatagacagct

SEQ ID NO: 924 - RNA, 23 nt, synthetic construct
  source 1..23: mol_type = other RNA
tttaaataataaataaaggcccc

SEQ ID NO: 925 - RNA, 23 nt, synthetic construct
  source 1..23: mol_type = other RNA
gacaccctaaaagtgagtcacag

SEQ ID NO: 926 - RNA, 23 nt, synthetic construct
  source 1..23: mol_type = other RNA
gcatttaacaatgtcccaaaccc

SEQ ID NO: 927 - RNA, 23 nt, synthetic construct
  source 1..23: mol_type = other RNA
gtaataggaaatgaagagagcat

SEQ ID NO: 928 - RNA, 23 nt, synthetic construct
  source 1..23: mol_type = other RNA
aagaccaaggtatagcagcactg

SEQ ID NO: 929 - RNA, 23 nt, synthetic construct
  source 1..23: mol_type = other RNA
ccaaatactatggatgagctgca

SEQ ID NO: 930 - RNA, 23 nt, synthetic construct
  source 1..23: mol_type = other RNA
caaaagtagcataggatggctga

SEQ ID NO: 931 - RNA, 23 nt, synthetic construct
  source 1..23: mol_type = other RNA
atgagaaaaagcaggaggaagaa

SEQ ID NO: 932 - RNA, 23 nt, synthetic construct
  source 1..23: mol_type = other RNA
attaattaaatagagtccactgg

SEQ ID NO: 933 - RNA, 23 nt, synthetic construct
  source 1..23: mol_type = other RNA
agttactagaatctgccagcaag

SEQ ID NO: 934 - RNA, 23 nt, synthetic construct
  source 1..23: mol_type = other RNA
aaaatgtagttaattgaagcagg

SEQ ID NO: 935 - RNA, 23 nt, synthetic construct
  source 1..23: mol_type = other RNA
caatgaagcataagacagctagc

SEQ ID NO: 936 - RNA, 23 nt, synthetic construct
  source 1..23: mol_type = other RNA
tcatcaaaactgaaggcccagaa

SEQ ID NO: 937 - RNA, 23 nt, synthetic construct
  source 1..23: mol_type = other RNA
caaagcgaaattcctgagccgag

SEQ ID NO: 938 - RNA, 23 nt, synthetic construct
  source 1..23: mol_type = other RNA
ttgaacagttcagagccaagccg

SEQ ID NO: 939 - RNA, 23 nt, synthetic construct
  source 1..23: mol_type = other RNA
caaacttgagacattcaacccag

SEQ ID NO: 940 - RNA, 23 nt, synthetic construct
  source 1..23: mol_type = other RNA
atcgacaactagaagcaaccaag

SEQ ID NO: 941 - RNA, 23 nt, synthetic construct
  source 1..23: mol_type = other RNA
gcaatcacaatggagtgagccat

SEQ ID NO: 942 - RNA, 23 nt, synthetic construct
  source 1..23: mol_type = other RNA
cagtgacaagtctttgaagcgcc

SEQ ID NO: 943 - RNA, 23 nt, synthetic construct
  source 1..23: mol_type = other RNA
atgagcatcagaaagagacagct

SEQ ID NO: 944 - RNA, 23 nt, synthetic construct
  source 1..23: mol_type = other RNA
agacgaatagacgacagccgcca

SEQ ID NO: 945 - RNA, 23 nt, synthetic construct
  source 1..23: mol_type = other RNA
cagcaccattatcatcagcaccc

SEQ ID NO: 946 - RNA, 23 nt, synthetic construct
  source 1..23: mol_type = other RNA
ttgcacatgtacacgctcaccac

SEQ ID NO: 947 - RNA, 23 nt, synthetic construct
  source 1..23: mol_type = other RNA
ggaacaacaaggtcagagcatga

SEQ ID NO: 948 - RNA, 23 nt, synthetic construct
  source 1..23: mol_type = other RNA
cgaagcgctgtatagaagcccag

SEQ ID NO: 949 - RNA, 23 nt, synthetic construct
  source 1..23: mol_type = other RNA
acaaggtacttagcctcctcaga

SEQ ID NO: 950 - RNA, 23 nt, synthetic construct
  source 1..23: mol_type = other RNA
cttggatgtccagtgagagcacc

SEQ ID NO: 951 - RNA, 23 nt, synthetic construct
  source 1..23: mol_type = other RNA
gttcaccgtcctacagggcactt

SEQ ID NO: 952 - RNA, 23 nt, synthetic construct
  source 1..23: mol_type = other RNA
cagcatggccaggagtagcagga

SEQ ID NO: 953 - RNA, 23 nt, synthetic construct
  source 1..23: mol_type = other RNA
caaaaattcgtgtgacggccaac

SEQ ID NO: 954 - RNA, 23 nt, synthetic construct
  source 1..23: mol_type = other RNA
aaatttaattagaaggatccagc

SEQ ID NO: 955 - RNA, 23 nt, synthetic construct
  source 1..23: mol_type = other RNA
agaactacgagcaaggccactag

SEQ ID NO: 956 - RNA, 23 nt, synthetic construct
  source 1..23: mol_type = other RNA
cacttataaaataggaagggcat

SEQ ID NO: 957 - RNA, 23 nt, synthetic construct
  source 1..23: mol_type = other RNA
tttaattagaaggatccagcagg

SEQ ID NO: 958 - RNA, 23 nt, synthetic construct
  source 1..23: mol_type = other RNA
aaaacccattatttggcagccag

SEQ ID NO: 959 - RNA, 23 nt, synthetic construct
  source 1..23: mol_type = other RNA
ttccagtagacatagccacgcac

SEQ ID NO: 960 - RNA, 23 nt, synthetic construct
  source 1..23: mol_type = other RNA
ccatcatgacttgagagcccaac

SEQ ID NO: 961 - RNA, 23 nt, synthetic construct
  source 1..23: mol_type = other RNA
tgagattaagaatcaaggcgcag

SEQ ID NO: 962 - RNA, 23 nt, synthetic construct
  source 1..23: mol_type = other RNA
aaaacaaaaccaaaaaccccagc

SEQ ID NO: 963 - RNA, 23 nt, synthetic construct
  source 1..23: mol_type = other RNA
tcagatttgatagtgaagcagcc

SEQ ID NO: 964 - RNA, 23 nt, synthetic construct
  source 1..23: mol_type = other RNA
ccatttaagaaacagcatccagg

SEQ ID NO: 965 - RNA, 23 nt, synthetic construct
  source 1..23: mol_type = other RNA
tgtatagaagtcatccaggacac

SEQ ID NO: 966 - RNA, 23 nt, synthetic construct
  source 1..23: mol_type = other RNA
gttgttatagtaacccatccaga

SEQ ID NO: 967 - RNA, 23 nt, synthetic construct
  source 1..23: mol_type = other RNA
agtgcttaatgacatcaccatgc

SEQ ID NO: 968 - RNA, 23 nt, synthetic construct
  source 1..23: mol_type = other RNA
attaacatatttaatccaagcgc

SEQ ID NO: 969 - RNA, 23 nt, synthetic construct
  source 1..23: mol_type = other RNA
tcccctaaatcatttgagcagag

SEQ ID NO: 970 - RNA, 23 nt, synthetic construct
  source 1..23: mol_type = other RNA
ctaaaagctagagtgtgtgaccc

SEQ ID NO: 971 - RNA, 23 nt, synthetic construct
  source 1..23: mol_type = other RNA
atgttaattctaactccgaccaa

SEQ ID NO: 972 - RNA, 23 nt, synthetic construct
  source 1..23: mol_type = other RNA
attaacatattccatggccaggg

SEQ ID NO: 973 - RNA, 23 nt, synthetic construct
  source 1..23: mol_type = other RNA
tcatttaataaggttagcacagc

SEQ ID NO: 974 - RNA, 23 nt, synthetic construct
  source 1..23: mol_type = other RNA
caaagcgatgaaagcctgcagca

SEQ ID NO: 975 - RNA, 23 nt, synthetic construct
  source 1..23: mol_type = other RNA
gcagtttaacaattagcactggt

SEQ ID NO: 976 - RNA, 23 nt, synthetic construct
  source 1..23: mol_type = other RNA
tgttaatatcaaatgccaacgac

SEQ ID NO: 977 - RNA, 23 nt, synthetic construct
  source 1..23: mol_type = other RNA
acaaccttttgaattcaggaccc

SEQ ID NO: 978 - RNA, 23 nt, synthetic construct
  source 1..23: mol_type = other RNA
taaaaataaaaccatcaggccca

SEQ ID NO: 979 - RNA, 23 nt, synthetic construct
  source 1..23: mol_type = other RNA
ccagaaatgacaaggagcagcaa

SEQ ID NO: 980 - RNA, 23 nt, synthetic construct
  source 1..23: mol_type = other RNA
aacaggacagttaagagccacag

SEQ ID NO: 981 - RNA, 23 nt, synthetic construct
  source 1..23: mol_type = other RNA
cagtgctttcacaatcgccacag

SEQ ID NO: 982 - RNA, 23 nt, synthetic construct
  source 1..23: mol_type = other RNA
acatccttaaaccaggacgcgac

SEQ ID NO: 983 - RNA, 23 nt, synthetic construct
  source 1..23: mol_type = other RNA
tcacttaacaagaaccgcggcca

SEQ ID NO: 984 - RNA, 23 nt, synthetic construct
  source 1..23: mol_type = other RNA
gcagagtaactcaagccaagcct

SEQ ID NO: 985 - RNA, 23 nt, synthetic construct
  source 1..23: mol_type = other RNA
ctcaccaaagatgcagcccacag

SEQ ID NO: 986 - RNA, 23 nt, synthetic construct
  source 1..23: mol_type = other RNA
aaaaccttgtatttcacaggcct

SEQ ID NO: 987 - RNA, 23 nt, synthetic construct
  source 1..23: mol_type = other RNA
tcctccagatcaaagccaactcc

SEQ ID NO: 988 - RNA, 23 nt, synthetic construct
  source 1..23: mol_type = other RNA
cgaagcgctgtatagaagcccag

SEQ ID NO: 989 - RNA, 23 nt, synthetic construct
  source 1..23: mol_type = other RNA
acaaggtacttagcctcctcaga

SEQ ID NO: 990 - RNA, 23 nt, synthetic construct
  source 1..23: mol_type = other RNA
cttggatgtccagtgagagcacc

SEQ ID NO: 991 - RNA, 23 nt, synthetic construct
  source 1..23: mol_type = other RNA
gttcaccgtcctacagggcactt

SEQ ID NO: 992 - RNA, 23 nt, synthetic construct
  source 1..23: mol_type = other RNA
cagcatggccaggagtagcagga

SEQ ID NO: 993 - RNA, 23 nt, synthetic construct
  source 1..23: mol_type = other RNA
gttttcacttacataccaccaga

SEQ ID NO: 994 - RNA, 23 nt, synthetic construct
  source 1..23: mol_type = other RNA
taaggaaaatataaacagccaat

SEQ ID NO: 995 - RNA, 23 nt, synthetic construct
  source 1..23: mol_type = other RNA
caaagcagagttctgtgacgagc

SEQ ID NO: 996 - RNA, 23 nt, synthetic construct
  source 1..23: mol_type = other RNA
ttgaacaatacacaatgtgctgc

SEQ ID NO: 997 - RNA, 23 nt, synthetic construct
  source 1..23: mol_type = other RNA
cttagtcataatatactggccaa

SEQ ID NO: 998 - RNA, 23 nt, synthetic construct
  source 1..23: mol_type = other RNA
aaacctagcatattgcagaccat

SEQ ID NO: 999 - RNA, 23 nt, synthetic construct
  source 1..23: mol_type = other RNA
tccagaaatgaactgcagcagca

SEQ ID NO: 1000 - RNA, 23 nt, synthetic construct
  source 1..23: mol_type = other RNA
cttgagtatagaaacgcccacat

SEQ ID NO: 1001 - RNA, 23 nt, synthetic construct
  source 1..23: mol_type = other RNA
cctccaatgattttccgaaccag

SEQ ID NO: 1002 - RNA, 23 nt, synthetic construct
  source 1..23: mol_type = other RNA
catgtcataaaatgccacgccaa

SEQ ID NO: 1003 - RNA, 23 nt, synthetic construct
  source 1..23: mol_type = other RNA
aacaaagtatctaagaccaccag

SEQ ID NO: 1004 - RNA, 23 nt, synthetic construct
  source 1..23: mol_type = other RNA
aaaatgaatccacagaagcagcc

SEQ ID NO: 1005 - RNA, 23 nt, synthetic construct
  source 1..23: mol_type = other RNA
catcttacagaagtacgacccat

SEQ ID NO: 1006 - RNA, 23 nt, synthetic construct
  source 1..23: mol_type = other RNA
ttaaagatataagcactcagctg

SEQ ID NO: 1007 - RNA, 23 nt, synthetic construct
  source 1..23: mol_type = other RNA
ctcatacagatgtggcgaagcag

SEQ ID NO: 1008 - RNA, 23 nt, synthetic construct
  source 1..23: mol_type = other RNA
ctactgatattgagaccgcacca

SEQ ID NO: 1009 - RNA, 23 nt, synthetic construct
  source 1..23: mol_type = other RNA
tacatcatctgtatgcagccaag

SEQ ID NO: 1010 - RNA, 23 nt, synthetic construct
  source 1..23: mol_type = other RNA
agtaagcacaaaattccacagag

SEQ ID NO: 1011 - RNA, 23 nt, synthetic construct
  source 1..23: mol_type = other RNA
acaccttataaaccgccggcaga

SEQ ID NO: 1012 - RNA, 23 nt, synthetic construct
  source 1..23: mol_type = other RNA
cacaaagaaattgatgaaccggt

SEQ ID NO: 1013 - RNA, 23 nt, synthetic construct
  source 1..23: mol_type = other RNA
aaaataaaaagatttaacccagc

SEQ ID NO: 1014 - RNA, 23 nt, synthetic construct
  source 1..23: mol_type = other RNA
acactaaaactatagcggtgcaa

SEQ ID NO: 1015 - RNA, 23 nt, synthetic construct
  source 1..23: mol_type = other RNA
atgaacattcaaaaaggatccac

SEQ ID NO: 1016 - RNA, 23 nt, synthetic construct
  source 1..23: mol_type = other RNA
tctgtcagctcgatgatgccacc

SEQ ID NO: 1017 - RNA, 23 nt, synthetic construct
  source 1..23: mol_type = other RNA
cgtcacaacaaagacaggctcct

SEQ ID NO: 1018 - RNA, 23 nt, synthetic construct
  source 1..23: mol_type = other RNA
agtacaatcagaagggaaggcca

SEQ ID NO: 1019 - RNA, 23 nt, synthetic construct
  source 1..23: mol_type = other RNA
gaaacccatatacacagacgagg

SEQ ID NO: 1020 - RNA, 23 nt, synthetic construct
  source 1..23: mol_type = other RNA
agtcacagaagtcaccagacgcg

SEQ ID NO: 1021 - RNA, 23 nt, synthetic construct
  source 1..23: mol_type = other RNA
tcctcatatacagtccactgcac

SEQ ID NO: 1022 - RNA, 23 nt, synthetic construct
  source 1..23: mol_type = other RNA
aattgtgttattcttcaggccca

SEQ ID NO: 1023 - RNA, 23 nt, synthetic construct
  source 1..23: mol_type = other RNA
acaaagatgatagcaccccaaag

SEQ ID NO: 1024 - RNA, 23 nt, synthetic construct
  source 1..23: mol_type = other RNA
aacaacttatccaagcagcgccg

SEQ ID NO: 1025 - RNA, 23 nt, synthetic construct
  source 1..23: mol_type = other RNA
ccatgaaactaagatcaggcaac

SEQ ID NO: 1026 - RNA, 23 nt, synthetic construct
  source 1..23: mol_type = other RNA
tccctgaagatcatcaaccccgc

SEQ ID NO: 1027 - RNA, 23 nt, synthetic construct
  source 1..23: mol_type = other RNA
cacgcatagactttccatctccc

SEQ ID NO: 1028 - RNA, 23 nt, synthetic construct
  source 1..23: mol_type = other RNA
aatatgtagtctacatcctcacg

SEQ ID NO: 1029 - RNA, 23 nt, synthetic construct
  source 1..23: mol_type = other RNA
aggcagatacaaaatacacagac

SEQ ID NO: 1030 - RNA, 23 nt, synthetic construct
  source 1..23: mol_type = other RNA
tagatcctgtaagtgaggtaccg

SEQ ID NO: 1031 - RNA, 23 nt, synthetic construct
  source 1..23: mol_type = other RNA
ttgcccagttgcttctagcccaa

SEQ ID NO: 1032 - RNA, 23 nt, synthetic construct
  source 1..23: mol_type = other RNA
attgccagagttgagttgtccag

SEQ ID NO: 1033 - RNA, 23 nt, synthetic construct
  source 1..23: mol_type = other RNA
agagccttctagaacacacgccg

SEQ ID NO: 1034 - RNA, 23 nt, synthetic construct
  source 1..23: mol_type = other RNA
agatatcagagtgcagagaccgc

SEQ ID NO: 1035 - RNA, 23 nt, synthetic construct
  source 1..23: mol_type = other RNA
tagatttatctacatcagctcag

SEQ ID NO: 1036 - RNA, 23 nt, synthetic construct
  source 1..23: mol_type = other RNA
aaaacatgtcaaaatgtcctgag

SEQ ID NO: 1037 - RNA, 23 nt, synthetic construct
  source 1..23: mol_type = other RNA
gcaatcatgttctggcaagcagt

SEQ ID NO: 1038 - RNA, 23 nt, synthetic construct
  source 1..23: mol_type = other RNA
ccatagaaaggaatccacgcagt

SEQ ID NO: 1039 - RNA, 23 nt, synthetic construct
  source 1..23: mol_type = other RNA
aaggacaaaccaatgcaacccag

SEQ ID NO: 1040 - RNA, 23 nt, synthetic construct
  source 1..23: mol_type = other RNA
gtctttgtaaataacccacccat

SEQ ID NO: 1041 - RNA, 23 nt, synthetic construct
  source 1..23: mol_type = other RNA
ccttgcctttcataaccacgcag

SEQ ID NO: 1042 - RNA, 23 nt, synthetic construct
  source 1..23: mol_type = other RNA
acagagataaaagccagaagcag

SEQ ID NO: 1043 - RNA, 23 nt, synthetic construct
  source 1..23: mol_type = other RNA
atgaagtacaagtgaccccagca

SEQ ID NO: 1044 - RNA, 23 nt, synthetic construct
  source 1..23: mol_type = other RNA
atcatcacagattgtgccccact

SEQ ID NO: 1045 - RNA, 23 nt, synthetic construct
  source 1..23: mol_type = other RNA
cattcagccatattggaccagta

SEQ ID NO: 1046 - RNA, 23 nt, synthetic construct
  source 1..23: mol_type = other RNA
atgctgtcattgaacgtgcgtca

SEQ ID NO: 1047 - RNA, 23 nt, synthetic construct
  source 1..23: mol_type = other RNA
ttcccaattcaaaagctgagctg

SEQ ID NO: 1048 - RNA, 23 nt, synthetic construct
  source 1..23: mol_type = other RNA
tctgaaaagctgattgagcgcat

SEQ ID NO: 1049 - RNA, 23 nt, synthetic construct
  source 1..23: mol_type = other RNA
tgagtaccagtagtaccgccaag

SEQ ID NO: 1050 - RNA, 23 nt, synthetic construct
  source 1..23: mol_type = other RNA
gagacaaataataagctagacga

SEQ ID NO: 1051 - RNA, 23 nt, synthetic construct
  source 1..23: mol_type = other RNA
ctgttttgagaatacccatccac

SEQ ID NO: 1052 - RNA, 23 nt, synthetic construct
  source 1..23: mol_type = other RNA
gtatggttatattcagtggcaca

SEQ ID NO: 1053 - RNA, 23 nt, synthetic construct
  source 1..23: mol_type = other RNA
cagtagtcaaataagcagccttg

SEQ ID NO: 1054 - RNA, 23 nt, synthetic construct
  source 1..23: mol_type = other RNA
agaacacatagttgcagagacca

SEQ ID NO: 1055 - RNA, 23 nt, synthetic construct
  source 1..23: mol_type = other RNA
gccaatgtcagtttccaccacca

SEQ ID NO: 1056 - RNA, 23 nt, synthetic construct
  source 1..23: mol_type = other RNA
gcatagtaacagtggccatcaag

SEQ ID NO: 1057 - RNA, 23 nt, synthetic construct
  source 1..23: mol_type = other RNA
atcaaaagtgatgtagtggccat

SEQ ID NO: 1058 - RNA, 23 nt, synthetic construct
  source 1..23: mol_type = other RNA
atactatttaagtttgaggcagt

SEQ ID NO: 1059 - RNA, 23 nt, synthetic construct
  source 1..23: mol_type = other RNA
catgcattttaattccagcgcat

SEQ ID NO: 1060 - RNA, 23 nt, synthetic construct
  source 1..23: mol_type = other RNA
aagttatttacatttcaaggccc

SEQ ID NO: 1061 - RNA, 23 nt, synthetic construct
  source 1..23: mol_type = other RNA
ctggaattactacagcgagtcag

SEQ ID NO: 1062 - RNA, 23 nt, synthetic construct
  source 1..23: mol_type = other RNA
ccgcaacataggatggagagcag

SEQ ID NO: 1063 - RNA, 23 nt, synthetic construct
  source 1..23: mol_type = other RNA
tcatactaaagtatacaggccgt

SEQ ID NO: 1064 - RNA, 23 nt, synthetic construct
  source 1..23: mol_type = other RNA
aaacgtttagcaagtccgagcgt

SEQ ID NO: 1065 - RNA, 23 nt, synthetic construct
  source 1..23: mol_type = other RNA
tcaaaatgtgcaaagtggcagtg

SEQ ID NO: 1066 - RNA, 23 nt, synthetic construct
  source 1..23: mol_type = other RNA
acagacacactagtgaccgcagc

SEQ ID NO: 1067 - RNA, 23 nt, synthetic construct
  source 1..23: mol_type = other RNA
aaacaaggaagaaacaggctagg

SEQ ID NO: 1068 - RNA, 23 nt, synthetic construct
  source 1..23: mol_type = other RNA
ctctgaaaacaaaatgacccagc

SEQ ID NO: 1069 - RNA, 23 nt, synthetic construct
  source 1..23: mol_type = other RNA
acggtaactaagtagaacagccc

SEQ ID NO: 1070 - RNA, 23 nt, synthetic construct
  source 1..23: mol_type = other RNA
taaggctaaatcaaacgtgcctt

SEQ ID NO: 1071 - RNA, 23 nt, synthetic construct
  source 1..23: mol_type = other RNA
ttatttattgtatatgagcacac

SEQ ID NO: 1072 - RNA, 23 nt, synthetic construct
  source 1..23: mol_type = other RNA
aataaaatttcttggccaggcaa

SEQ ID NO: 1073 - RNA, 23 nt, synthetic construct
  source 1..23: mol_type = other RNA
accaagttgtaaatagagctcag

SEQ ID NO: 1074 - RNA, 23 nt, synthetic construct
  source 1..23: mol_type = other RNA
acagtgtataccaagacccacgc

SEQ ID NO: 1075 - RNA, 23 nt, synthetic construct
  source 1..23: mol_type = other RNA
catacgtaacaagatctgacgcc

SEQ ID NO: 1076 - RNA, 23 nt, synthetic construct
  source 1..23: mol_type = other RNA
caaagacaattagagctatgcag

SEQ ID NO: 1077 - RNA, 23 nt, synthetic construct
  source 1..23: mol_type = other RNA
agagcatctcagtagcagaccct

SEQ ID NO: 1078 - RNA, 23 nt, synthetic construct
  source 1..23: mol_type = other RNA
cactttgaagaagtccagcaggt

SEQ ID NO: 1079 - RNA, 23 nt, synthetic construct
  source 1..23: mol_type = other RNA
gacatgaaagcaaagtctccgca

SEQ ID NO: 1080 - RNA, 23 nt, synthetic construct
  source 1..23: mol_type = other RNA
tcaagttgacgtagaccagccag

SEQ ID NO: 1081 - RNA, 23 nt, synthetic construct
  source 1..23: mol_type = other RNA
gaacatgtacacatagtgctggc

SEQ ID NO: 1082 - RNA, 23 nt, synthetic construct
  source 1..23: mol_type = other RNA
tgaaattacagatgtgagcctcg

SEQ ID NO: 1083 - RNA, 23 nt, synthetic construct
  source 1..23: mol_type = other RNA
gctaattacacagagcagacacc

SEQ ID NO: 1084 - RNA, 23 nt, synthetic construct
  source 1..23: mol_type = other RNA
gtaacttaaaacttccagccaca

SEQ ID NO: 1085 - RNA, 23 nt, synthetic construct
  source 1..23: mol_type = other RNA
tgcagaaagttatggccagacag

SEQ ID NO: 1086 - RNA, 23 nt, synthetic construct
  source 1..23: mol_type = other RNA
taactacaaagatagctgacaca

SEQ ID NO: 1087 - RNA, 23 nt, synthetic construct
  source 1..23: mol_type = other RNA
cccaatgaacagctgtagcgtga

SEQ ID NO: 1088 - RNA, 23 nt, synthetic construct
  source 1..23: mol_type = other RNA
tcatcctcatagaaccgaacctc

SEQ ID NO: 1089 - RNA, 23 nt, synthetic construct
  source 1..23: mol_type = other RNA
ccagattattaaattgagcagtc

SEQ ID NO: 1090 - RNA, 23 nt, synthetic construct
  source 1..23: mol_type = other RNA
catatcgaaatctgaagcctcgc

SEQ ID NO: 1091 - RNA, 23 nt, synthetic construct
  source 1..23: mol_type = other RNA
agtcaactaacagatccaggcac

SEQ ID NO: 1092 - RNA, 23 nt, synthetic construct
  source 1..23: mol_type = other RNA
aataagatttcgattagacaggg

SEQ ID NO: 1093 - RNA, 23 nt, synthetic construct
  source 1..23: mol_type = other RNA
acatttacaagaaggcgacacag

SEQ ID NO: 1094 - RNA, 23 nt, synthetic construct
  source 1..23: mol_type = other RNA
gatttcacaaagacaacagccga

SEQ ID NO: 1095 - RNA, 23 nt, synthetic construct
  source 1..23: mol_type = other RNA
caacaagagcgaaaccaggtcag

SEQ ID NO: 1096 - RNA, 23 nt, synthetic construct
  source 1..23: mol_type = other RNA
tttccacttataatgtcagacgc

SEQ ID NO: 1097 - RNA, 23 nt, synthetic construct
  source 1..23: mol_type = other RNA
caaagtcaccaagtgctccacga

SEQ ID NO: 1098 - RNA, 23 nt, synthetic construct
  source 1..23: mol_type = other RNA
atcagatctgagatgatcaccgg

SEQ ID NO: 1099 - RNA, 23 nt, synthetic construct
  source 1..23: mol_type = other RNA
gacgcacaaacatgggaactagg

SEQ ID NO: 1100 - RNA, 23 nt, synthetic construct
  source 1..23: mol_type = other RNA
agaagacctccaagccaagacga

SEQ ID NO: 1101 - RNA, 23 nt, synthetic construct
  source 1..23: mol_type = other RNA
ccacaggaacctgagctactccg

SEQ ID NO: 1102 - RNA, 23 nt, synthetic construct
  source 1..23: mol_type = other RNA
tctaaggaagtcaaggttgacca

SEQ ID NO: 1103 - RNA, 23 nt, synthetic construct
  source 1..23: mol_type = other RNA
ccgagcatcaaatacctgcagct

SEQ ID NO: 1104 - RNA, 23 nt, synthetic construct
  source 1..23: mol_type = other RNA
gaaccttcaagatttaggccgac

SEQ ID NO: 1105 - RNA, 23 nt, synthetic construct
  source 1..23: mol_type = other RNA
caaagcatatgaagagggcagca

SEQ ID NO: 1106 - RNA, 23 nt, synthetic construct
  source 1..23: mol_type = other RNA
aagaaagatcttaaattggcagc

SEQ ID NO: 1107 - RNA, 23 nt, synthetic construct
  source 1..23: mol_type = other RNA
gaactcatgtaccagccgctgaa

SEQ ID NO: 1108 - RNA, 23 nt, synthetic construct
  source 1..23: mol_type = other RNA
tgccttcaattcatgccacccac

SEQ ID NO: 1109 - RNA, 23 nt, synthetic construct
  source 1..23: mol_type = other RNA
tcttcattcattcaactccgccg

SEQ ID NO: 1110 - RNA, 23 nt, synthetic construct
  source 1..23: mol_type = other RNA
ggaaacagtcaaaagctgctgca

SEQ ID NO: 1111 - RNA, 23 nt, synthetic construct
  source 1..23: mol_type = other RNA
gaaccacttctttaaccatccca

SEQ ID NO: 1112 - RNA, 23 nt, synthetic construct
  source 1..23: mol_type = other RNA
agctgattcaaaaagcaggtcct

SEQ ID NO: 1113 - RNA, 23 nt, synthetic construct
  source 1..23: mol_type = other RNA
cttttaaatagaaaccacccagt

SEQ ID NO: 1114 - RNA, 23 nt, synthetic construct
  source 1..23: mol_type = other RNA
accgagataactgttcaagcccc

SEQ ID NO: 1115 - RNA, 23 nt, synthetic construct
  source 1..23: mol_type = other RNA
ttgcatttatgaatacagagcag

SEQ ID NO: 1116 - RNA, 23 nt, synthetic construct
  source 1..23: mol_type = other RNA
tacgtaatcactaggcgcccaag

SEQ ID NO: 1117 - RNA, 23 nt, synthetic construct
  source 1..23: mol_type = other RNA
gtactcagtcatttcacaccagc

SEQ ID NO: 1118 - RNA, 23 nt, synthetic construct
  source 1..23: mol_type = other RNA
tttccgagaagaatccaccacag

SEQ ID NO: 1119 - RNA, 23 nt, synthetic construct
  source 1..23: mol_type = other RNA
agtttcagagatttgtgcagcaa

SEQ ID NO: 1120 - RNA, 23 nt, synthetic construct
  source 1..23: mol_type = other RNA
aaagcagagtaacagagtcccca

SEQ ID NO: 1121 - RNA, 23 nt, synthetic construct
  source 1..23: mol_type = other RNA
catagatataccagtttcccagg

SEQ ID NO: 1122 - RNA, 23 nt, synthetic construct
  source 1..23: mol_type = other RNA
caaagatgatgtaggtgcactcc

SEQ ID NO: 1123 - RNA, 23 nt, synthetic construct
  source 1..23: mol_type = other RNA
tacaattacaagatagtccaagg

SEQ ID NO: 1124 - RNA, 23 nt, synthetic construct
  source 1..23: mol_type = other RNA
agatgtattcagaaaccacgctg

SEQ ID NO: 1125 - RNA, 23 nt, synthetic construct
  source 1..23: mol_type = other RNA
ctttcggttaatatcgtacacca

SEQ ID NO: 1126 - RNA, 23 nt, synthetic construct
  source 1..23: mol_type = other RNA
atctcaaaaccagagtgccccag

SEQ ID NO: 1127 - RNA, 23 nt, synthetic construct
  source 1..23: mol_type = other RNA
ctgagagacaacaagagcccaaa

SEQ ID NO: 1128 - RNA, 23 nt, synthetic construct
  source 1..23: mol_type = other RNA
cagaagtaaccatggcatccgtg

SEQ ID NO: 1129 - RNA, 23 nt, synthetic construct
  source 1..23: mol_type = other RNA
aataatttatatgtggggacaga

SEQ ID NO: 1130 - RNA, 23 nt, synthetic construct
  source 1..23: mol_type = other RNA
cataaaacaagatggacatcacc

SEQ ID NO: 1131 - RNA, 23 nt, synthetic construct
  source 1..23: mol_type = other RNA
ataatttatatgtggggacagat

SEQ ID NO: 1132 - RNA, 23 nt, synthetic construct
  source 1..23: mol_type = other RNA
ccagtgtttgtcatcaatccccc

SEQ ID NO: 1133 - RNA, 23 nt, synthetic construct
  source 1..23: mol_type = other RNA
agcacagagaagaacggcacttc

SEQ ID NO: 1134 - RNA, 23 nt, synthetic construct
  source 1..23: mol_type = other RNA
tcagcagttttcattgcagccag

SEQ ID NO: 1135 - RNA, 23 nt, synthetic construct
  source 1..23: mol_type = other RNA
tataattccagctttggcagcaa

SEQ ID NO: 1136 - RNA, 23 nt, synthetic construct
  source 1..23: mol_type = other RNA
acatcaggaacttcacagcttcg

SEQ ID NO: 1137 - RNA, 23 nt, synthetic construct
  source 1..23: mol_type = other RNA
tgctccataagcgtccgccacct

SEQ ID NO: 1138 - RNA, 23 nt, synthetic construct
  source 1..23: mol_type = other RNA
gtggaaatttaagtccagcacat

SEQ ID NO: 1139 - RNA, 23 nt, synthetic construct
  source 1..23: mol_type = other RNA
aaacaaagagaagtccaagacag

SEQ ID NO: 1140 - RNA, 23 nt, synthetic construct
  source 1..23: mol_type = other RNA
tccatgtactcagagtagacccg

SEQ ID NO: 1141 - RNA, 23 nt, synthetic construct
  source 1..23: mol_type = other RNA
ctactcaaggaattccgcccata

SEQ ID NO: 1142 - RNA, 23 nt, synthetic construct
  source 1..23: mol_type = other RNA
gcctctcattacctgcagccgag

SEQ ID NO: 1143 - RNA, 23 nt, synthetic construct
  source 1..23: mol_type = other RNA
cagagcaaagataccagcagcca

SEQ ID NO: 1144 - RNA, 23 nt, synthetic construct
  source 1..23: mol_type = other RNA
acaggataaactgtagcagccaa

SEQ ID NO: 1145 - RNA, 23 nt, synthetic construct
  source 1..23: mol_type = other RNA
aagctcaattattagtggcagag

SEQ ID NO: 1146 - RNA, 23 nt, synthetic construct
  source 1..23: mol_type = other RNA
agcaagatcagagacacaccagc

SEQ ID NO: 1147 - RNA, 23 nt, synthetic construct
  source 1..23: mol_type = other RNA
tccagcattcttatgcctccaac

SEQ ID NO: 1148 - RNA, 23 nt, synthetic construct
  source 1..23: mol_type = other RNA
agttcagcataagatcctccgac

SEQ ID NO: 1149 - RNA, 23 nt, synthetic construct
  source 1..23: mol_type = other RNA
caaaccattacagaaggcggcct

SEQ ID NO: 1150 - RNA, 23 nt, synthetic construct
  source 1..23: mol_type = other RNA
aagtccctagaagtgcccaacag

SEQ ID NO: 1151 - RNA, 23 nt, synthetic construct
  source 1..23: mol_type = other RNA
agcagcaatacagggatacccac

SEQ ID NO: 1152 - RNA, 23 nt, synthetic construct
  source 1..23: mol_type = other RNA
ccagtctacgaatttcccacctg

SEQ ID NO: 1153 - RNA, 23 nt, synthetic construct
  source 1..23: mol_type = other RNA
atgaaaacatgaagtggccacgt

SEQ ID NO: 1154 - RNA, 23 nt, synthetic construct
  source 1..23: mol_type = other RNA
aagtagaaacaaataccacagtg

SEQ ID NO: 1155 - RNA, 23 nt, synthetic construct
  source 1..23: mol_type = other RNA
caaaagtgcaaatggcaggtcct

SEQ ID NO: 1156 - RNA, 23 nt, synthetic construct
  source 1..23: mol_type = other RNA
gcattccagaacatgcagctgaa

SEQ ID NO: 1157 - RNA, 23 nt, synthetic construct
  source 1..23: mol_type = other RNA
tacaacaattaccttgtgactca

SEQ ID NO: 1158 - RNA, 23 nt, synthetic construct
  source 1..23: mol_type = other RNA
tccaaaatcaaaatgacgaccac

SEQ ID NO: 1159 - RNA, 23 nt, synthetic construct
  source 1..23: mol_type = other RNA
gtccacgaaatttcgcagagcat

SEQ ID NO: 1160 - RNA, 23 nt, synthetic construct
  source 1..23: mol_type = other RNA
atttcctaaattacccacccacc

SEQ ID NO: 1161 - RNA, 23 nt, synthetic construct
  source 1..23: mol_type = other RNA
actgatacatgaaaggacccagc

SEQ ID NO: 1162 - RNA, 23 nt, synthetic construct
  source 1..23: mol_type = other RNA
aggaattcaaatttgggcccaca

SEQ ID NO: 1163 - RNA, 23 nt, synthetic construct
  source 1..23: mol_type = other RNA
ccaagataaaggttgccacgcag

SEQ ID NO: 1164 - RNA, 23 nt, synthetic construct
  source 1..23: mol_type = other RNA
agcgcactatttatgacccagag

SEQ ID NO: 1165 - RNA, 23 nt, synthetic construct
  source 1..23: mol_type = other RNA
agtgtttcaattatagagcacgt

SEQ ID NO: 1166 - RNA, 23 nt, synthetic construct
  source 1..23: mol_type = other RNA
agtagaaatctagagcgaccact

SEQ ID NO: 1167 - RNA, 23 nt, synthetic construct
  source 1..23: mol_type = other RNA
aagcagatattgatcaccccagc

SEQ ID NO: 1168 - RNA, 23 nt, synthetic construct
  source 1..23: mol_type = other RNA
ccaagaacaaatgtggccacaca

SEQ ID NO: 1169 - RNA, 23 nt, synthetic construct
  source 1..23: mol_type = other RNA
accctttaaagaatgcagcagag

SEQ ID NO: 1170 - RNA, 23 nt, synthetic construct
  source 1..23: mol_type = other RNA
agcaattcaaatgaaaggcagag

SEQ ID NO: 1171 - RNA, 23 nt, synthetic construct
  source 1..23: mol_type = other RNA
caagaacaagatgtaggaaccag

SEQ ID NO: 1172 - RNA, 23 nt, synthetic construct
  source 1..23: mol_type = other RNA
tcatatcgaaaatgccagagcaa

SEQ ID NO: 1173 - RNA, 23 nt, synthetic construct
  source 1..23: mol_type = other RNA
cacagcaaggaattcagcctgta

SEQ ID NO: 1174 - RNA, 23 nt, synthetic construct
  source 1..23: mol_type = other RNA
acaaaccaaagatggacctcagc

SEQ ID NO: 1175 - RNA, 23 nt, synthetic construct
  source 1..23: mol_type = other RNA
gtagaacaatatgctcacacagc

SEQ ID NO: 1176 - RNA, 23 nt, synthetic construct
  source 1..23: mol_type = other RNA
tgttcaagaacaaaggagggcat

SEQ ID NO: 1177 - RNA, 23 nt, synthetic construct
  source 1..23: mol_type = other RNA
gcatcaatgaaggtgagcatctc

SEQ ID NO: 1178 - RNA, 23 nt, synthetic construct
  source 1..23: mol_type = other RNA
ctttactcagataaaaccagcgg

SEQ ID NO: 1179 - RNA, 23 nt, synthetic construct
  source 1..23: mol_type = other RNA
ttttatgtacagtcagcagaccc

SEQ ID NO: 1180 - RNA, 23 nt, synthetic construct
  source 1..23: mol_type = other RNA
aaaaaccaaaccaaacccccaac

SEQ ID NO: 1181 - RNA, 23 nt, synthetic construct
  source 1..23: mol_type = other RNA
ccatttaataaggtgctgacact

SEQ ID NO: 1182 - RNA, 23 nt, synthetic construct
  source 1..23: mol_type = other RNA
cagaagtctaatgacgcccgaga

SEQ ID NO: 1183 - RNA, 23 nt, synthetic construct
  source 1..23: mol_type = other RNA
agagtacaataacaacaggccaa

SEQ ID NO: 1184 - RNA, 23 nt, synthetic construct
  source 1..23: mol_type = other RNA
acgagtaatagcatccaaagccc

SEQ ID NO: 1185 - RNA, 23 nt, synthetic construct
  source 1..23: mol_type = other RNA
gttcatgtaagtattgcaaccca

SEQ ID NO: 1186 - RNA, 23 nt, synthetic construct
  source 1..23: mol_type = other RNA
catatataagtacaccaaggccc

SEQ ID NO: 1187 - RNA, 23 nt, synthetic construct
  source 1..23: mol_type = other RNA
ttttgcattatcaaaagccagct

SEQ ID NO: 1188 - RNA, 23 nt, synthetic construct
  source 1..23: mol_type = other RNA
actcagacagaaaaacagcagca

SEQ ID NO: 1189 - RNA, 23 nt, synthetic construct
  source 1..23: mol_type = other RNA
tttacagtagaaaggcaccaaga

SEQ ID NO: 1190 - RNA, 23 nt, synthetic construct
  source 1..23: mol_type = other RNA
attatgcagcaagagatcccagc

SEQ ID NO: 1191 - RNA, 23 nt, synthetic construct
  source 1..23: mol_type = other RNA
ttccagagaaagaaaccaggcct

SEQ ID NO: 1192 - RNA, 23 nt, synthetic construct
  source 1..23: mol_type = other RNA
acaatcatgcttctgacagcacc

SEQ ID NO: 1193 - RNA, 23 nt, synthetic construct
  source 1..23: mol_type = other RNA
agaaacagaattttcagaagccc

SEQ ID NO: 1194 - RNA, 23 nt, synthetic construct
  source 1..23: mol_type = other RNA
tgactacacaagaaggacctcca

SEQ ID NO: 1195 - RNA, 23 nt, synthetic construct
  source 1..23: mol_type = other RNA
tgaaccaatctgtcgagcacagc

SEQ ID NO: 1196 - RNA, 23 nt, synthetic construct
  source 1..23: mol_type = other RNA
cccagaaacatttgcacagtcag

SEQ ID NO: 1197 - RNA, 23 nt, synthetic construct
  source 1..23: mol_type = other RNA
cacatatttcgttgatgagccct

SEQ ID NO: 1198 - RNA, 23 nt, synthetic construct
  source 1..23: mol_type = other RNA
gttagatacagaaatacaccagc

SEQ ID NO: 1199 - RNA, 23 nt, synthetic construct
  source 1..23: mol_type = other RNA
tttataattaatttcagagccat

SEQ ID NO: 1200 - RNA, 23 nt, synthetic construct
  source 1..23: mol_type = other RNA
caatatcattatggtaggccagg

SEQ ID NO: 1201 - RNA, 23 nt, synthetic construct
  source 1..23: mol_type = other RNA
gaaatcatttatttcccaacagc

SEQ ID NO: 1202 - RNA, 23 nt, synthetic construct
  source 1..23: mol_type = other RNA
agaagtacttgtaggacacgcat

SEQ ID NO: 1203 - RNA, 23 nt, synthetic construct
  source 1..23: mol_type = other RNA
atccagagatagtaccttgcaca

SEQ ID NO: 1204 - RNA, 23 nt, synthetic construct
  source 1..23: mol_type = other RNA
cagaaattacagctgtggcagat

SEQ ID NO: 1205 - RNA, 23 nt, synthetic construct
  source 1..23: mol_type = other RNA
cttgacaattggagcagagccac

SEQ ID NO: 1206 - RNA, 23 nt, synthetic construct
  source 1..23: mol_type = other RNA
aaaaccgtgtttacttagcaggc

SEQ ID NO: 1207 - RNA, 23 nt, synthetic construct
  source 1..23: mol_type = other RNA
tccagagaaggaattcccagcat

SEQ ID NO: 1208 - RNA, 23 nt, synthetic construct
  source 1..23: mol_type = other RNA
cacaaagtagaggtagcccatga

SEQ ID NO: 1209 - RNA, 23 nt, synthetic construct
  source 1..23: mol_type = other RNA
ccagcatccaaaagtgacccagt

SEQ ID NO: 1210 - RNA, 23 nt, synthetic construct
  source 1..23: mol_type = other RNA
caaagagcaagtttaaggctcgt

SEQ ID NO: 1211 - RNA, 23 nt, synthetic construct
  source 1..23: mol_type = other RNA
gcctcagacagaaagcccacaaa

SEQ ID NO: 1212 - RNA, 23 nt, synthetic construct
  source 1..23: mol_type = other RNA
aagcagatggtaagaagcagcca

SEQ ID NO: 1213 - RNA, 23 nt, synthetic construct
  source 1..23: mol_type = other RNA
aacaaaatttagcaggaaaccca

SEQ ID NO: 1214 - RNA, 23 nt, synthetic construct
  source 1..23: mol_type = other RNA
atggttaataaaaagccccagat

SEQ ID NO: 1215 - RNA, 23 nt, synthetic construct
  source 1..23: mol_type = other RNA
agcacagttcacagtgacaaccc

SEQ ID NO: 1216 - RNA, 23 nt, synthetic construct
  source 1..23: mol_type = other RNA
gcagaattagcttaagaaggcgt

SEQ ID NO: 1217 - RNA, 23 nt, synthetic construct
  source 1..23: mol_type = other RNA
ggcagcattcatatagcacagtg

SEQ ID NO: 1218 - RNA, 23 nt, synthetic construct
  source 1..23: mol_type = other RNA
acctttccaaagagaacgcccag

SEQ ID NO: 1219 - RNA, 23 nt, synthetic construct
  source 1..23: mol_type = other RNA
acaaaatgatgagtgacagacag

SEQ ID NO: 1220 - RNA, 23 nt, synthetic construct
  source 1..23: mol_type = other RNA
ttacagaaccatactcgaaccac

SEQ ID NO: 1221 - RNA, 23 nt, synthetic construct
  source 1..23: mol_type = other RNA
cacttcagaaacatggtctccag

SEQ ID NO: 1222 - RNA, 23 nt, synthetic construct
  source 1..23: mol_type = other RNA
agtaaaagaatttaaccccaaag

SEQ ID NO: 1223 - RNA, 23 nt, synthetic construct
  source 1..23: mol_type = other RNA
gcattaatcaaatgtccgcgaac

SEQ ID NO: 1224 - RNA, 23 nt, synthetic construct
  source 1..23: mol_type = other RNA
cagttaatagaacctgcccaagg

SEQ ID NO: 1225 - RNA, 23 nt, synthetic construct
  source 1..23: mol_type = other RNA
gcaacagacttaatgctgcagtt

SEQ ID NO: 1226 - RNA, 23 nt, synthetic construct
  source 1..23: mol_type = other RNA
atccacttctaatttcagcgcat

SEQ ID NO: 1227 - RNA, 23 nt, synthetic construct
  source 1..23: mol_type = other RNA
acatatatgtgtgtgaagagcag

SEQ ID NO: 1228 - RNA, 23 nt, synthetic construct
  source 1..23: mol_type = other RNA
ccaataagaaatatggccacaag

SEQ ID NO: 1229 - RNA, 23 nt, synthetic construct
  source 1..23: mol_type = other RNA
agatttaaaaacaaagaccacca

SEQ ID NO: 1230 - RNA, 23 nt, synthetic construct
  source 1..23: mol_type = other RNA
aagcaagaatgcagcccgacaag

SEQ ID NO: 1231 - RNA, 23 nt, synthetic construct
  source 1..23: mol_type = other RNA
gacacagataactagacacccat

SEQ ID NO: 1232 - RNA, 23 nt, synthetic construct
  source 1..23: mol_type = other RNA
ccaacgatatcaaacggagacct

SEQ ID NO: 1233 - RNA, 23 nt, synthetic construct
  source 1..23: mol_type = other RNA
aaacaaacatttattgagccagg

SEQ ID NO: 1234 - RNA, 23 nt, synthetic construct
  source 1..23: mol_type = other RNA
taatcaaacagatgacagcagag

SEQ ID NO: 1235 - RNA, 23 nt, synthetic construct
  source 1..23: mol_type = other RNA
cattttacaaatgatgagcagct

SEQ ID NO: 1236 - RNA, 23 nt, synthetic construct
  source 1..23: mol_type = other RNA
gcaaaacagaactagaacccagg

SEQ ID NO: 1237 - RNA, 23 nt, synthetic construct
  source 1..23: mol_type = other RNA
ttcagcagaataacccaggctta

SEQ ID NO: 1238 - RNA, 23 nt, synthetic construct
  source 1..23: mol_type = other RNA
catcacagtaacaagagtagcct

SEQ ID NO: 1239 - RNA, 23 nt, synthetic construct
  source 1..23: mol_type = other RNA
gtcacattcatttgccaaccctc

SEQ ID NO: 1240 - RNA, 23 nt, synthetic construct
  source 1..23: mol_type = other RNA
cagaagcaagtaaaagcagcttg

SEQ ID NO: 1241 - RNA, 23 nt, synthetic construct
  source 1..23: mol_type = other RNA
aaagagatattagaagaacagga

SEQ ID NO: 1242 - RNA, 23 nt, synthetic construct
  source 1..23: mol_type = other RNA
agcctcgtatacatccatccaga

SEQ ID NO: 1243 - RNA, 23 nt, synthetic construct
  source 1..23: mol_type = other RNA
tgcaattataaacttggagcaag

SEQ ID NO: 1244 - RNA, 23 nt, synthetic construct
  source 1..23: mol_type = other RNA
gtagcacagatacagaaccgcag

SEQ ID NO: 1245 - RNA, 23 nt, synthetic construct
  source 1..23: mol_type = other RNA
cattccagagcttagcacaaccg

SEQ ID NO: 1246 - RNA, 23 nt, synthetic construct
  source 1..23: mol_type = other RNA
acaaaacatcagtgactgccccc

SEQ ID NO: 1247 - RNA, 23 nt, synthetic construct
  source 1..23: mol_type = other RNA
cagaaaacatctgtaggggacag

SEQ ID NO: 1248 - RNA, 23 nt, synthetic construct
  source 1..23: mol_type = other RNA
aacagatgatgaataccgccaag

SEQ ID NO: 1249 - RNA, 23 nt, synthetic construct
  source 1..23: mol_type = other RNA
tacaagttagcaaagacccatag

SEQ ID NO: 1250 - RNA, 23 nt, synthetic construct
  source 1..23: mol_type = other RNA
tacaaagttgagttccaggacag

SEQ ID NO: 1251 - RNA, 23 nt, synthetic construct
  source 1..23: mol_type = other RNA
aaaccttaagttgatgccccagt

SEQ ID NO: 1252 - RNA, 23 nt, synthetic construct
  source 1..23: mol_type = other RNA
tcattgagagcactagacaccca

SEQ ID NO: 1253 - RNA, 23 nt, synthetic construct
  source 1..23: mol_type = other RNA
cgcacttattcagatccacctcg

SEQ ID NO: 1254 - RNA, 23 nt, synthetic construct
  source 1..23: mol_type = other RNA
cttgattaaagcatccaacacca

SEQ ID NO: 1255 - RNA, 23 nt, synthetic construct
  source 1..23: mol_type = other RNA
caccagagttgacaggcacactc

SEQ ID NO: 1256 - RNA, 23 nt, synthetic construct
  source 1..23: mol_type = other RNA
tcatcttcttgatttccgcaatc

SEQ ID NO: 1257 - RNA, 23 nt, synthetic construct
  source 1..23: mol_type = other RNA
accagagttgacaggcacactca

SEQ ID NO: 1258 - RNA, 23 nt, synthetic construct
  source 1..23: mol_type = other RNA
ttaaagagctttgtagccccaca

SEQ ID NO: 1259 - RNA, 23 nt, synthetic construct
  source 1..23: mol_type = other RNA
tagagaacatgcatgtgcctcag

SEQ ID NO: 1260 - RNA, 23 nt, synthetic construct
  source 1..23: mol_type = other RNA
agcttcagaatagttcaccagct

SEQ ID NO: 1261 - RNA, 23 nt, synthetic construct
  source 1..23: mol_type = other RNA
aatcgctcaaaattgcacagcgg

SEQ ID NO: 1262 - RNA, 23 nt, synthetic construct
  source 1..23: mol_type = other RNA
tattgcacactaatgccgcaagg

SEQ ID NO: 1263 - RNA, 23 nt, synthetic construct
  source 1..23: mol_type = other RNA
tcttgaatatgaagccagcacca

SEQ ID NO: 1264 - RNA, 23 nt, synthetic construct
  source 1..23: mol_type = other RNA
tccattaagcaatcacgagacag

SEQ ID NO: 1265 - RNA, 23 nt, synthetic construct
  source 1..23: mol_type = other RNA
atccaagaaatggaaccgtgcac

SEQ ID NO: 1266 - RNA, 23 nt, synthetic construct
  source 1..23: mol_type = other RNA
gcaccaattccgttaccagcacg

SEQ ID NO: 1267 - RNA, 23 nt, synthetic construct
  source 1..23: mol_type = other RNA
aagcgaaataggtacacgcgtga

SEQ ID NO: 1268 - RNA, 23 nt, synthetic construct
  source 1..23: mol_type = other RNA
gcacaatactacgatggccacca

SEQ ID NO: 1269 - RNA, 23 nt, synthetic construct
  source 1..23: mol_type = other RNA
agtcacatcagaatgacagccaa

SEQ ID NO: 1270 - RNA, 23 nt, synthetic construct
  source 1..23: mol_type = other RNA
gttaatgaacactcgcaccacga

SEQ ID NO: 1271 - RNA, 23 nt, synthetic construct
  source 1..23: mol_type = other RNA
cacagatgatgctgagacccgac

SEQ ID NO: 1272 - RNA, 23 nt, synthetic construct
  source 1..23: mol_type = other RNA
ccaagcaattcacaaggacacgt

SEQ ID NO: 1273 - RNA, 23 nt, synthetic construct
  source 1..23: mol_type = other RNA
ccattcctattcaagccaccaca

SEQ ID NO: 1274 - RNA, 23 nt, synthetic construct
  source 1..23: mol_type = other RNA
ccacagaaaattaaaacgcagga

SEQ ID NO: 1275 - RNA, 23 nt, synthetic construct
  source 1..23: mol_type = other RNA
ccacctctaaaaatagcaagcac

SEQ ID NO: 1276 - RNA, 23 nt, synthetic construct
  source 1..23: mol_type = other RNA
gcgtacatcttgatgaccagcag

SEQ ID NO: 1277 - RNA, 23 nt, synthetic construct
  source 1..23: mol_type = other RNA
cctattctagaagagacaccaag

SEQ ID NO: 1278 - RNA, 23 nt, synthetic construct
  source 1..23: mol_type = other RNA
cttcatgagaaacagctgccaca

SEQ ID NO: 1279 - RNA, 23 nt, synthetic construct
  source 1..23: mol_type = other RNA
aagtttcaaatgtgagcatcagc

SEQ ID NO: 1280 - RNA, 23 nt, synthetic construct
  source 1..23: mol_type = other RNA
caacattctgaagaggcctccaa

SEQ ID NO: 1281 - RNA, 23 nt, synthetic construct
  source 1..23: mol_type = other RNA
cattcacaaattcccagagccag

SEQ ID NO: 1282 - RNA, 23 nt, synthetic construct
  source 1..23: mol_type = other RNA
agaactcactatataggccaaac

SEQ ID NO: 1283 - RNA, 23 nt, synthetic construct
  source 1..23: mol_type = other RNA
tcatagttaagacagagcagcac

SEQ ID NO: 1284 - RNA, 23 nt, synthetic construct
  source 1..23: mol_type = other RNA
atatatttcttcattagacaccc

SEQ ID NO: 1285 - RNA, 23 nt, synthetic construct
  source 1..23: mol_type = other RNA
tatatttcttcattagacaccct

SEQ ID NO: 1286 - RNA, 23 nt, synthetic construct
  source 1..23: mol_type = other RNA
gcaaacatcatatttgagccttg

SEQ ID NO: 1287 - RNA, 23 nt, synthetic construct
  source 1..23: mol_type = other RNA
tttatgcgtaaattccaacagcc

SEQ ID NO: 1288 - RNA, 23 nt, synthetic construct
  source 1..23: mol_type = other RNA
catataatatcaaagatgccagg

SEQ ID NO: 1289 - RNA, 23 nt, synthetic construct
  source 1..23: mol_type = other RNA
ttctacagatagttcgcaaccca

SEQ ID NO: 1290 - RNA, 23 nt, synthetic construct
  source 1..23: mol_type = other RNA
cacaatcatttgaagacaccaga

SEQ ID NO: 1291 - RNA, 23 nt, synthetic construct
  source 1..23: mol_type = other RNA
gcaataactcagaaggcagcagt

SEQ ID NO: 1292 - RNA, 23 nt, synthetic construct
  source 1..23: mol_type = other RNA
tgtcatattctttagcccaacac

SEQ ID NO: 1293 - RNA, 23 nt, synthetic construct
  source 1..23: mol_type = other RNA
acaacaataaagtatgaggccag

SEQ ID NO: 1294 - RNA, 23 nt, synthetic construct
  source 1..23: mol_type = other RNA
tgcaataatacaaaggccgccca

SEQ ID NO: 1295 - RNA, 23 nt, synthetic construct
  source 1..23: mol_type = other RNA
accattctgcaaaggcccaccat

SEQ ID NO: 1296 - RNA, 23 nt, synthetic construct
  source 1..23: mol_type = other RNA
acctccacactattgatgcgcaa

SEQ ID NO: 1297 - RNA, 23 nt, synthetic construct
  source 1..23: mol_type = other RNA
aatgcaagtgtagttgccagcat

SEQ ID NO: 1298 - RNA, 23 nt, synthetic construct
  source 1..23: mol_type = other RNA
atacagattcagatgaccacagt

SEQ ID NO: 1299 - RNA, 23 nt, synthetic construct
  source 1..23: mol_type = other RNA
tgatccagaaagctgaggacacc

SEQ ID NO: 1300 - RNA, 23 nt, synthetic construct
  source 1..23: mol_type = other RNA
acaaactctagagatgagcctca

SEQ ID NO: 1301 - RNA, 23 nt, synthetic construct
  source 1..23: mol_type = other RNA
cagagagcacaaaatcaccaggt

SEQ ID NO: 1302 - RNA, 23 nt, synthetic construct
  source 1..23: mol_type = other RNA
tacaggtcatagaagtcccctga

SEQ ID NO: 1303 - RNA, 23 nt, synthetic construct
  source 1..23: mol_type = other RNA
cccctttatccattagcaccagc

SEQ ID NO: 1304 - RNA, 23 nt, synthetic construct
  source 1..23: mol_type = other RNA
atgccaattagaaacaggccagc

SEQ ID NO: 1305 - RNA, 23 nt, synthetic construct
  source 1..23: mol_type = other RNA
agcacgatacgataatgctccca

SEQ ID NO: 1306 - RNA, 23 nt, synthetic construct
  source 1..23: mol_type = other RNA
tatcgtatctttctgccacacca

SEQ ID NO: 1307 - RNA, 23 nt, synthetic construct
  source 1..23: mol_type = other RNA
ccatcctcagttgacagccactg

SEQ ID NO: 1308 - RNA, 23 nt, synthetic construct
  source 1..23: mol_type = other RNA
gcagacattaatctgaaagcacc

SEQ ID NO: 1309 - RNA, 23 nt, synthetic construct
  source 1..23: mol_type = other RNA
tcattcgtctattacgcaccgaa

SEQ ID NO: 1310 - RNA, 23 nt, synthetic construct
  source 1..23: mol_type = other RNA
aaagaatgaacaatgggcaacag

SEQ ID NO: 1311 - RNA, 23 nt, synthetic construct
  source 1..23: mol_type = other RNA
aattcacatacatgagctcgtag

SEQ ID NO: 1312 - RNA, 23 nt, synthetic construct
  source 1..23: mol_type = other RNA
atgctaatgattcgtcaaagccc

SEQ ID NO: 1313 - RNA, 23 nt, synthetic construct
  source 1..23: mol_type = other RNA
agaatatatgatgtagcacaccc

SEQ ID NO: 1314 - RNA, 23 nt, synthetic construct
  source 1..23: mol_type = other RNA
cataagtataaaccagaggccac

SEQ ID NO: 1315 - RNA, 23 nt, synthetic construct
  source 1..23: mol_type = other RNA
agaaacatgaaaagacagcccat

SEQ ID NO: 1316 - RNA, 23 nt, synthetic construct
  source 1..23: mol_type = other RNA
caaaattcataagtgagaagcct

SEQ ID NO: 1317 - RNA, 23 nt, synthetic construct
  source 1..23: mol_type = other RNA
tatgagataaaatctagcccagg

SEQ ID NO: 1318 - RNA, 23 nt, synthetic construct
  source 1..23: mol_type = other RNA
ccacagtcagaagagcaggcaaa

SEQ ID NO: 1319 - RNA, 23 nt, synthetic construct
  source 1..23: mol_type = other RNA
ctccatcttcaatgtgatcaccc

SEQ ID NO: 1320 - RNA, 23 nt, synthetic construct
  source 1..23: mol_type = other RNA
cacgtcgaaacatttctgcagcc

SEQ ID NO: 1321 - RNA, 23 nt, synthetic construct
  source 1..23: mol_type = other RNA
tcttgacaaacaaagcaaggctt

SEQ ID NO: 1322 - RNA, 23 nt, synthetic construct
  source 1..23: mol_type = other RNA
tatcaaagaggatgccactccca

SEQ ID NO: 1323 - RNA, 23 nt, synthetic construct
  source 1..23: mol_type = other RNA
tacagagtaagtgtccagcccaa

SEQ ID NO: 1324 - RNA, 23 nt, synthetic construct
  source 1..23: mol_type = other RNA
atgtgaaatacaaacgcagccat

SEQ ID NO: 1325 - RNA, 23 nt, synthetic construct
  source 1..23: mol_type = other RNA
tagctactatttatacagcacca

SEQ ID NO: 1326 - RNA, 23 nt, synthetic construct
  source 1..23: mol_type = other RNA
catgaagaatagtaaggttcagc

SEQ ID NO: 1327 - RNA, 23 nt, synthetic construct
  source 1..23: mol_type = other RNA
aaaacttgaaaacacagcctcac

SEQ ID NO: 1328 - RNA, 23 nt, synthetic construct
  source 1..23: mol_type = other RNA
tgtatatagcaaaggcaacacca

SEQ ID NO: 1329 - RNA, 23 nt, synthetic construct
  source 1..23: mol_type = other RNA
gattaaaacaagacagacccacc

SEQ ID NO: 1330 - RNA, 23 nt, synthetic construct
  source 1..23: mol_type = other RNA
tctttcagcattttcacagccac

SEQ ID NO: 1331 - RNA, 23 nt, synthetic construct
  source 1..23: mol_type = other RNA
ccaagtcatgaatggcagacccc

SEQ ID NO: 1332 - RNA, 23 nt, synthetic construct
  source 1..23: mol_type = other RNA
cagacacagaagatggacagcag

SEQ ID NO: 1333 - RNA, 23 nt, synthetic construct
  source 1..23: mol_type = other RNA
aatcaagactgaattacgcaagc

SEQ ID NO: 1334 - RNA, 23 nt, synthetic construct
  source 1..23: mol_type = other RNA
catagaatgtcatagaacccctg

SEQ ID NO: 1335 - RNA, 23 nt, synthetic construct
  source 1..23: mol_type = other RNA
gaaagaaatctagcaagccccga

SEQ ID NO: 1336 - RNA, 23 nt, synthetic construct
  source 1..23: mol_type = other RNA
tctcaagcattattgagccacat

SEQ ID NO: 1337 - RNA, 23 nt, synthetic construct
  source 1..23: mol_type = other RNA
acatccacacaaacagcaccaga

SEQ ID NO: 1338 - RNA, 23 nt, synthetic construct
  source 1..23: mol_type = other RNA
gtagcacttaaaataggagcaca

SEQ ID NO: 1339 - RNA, 23 nt, synthetic construct
  source 1..23: mol_type = other RNA
ctacttagacaaataccagcagc

SEQ ID NO: 1340 - RNA, 23 nt, synthetic construct
  source 1..23: mol_type = other RNA
aatagcaataaaataggagacat

SEQ ID NO: 1341 - RNA, 23 nt, synthetic construct
  source 1..23: mol_type = other RNA
tatcaagtgatgttaccacaggc

SEQ ID NO: 1342 - RNA, 23 nt, synthetic construct
  source 1..23: mol_type = other RNA
cccctttcaaagatgccaccatt

SEQ ID NO: 1343 - RNA, 23 nt, synthetic construct
  source 1..23: mol_type = other RNA
gtacacatacagaagcagcagca

SEQ ID NO: 1344 - RNA, 23 nt, synthetic construct
  source 1..23: mol_type = other RNA
gattgtataaacctgcacacagc

SEQ ID NO: 1345 - RNA, 23 nt, synthetic construct
  source 1..23: mol_type = other RNA
tgcaaaaaggtaattccacagag

SEQ ID NO: 1346 - RNA, 23 nt, synthetic construct
  source 1..23: mol_type = other RNA
cagcataattgataagaaccccc

SEQ ID NO: 1347 - RNA, 23 nt, synthetic construct
  source 1..23: mol_type = other RNA
acttaaataaaaatagcaccaga

SEQ ID NO: 1348 - RNA, 23 nt, synthetic construct
  source 1..23: mol_type = other RNA
caaaaagttaagaagaggccact

SEQ ID NO: 1349 - RNA, 23 nt, synthetic construct
  source 1..23: mol_type = other RNA
gaaacacagcgtatgccccacat

SEQ ID NO: 1350 - RNA, 23 nt, synthetic construct
  source 1..23: mol_type = other RNA
ctgacagtatgtagccgacccaa

SEQ ID NO: 1351 - RNA, 23 nt, synthetic construct
  source 1..23: mol_type = other RNA
acatccaaacacttgaggggcaa

SEQ ID NO: 1352 - RNA, 23 nt, synthetic construct
  source 1..23: mol_type = other RNA
tgagcacaagattacacaggcat

SEQ ID NO: 1353 - RNA, 23 nt, synthetic construct
  source 1..23: mol_type = other RNA
aataaatttaaaaggcagcacct

SEQ ID NO: 1354 - RNA, 23 nt, synthetic construct
  source 1..23: mol_type = other RNA
aggtccaaaagttttgcccgcaa

SEQ ID NO: 1355 - RNA, 23 nt, synthetic construct
  source 1..23: mol_type = other RNA
tttcatatagaaataccccaact

SEQ ID NO: 1356 - RNA, 23 nt, synthetic construct
  source 1..23: mol_type = other RNA
atgtccagacttatccagcagca

SEQ ID NO: 1357 - RNA, 23 nt, synthetic construct
  source 1..23: mol_type = other RNA
tatttctcattaaatgagcacag

SEQ ID NO: 1358 - RNA, 23 nt, synthetic construct
  source 1..23: mol_type = other RNA
tccagatcactttgacagcccct

SEQ ID NO: 1359 - RNA, 23 nt, synthetic construct
  source 1..23: mol_type = other RNA
ccaagagtaagttggccgtcagt

SEQ ID NO: 1360 - RNA, 23 nt, synthetic construct
  source 1..23: mol_type = other RNA
cccagatgacttgtccccacaga

SEQ ID NO: 1361 - RNA, 23 nt, synthetic construct
  source 1..23: mol_type = other RNA
accacatactgaatggcctcagt

SEQ ID NO: 1362 - RNA, 23 nt, synthetic construct
  source 1..23: mol_type = other RNA
atcctcagaaaaacacagggcgc

SEQ ID NO: 1363 - RNA, 23 nt, synthetic construct
  source 1..23: mol_type = other RNA
gtccaatagagatagccaccaac

SEQ ID NO: 1364 - RNA, 23 nt, synthetic construct
  source 1..23: mol_type = other RNA
gaaatccatatgatgcacccaag

SEQ ID NO: 1365 - RNA, 23 nt, synthetic construct
  source 1..23: mol_type = other RNA
acattgaaaatggctgcagacct

SEQ ID NO: 1366 - RNA, 23 nt, synthetic construct
  source 1..23: mol_type = other RNA
agtccaatttcatttgcagcaac

SEQ ID NO: 1367 - RNA, 23 nt, synthetic construct
  source 1..23: mol_type = other RNA
acttcccaacaaaagacgcaggc

SEQ ID NO: 1368 - RNA, 23 nt, synthetic construct
  source 1..23: mol_type = other RNA
acccacttttatcaccatcgcaa

SEQ ID NO: 1369 - RNA, 23 nt, synthetic construct
  source 1..23: mol_type = other RNA
gaagtcattcttgtagagggcat

SEQ ID NO: 1370 - RNA, 23 nt, synthetic construct
  source 1..23: mol_type = other RNA
tctttgtgagatgccacacccac

SEQ ID NO: 1371 - RNA, 23 nt, synthetic construct
  source 1..23: mol_type = other RNA
gtagacatcaatgaggttgctca

SEQ ID NO: 1372 - RNA, 23 nt, synthetic construct
  source 1..23: mol_type = other RNA
tttatagaggaaagcttggccag

SEQ ID NO: 1373 - RNA, 23 nt, synthetic construct
  source 1..23: mol_type = other RNA
atttcccagaacaaaggccattg

SEQ ID NO: 1374 - RNA, 23 nt, synthetic construct
  source 1..23: mol_type = other RNA
cacagatgttggtaagagcagtg

SEQ ID NO: 1375 - RNA, 23 nt, synthetic construct
  source 1..23: mol_type = other RNA
catgatgtcatttatgagggctt

SEQ ID NO: 1376 - RNA, 23 nt, synthetic construct
  source 1..23: mol_type = other RNA
acctcttaattacttcccacagc

SEQ ID NO: 1377 - RNA, 23 nt, synthetic construct
  source 1..23: mol_type = other RNA
gccatcagcatcatacactcctc

SEQ ID NO: 1378 - RNA, 23 nt, synthetic construct
  source 1..23: mol_type = other RNA
tacaagacttagaggccaaaccc

SEQ ID NO: 1379 - RNA, 23 nt, synthetic construct
  source 1..23: mol_type = other RNA
cattaagacagtacacagagcct

SEQ ID NO: 1380 - RNA, 23 nt, synthetic construct
  source 1..23: mol_type = other RNA
gccacatataaaatgagacagag

SEQ ID NO: 1381 - RNA, 23 nt, synthetic construct
  source 1..23: mol_type = other RNA
gaaaaatcagaagtgcaagacca

SEQ ID NO: 1382 - RNA, 23 nt, synthetic construct
  source 1..23: mol_type = other RNA
gtctatactcattatgcccacca

SEQ ID NO: 1383 - RNA, 23 nt, synthetic construct
  source 1..23: mol_type = other RNA
catcaatacctagtgacagccga

SEQ ID NO: 1384 - RNA, 23 nt, synthetic construct
  source 1..23: mol_type = other RNA
cagtgcaaaatcaaagctccagg

SEQ ID NO: 1385 - RNA, 23 nt, synthetic construct
  source 1..23: mol_type = other RNA
catttgcaacaagatacgatccg

SEQ ID NO: 1386 - RNA, 23 nt, synthetic construct
  source 1..23: mol_type = other RNA
tctgatgaacaaaagtcaggcag

SEQ ID NO: 1387 - RNA, 23 nt, synthetic construct
  source 1..23: mol_type = other RNA
agccttcagttacagcaaagcca

SEQ ID NO: 1388 - RNA, 23 nt, synthetic construct
  source 1..23: mol_type = other RNA
agaaccaaagatgtcagccagga

SEQ ID NO: 1389 - RNA, 23 nt, synthetic construct
  source 1..23: mol_type = other RNA
ctataaagtgagttccaggacag

SEQ ID NO: 1390 - RNA, 23 nt, synthetic construct
  source 1..23: mol_type = other RNA
actgtacacagaaagcgtgccat

SEQ ID NO: 1391 - RNA, 23 nt, synthetic construct
  source 1..23: mol_type = other RNA
acatgtgactaagacagaaccac

SEQ ID NO: 1392 - RNA, 23 nt, synthetic construct
  source 1..23: mol_type = other RNA
acaaaagagcacatagcccttgg

SEQ ID NO: 1393 - RNA, 23 nt, synthetic construct
  source 1..23: mol_type = other RNA
aggccatttagaagagcacacct

SEQ ID NO: 1394 - RNA, 23 nt, synthetic construct
  source 1..23: mol_type = other RNA
catgcctgaaaaataaggaccct

SEQ ID NO: 1395 - RNA, 23 nt, synthetic construct
  source 1..23: mol_type = other RNA
ccacagagatgacagaagccgag

SEQ ID NO: 1396 - RNA, 23 nt, synthetic construct
  source 1..23: mol_type = other RNA
agacgatagaaaaagcgccagat

SEQ ID NO: 1397 - RNA, 23 nt, synthetic construct
  source 1..23: mol_type = other RNA
tcataaaagcacgctggcccatg

SEQ ID NO: 1398 - RNA, 23 nt, synthetic construct
  source 1..23: mol_type = other RNA
aatcagcagacattgcttcaccg

SEQ ID NO: 1399 - RNA, 23 nt, synthetic construct
  source 1..23: mol_type = other RNA
ccactggaatcagagaagcagac

SEQ ID NO: 1400 - RNA, 23 nt, synthetic construct
  source 1..23: mol_type = other RNA
ataatatgtttattggatgcctg

SEQ ID NO: 1401 - RNA, 23 nt, synthetic construct
  source 1..23: mol_type = other RNA
agaacgcaaggacaggtatccag

SEQ ID NO: 1402 - RNA, 23 nt, synthetic construct
  source 1..23: mol_type = other RNA
aagtgagtcacggatggcccaag

SEQ ID NO: 1403 - RNA, 23 nt, synthetic construct
  source 1..23: mol_type = other RNA
gaaaacagtttaatacggcacct

SEQ ID NO: 1404 - RNA, 23 nt, synthetic construct
  source 1..23: mol_type = other RNA
agacacaatagcttggtgcagta

SEQ ID NO: 1405 - RNA, 23 nt, synthetic construct
  source 1..23: mol_type = other RNA
aaaagattctcacatggacccca

SEQ ID NO: 1406 - RNA, 23 nt, synthetic construct
  source 1..23: mol_type = other RNA
acgtagcaagttcctggctccac

SEQ ID NO: 1407 - RNA, 23 nt, synthetic construct
  source 1..23: mol_type = other RNA
tgcataggtacacacgtctccag

SEQ ID NO: 1408 - RNA, 23 nt, synthetic construct
  source 1..23: mol_type = other RNA
tatcaagatagcaaagaggcagt

SEQ ID NO: 1409 - RNA, 23 nt, synthetic construct
  source 1..23: mol_type = other RNA
aagcaattacaagtacagcgaca

SEQ ID NO: 1410 - RNA, 23 nt, synthetic construct
  source 1..23: mol_type = other RNA
cagcaaaattctatgatggcaga

SEQ ID NO: 1411 - RNA, 23 nt, synthetic construct
  source 1..23: mol_type = other RNA
cattataagatcaggctgacaag

SEQ ID NO: 1412 - RNA, 23 nt, synthetic construct
  source 1..23: mol_type = other RNA
ccaagcgctgaaacagagactcc

SEQ ID NO: 1413 - RNA, 23 nt, synthetic construct
  source 1..23: mol_type = other RNA
agtcacatgtagtaatcccgaag

SEQ ID NO: 1414 - RNA, 23 nt, synthetic construct
  source 1..23: mol_type = other RNA
gtcagcaaattctgggcaccaat

SEQ ID NO: 1415 - RNA, 23 nt, synthetic construct
  source 1..23: mol_type = other RNA
ccagcaggtgatattcagagccc

SEQ ID NO: 1416 - RNA, 23 nt, synthetic construct
  source 1..23: mol_type = other RNA
actgcatctcctcagccgccatg

SEQ ID NO: 1417 - RNA, 23 nt, synthetic construct
  source 1..23: mol_type = other RNA
cttgctcccagcccagcgcctag

SEQ ID NO: 1418 - RNA, 23 nt, synthetic construct
  source 1..23: mol_type = other RNA
attttccattataagaacccacc

SEQ ID NO: 1419 - RNA, 23 nt, synthetic construct
  source 1..23: mol_type = other RNA
caaaccaaaaatttggcgaccat

SEQ ID NO: 1420 - RNA, 23 nt, synthetic construct
  source 1..23: mol_type = other RNA
cattgactgtaagacctccacac

SEQ ID NO: 1421 - RNA, 23 nt, synthetic construct
  source 1..23: mol_type = other RNA
aagaagtcaccattgagctgcag

SEQ ID NO: 1422 - RNA, 23 nt, synthetic construct
  source 1..23: mol_type = other RNA
gaagcctcatatctaccacccca

SEQ ID NO: 1423 - RNA, 23 nt, synthetic construct
  source 1..23: mol_type = other RNA
agaaaaatagactttcagcaggt

SEQ ID NO: 1424 - RNA, 23 nt, synthetic construct
  source 1..23: mol_type = other RNA
acgaaacaaataaatagccagga

SEQ ID NO: 1425 - RNA, 23 nt, synthetic construct
  source 1..23: mol_type = other RNA
aaatataatttgtggacccccat

SEQ ID NO: 1426 - RNA, 23 nt, synthetic construct
  source 1..23: mol_type = other RNA
gattcatagtagacccagtcgaa

SEQ ID NO: 1427 - RNA, 23 nt, synthetic construct
  source 1..23: mol_type = other RNA
ttaaaagacaaaaagagcctgat

SEQ ID NO: 1428 - RNA, 23 nt, synthetic construct
  source 1..23: mol_type = other RNA
cacacagtagtagatgccagcat

SEQ ID NO: 1429 - RNA, 23 nt, synthetic construct
  source 1..23: mol_type = other RNA
aaactgtagatcaacagccggct

SEQ ID NO: 1430 - RNA, 23 nt, synthetic construct
  source 1..23: mol_type = other RNA
aacatttctaattcgaggaacgt

SEQ ID NO: 1431 - RNA, 23 nt, synthetic construct
  source 1..23: mol_type = other RNA
cagttcagaacggtcgaatcccc

SEQ ID NO: 1432 - RNA, 23 nt, synthetic construct
  source 1..23: mol_type = other RNA
agtcaccttcagttccttccccg

SEQ ID NO: 1433 - RNA, 23 nt, synthetic construct
  source 1..23: mol_type = other RNA
agaaacttatttgatccacccag

SEQ ID NO: 1434 - RNA, 23 nt, synthetic construct
  source 1..23: mol_type = other RNA
tttatttctgctttgcaagccac

SEQ ID NO: 1435 - RNA, 23 nt, synthetic construct
  source 1..23: mol_type = other RNA
taataatcggcatagtccactcg

SEQ ID NO: 1436 - RNA, 23 nt, synthetic construct
  source 1..23: mol_type = other RNA
accaaaaggatgcttctgacgtg

SEQ ID NO: 1437 - RNA, 23 nt, synthetic construct
  source 1..23: mol_type = other RNA
cttaaagtcacaggaagcagcag

SEQ ID NO: 1438 - RNA, 23 nt, synthetic construct
  source 1..23: mol_type = other RNA
tacaaagagaagaagccaccatc

SEQ ID NO: 1439 - RNA, 23 nt, synthetic construct
  source 1..23: mol_type = other RNA
actgacatatgaaacccagcaca

SEQ ID NO: 1440 - RNA, 23 nt, synthetic construct
  source 1..23: mol_type = other RNA
gttcagaaaacaagggcccacaa

SEQ ID NO: 1441 - RNA, 23 nt, synthetic construct
  source 1..23: mol_type = other RNA
cactgtatttaataaccacaccc

SEQ ID NO: 1442 - RNA, 23 nt, synthetic construct
  source 1..23: mol_type = other RNA
gtctgtttatcgaagacagccaa

SEQ ID NO: 1443 - RNA, 23 nt, synthetic construct
  source 1..23: mol_type = other RNA
gccatttgatcttgagcagccat

SEQ ID NO: 1444 - RNA, 23 nt, synthetic construct
  source 1..23: mol_type = other RNA
ataaatacaagcatggtcagctt

SEQ ID NO: 1445 - RNA, 23 nt, synthetic construct
  source 1..23: mol_type = other RNA
aacactttgaaaaacaggcaggt

SEQ ID NO: 1446 - RNA, 23 nt, synthetic construct
  source 1..23: mol_type = other RNA
acagatcgccatttaaccccgag

SEQ ID NO: 1447 - RNA, 23 nt, synthetic construct
  source 1..23: mol_type = other RNA
gtagtcaatcagatgaaccacac

SEQ ID NO: 1448 - RNA, 23 nt, synthetic construct
  source 1..23: mol_type = other RNA
ctgcagaatcaaaacccagcaag

SEQ ID NO: 1449 - RNA, 23 nt, synthetic construct
  source 1..23: mol_type = other RNA
gcctgattccaataacagagccg

SEQ ID NO: 1450 - RNA, 23 nt, synthetic construct
  source 1..23: mol_type = other RNA
attaccaacgatatcaacctgca

SEQ ID NO: 1451 - RNA, 23 nt, synthetic construct
  source 1..23: mol_type = other RNA
ctgtacaaaaatagtcccccaaa

SEQ ID NO: 1452 - RNA, 23 nt, synthetic construct
  source 1..23: mol_type = other RNA
tcggacaaaagtttccactccgc

SEQ ID NO: 1453 - RNA, 23 nt, synthetic construct
  source 1..23: mol_type = other RNA
gaaaacattgcaaaggactcagt

SEQ ID NO: 1454 - RNA, 23 nt, synthetic construct
  source 1..23: mol_type = other RNA
aacagcatatactttcccagcag

SEQ ID NO: 1455 - RNA, 23 nt, synthetic construct
  source 1..23: mol_type = other RNA
ttaacaacagatgaccataccct

SEQ ID NO: 1456 - RNA, 23 nt, synthetic construct
  source 1..23: mol_type = other RNA
tgcaaagaaaagttcctggactg

SEQ ID NO: 1457 - RNA, 23 nt, synthetic construct
  source 1..23: mol_type = other RNA
tcgctctcgttcagcagcctcca

SEQ ID NO: 1458 - RNA, 23 nt, synthetic construct
  source 1..23: mol_type = other RNA
cacaacagcagtagcgccacgag

SEQ ID NO: 1459 - RNA, 23 nt, synthetic construct
  source 1..23: mol_type = other RNA
aaccacatgaatacctggtgccc

SEQ ID NO: 1460 - RNA, 23 nt, synthetic construct
  source 1..23: mol_type = other RNA
acagcttcatcctcactgcccac

SEQ ID NO: 1461 - RNA, 23 nt, synthetic construct
  source 1..23: mol_type = other RNA
tctccagtacctcagagcccttg

SEQ ID NO: 1462 - RNA, 23 nt, synthetic construct
  source 1..23: mol_type = other RNA
tcttctagtactaaagagccctg

SEQ ID NO: 1463 - RNA, 23 nt, synthetic construct
  source 1..23: mol_type = other RNA
acttatagcgagttcccaccaca

SEQ ID NO: 1464 - RNA, 23 nt, synthetic construct
  source 1..23: mol_type = other RNA
acaaacactaaagagtggccatt

SEQ ID NO: 1465 - RNA, 23 nt, synthetic construct
  source 1..23: mol_type = other RNA
caaaccaagtcagaggaagctac

SEQ ID NO: 1466 - RNA, 23 nt, synthetic construct
  source 1..23: mol_type = other RNA
gttacacaagttaatgccccatc

SEQ ID NO: 1467 - RNA, 23 nt, synthetic construct
  source 1..23: mol_type = other RNA
ttttgaagacaaatgtgtccagt

SEQ ID NO: 1468 - RNA, 23 nt, synthetic construct
  source 1..23: mol_type = other RNA
tccaactacaaaacaagaccccc

SEQ ID NO: 1469 - RNA, 23 nt, synthetic construct
  source 1..23: mol_type = other RNA
aaagttattgaagaacgccacct

SEQ ID NO: 1470 - RNA, 23 nt, synthetic construct
  source 1..23: mol_type = other RNA
agaaggttatcaaaccagccact

SEQ ID NO: 1471 - RNA, 23 nt, synthetic construct
  source 1..23: mol_type = other RNA
cgaaaagcatcatagttcccacg

SEQ ID NO: 1472 - RNA, 23 nt, synthetic construct
  source 1..23: mol_type = other RNA
tgccatttatgttgcggacacga

SEQ ID NO: 1473 - RNA, 23 nt, synthetic construct
  source 1..23: mol_type = other RNA
ccactttaaatgttgcaccacga

SEQ ID NO: 1474 - RNA, 23 nt, synthetic construct
  source 1..23: mol_type = other RNA
accaatagacaaaggaagtgcag

SEQ ID NO: 1475 - RNA, 23 nt, synthetic construct
  source 1..23: mol_type = other RNA
catagagccaatgagacatgccg

SEQ ID NO: 1476 - RNA, 23 nt, synthetic construct
  source 1..23: mol_type = other RNA
agcagcatcaaaatccaaggcag

SEQ ID NO: 1477 - RNA, 23 nt, synthetic construct
  source 1..23: mol_type = other RNA
aagatgatactcagtagcaccgt

SEQ ID NO: 1478 - RNA, 23 nt, synthetic construct
  source 1..23: mol_type = other RNA
caagcagaaaaatagatgcagtc

SEQ ID NO: 1479 - RNA, 23 nt, synthetic construct
  source 1..23: mol_type = other RNA
ctgagaattaacaggccaaccca

SEQ ID NO: 1480 - RNA, 23 nt, synthetic construct
  source 1..23: mol_type = other RNA
aggaatttcagaaatgcgtcctt

SEQ ID NO: 1481 - RNA, 23 nt, synthetic construct
  source 1..23: mol_type = other RNA
cccaccgagtttacgcaccagca

SEQ ID NO: 1482 - RNA, 23 nt, synthetic construct
  source 1..23: mol_type = other RNA
cccccagctccttaagctccacc

SEQ ID NO: 1483 - RNA, 23 nt, synthetic construct
  source 1..23: mol_type = other RNA
caaataaatcaatagaggccaag

SEQ ID NO: 1484 - RNA, 23 nt, synthetic construct
  source 1..23: mol_type = other RNA
gaacaaaatgctttacgacagcc

SEQ ID NO: 1485 - RNA, 23 nt, synthetic construct
  source 1..23: mol_type = other RNA
atcaaacaagcagagcagcacct

SEQ ID NO: 1486 - RNA, 23 nt, synthetic construct
  source 1..23: mol_type = other RNA
acaagcaatgaacagccaaccac

SEQ ID NO: 1487 - RNA, 23 nt, synthetic construct
  source 1..23: mol_type = other RNA
catgcgtattacagtgcagcgaa

SEQ ID NO: 1488 - RNA, 23 nt, synthetic construct
  source 1..23: mol_type = other RNA
acataatcagaaacgccaacagc

SEQ ID NO: 1489 - RNA, 23 nt, synthetic construct
  source 1..23: mol_type = other RNA
ccacagagaactaagccggctaa

SEQ ID NO: 1490 - RNA, 23 nt, synthetic construct
  source 1..23: mol_type = other RNA
actaaaatgtttctggcagccag

SEQ ID NO: 1491 - RNA, 23 nt, synthetic construct
  source 1..23: mol_type = other RNA
gaagaaatcgaatagccatccac

SEQ ID NO: 1492 - RNA, 23 nt, synthetic construct
  source 1..23: mol_type = other RNA
ccccaaagtaaggctcagagctg

SEQ ID NO: 1493 - RNA, 23 nt, synthetic construct
  source 1..23: mol_type = other RNA
caatacataaaatacaggcagag

SEQ ID NO: 1494 - RNA, 23 nt, synthetic construct
  source 1..23: mol_type = other RNA
atttctaaagcagtaggcagcat

SEQ ID NO: 1495 - RNA, 23 nt, synthetic construct
  source 1..23: mol_type = other RNA
tgtattgtagatcaacagccact

SEQ ID NO: 1496 - RNA, 23 nt, synthetic construct
  source 1..23: mol_type = other RNA
agaacccttaaaatagcagtcag

SEQ ID NO: 1497 - RNA, 23 nt, synthetic construct
  source 1..23: mol_type = other RNA
aaagaaaatgcaacgcgttccca

SEQ ID NO: 1498 - RNA, 23 nt, synthetic construct
  source 1..23: mol_type = other RNA
aataagacattaacagagcccag

SEQ ID NO: 1499 - RNA, 23 nt, synthetic construct
  source 1..23: mol_type = other RNA
ccaccaatagaacagcgtcgagc

SEQ ID NO: 1500 - RNA, 23 nt, synthetic construct
  source 1..23: mol_type = other RNA
agcataagtgcaatgcagacgaa

SEQ ID NO: 1501 - RNA, 23 nt, synthetic construct
  source 1..23: mol_type = other RNA
aagaagtatccatagtgcacaga

SEQ ID NO: 1502 - RNA, 23 nt, synthetic construct
  source 1..23: mol_type = other RNA
gcttcatttagtgccacacccca

SEQ ID NO: 1503 - RNA, 23 nt, synthetic construct
  source 1..23: mol_type = other RNA
ctacatagcaaatttcaggccag

SEQ ID NO: 1504 - RNA, 23 nt, synthetic construct
  source 1..23: mol_type = other RNA
aaaatgctatgaaagcagtcacc

SEQ ID NO: 1505 - RNA, 23 nt, synthetic construct
  source 1..23: mol_type = other RNA
taaaattcatgaaagaagcagga

SEQ ID NO: 1506 - RNA, 23 nt, synthetic construct
  source 1..23: mol_type = other RNA
cccagatccaagatgacacccat

SEQ ID NO: 1507 - RNA, 23 nt, synthetic construct
  source 1..23: mol_type = other RNA
aatgaactgtttctgcgacacac

SEQ ID NO: 1508 - RNA, 23 nt, synthetic construct
  source 1..23: mol_type = other RNA
ttaagcaaatgagtcccagcaca

SEQ ID NO: 1509 - RNA, 23 nt, synthetic construct
  source 1..23: mol_type = other RNA
gtcaacactataaatggccagaa

SEQ ID NO: 1510 - RNA, 23 nt, synthetic construct
  source 1..23: mol_type = other RNA
acactgtaagatgacagagccac

SEQ ID NO: 1511 - RNA, 23 nt, synthetic construct
  source 1..23: mol_type = other RNA
cactgaagactgaagcgacatgc

SEQ ID NO: 1512 - RNA, 23 nt, synthetic construct
  source 1..23: mol_type = other RNA
gatacagcaatgaagctctctag

SEQ ID NO: 1513 - RNA, 23 nt, synthetic construct
  source 1..23: mol_type = other RNA
aggtacatatagaagccccaagt

SEQ ID NO: 1514 - RNA, 23 nt, synthetic construct
  source 1..23: mol_type = other RNA
agacacatatctctgaaggccca

SEQ ID NO: 1515 - RNA, 23 nt, synthetic construct
  source 1..23: mol_type = other RNA
ttccagacttgatgcgaccacca

SEQ ID NO: 1516 - RNA, 23 nt, synthetic construct
  source 1..23: mol_type = other RNA
tcaccagctgaattccacacagc

SEQ ID NO: 1517 - RNA, 23 nt, synthetic construct
  source 1..23: mol_type = other RNA
ccccctccaatgaagccacccat

SEQ ID NO: 1518 - RNA, 23 nt, synthetic construct
  source 1..23: mol_type = other RNA
atgacaaagtactcataggccag

SEQ ID NO: 1519 - RNA, 23 nt, synthetic construct
  source 1..23: mol_type = other RNA
acgacaaagtgcttataggccag

SEQ ID NO: 1520 - RNA, 23 nt, synthetic construct
  source 1..23: mol_type = other RNA
ccttagagacgataccagcaact

SEQ ID NO: 1521 - RNA, 23 nt, synthetic construct
  source 1..23: mol_type = other RNA
aagcaatgtctagaggatcccac

SEQ ID NO: 1522 - RNA, 23 nt, synthetic construct
  source 1..23: mol_type = other RNA
caatgctatcaatttcagccaag

SEQ ID NO: 1523 - RNA, 23 nt, synthetic construct
  source 1..23: mol_type = other RNA
ccatcttgaagaaacagcccagt

SEQ ID NO: 1524 - RNA, 23 nt, synthetic construct
  source 1..23: mol_type = other RNA
gacacataaaatagtagaagcca

SEQ ID NO: 1525 - RNA, 23 nt, synthetic construct
  source 1..23: mol_type = other RNA
tgctatgaaaccaagccagccat

SEQ ID NO: 1526 - RNA, 23 nt, synthetic construct
  source 1..23: mol_type = other RNA
ttcaagcagaattcgatctccag

SEQ ID NO: 1527 - RNA, 23 nt, synthetic construct
  source 1..23: mol_type = other RNA
acagcaaacctgaagaaggcctc

SEQ ID NO: 1528 - RNA, 23 nt, synthetic construct
  source 1..23: mol_type = other RNA
cccaagaagatccagagcaccgt

SEQ ID NO: 1529 - RNA, 23 nt, synthetic construct
  source 1..23: mol_type = other RNA
taacgacatctaaaacaccagct

SEQ ID NO: 1530 - RNA, 23 nt, synthetic construct
  source 1..23: mol_type = other RNA
agaaaatcgaggaagaacagcag

SEQ ID NO: 1531 - RNA, 23 nt, synthetic construct
  source 1..23: mol_type = other RNA
aaaagtgccaaagagaggaccaa

SEQ ID NO: 1532 - RNA, 23 nt, synthetic construct
  source 1..23: mol_type = other RNA
acactttgcaaagcagttggcac

SEQ ID NO: 1533 - RNA, 23 nt, synthetic construct
  source 1..23: mol_type = other RNA
atgaagtaagatgatcggaccag

SEQ ID NO: 1534 - RNA, 23 nt, synthetic construct
  source 1..23: mol_type = other RNA
gcagcaattgacaacgctcgtga

SEQ ID NO: 1535 - RNA, 23 nt, synthetic construct
  source 1..23: mol_type = other RNA
caatttcacggaaggaagccagc

SEQ ID NO: 1536 - RNA, 23 nt, synthetic construct
  source 1..23: mol_type = other RNA
acatactttccttggggacacaa

SEQ ID NO: 1537 - RNA, 23 nt, synthetic construct
  source 1..23: mol_type = other RNA
atcagcagagccaggagcaccag

SEQ ID NO: 1538 - RNA, 23 nt, synthetic construct
  source 1..23: mol_type = other RNA
acagcaagtacagtaccaagccg

SEQ ID NO: 1539 - RNA, 23 nt, synthetic construct
  source 1..23: mol_type = other RNA
cagagtaaaagtcaaaggcagag

SEQ ID NO: 1540 - RNA, 23 nt, synthetic construct
  source 1..23: mol_type = other RNA
cagtcctaacatcagcagaccca

SEQ ID NO: 1541 - RNA, 23 nt, synthetic construct
  source 1..23: mol_type = other RNA
ctcagaagcaagaatcaggcaag

SEQ ID NO: 1542 - RNA, 23 nt, synthetic construct
  source 1..23: mol_type = other RNA
gtacaggaagaactgcagctcaa

SEQ ID NO: 1543 - RNA, 23 nt, synthetic construct
  source 1..23: mol_type = other RNA
caaaaattgtgtgaagagccact

SEQ ID NO: 1544 - RNA, 23 nt, synthetic construct
  source 1..23: mol_type = other RNA
tacaagagtatgcagctgccagt

SEQ ID NO: 1545 - RNA, 23 nt, synthetic construct
  source 1..23: mol_type = other RNA
ggagaaatttaaagggcacacag

SEQ ID NO: 1546 - RNA, 23 nt, synthetic construct
  source 1..23: mol_type = other RNA
gcaaagcatagtcttgagcagtg

SEQ ID NO: 1547 - RNA, 23 nt, synthetic construct
  source 1..23: mol_type = other RNA
agaacatgacctcagtgcagctg

SEQ ID NO: 1548 - RNA, 23 nt, synthetic construct
  source 1..23: mol_type = other RNA
tggaattcagaattgcccgacca

SEQ ID NO: 1549 - RNA, 23 nt, synthetic construct
  source 1..23: mol_type = other RNA
aagaacttatttgcaggtcccag

SEQ ID NO: 1550 - RNA, 23 nt, synthetic construct
  source 1..23: mol_type = other RNA
cgaaagcaaatgttggcgtacag

SEQ ID NO: 1551 - RNA, 23 nt, synthetic construct
  source 1..23: mol_type = other RNA
aataaactacatgtggtcaccag

SEQ ID NO: 1552 - RNA, 23 nt, synthetic construct
  source 1..23: mol_type = other RNA
tcgaaagtacaggaacagagcga

SEQ ID NO: 1553 - RNA, 23 nt, synthetic construct
  source 1..23: mol_type = other RNA
gtcaataataaaatgcacacagc

SEQ ID NO: 1554 - RNA, 23 nt, synthetic construct
  source 1..23: mol_type = other RNA
ctgagacaaagaacacgcacagc

SEQ ID NO: 1555 - RNA, 23 nt, synthetic construct
  source 1..23: mol_type = other RNA
taactagtcactaagccccagag

SEQ ID NO: 1556 - RNA, 23 nt, synthetic construct
  source 1..23: mol_type = other RNA
cctagagaaagcacaggagccac

SEQ ID NO: 1557 - RNA, 23 nt, synthetic construct
  source 1..23: mol_type = other RNA
atataagatacgtggttcagcca

SEQ ID NO: 1558 - RNA, 23 nt, synthetic construct
  source 1..23: mol_type = other RNA
cacagagagagcttgtccaccac

SEQ ID NO: 1559 - RNA, 23 nt, synthetic construct
  source 1..23: mol_type = other RNA
ctgtccctccctctcagccacac

SEQ ID NO: 1560 - RNA, 23 nt, synthetic construct
  source 1..23: mol_type = other RNA
ccacacagaaggtaccatctagg

SEQ ID NO: 1561 - RNA, 23 nt, synthetic construct
  source 1..23: mol_type = other RNA
cagaggaaagtcatagcggtcac

SEQ ID NO: 1562 - RNA, 23 nt, synthetic construct
  source 1..23: mol_type = other RNA
ttccactgacagatgctgagcag

SEQ ID NO: 1563 - RNA, 23 nt, synthetic construct
  source 1..23: mol_type = other RNA
cccataagctacaagccacacgc

SEQ ID NO: 1564 - RNA, 23 nt, synthetic construct
  source 1..23: mol_type = other RNA
aagcagaatagttatagtccagg

SEQ ID NO: 1565 - RNA, 23 nt, synthetic construct
  source 1..23: mol_type = other RNA
caagaagacgataaggagcaaca

SEQ ID NO: 1566 - RNA, 23 nt, synthetic construct
  source 1..23: mol_type = other RNA
cccagaagtacagcagcagctcg

SEQ ID NO: 1567 - RNA, 23 nt, synthetic construct
  source 1..23: mol_type = other RNA
aagatcatgaagtcaaggcagag

SEQ ID NO: 1568 - RNA, 23 nt, synthetic construct
  source 1..23: mol_type = other RNA
aatgtagaacatattggccacca

SEQ ID NO: 1569 - RNA, 23 nt, synthetic construct
  source 1..23: mol_type = other RNA
aaaaactcaattttggcacagag

SEQ ID NO: 1570 - RNA, 23 nt, synthetic construct
  source 1..23: mol_type = other RNA
catcaataagattctgagccaag

SEQ ID NO: 1571 - RNA, 23 nt, synthetic construct
  source 1..23: mol_type = other RNA
tacaagagcatcaagcatagcct

SEQ ID NO: 1572 - RNA, 23 nt, synthetic construct
  source 1..23: mol_type = other RNA
ccttcaaatatcaaagccaccac

SEQ ID NO: 1573 - RNA, 23 nt, synthetic construct
  source 1..23: mol_type = other RNA
ctataaacatttatacagcccca

SEQ ID NO: 1574 - RNA, 23 nt, synthetic construct
  source 1..23: mol_type = other RNA
aaagagaaatgtcagcgctcaag

SEQ ID NO: 1575 - RNA, 23 nt, synthetic construct
  source 1..23: mol_type = other RNA
cccacattaaaactccgaacgac

SEQ ID NO: 1576 - RNA, 23 nt, synthetic construct
  source 1..23: mol_type = other RNA
gagatctttactacgcaggcaga

SEQ ID NO: 1577 - RNA, 23 nt, synthetic construct
  source 1..23: mol_type = other RNA
acgagatatgagaccagctgcat

SEQ ID NO: 1578 - RNA, 23 nt, synthetic construct
  source 1..23: mol_type = other RNA
aaaaccgaagtacatcagcaccc

SEQ ID NO: 1579 - RNA, 23 nt, synthetic construct
  source 1..23: mol_type = other RNA
caaacttaacatctaccaccaga

SEQ ID NO: 1580 - RNA, 23 nt, synthetic construct
  source 1..23: mol_type = other RNA
cgctaatccaaagaatggcgcct

SEQ ID NO: 1581 - RNA, 23 nt, synthetic construct
  source 1..23: mol_type = other RNA
cacagtgatgaagacaggctgca

SEQ ID NO: 1582 - RNA, 23 nt, synthetic construct
  source 1..23: mol_type = other RNA
tccatctatcaccgcgtgcacga

SEQ ID NO: 1583 - RNA, 23 nt, synthetic construct
  source 1..23: mol_type = other RNA
cagtatacaagttgtcagccaag

SEQ ID NO: 1584 - RNA, 23 nt, synthetic construct
  source 1..23: mol_type = other RNA
catgcactaaagtaggagcagct

SEQ ID NO: 1585 - RNA, 23 nt, synthetic construct
  source 1..23: mol_type = other RNA
agagtccaaaatcagccacacac

SEQ ID NO: 1586 - RNA, 23 nt, synthetic construct
  source 1..23: mol_type = other RNA
tacagtacaggaatgcagcagag

SEQ ID NO: 1587 - RNA, 23 nt, synthetic construct
  source 1..23: mol_type = other RNA
tcttaatgataacagcagtgctg

SEQ ID NO: 1588 - RNA, 23 nt, synthetic construct
  source 1..23: mol_type = other RNA
caaaacaaatgccaggtgcctga

SEQ ID NO: 1589 - RNA, 23 nt, synthetic construct
  source 1..23: mol_type = other RNA
ccactagaatgtcagcatcgcac

SEQ ID NO: 1590 - RNA, 23 nt, synthetic construct
  source 1..23: mol_type = other RNA
cccatgaaattcatgcagacaga

SEQ ID NO: 1591 - RNA, 23 nt, synthetic construct
  source 1..23: mol_type = other RNA
ccgaacacagactcacagagcgg

SEQ ID NO: 1592 - RNA, 23 nt, synthetic construct
  source 1..23: mol_type = other RNA
cagaagaaagtctgcttgacgcg

SEQ ID NO: 1593 - RNA, 23 nt, synthetic construct
  source 1..23: mol_type = other RNA
tattaaagtcagatgccaacagt

SEQ ID NO: 1594 - RNA, 23 nt, synthetic construct
  source 1..23: mol_type = other RNA
ccccattcattagaaagccccac

SEQ ID NO: 1595 - RNA, 23 nt, synthetic construct
  source 1..23: mol_type = other RNA
agccaagacagttttgaccccac

SEQ ID NO: 1596 - RNA, 23 nt, synthetic construct
  source 1..23: mol_type = other RNA
gaaaagaaaagaaaagcccacac

SEQ ID NO: 1597 - RNA, 23 nt, synthetic construct
  source 1..23: mol_type = other RNA
caaaaagaaagataaggaccagt

SEQ ID NO: 1598 - RNA, 23 nt, synthetic construct
  source 1..23: mol_type = other RNA
tcaaagtcaacatcacagccgag

SEQ ID NO: 1599 - RNA, 23 nt, synthetic construct
  source 1..23: mol_type = other RNA
acataaaacaaacatgcacacca

SEQ ID NO: 1600 - RNA, 23 nt, synthetic construct
  source 1..23: mol_type = other RNA
cgtaaccaaagacagctgaccca

SEQ ID NO: 1601 - RNA, 23 nt, synthetic construct
  source 1..23: mol_type = other RNA
cagatgttgaaaattccacaccg

SEQ ID NO: 1602 - RNA, 23 nt, synthetic construct
  source 1..23: mol_type = other RNA
ttacctcatcttaaaacagccac

SEQ ID NO: 1603 - RNA, 23 nt, synthetic construct
  source 1..23: mol_type = other RNA
tatgtatagcataagcccaccac

SEQ ID NO: 1604 - RNA, 23 nt, synthetic construct
  source 1..23: mol_type = other RNA
tttaaaagcacataagcgcacag

SEQ ID NO: 1605 - RNA, 23 nt, synthetic construct
  source 1..23: mol_type = other RNA
gcctctttccaattatcacccac

SEQ ID NO: 1606 - RNA, 23 nt, synthetic construct
  source 1..23: mol_type = other RNA
aataactgtagagtgctcagcca

SEQ ID NO: 1607 - RNA, 23 nt, synthetic construct
  source 1..23: mol_type = other RNA
cactacaacgtttaacagctccg

SEQ ID NO: 1608 - RNA, 23 nt, synthetic construct
  source 1..23: mol_type = other RNA
aagtaaattgtttgccaaggccc

SEQ ID NO: 1609 - RNA, 23 nt, synthetic construct
  source 1..23: mol_type = other RNA
aaagagaaagtacagagacagga

SEQ ID NO: 1610 - RNA, 23 nt, synthetic construct
  source 1..23: mol_type = other RNA
ttccactaagttcatgaactccg

SEQ ID NO: 1611 - RNA, 23 nt, synthetic construct
  source 1..23: mol_type = other RNA
ccttttgccaaaactccagccac

SEQ ID NO: 1612 - RNA, 23 nt, synthetic construct
  source 1..23: mol_type = other RNA
catattgatgtttcaggttccag

SEQ ID NO: 1613 - RNA, 23 nt, synthetic construct
  source 1..23: mol_type = other RNA
acattagattacagagtccaagc

SEQ ID NO: 1614 - RNA, 23 nt, synthetic construct
  source 1..23: mol_type = other RNA
gcacaaacaaggttaaccccgac

SEQ ID NO: 1615 - RNA, 23 nt, synthetic construct
  source 1..23: mol_type = other RNA
agaactagttatgcacggagcaa

SEQ ID NO: 1616 - RNA, 23 nt, synthetic construct
  source 1..23: mol_type = other RNA
aacttagtagacctgtagcagca

SEQ ID NO: 1617 - RNA, 23 nt, synthetic construct
  source 1..23: mol_type = other RNA
cgtaatcactgagagccacacaa

SEQ ID NO: 1618 - RNA, 23 nt, synthetic construct
  source 1..23: mol_type = other RNA
gatgtcacacaattgcagagccc

SEQ ID NO: 1619 - RNA, 23 nt, synthetic construct
  source 1..23: mol_type = other RNA
tccaaaagatgatctgccccaag

SEQ ID NO: 1620 - RNA, 23 nt, synthetic construct
  source 1..23: mol_type = other RNA
agagtgacatcataacagcccat

SEQ ID NO: 1621 - RNA, 23 nt, synthetic construct
  source 1..23: mol_type = other RNA
catttcaaagagcatggccgtat

SEQ ID NO: 1622 - RNA, 23 nt, synthetic construct
  source 1..23: mol_type = other RNA
tgtgtaaattcagtgagacacgt

SEQ ID NO: 1623 - RNA, 23 nt, synthetic construct
  source 1..23: mol_type = other RNA
aaatcatagcatgaactgcccag

SEQ ID NO: 1624 - RNA, 23 nt, synthetic construct
  source 1..23: mol_type = other RNA
gttaaagcaaaatagcggccaga

SEQ ID NO: 1625 - RNA, 23 nt, synthetic construct
  source 1..23: mol_type = other RNA
aagatacaagtttaatgaggcag

SEQ ID NO: 1626 - RNA, 23 nt, synthetic construct
  source 1..23: mol_type = other RNA
aacacgtgacattagctcctaga

SEQ ID NO: 1627 - RNA, 23 nt, synthetic construct
  source 1..23: mol_type = other RNA
aaaaactgctgtgtggcatcaga

SEQ ID NO: 1628 - RNA, 23 nt, synthetic construct
  source 1..23: mol_type = other RNA
aaatcataccagatgccgctaca

SEQ ID NO: 1629 - RNA, 23 nt, synthetic construct
  source 1..23: mol_type = other RNA
atacaaacagatcggagcccgct

SEQ ID NO: 1630 - RNA, 23 nt, synthetic construct
  source 1..23: mol_type = other RNA
agtttacaaacagagagcacaag

SEQ ID NO: 1631 - RNA, 23 nt, synthetic construct
  source 1..23: mol_type = other RNA
actacagagcagttagccagcta

SEQ ID NO: 1632 - RNA, 23 nt, synthetic construct
  source 1..23: mol_type = other RNA
atgccatcaacatagtcccacag

SEQ ID NO: 1633 - RNA, 23 nt, synthetic construct
  source 1..23: mol_type = other RNA
tatttattactttacatgccagc

SEQ ID NO: 1634 - RNA, 23 nt, synthetic construct
  source 1..23: mol_type = other RNA
gttatcaaaggagccagccgcat

SEQ ID NO: 1635 - RNA, 23 nt, synthetic construct
  source 1..23: mol_type = other RNA
tgtaccacattagaacccacagg

SEQ ID NO: 1636 - RNA, 23 nt, synthetic construct
  source 1..23: mol_type = other RNA
aacattattattgtcccccagga

SEQ ID NO: 1637 - RNA, 23 nt, synthetic construct
  source 1..23: mol_type = other RNA
tacttttaaatgcatcagtccgc

SEQ ID NO: 1638 - RNA, 23 nt, synthetic construct
  source 1..23: mol_type = other RNA
accagagagaaaggaggccacag

SEQ ID NO: 1639 - RNA, 23 nt, synthetic construct
  source 1..23: mol_type = other RNA
ctcagaatttgggtgtggtgcag

SEQ ID NO: 1640 - RNA, 23 nt, synthetic construct
  source 1..23: mol_type = other RNA
ccgctgattgagtagtagtccca

SEQ ID NO: 1641 - RNA, 23 nt, synthetic construct
  source 1..23: mol_type = other RNA
gtccacctgacactggtcctcac

SEQ ID NO: 1642 - RNA, 23 nt, synthetic construct
  source 1..23: mol_type = other RNA
gtccgtaattggttcaagctggg

SEQ ID NO: 1643 - RNA, 23 nt, synthetic construct
  source 1..23: mol_type = other RNA
taaagtttcaaaagacgagccca

SEQ ID NO: 1644 - RNA, 23 nt, synthetic construct
  source 1..23: mol_type = other RNA
tttccaaaaagaaatcagcagga

SEQ ID NO: 1645 - RNA, 23 nt, synthetic construct
  source 1..23: mol_type = other RNA
gtcaatattgtcactgcagccac

SEQ ID NO: 1646 - RNA, 23 nt, synthetic construct
  source 1..23: mol_type = other RNA
cacatttatgatattccactcac

SEQ ID NO: 1647 - RNA, 23 nt, synthetic construct
  source 1..23: mol_type = other RNA
cacaacacataacttcgcagctg

SEQ ID NO: 1648 - RNA, 23 nt, synthetic construct
  source 1..23: mol_type = other RNA
actagcaagaagatccatcaagc

SEQ ID NO: 1649 - RNA, 23 nt, synthetic construct
  source 1..23: mol_type = other RNA
tcagaatctgaagaggcaacagt

SEQ ID NO: 1650 - RNA, 23 nt, synthetic construct
  source 1..23: mol_type = other RNA
ttcctcaattttcactaccacgt

SEQ ID NO: 1651 - RNA, 23 nt, synthetic construct
  source 1..23: mol_type = other RNA
cttttaacgaaagagacaggcct

SEQ ID NO: 1652 - RNA, 23 nt, synthetic construct
  source 1..23: mol_type = other RNA
cacataagggaaaacaagccccc

SEQ ID NO: 1653 - RNA, 23 nt, synthetic construct
  source 1..23: mol_type = other RNA
aatacatgttagatggcaacacg

SEQ ID NO: 1654 - RNA, 23 nt, synthetic construct
  source 1..23: mol_type = other RNA
ctgctagttaatataggccacac

SEQ ID NO: 1655 - RNA, 23 nt, synthetic construct
  source 1..23: mol_type = other RNA
taagactcaaacaagaccaccgg

SEQ ID NO: 1656 - RNA, 23 nt, synthetic construct
  source 1..23: mol_type = other RNA
cagaaatgacagaatggccaccc

SEQ ID NO: 1657 - RNA, 23 nt, synthetic construct
  source 1..23: mol_type = other RNA
tatatgtgagctataggagccag

SEQ ID NO: 1658 - RNA, 21 nt, synthetic construct
  source 1..21: mol_type = other RNA
cagagtctggacaaaggattt

SEQ ID NO: 1659 - RNA, 21 nt, synthetic construct
  source 1..21: mol_type = other RNA
gcccatagtacaccttcacta

SEQ ID NO: 1660 - RNA, 21 nt, synthetic construct
  source 1..21: mol_type = other RNA
gcgactgtgcaatttcatgta

SEQ ID NO: 1661 - RNA, 21 nt, synthetic construct
  source 1..21: mol_type = other RNA
cctgtgtctacaaacgctgaa

SEQ ID NO: 1662 - RNA, 21 nt, synthetic construct
  source 1..21: mol_type = other RNA
ctttgcttggtacaagggaaa

SEQ ID NO: 1663 - RNA, 21 nt, synthetic construct
  source 1..21: mol_type = other RNA
ccgcatgctgatcacaatcat

SEQ ID NO: 1664 - RNA, 21 nt, synthetic construct
  source 1..21: mol_type = other RNA
gaagttgaacaaatcgtatat

SEQ ID NO: 1665 - RNA, 21 nt, synthetic construct
  source 1..21: mol_type = other RNA
atctcagtctcagacttaatc

SEQ ID NO: 1666 - RNA, 59 nt, synthetic construct
  source 1..59: mol_type = other RNA
accggcagagtctggacaaaggatttctcgagaaatcctttgtccagactctgtttttg

SEQ ID NO: 1667 - RNA, 60 nt, synthetic construct
  source 1..60: mol_type = other RNA
gatccaaaaacagagtctggacaaaggatttctcgagaaatcctttgtccagactctgcc

SEQ ID NO: 1668 - RNA, 59 nt, synthetic construct
  source 1..59: mol_type = other RNA
accgggcccatagtacaccttcactactcgagtagtgaaggtgtactatgggctttttg

SEQ ID NO: 1669 - RNA, 60 nt, synthetic construct
  source 1..60: mol_type = other RNA
gatccaaaaagcccatagtacaccttcactactcgagtagtgaaggtgtactatgggccc

SEQ ID NO: 1670 - RNA, 59 nt, synthetic construct
  source 1..59: mol_type = other RNA
accgggcgactgtgcaatttcatgtactcgagtacatgaaattgcacagtcgctttttg

SEQ ID NO: 1671 - RNA, 60 nt, synthetic construct
  source 1..60: mol_type = other RNA
gatccaaaaagcgactgtgcaatttcatgtactcgagtacatgaaattgcacagtcgccc

SEQ ID NO: 1672 - RNA, 59 nt, synthetic construct
  source 1..59: mol_type = other RNA
accggcctgtgtctacaaacgctgaactcgagttcagcgtttgtagacacaggtttttg

SEQ ID NO: 1673 - RNA, 60 nt, synthetic construct
  source 1..60: mol_type = other RNA
gatccaaaaacctgtgtctacaaacgctgaactcgagttcagcgtttgtagacacaggcc

SEQ ID NO: 1674 - RNA, 59 nt, synthetic construct
  source 1..59: mol_type = other RNA
accggctttgcttggtacaagggaaactcgagtttcccttgtaccaagcaaagtttttg

SEQ ID NO: 1675 - RNA, 60 nt, synthetic construct
  source 1..60: mol_type = other RNA
gatccaaaaactttgcttggtacaagggaaactcgagtttcccttgtaccaagcaaagcc

SEQ ID NO: 1676 - RNA, 59 nt, synthetic construct
  source 1..59: mol_type = other RNA
accggccgcatgctgatcacaatcatctcgagatgattgtgatcagcatgcggtttttg

SEQ ID NO: 1677 - RNA, 60 nt, synthetic construct
  source 1..60: mol_type = other RNA
gatccaaaaaccgcatgctgatcacaatcatctcgagatgattgtgatcagcatgcggcc

SEQ ID NO: 1678 - RNA, 59 nt, synthetic construct
  source 1..59: mol_type = other RNA
accgggaagttgaacaaatcgtatatctcgagatatacgatttgttcaacttctttttg

SEQ ID NO: 1679 - RNA, 60 nt, synthetic construct
  source 1..60: mol_type = other RNA
gatccaaaaagaagttgaacaaatcgtatatctcgagatatacgatttgttcaacttccc

SEQ ID NO: 1680 - RNA, 59 nt, synthetic construct
  source 1..59: mol_type = other RNA
accggatctcagtctcagacttaatcctcgaggattaagtctgagactgagattttttg

SEQ ID NO: 1681 - RNA, 60 nt, synthetic construct
  source 1..60: mol_type = other RNA
gatccaaaaaatctcagtctcagacttaatcctcgaggattaagtctgagactgagatcc